SEQUENCE LISTING (558 sequences)

SEQ ID NO: 1 - DNA, 83 bp, synthetic construct
  source 1..83: mol_type = other DNA
tgtgattgacagctagctcagtcctaggtataatgctagcaaagaggagaaaggatctatggtgtcgaaaggtgaggtgcttt

SEQ ID NO: 2 - DNA, 60 bp, synthetic construct
  source 1..60: mol_type = other DNA
tcacgggggtggttccaatcttagtcgagcttgatggtgacgtgaatggccataagttta

SEQ ID NO: 3 - DNA, 89 bp, synthetic construct
  source 1..89: mol_type = other DNA
ggacacggtttgacttacctttcgacaccatagatcctttctcctctttgctagcattatacctaggactgagctagctgtcaatcaca

SEQ ID NO: 4 - DNA, 86 bp, synthetic construct
  source 1..86: mol_type = other DNA
taaacttatggccattcacgtcaccatcaagctcgactaagattggaaccacccccgtgaaaagcacctctagtcaaaccgtgtcc

SEQ ID NO: 5 - DNA, 86 bp, synthetic construct
  source 1..86: mol_type = other DNA
taaacttatggccattcacgtcaccatcaagctcgactaagattggaaccacccccgtgagagggtcccatagtcaaaccgtgtcc

SEQ ID NO: 6 - DNA, 86 bp, synthetic construct
  source 1..86: mol_type = other DNA
taaacttatggccattcacgtcaccatcaagctcgactaagattggaaccacccccgtgaaaagcacctctatcactcgcatccca

SEQ ID NO: 7 - DNA, 86 bp, synthetic construct
  source 1..86: mol_type = other DNA
taaacttatggccattcacgtcaccatcaagctcgactaagattggaaccacccccgtgagagggtcccatatcactcgcatccca

SEQ ID NO: 8 - DNA, 110 bp, synthetic construct
  source 1..110: mol_type = other DNA
atccatgcctgaattcgcggccgcttctagaggaaatcataaaaaatttatttgctttgtgagcggataacaattataatagattcaattgtcaagcgagccaccagcgc

SEQ ID NO: 9 - DNA, 82 bp, synthetic construct
  source 1..82: mol_type = other DNA
ctattataattgttatccgctcacaaagcaaataaattttttatgatttcctctagaagcggccgcgaattcaggcatggat

SEQ ID NO: 10 - DNA, 120 bp, synthetic construct
  source 1..120: mol_type = other DNA
gcgctggtggctcgcttggagcggataacaattactagaggagtcacacaggaaagtacatgaaatttggaaactttttgcttacataccaacctccccaatacgatgaccggagcatat

SEQ ID NO: 11 - DNA, 84 bp, synthetic construct
  source 1..84: mol_type = other DNA
ttggtatgtaagcaaaaagtttccaaatttcatgtactttcctgtgtgactcctctagtaattgttatccgctcacaattgaat

SEQ ID NO: 12 - DNA, 109 bp, synthetic construct
  source 1..109: mol_type = other DNA
atatgctccggtcatcgttttctcaaacagaggtaatgaaacgtttggttaaattaggtcgcatctctgaggagtgtggttttgataccgttggaacaagaggaggaat

SEQ ID NO: 13 - DNA, 73 bp, synthetic construct
  source 1..73: mol_type = other DNA
aaccacactcctcagagatgcgacctaatttaaccaaacgtttcattacctctgtttgagaaaattggggagg

SEQ ID NO: 14 - DNA, 117 bp, synthetic construct
  source 1..117: mol_type = other DNA
attcctcctcttgttccaatggttactggagcatcatttcacggagtttggtttgcttggtaacccttatgtcgctgctgcatatttacttggcgcgacagtattcctgctagagaa

SEQ ID NO: 15 - DNA, 81 bp, synthetic construct
  source 1..81: mol_type = other DNA
gtaaatatgcagcagcgacataagggttaccaagcaaaccaaactccgtgaaatgatgctccagtaaccatacggtatcaa

SEQ ID NO: 16 - DNA, 116 bp, synthetic construct
  source 1..116: mol_type = other DNA
ttctctagcaggaatacttaaaaaattgaatgtaggaactgccgctattgttcttcccacagcccatccagtacgccaacttgaagatgtgaatttatgacgtatttgaatctctg

SEQ ID NO: 17 - DNA, 80 bp, synthetic construct
  source 1..80: mol_type = other DNA
atcttcaagttggcgtactggatgggctgtgggaagaacaatagcggcagttcctacattcaattttttagtcgcgccaa

SEQ ID NO: 18 - DNA, 114 bp, synthetic construct
  source 1..114: mol_type = other DNA
cagagattcaaatacgtctggatcaaatgtcaaaaggacgatttcggtttggtatttgccgagggctttacaacaaggactttcgcgtattcggcacattaacggggaccaaat

SEQ ID NO: 19 - DNA, 78 bp, synthetic construct
  source 1..78: mol_type = other DNA
gcgaaagtccttgttgtaaagccctcggcaaataccaaaccgaaatcgtccttttgacatttgatccaataaattcac

SEQ ID NO: 20 - DNA, 115 bp, synthetic construct
  source 1..115: mol_type = other DNA
atttggtccccgttaatgcagatatgaataacagtcgcgccttagcggaatgctggtacgggctgataaagaatggcatgacagagggatatatggatattcgttcggaatctgt

SEQ ID NO: 21 - DNA, 79 bp, synthetic construct
  source 1..79: mol_type = other DNA
cctctgtcatgccattctttatcagcccgtaccagcattccgctaaggcgcgactgttattcatatctgtgccgaatac

SEQ ID NO: 22 - DNA, 119 bp, synthetic construct
  source 1..119: mol_type = other DNA
acagattccgaacgaataagctgataatgaacatatcaagttccataaggtaaaagtaaaccccgcggcgtatagcagaggtggcgcaccggtttatgtggtagtgttggttcatccaa

SEQ ID NO: 23 - DNA, 83 bp, synthetic construct
  source 1..83: mol_type = other DNA
cggtgcgccacctctgctatacgccgcggggtttacttttaccttatggaacttgatatgttcattatcagcttccatatatc

SEQ ID NO: 24 - DNA, 108 bp, synthetic construct
  source 1..108: mol_type = other DNA
ttggatgaaccaacactatggctgaatcagcttcgacgactgagtgggctgctcaatttggcctaccgatgatattaagttggattataaaaagccgtcaattaaaat

SEQ ID NO: 25 - DNA, 72 bp, synthetic construct
  source 1..72: mol_type = other DNA
acttaatatcatcggtaggccaaattgagcagcccactcagtcgtcgaagctgattcagccaccacataaac

SEQ ID NO: 26 - DNA, 118 bp, synthetic construct
  source 1..118: mol_type = other DNA
attttaattgacggctttatactaacgaaaagaaagcacaacttgagctttataatgaagtggctcaagaatatgggcacgatattcataatatcgaccaatgacacgtaatccaaat

SEQ ID NO: 27 - DNA, 82 bp, synthetic construct
  source 1..82: mol_type = other DNA
tatgaatatcgtgcccatattcttgagccacttcattataaagctcaagttgtgctttcttttcgttagtatttataatcca

SEQ ID NO: 28 - DNA, 116 bp, synthetic construct
  source 1..116: mol_type = other DNA
atttggattacgtgtcatttgcttatcatatataacatctgtagatcatgactcaattaaagcgaaagagatttgccggaaatttctggggcattggttatgtttacccgggtcga

SEQ ID NO: 29 - DNA, 80 bp, synthetic construct
  source 1..80: mol_type = other DNA
cagaaatttccggcaaatctctttcgctttaattgagtcatgatctacagatgttatatatgataagcaatggtcgatat

SEQ ID NO: 30 - DNA, 116 bp, synthetic construct
  source 1..116: mol_type = other DNA
tcgacccgggtaaacataatgattcttatgtgaatgctacgactatttttgatgattcagaccaaacaagaggttatgatttcaataaagggcagtggcacaatcgctccccgagt

SEQ ID NO: 31 - DNA, 80 bp, synthetic construct
  source 1..80: mol_type = other DNA
ttattgaaatcataacctcttgtttggtctgaatcatcaaaaatagtcgtagcattcacataagaatcataccaatgccc

SEQ ID NO: 32 - DNA, 119 bp, synthetic construct
  source 1..119: mol_type = other DNA
actcggggagcgattgtgcgtgactttgtattaaaaggacataaagatactaatcgccgtattgattacagttacgaaatcaatcccgtgggaacgccgcatacccaactaggtcaatt

SEQ ID NO: 33 - DNA, 83 bp, synthetic construct
  source 1..83: mol_type = other DNA
ccacgggattgatttcgtaactgtaatcaatacggcgattagtatctttatgtccttttaatacaaagtcacgccactgccct

SEQ ID NO: 34 - DNA, 112 bp, synthetic construct
  source 1..112: mol_type = other DNA
aattgacctagttgggtaggaatgtattgacataattcaaaaagacattgatgctacaggaatatcaaatatttgttgtggatttgaagctaatgttaattcatcaagacag

SEQ ID NO: 35 - DNA, 76 bp, synthetic construct
  source 1..76: mol_type = other DNA
aatccacaacaaatatttgatattcctgtagcatcaatgtctttttgaattatgtcaatacattcctgcggcgttc

SEQ ID NO: 36 - DNA, 113 bp, synthetic construct
  source 1..113: mol_type = other DNA
ctgtcttgatgaattaacggaacagtagacgaaattattgcttccatgaagctcttccagtctgatgtcatgccatttcttaaagaaaaacaacgggtcataatttgcataac

SEQ ID NO: 37 - DNA, 77 bp, synthetic construct
  source 1..77: mol_type = other DNA
ctttaagaaatggcatgacatcagactggaagagcttcatggaagcaataatttcgtctactgttccattagcttca

SEQ ID NO: 38 - DNA, 115 bp, synthetic construct
  source 1..115: mol_type = other DNA
gttatgcaaattatgaccttcgctattatattaataatactagagagagaaagacaggacccactatgaaatttggattgttcttccttaacttcatgatcactaagttggccta

SEQ ID NO: 39 - DNA, 79 bp, synthetic construct
  source 1..79: mol_type = other DNA
ggaagaacaatccaaatttcatagtgggtcctgtctttctctctctagtattattaatataatagcgaacgttgttttt

SEQ ID NO: 40 - DNA, 119 bp, synthetic construct
  source 1..119: mol_type = other DNA
taggccaacttagtgatccaattcaacaactgttcaagaacaaagtatagttcgcatgcaggaaataacggagtatgttgataagttgaattttgaacagactctgcgaaaatacacta

SEQ ID NO: 41 - DNA, 83 bp, synthetic construct
  source 1..83: mol_type = other DNA
attcaacttatcaacatactccgttatttcctgcatgcgaactatactttgttcttgaacagttgttgaattgatgaagttaa

SEQ ID NO: 42 - DNA, 117 bp, synthetic construct
  source 1..117: mol_type = other DNA
tagtgtattttcgcagagttttagtgtatgaaaatcatttttcagataatggtgttgtcggcgctcctctgactgtttctggttttctgctcggtttaaatagggtgccaacgacgc

SEQ ID NO: 43 - DNA, 81 bp, synthetic construct
  source 1..81: mol_type = other DNA
cagaaaaccagaaacagtcagaggagcgccgacaacaccattatctgaaaaatgattttcatacactaaaatctgttcaaa

SEQ ID NO: 44 - DNA, 119 bp, synthetic construct
  source 1..119: mol_type = other DNA
gcgtcgttggcaccctatcagagaaaattaaaattggttcattaaatcacatcattacaactcatcatcctgtccgcatagcggaggaagcttgcttattgttgtaatcagcaggtata

SEQ ID NO: 45 - DNA, 83 bp, synthetic construct
  source 1..83: mol_type = other DNA
gcttcctccgctatgcggacaggatgatgagttgtaatgatgtgatttaatgaaccaattttaattttctctgttaaaccgag

SEQ ID NO: 46 - DNA, 117 bp, synthetic construct
  source 1..117: mol_type = other DNA
tatacctgctgattacaagatcagttaagtgaagggagatttattttagggtttagtgattgcgaaaaaaaagatgaaatgcatttttttaatcgcccggaaggttggtcttagcaa

SEQ ID NO: 47 - DNA, 81 bp, synthetic construct
  source 1..81: mol_type = other DNA
aaaaaatgcatttcatcttttttttcgcaatcactaaaccctaaaataaatctcccttcacttaactgatccaataagcaa

SEQ ID NO: 48 - DNA, 116 bp, synthetic construct
  source 1..116: mol_type = other DNA
ttgctaagaccaaccttcgttgaatatcaacagcaactatttgaagagtgttatgaaatcattaacgatgctttaacaacaggctattgtaatccagacgagtcactagtaaaagt

SEQ ID NO: 49 - DNA, 80 bp, synthetic construct
  source 1..80: mol_type = other DNA
aatagcctgttgttaaagcatcgttaatgatttcataacactcttcaaatagttgctgttgatattcaaccgggcgatta

SEQ ID NO: 50 - DNA, 119 bp, synthetic construct
  source 1..119: mol_type = other DNA
acttttactagtgactcgtaacgatttttatagcttccctaaaatatctgtaaatccccatgcttatacgccaggcggacctcggaaatatgtaacagcaaacaaagtgaactatggaa

SEQ ID NO: 51 - DNA, 83 bp, synthetic construct
  source 1..83: mol_type = other DNA
atatttccgaggtccgcctggcgtataagcatggggatttacagatattttagggaagctataaaaatcgttatctggattac

SEQ ID NO: 52 - DNA, 116 bp, synthetic construct
  source 1..116: mol_type = other DNA
ttccatagttcactttgtccagtcatcatattgttgagtgggcggccaaaaaaggtattcctctcatctttaagtgggatgattctaatgatgttagaacttgatttccccgcaac

SEQ ID NO: 53 - DNA, 80 bp, synthetic construct
  source 1..80: mol_type = other DNA
ttagaatcatcccacttaaagatgagaggaataccttttttggccgcccactcaacaatatgatgactggttgctgttac

SEQ ID NO: 54 - DNA, 116 bp, synthetic construct
  source 1..116: mol_type = other DNA
gttgcggggaaatcaagttatgaatatgctgaaagatataaagccgttgcggataaatatgacgttgacctatcagagatagaccatcagttaatgatcagctggccgagtctgat

SEQ ID NO: 55 - DNA, 80 bp, synthetic construct
  source 1..80: mol_type = other DNA
gatggtctatctctgataggtcaacgtcatatttatccgcaacggctttatatctttcagcatattcatatctaacatca

SEQ ID NO: 56 - DNA, 119 bp, synthetic construct
  source 1..119: mol_type = other DNA
atcagactcggccagctgattagttaactataacgaagatagtaataaagctaaacaagagacgcgtgcatttattagtgattatgttcttgaaatgcacccagctagtgatcgatgtg

SEQ ID NO: 57 - DNA, 83 bp, synthetic construct
  source 1..83: mol_type = other DNA
aagaacataatcactaataaatgcacgcgtctcttgtttagctttattactatcttcgttatagttaactaatatcattaact

SEQ ID NO: 58 - DNA, 117 bp, synthetic construct
  source 1..117: mol_type = other DNA
cacatcgatcactagctgctaatgaaaatttcgaaaataaacttgaagaaataattgcagaaaacgctgtcggaaattatacggagtgtataactgcggggatgctgggttgtcact

SEQ ID NO: 59 - DNA, 81 bp, synthetic construct
  source 1..81: mol_type = other DNA
acactccgtataatttccgacagcgttttctgcaattatttcttcaagtttattttcgaaattttcattagggtgcatttc

SEQ ID NO: 60 - DNA, 119 bp, synthetic construct
  source 1..119: mol_type = other DNA
agtgacaacccagcatccctaagttggcaattgaaaagtgtggtgcgaaaagtgtattgctgtcctttgaaccaatgaatgatttgatgagccaaaaaaatacaagaaaccaagtacaa

SEQ ID NO: 61 - DNA, 83 bp, synthetic construct
  source 1..83: mol_type = other DNA
ctcatcaaatcattcattggttcaaaggacagcaatacacttttcgcaccacacttttcaattgccaacttagccgcagttat

SEQ ID NO: 62 - DNA, 113 bp, synthetic construct
  source 1..113: mol_type = other DNA
ttgtacttggtttcttgtgtaatcaatattgttgatgataatattaagaagtaccacatggaatatacctaataatactagagaaagaggagaaagtcaagcacgagcatggc

SEQ ID NO: 63 - DNA, 77 bp, synthetic construct
  source 1..77: mol_type = other DNA
ttctctagtattattaggtatattccatgtggtacttcttaatattatcatcaacaatattgattacatttttttgg

SEQ ID NO: 64 - DNA, 116 bp, synthetic construct
  source 1..116: mol_type = other DNA
gccatgctcgtgcttgactactagatgactaaaaaaatttcattcattattaacggccaggttgaaatctttcccgaaagtgatgatttagtgcaatcttggctgcgtaccggttt

SEQ ID NO: 65 - DNA, 80 bp, synthetic construct
  source 1..80: mol_type = other DNA
aatcatcactttcgggaaagatttcaacctggccgttaataatgaatgaaatttttttagtcatctagtatttctcctct

SEQ ID NO: 66 - DNA, 116 bp, synthetic construct
  source 1..116: mol_type = other DNA
aaaccggtacgcagccaacattaattttggtgataatagtgtttacctgccaatattgaatgactctcatgtaaaaaacattattgattgtaatggaacgtaaagaccacggcccc

SEQ ID NO: 67 - DNA, 80 bp, synthetic construct
  source 1..80: mol_type = other DNA
atcaataatgttttttacatgagagtcattcaatattggcaggtaaacactattatcaccaaaattaatggattgcacta

SEQ ID NO: 68 - DNA, 107 bp, synthetic construct
  source 1..107: mol_type = other DNA
ggggccgtggtctttacgataacgaattacggttgcataacattgtcaattttctctatacggtagggcaaagatggaaaaatgaagaaagattacctcttggctct

SEQ ID NO: 69 - DNA, 71 bp, synthetic construct
  source 1..71: mol_type = other DNA
ttccatctttgccctaccgtatagagaaaattgacaatgttatgcaaccgtaattcgttatttccattaca

SEQ ID NO: 70 - DNA, 119 bp, synthetic construct
  source 1..119: mol_type = other DNA
agagccaagaggtaatcttactcaagacgcaggacatacattcgtgacttaaaaaaatatatgggatattcagaagaaatggctaagctagaggccaattgtatctacacctccggcgt

SEQ ID NO: 71 - DNA, 83 bp, synthetic construct
  source 1..83: mol_type = other DNA
ctagcttagccatttcttctgaatatcccatatatttttttaagtcacgaatgtatgtcctgcgtcttgagtattcttcattt

SEQ ID NO: 72 - DNA, 110 bp, synthetic construct
  source 1..110: mol_type = other DNA
acgccggaggtgtagatagatatctatgattttatgttctaaaggcggcctttatgatgttgtagaaaatgaacttggttctcgccatatcattctcgccctatcagtcg

SEQ ID NO: 73 - DNA, 74 bp, synthetic construct
  source 1..74: mol_type = other DNA
agaaccaagttcattttctacaacatcataaaggccgcctttagaacataaaatcatagatatccaattggcct

SEQ ID NO: 74 - DNA, 119 bp, synthetic construct
  source 1..119: mol_type = other DNA
cgactgatagggcgagaatggatgaatggctacctcaggatgaaagttatgttcgggcttttccgaaaggtaaatctgtacatctgttggcaggtaatgttaaggacggatagtttaag

SEQ ID NO: 75 - DNA, 83 bp, synthetic construct
  source 1..83: mol_type = other DNA
gccaacagatgtacagatttacctttcggaaaagcccgaacataactttcatcctgaggtagccattcatccatgatatggcg

SEQ ID NO: 76 - DNA, 118 bp, synthetic construct
  source 1..118: mol_type = other DNA
cttaaactatccgtccttccattatctgggatcatgtctatattacgcgcaattttaactaagaatcagtgtattataaaaacatcgtcaaccgatcctttgccgcctccttttgggt

SEQ ID NO: 77 - DNA, 82 bp, synthetic construct
  source 1..82: mol_type = other DNA
tgacgatgtttttataatacactgattcttagttaaaattgcgcgtaatatagacatgatcccagataatggaacattacct

SEQ ID NO: 78 - DNA, 116 bp, synthetic construct
  source 1..116: mol_type = other DNA
acccaaaaggaggcggcattaccgctaatgcattagcgttaagttttattgatgtagaccctaatcatccgataacgcgctctttatctgttatatattgacaagtcgctgtttaa

SEQ ID NO: 79 - DNA, 80 bp, synthetic construct
  source 1..80: mol_type = other DNA
gataaagagcgcgttatcggatgattagggtctacatcaataaaacttaacgctaatgcattagcggtaaaaggatcggt

SEQ ID NO: 80 - DNA, 118 bp, synthetic construct
  source 1..118: mol_type = other DNA
ttaaacagcgacttgtcatggccccaccaaggtgatacatcactcgcaaaagaaattatgcgacatgcggatgttattgtcgcttggggagggccagatgtctgattactgggaaaag

SEQ ID NO: 81 - DNA, 82 bp, synthetic construct
  source 1..82: mol_type = other DNA
tccccaagcgacaataacatccgcatgtcgcataatttcttttgcgagtgatgtatcaccttggtggggccaatatataaca

SEQ ID NO: 82 - DNA, 117 bp, synthetic construct
  source 1..117: mol_type = other DNA
cttttcccagtaatcagacgattaattgggcggtagagcatgcgccatcttatgctgatgtgattaaatttggttctaaaaagagtctttgcattatcgttagagtttcgccaccag

SEQ ID NO: 83 - DNA, 81 bp, synthetic construct
  source 1..81: mol_type = other DNA
aagactctttttagaaccaaatttaatcacatcagcataagatggcgcatgctctaccgcccaattaatcgcatctggccc

SEQ ID NO: 84 - DNA, 119 bp, synthetic construct
  source 1..119: mol_type = other DNA
ctggtggcgaaactctaaataatcctgttgatttgacgtccgcagcgacaggtgcggctcatgatgtttgtttttacgatcagcgagcttgtttttctgccgacatcttatgggaaagc

SEQ ID NO: 85 - DNA, 83 bp, synthetic construct
  source 1..83: mol_type = other DNA
caagctcgctgatcgtaaaaacaaacatcatgagccgcacctgtcgctgcggacgtcaaatcaacaggattatcgataatgca

SEQ ID NO: 86 - DNA, 120 bp, synthetic construct
  source 1..120: mol_type = other DNA
gctttcccataagatgtccaaaacatatattacatgggaaatcattatgaggaatttaagttagcgttgatagaaaaacttaatctatatgcgcatatattaccgaatgccaaaaaagat

SEQ ID NO: 87 - DNA, 112 bp, synthetic construct
  source 1..112: mol_type = other DNA
atcttttttggcattcggtaatatatgcgcatatagattaagtttttctatcaacgctaacttaaattcctcataatgatttcccatgtaatatatgttttgggcagaaaaa

SEQ ID NO: 88 - DNA, 100 bp, synthetic construct
  source 1..100: mol_type = other DNA
atccatgcctgaattcgcggccgcttctagaggaaatcataaaaaatttatttgctttgtgagcggataacaattataatagattcaattgtgagcggat

SEQ ID NO: 89 - DNA, 101 bp, synthetic construct
  source 1..101: mol_type = other DNA
agaaaattggggaggttggtatgtaagcaaaaagtttccaaatttcatgtactttcctgtgtgactcctctagtaattgttatccgctcacaattgaatct

SEQ ID NO: 90 - DNA, 100 bp, synthetic construct
  source 1..100: mol_type = other DNA
accaacctccccaattttctcaaacagaggtaatgaaacgtttggttaaattaggtcgcatctctgaggagtgtggttttgataccgtatggttactgga

SEQ ID NO: 91 - DNA, 100 bp, synthetic construct
  source 1..100: mol_type = other DNA
tcaattttttagtcgcgccaagtaaatatgcagcagcgacataagggttaccaagcaaaccaaactccgtgaaatgatgctccagtaaccatacggtatc

SEQ ID NO: 92 - DNA, 79 bp, synthetic construct
  source 1..79: mol_type = other DNA
tggcgcgactaaaaaattgaatgtaggaactgccgctattgttcttcccacagcccatccagtacgccaacttgaagat

SEQ ID NO: 93 - DNA, 100 bp, synthetic construct
  source 1..100: mol_type = other DNA
acgcgaaagtccttgttgtaaagccctcggcaaataccaaaccgaaatcgtccttttgacatttgatccaataaattcacatcttcaagttggcgtactg

SEQ ID NO: 94 - DNA, 100 bp, synthetic construct
  source 1..100: mol_type = other DNA
tacaacaaggactttcgcgtattcggcacagatatgaataacagtcgcgccttagcggaatgctggtacgggctgataaagaatggcatgacagagggat

SEQ ID NO: 95 - DNA, 100 bp, synthetic construct
  source 1..100: mol_type = other DNA
cggtgcgccacctctgctatacgccgcggggtttacttttaccttatggaacttgatatgttcattatcagcttccatatatccctctgtcatgccattc

SEQ ID NO: 96 - DNA, 100 bp, synthetic construct
  source 1..100: mol_type = other DNA
atagcagaggtggcgcaccggtttatgtggtggctgaatcagcttcgacgactgagtgggctgctcaatttggcctaccgatgatattaagttggattat

SEQ ID NO: 97 - DNA, 94 bp, synthetic construct
  source 1..94: mol_type = other DNA
tatgaatatcgtgcccatattcttgagccacttcattataaagctcaagttgtgctttcttttcgttagtatttataatccaacttaatatcat

SEQ ID NO: 98 - DNA, 100 bp, synthetic construct
  source 1..100: mol_type = other DNA
atatgggcacgatattcataatatcgaccattgcttatcatatataacatctgtagatcatgactcaattaaagcgaaagagatttgccggaaatttctg

SEQ ID NO: 99 - DNA, 100 bp, synthetic construct
  source 1..100: mol_type = other DNA
ttattgaaatcataacctcttgtttggtctgaatcatcaaaaatagtcgtagcattcacataagaatcataccaatgccccagaaatttccggcaaatct

SEQ ID NO: 100 - DNA, 100 bp, synthetic construct
  source 1..100: mol_type = other DNA
agaggttatgatttcaataaagggcagtggcgtgactttgtattaaaaggacataaagatactaatcgccgtattgattacagttacgaaatcaatcccg

SEQ ID NO: 101 - DNA, 100 bp, synthetic construct
  source 1..100: mol_type = other DNA
aaatccacaacaaatatttgatattcctgtagcatcaatgtctttttgaattatgtcaatacattcctgcggcgttcccacgggattgatttcgtaactg

SEQ ID NO: 102 - DNA, 100 bp, synthetic construct
  source 1..100: mol_type = other DNA
caaatatttgttgtggatttgaagctaatggaacagtagacgaaattattgcttccatgaagctcttccagtctgatgtcatgccatttcttaaagaaaa

SEQ ID NO: 103 - DNA, 100 bp, synthetic construct
  source 1..100: mol_type = other DNA
gttaaggaagaacaatccaaatttcatagtgggtcctgtctttctctctctagtattattaatataatagcgaacgttgtttttctttaagaaatggcat

SEQ ID NO: 104 - DNA, 100 bp, synthetic construct
  source 1..100: mol_type = other DNA
ttggattgttcttccttaacttcatcaattcaacaactgttcaagaacaaagtatagttcgcatgcaggaaataacggagtatgttgataagttgaattt

SEQ ID NO: 105 - DNA, 100 bp, synthetic construct
  source 1..100: mol_type = other DNA
gcagaaaaccagaaacagtcagaggagcgccgacaacaccattatctgaaaaatgattttcatacactaaaatctgttcaaaattcaacttatcaacata

SEQ ID NO: 106 - DNA, 100 bp, synthetic construct
  source 1..100: mol_type = other DNA
gactgtttctggttttctgctcggtttaacagagaaaattaaaattggttcattaaatcacatcattacaactcatcatcctgtccgcatagcggaggaa

SEQ ID NO: 107 - DNA, 103 bp, synthetic construct
  source 1..103: mol_type = other DNA
aaaaaatgcatttcatcttttttttcgcaatcactaaaccctaaaataaatctcccttcacttaactgatccaataagcaagcttcctccgctatgcggacag

SEQ ID NO: 108 - DNA, 84 bp, synthetic construct
  source 1..84: mol_type = other DNA
caacgactttgcgtgttgtcttactattgctggcaggaggtcagcgaccttgatgtttccagtgcgattgaggaccttcagtgc

SEQ ID NO: 109 - DNA, 88 bp, synthetic construct
  source 1..88: mol_type = other DNA
aaaagctcctcacctttcgacaccatggtttagttcctcaccttgtcgtattatactatgccgatatactatgccgatgattaattgt

SEQ ID NO: 110 - DNA, 88 bp, synthetic construct
  source 1..88: mol_type = other DNA
cgcatcgccctcaccctcgccgcgcacgctaaacttatggccattcacgtcaccatcaagctcgactaagattggaaccacccccgtg

SEQ ID NO: 111 - DNA, 85 bp, synthetic construct
  source 1..85: mol_type = other DNA
acccaagcgtagtgaccagggtcggccacgggaccggtaatttgcctgtagtacaaataaactttaaagtaagtttaccgttcgt

SEQ ID NO: 112 - DNA, 88 bp, synthetic construct
  source 1..88: mol_type = other DNA
tcctgaacatacccttcgggcatggcagatttaaagaaatcgtgctgtttcatgtggtcagggtaacgggcaaaacatgctaccccat

SEQ ID NO: 113 - DNA, 87 bp, synthetic construct
  source 1..87: mol_type = other DNA
acgatacggttcacaagggtatcaccttcgaactttacttcagcacgagttttgtacgttccgtcgtctttaaaactgatagtgcgc

SEQ ID NO: 114 - DNA, 88 bp, synthetic construct
  source 1..88: mol_type = other DNA
tgtgatataaaccttatgtgaattgaagttgtactctaatttgtggcctaaaatgttaccgtcctctttgaagtcgatgcctttaagg

SEQ ID NO: 115 - DNA, 88 bp, synthetic construct
  source 1..88: mol_type = other DNA
ggtagtggtccgccaactggactccaccatcttctacgttgtggcgggtcttaaaatttgccttgatgccatttttttgcttatctgc

SEQ ID NO: 116 - DNA, 80 bp, synthetic construct
  source 1..80: mol_type = other DNA
cttactcagtttactctggtggctaagatagtgattgtcgggaagcaaaaccggaccatcgccaatcggtgtattttgct

SEQ ID NO: 117 - DNA, 86 bp, synthetic construct
  source 1..86: mol_type = other DNA
ttataaagttcgtccatatcgtgtgtaattccagctgctgtaacgcgctctttcaataccatgtggtcgcgcttttcatttgggtc

SEQ ID NO: 118 - DNA, 88 bp, synthetic construct
  source 1..88: mol_type = other DNA
tttgataactgcttcgcctttactgaccatatttctcctctttgctagcattatacctaggactgagctagctgtcaaatagatttac

SEQ ID NO: 119 - DNA, 88 bp, synthetic construct
  source 1..88: mol_type = other DNA
catatgggcggccttcgccttcgccttcaatttcaaattcgtgcccgttcatcgacccttccatatgaactttgaagcgcatgaactc

SEQ ID NO: 120 - DNA, 87 bp, synthetic construct
  source 1..87: mol_type = other DNA
cgtacataaactgtgggcttaaaatgtcccacgaaaagggtaatggaccgcctttagtaaccttaagctttgcggtttgggtccctt

SEQ ID NO: 121 - DNA, 88 bp, synthetic construct
  source 1..88: mol_type = other DNA
atcacgcgctcccacttgaacccctcggggaaagactgtttgtagtaatctgggatgtccgcagggtgcttgatgaaagcgcgactcc

SEQ ID NO: 122 - DNA, 88 bp, synthetic construct
  source 1..88: mol_type = other DNA
gccacgcaatttcactttgtagatcaacgtaccgtcctccaaagaggtatcttgggtgaccgtcacggctccgccatcctcgaagttc

SEQ ID NO: 123 - DNA, 88 bp, synthetic construct
  source 1..88: mol_type = other DNA
caccgtcctctgggtaaaggcgctccgtggaagcttcccatcccattgtctttttctgcatgacaggcccatcaggtggaaaattcgt

SEQ ID NO: 124 - DNA, 88 bp, synthetic construct
  source 1..88: mol_type = other DNA
tttttggccttgtaagtagtcttgaagtccgccaggtagcggcctccatctttaagacgcagcgccattttgatatcccctttaagaa

SEQ ID NO: 125 - DNA, 88 bp, synthetic construct
  source 1..88: mol_type = other DNA
atactgctctacgaccgtgtaatcttcattgtgacttgtaatatctaatttacgatctacattgtacgcacccggcatctgcactggt

SEQ ID NO: 126 - DNA, 88 bp, synthetic construct
  source 1..88: mol_type = other DNA
tgtatttagaaaaataaacaaactcgagggtaccttacttgtataactcgtccataccgcccgtagagtgacgcccctcactgcgctc

SEQ ID NO: 127 - DNA, 120 bp, synthetic construct
  source 1..120: mol_type = other DNA
gaatttaatcgcggcctagagcaagacgtttcccgttgaatatggctcatactcttcctttttcaatattattgaagcatttatcagggttattgtctcatgagcggatacatatttgaa

SEQ ID NO: 128 - DNA, 110 bp, synthetic construct
  source 1..110: mol_type = other DNA
gcactgaaggtcctcaatcgcactggaaacatcaaggtcgctgacctcctgccagcaatagtaagacaacacgcaaagtcgttgacaattaatctaggcacgctcaacct

SEQ ID NO: 129 - DNA, 120 bp, synthetic construct
  source 1..120: mol_type = other DNA
aggttgagcgtgccttatcggcatagtatatcggcatagtataatacgacaaggtgaggaactaaaccatggtgtcgaaaggtgaggagcttttcacgggggtgtaggcggtcgagagtg

SEQ ID NO: 130 - DNA, 120 bp, synthetic construct
  source 1..120: mol_type = other DNA
cactctcgaccgccttgttccaatcttagtcgagcttgatggtgacgtgaatggccataagtttagcgtgcgcggcgagggtgagggcgatgcgacgaacggtatatcactcgcatccca

SEQ ID NO: 131 - DNA, 117 bp, synthetic construct
  source 1..117: mol_type = other DNA
tgggatgcgagtgattaacttactttaaagtttatttgtactacaggcaaattaccggtcccgtggccgaccctggtcactacgcttgggtatggggtagctaggtccggtgcaggt

SEQ ID NO: 132 - DNA, 120 bp, synthetic construct
  source 1..120: mol_type = other DNA
acctgcaccggaccttatgttttgcccgttaccctgaccacatgaaacagcacgatttctttaaatctgccatgcccgaagggtatgttcaggagcgcactatctcgatcgcccttggtg

SEQ ID NO: 133 - DNA, 119 bp, synthetic construct
  source 1..119: mol_type = other DNA
caccaagggcgatcgtagttttaaagacgacggaacgtacaaaactcgtgctgaagtaaagttcgaaggtgatacccttgtgaaccgtatcgtccttaaaggctgcggcaccacaaact

SEQ ID NO: 134 - DNA, 120 bp, synthetic construct
  source 1..120: mol_type = other DNA
agtttgtggtgccgctatcgacttcaaagaggacggtaacattttaggccacaaattagagtacaacttcaattcacataaggtttatatcacagcagataagctagttgaccagcgcca

SEQ ID NO: 135 - DNA, 120 bp, synthetic construct
  source 1..120: mol_type = other DNA
tggcgctggtcaacttaaaaaaatggcatcaaggcaaattttaagacccgccacaacgtagaagatggtggagtccagttggcggaccactaccagcaaaatactaagagggacgcagca

SEQ ID NO: 136 - DNA, 112 bp, synthetic construct
  source 1..112: mol_type = other DNA
tgctgcgtccctctttaccgattggcgatggtccggttttgcttcccgacaatcactatcttagccaccagagtaaactgagtaaggacccaaatgttgcccgctgtcttca

SEQ ID NO: 137 - DNA, 118 bp, synthetic construct
  source 1..118: mol_type = other DNA
tgaagacagcgggcataaaagcgcgaccacatggtattgaaagagcgcgttacagcagctggaattacacacgatatggacgaactttataagtaaatctattaacgcccagcctgtc

SEQ ID NO: 138 - DNA, 120 bp, synthetic construct
  source 1..120: mol_type = other DNA
gacaggctgggcgtttttgacagctagctcagtcctaggtataatgctagcaaagaggagaaatatggtcagtaaaggcgaagcagttatcaaagagttcatgctcgcagggtccagagt

SEQ ID NO: 139 - DNA, 120 bp, synthetic construct
  source 1..120: mol_type = other DNA
agacgacgcaccagttgatggcggagccgtgacggtcacccaagatacctctttggaggacggtacgttgatctacaaagtgaaattgcgtggcacgaattttctgccgccgcttattgg

SEQ ID NO: 140 - DNA, 120 bp, synthetic construct
  source 1..120: mol_type = other DNA
ccaataagcggcggctcacctgatgggcctgtcatgcagaaaaagacaatgggatgggaagcttccacggagcgcctttacccagaggacggtgttcttaaaggtggtcgagccggaact

SEQ ID NO: 141 - DNA, 120 bp, synthetic construct
  source 1..120: mol_type = other DNA
actctggaccctgcgtgcttcaaagttcatatggaagggtcgatgaacgggcacgaatttgaaattgaaggcgaaggcgaaggccgcccatatgaagggacccatagcgaaaccgtgcgt

SEQ ID NO: 142 - DNA, 119 bp, synthetic construct
  source 1..119: mol_type = other DNA
acgcacggtttcgcttaaccgcaaagcttaaggttactaaaggcggtccattacccttttcgtgggacattttaagcccacagtttatgtacgggagtcgcgcttccaccgtcggcaag

SEQ ID NO: 143 - DNA, 120 bp, synthetic construct
  source 1..120: mol_type = other DNA
cttgccgacggtggattttcatcaagcaccctgcggacatcccagattactacaaacagtctttccccgaggggttcaagtgggagcgcgtgatgaacttcgagtactggtgcgtcgtct

SEQ ID NO: 144 - DNA, 120 bp, synthetic construct
  source 1..120: mol_type = other DNA
agttccggctcgacctggatatcaaaatggcgctgcgtcttaaagatggaggccgctacctggcggacttcaagactacttacaaggccaaaaaaccagtgcagtcccggatcccttgct

SEQ ID NO: 145 - DNA, 120 bp, synthetic construct
  source 1..120: mol_type = other DNA
agcaagggatccgggtatgccgggtgcgtacaatgtagatcgtaaattagatattacaagtcacaatgaagattacacggtcgtagagcagtatgagcgcagtgtgcacatgggaagcgg

SEQ ID NO: 146 - DNA, 120 bp, synthetic construct
  source 1..120: mol_type = other DNA
ccgcttcccatgtgctaggggcgtcactctacgggcggtatggacgagttatacaagtaaggtaccctcgagtttgtttatttttctaaatacattcaaatatgtacgccgaatcccaca

SEQ ID NO: 147 - DNA, 89 bp, synthetic construct
  source 1..89: mol_type = other DNA
tgtgggattcggcgtttatccgctcatgagacaataaccctgataaatgcttcaataatattgaaaaaggaagagtatgagccatattc

SEQ ID NO: 148 - DNA, 100 bp, synthetic construct
  source 1..100: mol_type = other DNA
gttcctcaccttgtcgtattatactatgccgatatactatgccgatgattaattgtcaacgactttgcgtgttgtcttactattgctggcaggaggtcag

SEQ ID NO: 149 - DNA, 100 bp, synthetic construct
  source 1..100: mol_type = other DNA
aatacgacaaggtgaggaactaaaccatggtgtcgaaaggtgaggagcttttcacgggggtggttccaatcttagtcgagcttgatggtgacgtgaatgg

SEQ ID NO: 150 - DNA, 100 bp, synthetic construct
  source 1..100: mol_type = other DNA
ctgtagtacaaataaactttaaagtaagtttaccgttcgtcgcatcgccctcaccctcgccgcgcacgctaaacttatggccattcacgtcaccatcaag

SEQ ID NO: 151 - DNA, 100 bp, synthetic construct
  source 1..100: mol_type = other DNA
aaagtttatttgtactacaggcaaattaccggtcccgtggccgaccctggtcactacgcttgggtatggggtagcatgttttgcccgttaccctgaccac

SEQ ID NO: 152 - DNA, 100 bp, synthetic construct
  source 1..100: mol_type = other DNA
ccgtcgtctttaaaactgatagtgcgctcctgaacatacccttcgggcatggcagatttaaagaaatcgtgctgtttcatgtggtcagggtaacgggcaa

SEQ ID NO: 153 - DNA, 100 bp, synthetic construct
  source 1..100: mol_type = other DNA
atcagttttaaagacgacggaacgtacaaaactcgtgctgaagtaaagttcgaaggtgatacccttgtgaaccgtatcgtccttaaaggcatcgacttca

SEQ ID NO: 154 - DNA, 100 bp, synthetic construct
  source 1..100: mol_type = other DNA
ttgcttatctgctgtgatataaaccttatgtgaattgaagttgtactctaatttgtggcctaaaatgttaccgtcctctttgaagtcgatgcctttaagg

SEQ ID NO: 155 - DNA, 100 bp, synthetic construct
  source 1..100: mol_type = other DNA
atatcacagcagataagcaaaaaaatggcatcaaggcaaattttaagacccgccacaacgtagaagatggtggagtccagttggcggaccactaccagca

SEQ ID NO: 156 - DNA, 100 bp, synthetic construct
  source 1..100: mol_type = other DNA
ggtccttactcagtttactctggtggctaagatagtgattgtcgggaagcaaaaccggaccatcgccaatcggtgtattttgctggtagtggtccgccaa

SEQ ID NO: 157 - DNA, 100 bp, synthetic construct
  source 1..100: mol_type = other DNA
gagtaaactgagtaaggacccaaatgaaaagcgcgaccacatggtattgaaagagcgcgttacagcagctggaattacacacgatatggacgaactttat

SEQ ID NO: 158 - DNA, 100 bp, synthetic construct
  source 1..100: mol_type = other DNA
gcttcgcctttactgaccatatttctcctctttgctagcattatacctaggactgagctagctgtcaaatagatttacttataaagttcgtccatatcgt

SEQ ID NO: 159 - DNA, 100 bp, synthetic construct
  source 1..100: mol_type = other DNA
atggtcagtaaaggcgaagcagttatcaaagagttcatgcgcttcaaagttcatatggaagggtcgatgaacgggcacgaatttgaaattgaaggcgaag

SEQ ID NO: 160 - DNA, 100 bp, synthetic construct
  source 1..100: mol_type = other DNA
gtcccacgaaaagggtaatggaccgcctttagtaaccttaagctttgcggtttgggtcccttcatatgggcggccttcgccttcgccttcaatttcaaat

SEQ ID NO: 161 - DNA, 100 bp, synthetic construct
  source 1..100: mol_type = other DNA
cattacccttttcgtgggacattttaagcccacagtttatgtacgggagtcgcgctttcatcaagcaccctgcggacatcccagattactacaaacagtc

SEQ ID NO: 162 - DNA, 100 bp, synthetic construct
  source 1..100: mol_type = other DNA
aagaggtatcttgggtgaccgtcacggctccgccatcctcgaagttcatcacgcgctcccacttgaacccctcggggaaagactgtttgtagtaatctgg

SEQ ID NO: 163 - DNA, 100 bp, synthetic construct
  source 1..100: mol_type = other DNA
ggtcacccaagatacctctttggaggacggtacgttgatctacaaagtgaaattgcgtggcacgaattttccacctgatgggcctgtcatgcagaaaaag

SEQ ID NO: 164 - DNA, 100 bp, synthetic construct
  source 1..100: mol_type = other DNA
cgcagcgccattttgatatcccctttaagaacaccgtcctctgggtaaaggcgctccgtggaagcttcccatcccattgtctttttctgcatgacaggcc

SEQ ID NO: 165 - DNA, 100 bp, synthetic construct
  source 1..100: mol_type = other DNA
gatatcaaaatggcgctgcgtcttaaagatggaggccgctacctggcggacttcaagactacttacaaggccaaaaaaccagtgcagatgccgggtgcgt

SEQ ID NO: 166 - DNA, 100 bp, synthetic construct
  source 1..100: mol_type = other DNA
cccctcactgcgctcatactgctctacgaccgtgtaatcttcattgtgacttgtaatatctaatttacgatctacattgtacgcacccggcatctgcact

SEQ ID NO: 167 - DNA, 100 bp, synthetic construct
  source 1..100: mol_type = other DNA
agtatgagcgcagtgaggggcgtcactctacgggcggtatggacgagttatacaagtaaggtaccctcgagtttgtttatttttctaaatacattcaaat

SEQ ID NO: 168 - DNA, 82 bp, synthetic construct; identical to SEQ ID NO: 9
  source 1..82: mol_type = other DNA

SEQ ID NO: 169 - DNA, 84 bp, synthetic construct; identical to SEQ ID NO: 11
  source 1..84: mol_type = other DNA

SEQ ID NO: 170 - DNA, 73 bp, synthetic construct; identical to SEQ ID NO: 13
  source 1..73: mol_type = other DNA

SEQ ID NO: 171 - DNA, 81 bp, synthetic construct; identical to SEQ ID NO: 15
  source 1..81: mol_type = other DNA

SEQ ID NO: 172 - DNA, 80 bp, synthetic construct; identical to SEQ ID NO: 17
  source 1..80: mol_type = other DNA

SEQ ID NO: 173 - DNA, 78 bp, synthetic construct; identical to SEQ ID NO: 19
  source 1..78: mol_type = other DNA

SEQ ID NO: 174 - DNA, 79 bp, synthetic construct; identical to SEQ ID NO: 21
  source 1..79: mol_type = other DNA

SEQ ID NO: 175 - DNA, 83 bp, synthetic construct; identical to SEQ ID NO: 23
  source 1..83: mol_type = other DNA

SEQ ID NO: 176 - DNA, 72 bp, synthetic construct; identical to SEQ ID NO: 25
  source 1..72: mol_type = other DNA

SEQ ID NO: 177 - DNA, 82 bp, synthetic construct; identical to SEQ ID NO: 27
  source 1..82: mol_type = other DNA

SEQ ID NO: 178 - DNA, 80 bp, synthetic construct; identical to SEQ ID NO: 29
  source 1..80: mol_type = other DNA

SEQ ID NO: 179 - DNA, 80 bp, synthetic construct; identical to SEQ ID NO: 31
  source 1..80: mol_type = other DNA

SEQ ID NO: 180 - DNA, 83 bp, synthetic construct; identical to SEQ ID NO: 33
  source 1..83: mol_type = other DNA

SEQ ID NO: 181 - DNA, 76 bp, synthetic construct; identical to SEQ ID NO: 35
  source 1..76: mol_type = other DNA

SEQ ID NO: 182 - DNA, 77 bp, synthetic construct; identical to SEQ ID NO: 37
  source 1..77: mol_type = other DNA

SEQ ID NO: 183 - DNA, 79 bp, synthetic construct; identical to SEQ ID NO: 39
  source 1..79: mol_type = other DNA

SEQ ID NO: 184 - DNA, 83 bp, synthetic construct; identical to SEQ ID NO: 41
  source 1..83: mol_type = other DNA

SEQ ID NO: 185 - DNA, 81 bp, synthetic construct; identical to SEQ ID NO: 43
  source 1..81: mol_type = other DNA

SEQ ID NO: 186 - DNA, 83 bp, synthetic construct; identical to SEQ ID NO: 45
  source 1..83: mol_type = other DNA

SEQ ID NO: 187 - DNA, 81 bp, synthetic construct; identical to SEQ ID NO: 47
  source 1..81: mol_type = other DNA

SEQ ID NO: 188 - DNA, 80 bp, synthetic construct; identical to SEQ ID NO: 49
  source 1..80: mol_type = other DNA

SEQ ID NO: 189 - DNA, 83 bp, synthetic construct; identical to SEQ ID NO: 51
  source 1..83: mol_type = other DNA

SEQ ID NO: 190 - DNA, 80 bp, synthetic construct; identical to SEQ ID NO: 53
  source 1..80: mol_type = other DNA

SEQ ID NO: 191 - DNA, 80 bp, synthetic construct; identical to SEQ ID NO: 55
  source 1..80: mol_type = other DNA

SEQ ID NO: 192 - DNA, 83 bp, synthetic construct; identical to SEQ ID NO: 57
  source 1..83: mol_type = other DNA

SEQ ID NO: 193 - DNA, 81 bp, synthetic construct; identical to SEQ ID NO: 59
  source 1..81: mol_type = other DNA

SEQ ID NO: 194 - DNA, 83 bp, synthetic construct; identical to SEQ ID NO: 61
  source 1..83: mol_type = other DNA

SEQ ID NO: 195 - DNA, 77 bp, synthetic construct; identical to SEQ ID NO: 63
  source 1..77: mol_type = other DNA

SEQ ID NO: 196 - DNA, 80 bp, synthetic construct; identical to SEQ ID NO: 65
  source 1..80: mol_type = other DNA

SEQ ID NO: 197 - DNA, 80 bp, synthetic construct; identical to SEQ ID NO: 67
  source 1..80: mol_type = other DNA

SEQ ID NO: 198 - DNA, 71 bp, synthetic construct; identical to SEQ ID NO: 69
  source 1..71: mol_type = other DNA

SEQ ID NO: 199 - DNA, 83 bp, synthetic construct; identical to SEQ ID NO: 71
  source 1..83: mol_type = other DNA

SEQ ID NO: 200 - DNA, 74 bp, synthetic construct; identical to SEQ ID NO: 73
  source 1..74: mol_type = other DNA

SEQ ID NO: 201 - DNA, 83 bp, synthetic construct; identical to SEQ ID NO: 75
  source 1..83: mol_type = other DNA

SEQ ID NO: 202 - DNA, 82 bp, synthetic construct; identical to SEQ ID NO: 77
  source 1..82: mol_type = other DNA

SEQ ID NO: 203 - DNA, 80 bp, synthetic construct; identical to SEQ ID NO: 79
  source 1..80: mol_type = other DNA

SEQ ID NO: 204 - DNA, 82 bp, synthetic construct; identical to SEQ ID NO: 81
  source 1..82: mol_type = other DNA

SEQ ID NO: 205 - DNA, 81 bp, synthetic construct; identical to SEQ ID NO: 83
  source 1..81: mol_type = other DNA

SEQ ID NO: 206 - DNA, 83 bp, synthetic construct; identical to SEQ ID NO: 85
  source 1..83: mol_type = other DNA

SEQ ID NO: 207 - DNA, 112 bp, synthetic construct; identical to SEQ ID NO: 87
  source 1..112: mol_type = other DNA

SEQ ID NO: 208 - DNA, 120 bp, synthetic construct
  source 1..120: mol_type = other DNA
aggtcctcaatcgcactggatgaaggttctgtgggctgcgttgctggtcacattcctggcaggatgccaggccaaggtggagcaagcggtggagacagagccggacgaccaacgcaagtc

SEQ ID NO: 209 - DNA, 92 bp, synthetic construct
  source 1..92: mol_type = other DNA
ccaccgcttgctccaccttggcctggcatcctgccaggaatgtgaccagcaacgcagcccacagaaccttcatccagtgcgattgaggacct

SEQ ID NO: 210 - DNA, 119 bp, synthetic construct
  source 1..119: mol_type = other DNA
gacttgcgttggtcgtccggagcccgagctgcgccagcagaccgagtggcagagcggccagcgctgggaactggcactgggtcgcttttgggattacctgcttatcatcgctgcaacac

SEQ ID NO: 211 - DNA, 83 bp, synthetic construct
  source 1..83: mol_type = other DNA
ccaaaagcgacccagtgccagttcccagcgctggccgctctgccactcggtctgctggcgcagctcgggctccggctctgtct

SEQ ID NO: 212 - DNA, 116 bp, synthetic construct
  source 1..116: mol_type = other DNA
gtgttgcagcgatgataagctgggtgcagacactgtctgagcaggtgcaggaggagctgctcagctcccaggtcacccaggaactgagggcgctgatgtgcttcccatatctaggt

SEQ ID NO: 213 - DNA, 80 bp, synthetic construct
  source 1..80: mol_type = other DNA
ctcagttcctgggtgacctgggagctgagcagctcctcctgcacctgctcagacagtgtctgcacccagcgcaggtaatc

SEQ ID NO: 214 - DNA, 115 bp, synthetic construct
  source 1..115: mol_type = other DNA
acctagatatgggaagcagacgagaccatgaaggagttgaaggcctacaaatcggaactggaggaacaactgaccccggtggcggaggagacgcgggctgtctctattagttgtt

SEQ ID NO: 215 - DNA, 79 bp, synthetic construct
  source 1..79: mol_type = other DNA
ctccgccaccggggtcagttgttcctccagttccgatttgtaggccttcaactccttcatggtctcgtccatcagcgcc

SEQ ID NO: 216 - DNA, 118 bp, synthetic construct
  source 1..118: mol_type = other DNA
aacaactaatagagacagcacggctgtccaaggagctgcaggcggcgcaggcccggctgggcgcggacatggaggacgtgtgcggccgcctggtgcagtagcgtttgggaaaaccctc

SEQ ID NO: 217 - DNA, 82 bp, synthetic construct
  source 1..82: mol_type = other DNA
ggcggccgcacacgtcctccatgtccgcgcccagccgggcctgcgccgcctgcagctccttggacagccgtgcccgcgtctc

SEQ ID NO: 218 - DNA, 119 bp, synthetic construct
  source 1..119: mol_type = other DNA
gagggttttcccaaacgcccgcggcgaggtgcaggccatgctcggccagagcaccgaggagctgcgggtgcgcctcgcctcccacctgcgcaagctgcgtaatggtgtagccatataca

SEQ ID NO: 219 - DNA, 83 bp, synthetic construct
  source 1..83: mol_type = other DNA
gcgcaggtgggaggcgaggcgcacccgcagctcctcggtgctctggccgagcatggcctgcacctcgccgcggtactgcacca

SEQ ID NO: 220 - DNA, 113 bp, synthetic construct
  source 1..113: mol_type = other DNA
tgtatatggctacaccatagcggctcctccgcgatgccgatgacctgcagaagcgcctggcagtgtaccaggccggggcccgcgagggcgccgagcttggtaaatagcagtgg

SEQ ID NO: 221 - DNA, 77 bp, synthetic construct
  source 1..77: mol_type = other DNA
tcgcgggccccggcctggtacactgccaggcgcttctgcaggtcatcggcatcgcggaggagccgcttacgcagctt

SEQ ID NO: 222 - DNA, 113 bp, synthetic construct
  source 1..113: mol_type = other DNA
ccactgctatttaccaagcgcggcctcagcgccatccgcgagcgcctggggcccctggtggaacagggccgcgtgcgggccgccactgtgggctccgcgcgtgcgagggcgaa

SEQ ID NO: 223 - DNA, 77 bp, synthetic construct
  source 1..77: mol_type = other DNA
tggcggcccgcacgcggccctgttccaccaggggccccaggcgctcgcggatggcgctgaggccgcgctcggcgccc

SEQ ID NO: 224 - DNA, 112 bp, synthetic construct
  source 1..112: mol_type = other DNA
ttcgccctcgcacgcgcgcctggccggccagccgctacaggagcgggcccaggcctggggcgagcggctgcgcgcgcggatggaggagatgggccggcaggaccattagggt

SEQ ID NO: 225 - DNA, 76 bp, synthetic construct
  source 1..76: mol_type = other DNA
tccatccgcgcgcgcagccgctcgccccaggcctgggcccgctcctgtagcggctggccggccagggagcccacag

SEQ ID NO: 226 - DNA, 114 bp, synthetic construct
  source 1..114: mol_type = other DNA
accctaatggtcctgccgagccggacccgcgaccgcctggacgaggtgaaggagcaggtggcggaggtgcgcgccaagctggaggagcaggcccagcgaggttatagttatcgt

SEQ ID NO: 227 - DNA, 78 bp, synthetic construct
  source 1..78: mol_type = other DNA
tcctccagcttggcgcgcacctccgccacctgctccttcacctcgtccaggcggtcgcgggtccggctgcccatctcc

SEQ ID NO: 228 - DNA, 119 bp, synthetic construct
  source 1..119: mol_type = other DNA
acgataactataacctcgcagatacgcctgcaggccgaggccttccaggcccgcctcaagagctggttcgagcccctggtggaagacatgcagcgccagtgtggtgaccctgtgagtct

SEQ ID NO: 229 - DNA, 83 bp, synthetic construct
  source 1..83: mol_type = other DNA
gcatgtcttccaccaggggctcgaaccagctcttgaggcgggcctggaaggcctcggcctgcaggcgtatctgctgggcctgc

SEQ ID NO: 230 - DNA, 113 bp, synthetic construct
  source 1..113: mol_type = other DNA
agactcacagggtcaccaggccgggctggtggagaaggtgcaggctgccgtgggcaccagcgccgcccctgtgcccagcgacaatcactgaggaaatgtcagagccagcgtct

SEQ ID NO: 231 - DNA, 105 bp, synthetic construct
  source 1..105: mol_type = other DNA
agacgctggctctgacatttcctcagtgattgtcgctgggcacaggggcggcgctggtgcccacggcagcctgcaccttctccaccagcccggcccactggcgct

SEQ ID NO: 232 - DNA, 110 bp, synthetic construct
  source 1..110: mol_type = other DNA
gcactgaaggtcctcaatcgcactggaaacatcaaggtcggtctacaccaagatctctcgatcatcatcagtctccgttgaaggaggacgaccggtacaagatcccaaga

SEQ ID NO: 233 - DNA, 102 bp, synthetic construct
  source 1..102: mol_type = other DNA
ggtcctcgtcgtcgtcctccttcaacggagactgatgatgatcgagagatcttggtgtagaccgaccttgatgtttccagtgcgattgaggaccttcagtgc

SEQ ID NO: 234 - DNA, 108 bp, synthetic construct
  source 1..108: mol_type = other DNA
tcttgggatcttgtaccggacgaggaccatggggaggacgaggaattggaggactcagtggctcaggagatctcgacgtacttggcggtccctgtttaggaaatgctg

SEQ ID NO: 235 - DNA, 72 bp, synthetic construct
  source 1..72: mol_type = other DNA
ggtcctcgtcgaccgccaagtacgtcgagatctcctgagccactgagtcctccaattcctcgtcctccccat

SEQ ID NO: 236 - DNA, 111 bp, synthetic construct
  source 1..111: mol_type = other DNA
cagcatttcctaaacagggacgaggaccatggggtcgaggttatggaccttctggctctgtatcagtctcagtctctgtcgaacgaggaatcctagtctttcggccaacca

SEQ ID NO: 237 - DNA, 75 bp, synthetic construct
  source 1..75: mol_type = other DNA
ggtcctcgtcggattcctcgttcgacagagactgagactgatacagagccagaaggtccataacctcgaccccat

SEQ ID NO: 238 - DNA, 165 bp, synthetic construct
  source 1..165: mol_type = other DNA
tggttggccgaaagactagacgaggaccatggggacgacgaggaaaggtttcaatcagccgatccgtttccattgaacgaattgtcaccccaggcgtctacaccaagatctctcgatcatcatcagtctccgttgaaggaggacgaccttagttattctccgccg

SEQ ID NO: 239 - DNA, 129 bp, synthetic construct
  source 1..129: mol_type = other DNA
ggtcctcgtcgtcgtcctccttcaacggagactgatgatgatcgagagatcttggtgtagacgcctggggtgacaattcgttcaatggaaacggatcggctgattgaaacctttcctcgtcgtccccat

SEQ ID NO: 240 - DNA, 120 bp, synthetic construct
  source 1..120: mol_type = other DNA
cggcggagaataactaaggacgaggaccatggggaggacgaggaattggaggactcagtggctcaggagatctcgacgtacttggcggtcgcttggattctgcgtttgtttccgtctacg

SEQ ID NO: 241 - DNA, 92 bp, synthetic construct
  source 1..92: mol_type = other DNA
cgtagacggaaacaaacgcagaatccaagcgaccgccaagtacgtcgagatctcctgagccactgagtcctccaattcctcgtcctccccat

SEQ ID NO: 242 - DNA, 105 bp, synthetic construct
  source 1..105: mol_type = other DNA
cattactcgcatccattctcaggctgtctcgtctcgtctgctgggagttcgtagacggaaacaaacgcagaatccaagctctatttgactaacgcccagcctgtc

SEQ ID NO: 243 - DNA, 79 bp, synthetic construct
  source 1..79: mol_type = other DNA
gcttggattctgcgtttgtttccgtctacgaactcccagcagacgagacgagacagcctgagaatggatgcgagtaatg

SEQ ID NO: 244 - DNA, 120 bp, synthetic construct
  source 1..120: mol_type = other DNA
gacaggctgggcgtttagctagctcagtcctaggtataatgctagcaaagaggagaaaggatctatggtcagtaaaggcgaagcagttatcaaagagttcatgctcgcagggtccagagt

SEQ ID NO: 245 - DNA, 88 bp, synthetic construct
  source 1..88: mol_type = other DNA
tttgataactgcttcgcctttactgaccatagatcctttctcctctttgctagcattatacctaggactgagctagctgtcaaataga

SEQ ID NO: 246 - DNA, 120 bp, synthetic construct; identical to SEQ ID NO: 141
  source 1..120: mol_type = other DNA

SEQ ID NO: 247 - DNA, 88 bp, synthetic construct; identical to SEQ ID NO: 119
  source 1..88: mol_type = other DNA

SEQ ID NO: 248 - DNA, 119 bp, synthetic construct; identical to SEQ ID NO: 142
  source 1..119: mol_type = other DNA

SEQ ID NO: 249 - DNA, 87 bp, synthetic construct; identical to SEQ ID NO: 120
  source 1..87: mol_type = other DNA

SEQ ID NO: 250 - DNA, 120 bp, synthetic construct; identical to SEQ ID NO: 143
  source 1..120: mol_type = other DNA

SEQ ID NO: 251 - DNA, 88 bp, synthetic construct; identical to SEQ ID NO: 121
  source 1..88: mol_type = other DNA

SEQ ID NO: 252 - DNA, 120 bp, synthetic construct; identical to SEQ ID NO: 139
  source 1..120: mol_type = other DNA

SEQ ID NO: 253 - DNA, 88 bp, synthetic construct; identical to SEQ ID NO: 122
  source 1..88: mol_type = other DNA

SEQ ID NO: 254 - DNA, 120 bp, synthetic construct; identical to SEQ ID NO: 140
  source 1..120: mol_type = other DNA

SEQ ID NO: 255 - DNA, 88 bp, synthetic construct; identical to SEQ ID NO: 123
  source 1..88: mol_type = other DNA

SEQ ID NO: 256 - DNA, 120 bp, synthetic construct; identical to SEQ ID NO: 144
  source 1..120: mol_type = other DNA

SEQ ID NO: 257 - DNA, 88 bp, synthetic construct; identical to SEQ ID NO: 124
  source 1..88: mol_type = other DNA

SEQ ID NO: 258 - DNA, 120 bp, synthetic construct; identical to SEQ ID NO: 145
  source 1..120: mol_type = other DNA

SEQ ID NO: 259 - DNA, 88 bp, synthetic construct; identical to SEQ ID NO: 125
  source 1..88: mol_type = other DNA

SEQ ID NO: 260 - DNA, 88 bp, synthetic construct
  source 1..88: mol_type = other DNA
ccgcttcccatgtgctaggggcgtcactctacgggcggtatggacgagttatacaagtaacgaccttgatgtttccagtgcgattgag

SEQ ID NO: 261 - DNA, 82 bp, synthetic construct
  source 1..82: mol_type = other DNA
ctcaatcgcactggaaacatcaaggtcgttacttgtataactcgtccataccgcccgtagagtgacgcccctcactgcgctc

SEQ ID NO: 262 - DNA, 96 bp, synthetic construct
  source 1..96: mol_type = other DNA
cattactcgcatccattctcaggctgtctcgtctcgtctctgacctcctgccagcaatagtaagacaacacgcaaagtcttaggcacgctcaacct

SEQ ID NO: 263 - DNA, 70 bp, synthetic construct
  source 1..70: mol_type = other DNA
tgttgtcttactattgctggcaggaggtcagagacgagacgagacagcctgagaatggatgcgagtaatg

SEQ ID NO: 264 - DNA, 120 bp, synthetic construct
  source 1..120: mol_type = other DNA
aggttgagcgtgcctttgacagctagctcagtcctaggtataatgctagcaaagaggagaaaggatctatggtgtcgaaaggtgaggagcttttcacgggggtgtaggcggtcgagagtg

SEQ ID NO: 265 - DNA, 88 bp, synthetic construct
  source 1..88: mol_type = other DNA
aaaagctcctcacctttcgacaccatagatcctttctcctctttgctagcattatacctaggactgagctagctgtcaagactttgcg

SEQ ID NO: 266 - DNA, 120 bp, synthetic construct; identical to SEQ ID NO: 130
  source 1..120: mol_type = other DNA

SEQ ID NO: 267 - DNA, 88 bp, synthetic construct; identical to SEQ ID NO: 110
  source 1..88: mol_type = other DNA

SEQ ID NO: 268 - DNA, 117 bp, synthetic construct; identical to SEQ ID NO: 131
  source 1..117: mol_type = other DNA

SEQ ID NO: 269 - DNA, 85 bp, synthetic construct; identical to SEQ ID NO: 111
  source 1..85: mol_type = other DNA

SEQ ID NO: 270 - DNA, 120 bp, synthetic construct; identical to SEQ ID NO: 132
  source 1..120: mol_type = other DNA

SEQ ID NO: 271 - DNA, 88 bp, synthetic construct; identical to SEQ ID NO: 112
  source 1..88: mol_type = other DNA

SEQ ID NO: 272 - DNA, 119 bp, synthetic construct; identical to SEQ ID NO: 133
  source 1..119: mol_type = other DNA

SEQ ID NO: 273 - DNA, 87 bp, synthetic construct; identical to SEQ ID NO: 113
  source 1..87: mol_type = other DNA

SEQ ID NO: 274 - DNA, 120 bp, synthetic construct; identical to SEQ ID NO: 134
  source 1..120: mol_type = other DNA

SEQ ID NO: 275 - DNA, 88 bp, synthetic construct; identical to SEQ ID NO: 114
  source 1..88: mol_type = other DNA

SEQ ID NO: 276 - DNA, 120 bp, synthetic construct; identical to SEQ ID NO: 135
  source 1..120: mol_type = other DNA

SEQ ID NO: 277 - DNA, 88 bp, synthetic construct; identical to SEQ ID NO: 115
  source 1..88: mol_type = other DNA

SEQ ID NO: 278 - DNA, 112 bp, synthetic construct; identical to SEQ ID NO: 136
  source 1..112: mol_type = other DNA

SEQ ID NO: 279 - DNA, 80 bp, synthetic construct; identical to SEQ ID NO: 116
  source 1..80: mol_type = other DNA

SEQ ID NO: 280 - DNA, 120 bp, synthetic construct
  source 1..120: mol_type = other DNA
tgaagacagcgggcataaaagcgcgaccacatggtattgaaagagcgcgttacagcagctggaattacacacgatatggacgaactttataagtaacgaccttgatgtttccagtgcgat

SEQ ID NO: 281 - DNA, 114 bp, synthetic construct
  source 1..114: mol_type = other DNA
atcgcactggaaacatcaaggtcgttacttataaagttcgtccatatcgtgtgtaattccagctgctgtaacgcgctctttcaataccatgtggtcgcgcttttcatttgggtc

SEQ ID NO: 282 - DNA, 118 bp, synthetic construct
  source 1..118: mol_type = other DNA
cattactcgcatccattctcaggctgtctcgtctcgtctcctcgtctcaaccaaagcaatcaacccatcaaccacctggtctatttgacagctagctcagtctgacctgtctcccgca

SEQ ID NO: 283 - DNA, 92 bp, synthetic construct
  source 1..92: mol_type = other DNA
gctgtcaaatagaccaggtggttgatgggttgattgctttggttgagacgaggagacgagacgagacagcctgagaatggatgcgagtaatg

SEQ ID NO: 284 - DNA, 120 bp, synthetic construct
  source 1..120: mol_type = other DNA
tgcgggagacaggtctctaggtataatgctagcaaagaggagaaaggatctatggcttcactggttaaaaaagacatgtgcatcaaaatgacgatggaaggaactcggcgctcagtacca

SEQ ID NO: 285 - DNA, 88 bp, synthetic construct
  source 1..88: mol_type = other DNA
tcgtcattttgatgcacatgtcttttttaaccagtgaagccatagatcctttctcctctttgctagcattatacctaggactgagcta

SEQ ID NO: 286 - DNA, 120 bp, synthetic construct
  source 1..120: mol_type = other DNA
tggtactgagcgccgtagtaaacggtcaccatttcaagtgtgtaggagaaggcgaaggcaaaccatttgaagggacccaggtggaaaagatacgcatcactgaatttgtctctggccgct

SEQ ID NO: 287 - DNA, 88 bp, synthetic construct
  source 1..88: mol_type = other DNA
cgtatcttttccacctgggtcccttcaaatggtttgccttcgccttctcctacacacttgaaatggtgaccgtttactgttccttcca

SEQ ID NO: 288 - DNA, 120 bp, synthetic construct
  source 1..120: mol_type = other DNA
agcggccagagacaatggtgggcccttaccatttgcgtatgatattttggccccttgttgcatgtatggcagtaaaaccttcattaagcatgtgtcgggtattcttctgagacggcgagg

SEQ ID NO: 289 - DNA, 88 bp, synthetic construct
  source 1..88: mol_type = other DNA
cacatgcttaatgaaggttttactgccatacatgcaacaaggggccaaaatatcatacgcaaatggtaagggcccaccttcagtgatg

SEQ ID NO: 290 - DNA, 120 bp, synthetic construct
  source 1..120: mol_type = other DNA
cctcgccgtctcagatcggattactttaaggagtcttttcctgagggctttacctgggaaagaacacaaatcttcgaggatggcggctatctcaccatacaccatagtcttgcggcgttg

SEQ ID NO: 291 - DNA, 88 bp, synthetic construct
  source 1..88: mol_type = other DNA
tgagatagccgccatcctcgaagatttgtgttctttcccaggtaaagccctcaggaaaagactccttaaagtaatccggaatacccga

SEQ ID NO: 292 - DNA, 120 bp, synthetic construct
  source 1..120: mol_type = other DNA
caacgccgcaagacttggacacgagccttcagggtaataattttattttcaaagttaatgtcatcggtgccaacttccctgcaaacggtcccgtgatgcagaaatagcggttcgggttga

SEQ ID NO: 293 - DNA, 88 bp, synthetic construct
  source 1..88: mol_type = other DNA
acgggaccgtttgcagggaagttggcaccgatgacattaactttgaaaataaaattattaccctgaaggctcgtgtcctggtgtatgg

SEQ ID NO: 294 - DNA, 120 bp, synthetic construct
  source 1..120: mol_type = other DNA
tcaacccgaaccgcttaaaacagctggatgggaaccgtgcgttgagatgctttatccgcgggacggcgtcctgtgtggtcagagcctgatggccctgaaatgcatggcaggtccgagtgt

SEQ ID NO: 295 - DNA, 88 bp, synthetic construct
  source 1..88: mol_type = other DNA
ggccatcaggctctgaccacacaggacgccgtcccgcggataaagcatctcaacgcacggttcccatccagctgtttttttctgcatc

SEQ ID NO: 296 - DNA, 120 bp, synthetic construct
  source 1..120: mol_type = other DNA
acactcggacctgcctctgatggcaatcatctgacgtcccacctgcgcactacctatcgttctcgcaagccatccaatgcagttaacatgccggaatttcattttcgcatggccttcacc

SEQ ID NO: 297 - DNA, 88 bp, synthetic construct
  source 1..88: mol_type = other DNA
ccggcatgttaactgcattggatggcttgcgagaacgataggtagtgcgcaggtgggacgtcagatgattgccatcagtgcatttcag

SEQ ID NO: 298 - DNA, 105 bp, synthetic construct
  source 1..105: mol_type = other DNA
ggtgaaggccatgcgttggggatcatcgcattgagattttgaaagctgaacaaggtaaattttatgaacaatacgagtcagcggtggcctcgtgcaatgtggcgt

SEQ ID NO: 299 - DNA, 73 bp, synthetic construct
  source 1..73: mol_type = other DNA
gactcgtattgttcataaaatttaccttgttcagctttcaaaatctcaatgcgatgatccccaaaatgaaatt

SEQ ID NO: 300 - DNA, 89 bp, synthetic construct
  source 1..89: mol_type = other DNA
acgccacattgcacgtcgttactgtgaggcggcaccgagtaaattagggcatcactaataacgaccttgatgtttccagtgcgattgag

SEQ ID NO: 301 - DNA, 83 bp, synthetic construct
  source 1..83: mol_type = other DNA
ctcaatcgcactggaaacatcaaggtcgttattagtgatgccctaatttactcggtgccgcctcacagtaacgggccaccgct

SEQ ID NO: 302 - DNA, 92 bp, synthetic construct
  source 1..92: mol_type = other DNA
atgagtaaaggagaagaacttttcactggagttgtcccaattcttgttgaattagatggtgatgttaatgggcaagcagcagattaaagtga

SEQ ID NO: 303 - DNA, 111 bp, synthetic construct
  source 1..111: mol_type = other DNA
tcactttaatctgctgctcaaattctctgtcagtggagagggtgaaggtgatgcaacatacggaaaacttacccttaaatttatttgcactacacttgctaacacgcgaca

SEQ ID NO: 304 - DNA, 118 bp, synthetic construct
  source 1..118: mol_type = other DNA
tgtcgcgtgttagcaagttggaaagctacctgttccatggccaacacttgtcactactttgacttatggtgttcaatgcttttcaagatacccagatcatacgagtgtagcatatcga

SEQ ID NO: 305 - DNA, 119 bp, synthetic construct
  source 1..119: mol_type = other DNA
tcgatatgctacactcgtatgaaacagcatgactttttcaagagtgccatgcccgaaggttatgtacaggaaagaactatattttacaaagatgacgggaatgatacggtccttccttg

SEQ ID NO: 306 - DNA, 113 bp, synthetic construct
  source 1..113: mol_type = other DNA
caaggaaggaccgtatcactacaaatcacgtgctgaagtcaagtttgaaggtgataccctcgttaatagaattgagttaaaaggtattgattttaaacgcggccctgtaccct

SEQ ID NO: 307 - DNA, 119 bp, synthetic construct
  source 1..119: mol_type = other DNA
agggtacagggccgcgttaagaagatggaaacattcttggacacaaaatggaatacaactataactcacacaatgtatacatcatggcagacaaacaaaagacgtgaattacgaccaag

SEQ ID NO: 308 - DNA, 117 bp, synthetic construct
  source 1..117: mol_type = other DNA
cttggtcgtaattcacgtaatggaatcaaagttaacttcaaaattagacacaacattgaagatggaagcgttcaactagcagaccattatcaacaaaattccgtttgaaaagagctg

SEQ ID NO: 309 - DNA, 107 bp, synthetic construct
  source 1..107: mol_type = other DNA
cagctcttttcaaacggaactccaattggcgatggccctgtccttttaccagacaaccattacctgtccacacaatctgccctttccaactggaatgttctcggggc

SEQ ID NO: 310 - DNA, 109 bp, synthetic construct
  source 1..109: mol_type = other DNA
gccccgagaacattccagagatcccaacgaaaagagagatcacatgatccttcttgagtttgtaacagctgctgggattacacatggcatggatgaactatacaaataa

SEQ ID NO: 311 - DNA, 115 bp, synthetic construct
  source 1..115: mol_type = other DNA
catcaccatctaattcaacaagaattgggacaactccagtgaaaagttcttctcctttactcatnnnnnnnnnnncgaccttgatgtttccagtgcgattgaggaccttcagtgc

SEQ ID NO: 312 - DNA, 118 bp, synthetic construct
  source 1..118: mol_type = other DNA
catcaccatctaattcaacaagaattgggacaactccagtgaaaagttcttctcctttactgaccatnnnnnnnnnnncgaccttgatgtttccagtgcgattgaggaccttcagtgc

SEQ ID NO: 313 - DNA, 75 bp, synthetic construct
  source 1..75: mol_type = other DNA
taaatttaagggtaagttttccgtatgttgcatcaccttcaccctctccactgacagagaatttgtgcccattaa

SEQ ID NO: 314 - DNA, 82 bp, synthetic construct
  source 1..82: mol_type = other DNA
tatcttgmaaagcattgaasaccatragtcaaagtagtgacaagtgttggccatggaacaggtagctttccagtagtgcaaa

SEQ ID NO: 315 - DNA, 82 bp, synthetic construct
  source 1..82: mol_type = other DNA
tatcttgmaaagcattgaasaccatracccaaagtagtgacaagtgttggccatggaacaggtagctttccagtagtgcaaa

SEQ ID NO: 316 - DNA, 82 bp, synthetic construct
  source 1..82: mol_type = other DNA
tatcttgmaaagcattgaasaccccaagtcaaagtagtgacaagtgttggccatggaacaggtagctttccagtagtgcaaa

SEQ ID NO: 317 - DNA, 82 bp, synthetic construct
  source 1..82: mol_type = other DNA
tatcttgmaaagcattgaasaccccaacccaaagtagtgacaagtgttggccatggaacaggtagctttccagtagtgcaaa

SEQ ID NO: 318 - DNA, 82 bp, synthetic construct
  source 1..82: mol_type = other DNA
tatcttgmaaagcattgattaccatragtcaaagtagtgacaagtgttggccatggaacaggtagctttccagtagtgcaaa

SEQ ID NO: 319 - DNA, 82 bp, synthetic construct
  source 1..82: mol_type = other DNA
tatcttgmaaagcattgattaccatracccaaagtagtgacaagtgttggccatggaacaggtagctttccagtagtgcaaa

SEQ ID NO: 320 - DNA, 82 bp, synthetic construct
  source 1..82: mol_type = other DNA
tatcttgmaaagcattgattaccccaagtcaaagtagtgacaagtgttggccatggaacaggtagctttccagtagtgcaaa

SEQ ID NO: 321 - DNA, 82 bp, synthetic construct
  source 1..82: mol_type = other DNA
tatcttgmaaagcattgattaccccaacccaaagtagtgacaagtgttggccatggaacaggtagctttccagtagtgcaaa

SEQ ID NO: 322 - DNA, 83 bp, synthetic construct
  source 1..83: mol_type = other DNA
ctttgtaaaatatagttctttcctgtacataaccttcgggcatggcactcttgaaaaagtcatgctgtttcatatgatctggg

SEQ ID NO: 323 - DNA, 77 bp, synthetic construct
  source 1..77: mol_type = other DNA
accttttaactcaaytctattaacgagggtatcaccttcaaacttgacttcagcacgtgatttgtagttcccgtcat

SEQ ID NO: 324 - DNA, 83 bp, synthetic construct
  source 1..83: mol_type = other DNA
tctgccrtgatgtatacattgygtgagwtatrgttgtattccattttgtgtccaagaatgtttccatcttctttaaaatcaat

SEQ ID NO: 325 - DNA, 83 bp, synthetic construct
  source 1..83: mol_type = other DNA
tctgccrtgatgtatacattgygtgagwtatggttgtattccattttgtgtccaagaatgtttccatcttctttaaaatcaat

SEQ ID NO: 326 - DNA, 83 bp, synthetic construct
  source 1..83: mol_type = other DNA
tctgccgcgatgtatacattgygtgagwtatrgttgtattccattttgtgtccaagaatgtttccatcttctttaaaatcaat

SEQ ID NO: 327 - DNA, 83 bp, synthetic construct
  source 1..83: mol_type = other DNA
tctgccgcgatgtatacattgygtgagwtatggttgtattccattttgtgtccaagaatgtttccatcttctttaaaatcaat

SEQ ID NO: 328 - DNA, 81 bp, synthetic construct
  source 1..81: mol_type = other DNA
taatggtctgctagttgaacgcttccatcttcaakgttgtgtctaaytttgaagttarcttkgattccattcttttgtttg

SEQ ID NO: 329 - DNA, 71 bp, synthetic construct
  source 1..71: mol_type = other DNA
cagattgtgtggacaggtaatggttgtctggtaaaaggacagggccatcgccaattggagtattttgttga

SEQ ID NO: 330 - DNA, 71 bp, synthetic construct
  source 1..71: mol_type = other DNA
cagattggtaggacaggtaatggttgtctggtaaaaggacagggccatcgccaattggagtattttgttga

SEQ ID NO: 331 - DNA, 120 bp, synthetic construct
  source 1..120: mol_type = other DNA
ggagttcgtagacggaaacttattygtatagttcatccatgccatgtgtaatcccagcagctgttacaaactcaagaaggatcatgtgatctctcttttcsttgggatctttggaaaggg

SEQ ID NO: 332 - DNA, 120 bp, synthetic construct
  source 1..120: mol_type = other DNA
cctcgtctcaaccaaagcaatcaacccatcaaccacctggcaagacgctggctctgacatttccgctactgaactactcgtcattactcgcatccattctcaggctgtctcgtctcgtct

SEQ ID NO: 333 - DNA, 120 bp, synthetic construct
  source 1..120: mol_type = other DNA
  modified_base 61: mod_base = OTHER; note = URACIL
agacgagacgagacagcctgagaatggatgcgagtaatgacgagtagttcagtagcggaatagccaactccctttacaacgctgggagttcgtagacggaaacaaacgcagaatccaagc

SEQ ID NO: 334 - DNA, 120 bp, synthetic construct
  source 1..120: mol_type = other DNA
  modified_base 60: mod_base = OTHER; note = URACIL
gcttggattctgcgtttgtttccgtctacgaactcccagcgttgtaaagggagttggcttatgtcagagccagcgtcttgccaggtggttgatgggttgattgctttggttgagacgagg

SEQ ID NO: 335 - DNA, 120 bp, synthetic construct; identical to SEQ ID NO: 333
  source 1..120: mol_type = other DNA
  modified_base 61: mod_base = OTHER; note = URACIL

SEQ ID NO: 336 - DNA, 120 bp, synthetic construct; identical to SEQ ID NO: 334
  source 1..120: mol_type = other DNA
  modified_base 60: mod_base = OTHER; note = URACIL

SEQ ID NO: 337 - DNA, 38 bp, synthetic construct
  source 1..38: mol_type = other DNA
atccatgcctgaattcgcggccgcttctagaggaaatc

SEQ ID NO: 338 - DNA, 25 bp, synthetic construct
  source 1..25: mol_type = other DNA
atcttcaagttggcgtactggatgg

SEQ ID NO: 339 - DNA, 31 bp, synthetic construct
  source 1..31: mol_type = other DNA
tatgaatatcgtgcccatattcttgagccac

SEQ ID NO: 340 - DNA, 39 bp, synthetic construct
  source 1..39: mol_type = other DNA
aaaaaatgcatttcatcttttttttcgcaatcactaaac

SEQ ID NO: 341 - DNA, 60 bp, synthetic construct
  source 1..60: mol_type = other DNA
tcttttttggcattcggtaatatatgcgcatatagattaagtttttctatcaacgctaac

SEQ ID NO: 342 - DNA, 25 bp, synthetic construct
  source 1..25: mol_type = other DNA
ctgacctcctgccagcaatagtaag

SEQ ID NO: 343 - DNA, 28 bp, synthetic construct
  source 1..28: mol_type = other DNA
gatagtgcgctcctgaacatacccttcg

SEQ ID NO: 344 - DNA, 33 bp, synthetic construct
  source 1..33: mol_type = other DNA
ataaagttcgtccatatcgtgtgtaattccagc

SEQ ID NO: 345 - DNA, 31 bp, synthetic construct
  source 1..31: mol_type = other DNA
ggtaccttacttgtataactcgtccataccg

SEQ ID NO: 346 - DNA, 22 bp, synthetic construct
  source 1..22: mol_type = other DNA
agacgctggctctgacatttcc

SEQ ID NO: 347 - DNA, 22 bp, synthetic construct
  source 1..22: mol_type = other DNA
aggtcctcaatcgcactggatg

SEQ ID NO: 348 - DNA, 25 bp, synthetic construct
  source 1..25: mol_type = other DNA
cactgaaggtcctcaatcgcactgg

SEQ ID NO: 349 - DNA, 30 bp, synthetic construct
  source 1..30: mol_type = other DNA
cgtagacggaaacaaacgcagaatccaagc

SEQ ID NO: 350 - DNA, 29 bp, synthetic construct
  source 1..29: mol_type = other DNA
  modified_base 11: mod_base = OTHER; note = URACIL
attactcgcatccattctcaggctgtctc

SEQ ID NO: 351 - DNA, 38 bp, synthetic construct
  source 1..38: mol_type = other DNA
  modified_base 12: mod_base = OTHER; note = URACIL
agatagatttctcaatcgcactggaaacatcaaggtcg

SEQ ID NO: 352 - DNA, 37 bp, synthetic construct
  source 1..37: mol_type = other DNA
  modified_base 10: mod_base = OTHER; note = URACIL
agtctcgtctgctgggagttcgtagacggaaacaaac

SEQ ID NO: 353 - DNA, 45 bp, synthetic construct
  source 1..45: mol_type = other DNA
  modified_base 11: mod_base = OTHER; note = URACIL
aacatcaaggtcgttacttgtataactcgtccataccgcccgtag

SEQ ID NO: 354 - DNA, 50 bp, synthetic construct
  source 1..50: mol_type = other DNA
  modified_base 10: mod_base = OTHER; note = URACIL
agtctcgtctcctcgtctcaaccaaagcaatcaacccatcaaccacctgg

SEQ ID NO: 355 - DNA, 43 bp, synthetic construct
  source 1..43: mol_type = other DNA
  modified_base 11: mod_base = OTHER; note = URACIL
aacatcaaggtcgttattagtgatgccctaatttactcggtgc

SEQ ID NO: 356 - DNA, 42 bp, synthetic construct
  source 1..42: mol_type = other DNA
  modified_base 10: mod_base = OTHER; note = URACIL
agtctcgtctcgtctctgacctcctgccagcaatagtaagac

SEQ ID NO: 357 - DNA, 52 bp, synthetic construct
  source 1..52: mol_type = other DNA
  modified_base 11: mod_base = OTHER; note = URACIL
aacatcaaggtcgttacttataaagttcgtccatatcgtgtgtaattccagc

SEQ ID NO: 358 - DNA, 32 bp, synthetic construct
  source 1..32: mol_type = other DNA
  modified_base 11: mod_base = OTHER; note = URACIL
atgcgagtaatgctggctcaccttcgggtggg

SEQ ID NO: 359 - DNA, 37 bp, synthetic construct
  source 1..37: mol_type = other DNA
  modified_base 12: mod_base = OTHER; note = URACIL
agaaatctatcttgatcggcacgtaagaggttccaac

SEQ ID NO: 360 - DNA, 30 bp, synthetic construct
  source 1..30: mol_type = other DNA
  modified_base 10: mod_base = OTHER; note = URACIL
agacgagactctggctcaccttcgggtggg

SEQ ID NO: 361 - DNA, 36 bp, synthetic construct
  source 1..36: mol_type = other DNA
  modified_base 11: mod_base = OTHER; note = URACIL
accttgatgtttgatcggcacgtaagaggttccaac

SEQ ID NO: 362 - DNA, 38 bp, synthetic construct
  source 1..38: mol_type = other DNA
  modified_base 10: mod_base = OTHER; note = URACIL
agtctcgtcttagcactgaaggtcctcaatcgcactgg

SEQ ID NO: 363 - DNA, 30 bp, synthetic construct
  source 1..30: mol_type = other DNA
  modified_base 11: mod_base = OTHER; note = URACIL
aacatcaaggtggagttcgtagacggaaac

SEQ ID NO: 364 - DNA, 46 bp, synthetic construct
  source 1..46: mol_type = other DNA
  modified_base 11: mod_base = OTHER; note = URACIL
accttgatgttgtttaaactcaggtcgaggtggcccggctccatgc

SEQ ID NO: 365 - DNA, 60 bp, synthetic construct
  source 1..60: mol_type = other DNA
  modified_base 12: mod_base = OTHER; note = URACIL
agtatttcccctctttctctagtattaaacaaaattatttgtagaggctgtttcgtcctc

SEQ ID NO: 366 - DNA, 35 bp, synthetic construct
  source 1..35: mol_type = other DNA
  modified_base 12: mod_base = OTHER; note = URACIL
aggggaaatactagaaagaggagaaaggatctatg

SEQ ID NO: 367 - DNA, 86 bp, synthetic construct
  source 1..86: mol_type = other DNA
atccatgcctgaattcgcggccgcttctagaggaaatcataaaaaatttatttgctttgtgagcggataacaattataatagattc

SEQ ID NO: 368 - DNA, 84 bp, synthetic construct
  source 1..84: mol_type = other DNA
aattgtgagcggataacaattactagaggagtcacacaggaaagtacatgaaatttggaaactttttgcttacataccaacctc

SEQ ID NO: 369 - DNA, 73 bp, synthetic construct
  source 1..73: mol_type = other DNA
cccaattttctcaaacagaggtaatgaaacgtttggttaaattaggtcgcatctctgaggagtgtggttttga

SEQ ID NO: 370 - DNA, 81 bp, synthetic construct
  source 1..81: mol_type = other DNA
taccgtatggttactggagcatcatttcacggagtttggtttgcttggtaacccttatgtcgctgctgcatatttacttgg

SEQ ID NO: 371 - DNA, 80 bp, synthetic construct
  source 1..80: mol_type = other DNA
cgcgactaaaaaattgaatgtaggaactgccgctattgttcttcccacagcccatccagtacgccaacttgaagatgtga

SEQ ID NO: 372 - DNA, 78 bp, synthetic construct
  source 1..78: mol_type = other DNA
atttattggatcaaatgtcaaaaggacgatttcggtttggtatttgccgagggctttacaacaaggactttcgcgtat

SEQ ID NO: 373 - DNA, 79 bp, synthetic construct
  source 1..79: mol_type = other DNA
tcggcacagatatgaataacagtcgcgccttagcggaatgctggtacgggctgataaagaatggcatgacagagggata

SEQ ID NO: 374 - DNA, 83 bp, synthetic construct
  source 1..83: mol_type = other DNA
tatggaagctgataatgaacatatcaagttccataaggtaaaagtaaaccccgcggcgtatagcagaggtggcgcaccggttt

SEQ ID NO: 375 - DNA, 72 bp, synthetic construct
  source 1..72: mol_type = other DNA
atgtggtggctgaatcagcttcgacgactgagtgggctgctcaatttggcctaccgatgatattaagttgga

SEQ ID NO: 376 - DNA, 82 bp, synthetic construct
  source 1..82: mol_type = other DNA
ttataaatactaacgaaaagaaagcacaacttgagctttataatgaagtggctcaagaatatgggcacgatattcataatat

SEQ ID NO: 377 - DNA, 80 bp, synthetic construct
  source 1..80: mol_type = other DNA
cgaccattgcttatcatatataacatctgtagatcatgactcaattaaagcgaaagagatttgccggaaatttctggggc

SEQ ID NO: 378 - DNA, 80 bp, synthetic construct
  source 1..80: mol_type = other DNA
attggtatgattcttatgtgaatgctacgactatttttgatgattcagaccaaacaagaggttatgatttcaataaaggg

SEQ ID NO: 379 - DNA, 83 bp, synthetic construct
  source 1..83: mol_type = other DNA
cagtggcgtgactttgtattaaaaggacataaagatactaatcgccgtattgattacagttacgaaatcaatcccgtgggaac

SEQ ID NO: 380 - DNA, 76 bp, synthetic construct
  source 1..76: mol_type = other DNA
gccgcaggaatgtattgacataattcaaaaagacattgatgctacaggaatatcaaatatttgttgtggatttgaa

SEQ ID NO: 381 - DNA, 77 bp, synthetic construct
  source 1..77: mol_type = other DNA
gctaatggaacagtagacgaaattattgcttccatgaagctcttccagtctgatgtcatgccatttcttaaagaaaa

SEQ ID NO: 382 - DNA, 79 bp, synthetic construct
  source 1..79: mol_type = other DNA
acaacgttcgctattatattaataatactagagagagaaagacaggacccactatgaaatttggattgttcttccttaa

SEQ ID NO: 383 - DNA, 83 bp, synthetic construct
  source 1..83: mol_type = other DNA
cttcatcaattcaacaactgttcaagaacaaagtatagttcgcatgcaggaaataacggagtatgttgataagttgaattttg

SEQ ID NO: 384 - DNA, 81 bp, synthetic construct
  source 1..81: mol_type = other DNA
aacagattttagtgtatgaaaatcatttttcagataatggtgttgtcggcgctcctctgactgtttctggttttctgctcg

SEQ ID NO: 385 - DNA, 83 bp, synthetic construct
  source 1..83: mol_type = other DNA
gtttaacagagaaaattaaaattggttcattaaatcacatcattacaactcatcatcctgtccgcatagcggaggaagcttgc

SEQ ID NO: 386 - DNA, 77 bp, synthetic construct
  source 1..77: mol_type = other DNA
ttattggatcagttaagtgaagggagatttattttagggtttagtgattgcgaaaaaaaagatgaaatgcatttttt

SEQ ID NO: 387 - DNA, 92 bp, synthetic construct
  source 1..92: mol_type = other DNA
atccatgcctgaattcgcggccgcttctagaggaaatcataaaaaatttatttgctttgtgagcggataacaattataatagattcaattgt

SEQ ID NO: 388 - DNA, 84 bp, synthetic construct
  source 1..84: mol_type = other DNA
gagcggataacaattactagaggagtcacacaggaaagtacatgaaatttggaaactttttgcttacataccaacctccccaat

SEQ ID NO: 389 - DNA, 73 bp, synthetic construct
  source 1..73: mol_type = other DNA
tttctcaaacagaggtaatgaaacgtttggttaaattaggtcgcatctctgaggagtgtggttttgataccgt

SEQ ID NO: 390 - DNA, 81 bp, synthetic construct
  source 1..81: mol_type = other DNA
atggttactggagcatcatttcacggagtttggtttgcttggtaacccttatgtcgctgctgcatatttacttggcgcgac

SEQ ID NO: 391 - DNA, 80 bp, synthetic construct
  source 1..80: mol_type = other DNA
taaaaaattgaatgtaggaactgccgctattgttcttcccacagcccatccagtacgccaacttgaagatgtgaatttat

SEQ ID NO: 392 - DNA, 78 bp, synthetic construct
  source 1..78: mol_type = other DNA
tggatcaaatgtcaaaaggacgatttcggtttggtatttgccgagggctttacaacaaggactttcgcgtattcggca

SEQ ID NO: 393 - DNA, 79 bp, synthetic construct
  source 1..79: mol_type = other DNA
cagatatgaataacagtcgcgccttagcggaatgctggtacgggctgataaagaatggcatgacagagggatatatgga

SEQ ID NO: 394 - DNA, 83 bp, synthetic construct
  source 1..83: mol_type = other DNA
agctgataatgaacatatcaagttccataaggtaaaagtaaaccccgcggcgtatagcagaggtggcgcaccggtttatgtgg

SEQ ID NO: 395 - DNA, 72 bp, synthetic construct
  source 1..72: mol_type = other DNA
tggctgaatcagcttcgacgactgagtgggctgctcaatttggcctaccgatgatattaagttggattataa

SEQ ID NO: 396 - DNA, 82 bp, synthetic construct
  source 1..82: mol_type = other DNA
atactaacgaaaagaaagcacaacttgagctttataatgaagtggctcaagaatatgggcacgatattcataatatcgacca

SEQ ID NO: 397 - DNA, 80 bp, synthetic construct
  source 1..80: mol_type = other DNA
ttgcttatcatatataacatctgtagatcatgactcaattaaagcgaaagagatttgccggaaatttctggggcattggt

SEQ ID NO: 398 - DNA, 80 bp, synthetic construct
  source 1..80: mol_type = other DNA
atgattcttatgtgaatgctacgactatttttgatgattcagaccaaacaagaggttatgatttcaataaagggcagtgg

SEQ ID NO: 399 - DNA, 83 bp, synthetic construct
  source 1..83: mol_type = other DNA
cgtgactttgtattaaaaggacataaagatactaatcgccgtattgattacagttacgaaatcaatcccgtgggaacgccgca

SEQ ID NO: 400 - DNA, 76 bp, synthetic construct
  source 1..76: mol_type = other DNA
ggaatgtattgacataattcaaaaagacattgatgctacaggaatatcaaatatttgttgtggatttgaagctaat

SEQ ID NO: 401 - DNA, 77 bp, synthetic construct
  source 1..77: mol_type = other DNA
ggaacagtagacgaaattattgcttccatgaagctcttccagtctgatgtcatgccatttcttaaagaaaaacaacg

SEQ ID NO: 402 - DNA, 79 bp, synthetic construct
  source 1..79: mol_type = other DNA
ttcgctattatattaataatactagagagagaaagacaggacccactatgaaatttggattgttcttccttaacttcat

SEQ ID NO: 403 - DNA, 83 bp, synthetic construct
  source 1..83: mol_type = other DNA
caattcaacaactgttcaagaacaaagtatagttcgcatgcaggaaataacggagtatgttgataagttgaattttgaacaga

SEQ ID NO: 404 - DNA, 81 bp, synthetic construct
  source 1..81: mol_type = other DNA
ttttagtgtatgaaaatcatttttcagataatggtgttgtcggcgctcctctgactgtttctggttttctgctcggtttaa

SEQ ID NO: 405 - DNA, 83 bp, synthetic construct
  source 1..83: mol_type = other DNA
cagagaaaattaaaattggttcattaaatcacatcattacaactcatcatcctgtccgcatagcggaggaagcttgcttattg

SEQ ID NO: 406 - DNA, 71 bp, synthetic construct
  source 1..71: mol_type = other DNA
gatcagttaagtgaagggagatttattttagggtttagtgattgcgaaaaaaaagatgaaatgcatttttt

SEQ ID NO: 407 - DNA, 92 bp, synthetic construct; identical to SEQ ID NO: 387
  source 1..92: mol_type = other DNA

SEQ ID NO: 408 - DNA, 92 bp, synthetic construct
  source 1..92: mol_type = other DNA
acaattgaatctattataattgttatccgctcacaaagcaaataaattttttatgatttcctctagaagcggccgcgaattcaggcatggat

SEQ ID NO: 409 - DNA, 104 bp, synthetic construct
  source 1..104: mol_type = other DNA
attataatagattcaattgtgagcggataacaattactagaggagtcacacaggaaagtacatgaaatttggaaactttttgcttacataccaacctccccaat

SEQ ID NO: 410 - DNA, 104 bp, synthetic construct
  source 1..104: mol_type = other DNA
attggggaggttggtatgtaagcaaaaagtttccaaatttcatgtactttcctgtgtgactcctctagtaattgttatccgctcacaattgaatctattataat

SEQ ID NO: 411 - DNA, 93 bp, synthetic construct
  source 1..93: mol_type = other DNA
tacataccaacctccccaattttctcaaacagaggtaatgaaacgtttggttaaattaggtcgcatctctgaggagtgtggttttgataccgt

SEQ ID NO: 412 - DNA, 93 bp, synthetic construct
  source 1..93: mol_type = other DNA
acggtatcaaaaccacactcctcagagatgcgacctaatttaaccaaacgtttcattacctctgtttgagaaaattggggaggttggtatgta

SEQ ID NO: 413 - DNA, 101 bp, synthetic construct
  source 1..101: mol_type = other DNA
gagtgtggttttgataccgtatggttactggagcatcatttcacggagtttggtttgcttggtaacccttatgtcgctgctgcatatttacttggcgcgac

SEQ ID NO: 414 - DNA, 101 bp, synthetic construct
  source 1..101: mol_type = other DNA
gtcgcgccaagtaaatatgcagcagcgacataagggttaccaagcaaaccaaactccgtgaaatgatgctccagtaaccatacggtatcaaaaccacactc

SEQ ID NO: 415 - DNA, 100 bp, synthetic construct
  source 1..100: mol_type = other DNA
gcatatttacttggcgcgactaaaaaattgaatgtaggaactgccgctattgttcttcccacagcccatccagtacgccaacttgaagatgtgaatttat

SEQ ID NO: 416 - DNA, 100 bp, synthetic construct
  source 1..100: mol_type = other DNA
ataaattcacatcttcaagttggcgtactggatgggctgtgggaagaacaatagcggcagttcctacattcaattttttagtcgcgccaagtaaatatgc

SEQ ID NO: 417 - DNA, 98 bp, synthetic construct
  source 1..98: mol_type = other DNA
acttgaagatgtgaatttattggatcaaatgtcaaaaggacgatttcggtttggtatttgccgagggctttacaacaaggactttcgcgtattcggca

SEQ ID NO: 418 - DNA, 98 bp, synthetic construct
  source 1..98: mol_type = other DNA
tgccgaatacgcgaaagtccttgttgtaaagccctcggcaaataccaaaccgaaatcgtccttttgacatttgatccaataaattcacatcttcaagt

SEQ ID NO: 419 - DNA, 99 bp, synthetic construct
  source 1..99: mol_type = other DNA
ggactttcgcgtattcggcacagatatgaataacagtcgcgccttagcggaatgctggtacgggctgataaagaatggcatgacagagggatatatgga

SEQ ID NO: 420 - DNA, 99 bp, synthetic construct
  source 1..99: mol_type = other DNA
tccatatatccctctgtcatgccattctttatcagcccgtaccagcattccgctaaggcgcgactgttattcatatctgtgccgaatacgcgaaagtcc

SEQ ID NO: 421 - DNA, 103 bp, synthetic construct
  source 1..103: mol_type = other DNA
atgacagagggatatatggaagctgataatgaacatatcaagttccataaggtaaaagtaaaccccgcggcgtatagcagaggtggcgcaccggtttatgtgg

SEQ ID NO: 422 - DNA, 103 bp, synthetic construct
  source 1..103: mol_type = other DNA
ccacataaaccggtgcgccacctctgctatacgccgcggggtttacttttaccttatggaacttgatatgttcattatcagcttccatatatccctctgtcat

SEQ ID NO: 423 - DNA, 92 bp, synthetic construct
  source 1..92: mol_type = other DNA
tggcgcaccggtttatgtggtggctgaatcagcttcgacgactgagtgggctgctcaatttggcctaccgatgatattaagttggattataa

SEQ ID NO: 424 - DNA, 92 bp, synthetic construct
  source 1..92: mol_type = other DNA
ttataatccaacttaatatcatcggtaggccaaattgagcagcccactcagtcgtcgaagctgattcagccaccacataaaccggtgcgcca

SEQ ID NO: 425 - DNA, 102 bp, synthetic construct
  source 1..102: mol_type = other DNA
gatattaagttggattataaatactaacgaaaagaaagcacaacttgagctttataatgaagtggctcaagaatatgggcacgatattcataatatcgacca

SEQ ID NO: 426 - DNA, 102 bp, synthetic construct
  source 1..102: mol_type = other DNA
tggtcgatattatgaatatcgtgcccatattcttgagccacttcattataaagctcaagttgtgctttcttttcgttagtatttataatccaacttaatatc

SEQ ID NO: 427 - DNA, 100 bp, synthetic construct
  source 1..100: mol_type = other DNA
gatattcataatatcgaccattgcttatcatatataacatctgtagatcatgactcaattaaagcgaaagagatttgccggaaatttctggggcattggt

SEQ ID NO: 428 - DNA, 100 bp, synthetic construct
  source 1..100: mol_type = other DNA
accaatgccccagaaatttccggcaaatctctttcgctttaattgagtcatgatctacagatgttatatatgataagcaatggtcgatattatgaatatc

SEQ ID NO: 429 - DNA, 100 bp, synthetic construct
  source 1..100: mol_type = other DNA
gaaatttctggggcattggtatgattcttatgtgaatgctacgactatttttgatgattcagaccaaacaagaggttatgatttcaataaagggcagtgg

SEQ ID NO: 430 - DNA, 100 bp, synthetic construct
  source 1..100: mol_type = other DNA
ccactgccctttattgaaatcataacctcttgtttggtctgaatcatcaaaaatagtcgtagcattcacataagaatcataccaatgccccagaaatttc

SEQ ID NO: 431 - DNA, 103 bp, synthetic construct
  source 1..103: mol_type = other DNA
atttcaataaagggcagtggcgtgactttgtattaaaaggacataaagatactaatcgccgtattgattacagttacgaaatcaatcccgtgggaacgccgca

SEQ ID NO: 432 - DNA, 103 bp, synthetic construct
  source 1..103: mol_type = other DNA
tgcggcgttcccacgggattgatttcgtaactgtaatcaatacggcgattagtatctttatgtccttttaatacaaagtcacgccactgccctttattgaaat

SEQ ID NO: 433 - DNA, 96 bp, synthetic construct
  source 1..96: mol_type = other DNA
aatcccgtgggaacgccgcaggaatgtattgacataattcaaaaagacattgatgctacaggaatatcaaatatttgttgtggatttgaagctaat

SEQ ID NO: 434 - DNA, 96 bp, synthetic construct
  source 1..96: mol_type = other DNA
attagcttcaaatccacaacaaatatttgatattcctgtagcatcaatgtctttttgaattatgtcaatacattcctgcggcgttcccacgggatt

SEQ ID NO: 435 - DNA, 97 bp, synthetic construct
  source 1..97: mol_type = other DNA
gttgtggatttgaagctaatggaacagtagacgaaattattgcttccatgaagctcttccagtctgatgtcatgccatttcttaaagaaaaacaacg

SEQ ID NO: 436 - DNA, 97 bp, synthetic construct
  source 1..97: mol_type = other DNA
cgttgtttttctttaagaaatggcatgacatcagactggaagagcttcatggaagcaataatttcgtctactgttccattagcttcaaatccacaac

SEQ ID NO: 437 - DNA, 99 bp, synthetic construct
  source 1..99: mol_type = other DNA
tttcttaaagaaaaacaacgttcgctattatattaataatactagagagagaaagacaggacccactatgaaatttggattgttcttccttaacttcat

SEQ ID NO: 438 - DNA, 99 bp, synthetic construct
  source 1..99: mol_type = other DNA
atgaagttaaggaagaacaatccaaatttcatagtgggtcctgtctttctctctctagtattattaatataatagcgaacgttgtttttctttaagaaa

SEQ ID NO: 439 - DNA, 103 bp, synthetic construct
  source 1..103: mol_type = other DNA
ttgttcttccttaacttcatcaattcaacaactgttcaagaacaaagtatagttcgcatgcaggaaataacggagtatgttgataagttgaattttgaacaga

SEQ ID NO: 440 - DNA, 103 bp, synthetic construct
  source 1..103: mol_type = other DNA
tctgttcaaaattcaacttatcaacatactccgttatttcctgcatgcgaactatactttgttcttgaacagttgttgaattgatgaagttaaggaagaacaa

SEQ ID NO: 441 - DNA, 101 bp, synthetic construct
  source 1..101: mol_type = other DNA
taagttgaattttgaacagattttagtgtatgaaaatcatttttcagataatggtgttgtcggcgctcctctgactgtttctggttttctgctcggtttaa

SEQ ID NO: 442 - DNA, 101 bp, synthetic construct
  source 1..101: mol_type = other DNA
ttaaaccgagcagaaaaccagaaacagtcagaggagcgccgacaacaccattatctgaaaaatgattttcatacactaaaatctgttcaaaattcaactta

SEQ ID NO: 443 - DNA, 103 bp, synthetic construct
  source 1..103: mol_type = other DNA
tggttttctgctcggtttaacagagaaaattaaaattggttcattaaatcacatcattacaactcatcatcctgtccgcatagcggaggaagcttgcttattg

SEQ ID NO: 444 - DNA, 103 bp, synthetic construct
  source 1..103: mol_type = other DNA
caataagcaagcttcctccgctatgcggacaggatgatgagttgtaatgatgtgatttaatgaaccaattttaattttctctgttaaaccgagcagaaaacca

SEQ ID NO: 445 - DNA, 91 bp, synthetic construct
  source 1..91: mol_type = other DNA
cggaggaagcttgcttattggatcagttaagtgaagggagatttattttagggtttagtgattgcgaaaaaaaagatgaaatgcatttttt

SEQ ID NO: 446 - DNA, 91 bp, synthetic construct
  source 1..91: mol_type = other DNA
aaaaaatgcatttcatcttttttttcgcaatcactaaaccctaaaataaatctcccttcacttaactgatccaataagcaagcttcctccg

SEQ ID NO: 447 - DNA, 92 bp, synthetic construct; identical to SEQ ID NO: 209
  source 1..92: mol_type = other DNA

SEQ ID NO: 448 - DNA, 83 bp, synthetic construct; identical to SEQ ID NO: 211
  source 1..83: mol_type = other DNA

SEQ ID NO: 449 - DNA, 80 bp, synthetic construct; identical to SEQ ID NO: 213
  source 1..80: mol_type = other DNA

SEQ ID NO: 450 - DNA, 79 bp, synthetic construct; identical to SEQ ID NO: 215
  source 1..79: mol_type = other DNA

SEQ ID NO: 451 - DNA, 82 bp, synthetic construct; identical to SEQ ID NO: 217
  source 1..82: mol_type = other DNA

SEQ ID NO: 452 - DNA, 83 bp, synthetic construct; identical to SEQ ID NO: 219
  source 1..83: mol_type = other DNA

SEQ ID NO: 453 - DNA, 77 bp, synthetic construct; identical to SEQ ID NO: 221
  source 1..77: mol_type = other DNA

SEQ ID NO: 454 - DNA, 77 bp, synthetic construct; identical to SEQ ID NO: 223
  source 1..77: mol_type = other DNA

SEQ ID NO: 455 - DNA, 76 bp, synthetic construct; identical to SEQ ID NO: 225
  source 1..76: mol_type = other DNA

SEQ ID NO: 456 - DNA, 78 bp, synthetic construct; identical to SEQ ID NO: 227
  source 1..78: mol_type = other DNA

SEQ ID NO: 457 - DNA, 83 bp, synthetic construct; identical to SEQ ID NO: 229
  source 1..83: mol_type = other DNA

SEQ ID NO: 458 - DNA, 105 bp, synthetic construct; identical to SEQ ID NO: 231
  source 1..105: mol_type = other DNA

SEQ ID NO: 459 - DNA, 96 bp, synthetic construct
  source 1..96: mol_type = other DNA
aggtcctcaatcgcactggatgaaggttctgtgggctgcgttgctggtcacattcctggcaggatgccaggccaaggtggagcaagcggtggagac

SEQ ID NO: 460 - DNA, 83 bp, synthetic construct
  source 1..83: mol_type = other DNA
agagccggagcccgagctgcgccagcagaccgagtggcagagcggccagcgctgggaactggcactgggtcgcttttgggatt

SEQ ID NO: 461 - DNA, 80 bp, synthetic construct
  source 1..80: mol_type = other DNA
acctgcgctgggtgcagacactgtctgagcaggtgcaggaggagctgctcagctcccaggtcacccaggaactgagggcg

SEQ ID NO: 462 - DNA, 79 bp, synthetic construct
  source 1..79: mol_type = other DNA
ctgatggacgagaccatgaaggagttgaaggcctacaaatcggaactggaggaacaactgaccccggtggcggaggaga

SEQ ID NO: 463 - DNA, 82 bp, synthetic construct
  source 1..82: mol_type = other DNA
cgcgggcacggctgtccaaggagctgcaggcggcgcaggcccggctgggcgcggacatggaggacgtgtgcggccgcctggt

SEQ ID NO: 464 - DNA, 83 bp, synthetic construct
  source 1..83: mol_type = other DNA
gcagtaccgcggcgaggtgcaggccatgctcggccagagcaccgaggagctgcgggtgcgcctcgcctcccacctgcgcaagc

SEQ ID NO: 465 - DNA, 77 bp, synthetic construct
  source 1..77: mol_type = other DNA
tgcgtaagcggctcctccgcgatgccgatgacctgcagaagcgcctggcagtgtaccaggccggggcccgcgagggc

SEQ ID NO: 466 - DNA, 77 bp, synthetic construct
  source 1..77: mol_type = other DNA
gccgagcgcggcctcagcgccatccgcgagcgcctggggcccctggtggaacagggccgcgtgcgggccgccactgt

SEQ ID NO: 467 - DNA, 76 bp, synthetic construct
  source 1..76: mol_type = other DNA
gggctccctggccggccagccgctacaggagcgggcccaggcctggggcgagcggctgcgcgcgcggatggaggag

SEQ ID NO: 468 - DNA, 78 bp, synthetic construct
  source 1..78: mol_type = other DNA
atgggcagccggacccgcgaccgcctggacgaggtgaaggagcaggtggcggaggtgcgcgccaagctggaggagcag

SEQ ID NO: 469 - DNA, 83 bp, synthetic construct
  source 1..83: mol_type = other DNA
gcccagcagatacgcctgcaggccgaggccttccaggcccgcctcaagagctggttcgagcccctggtggaagacatgcagcg

SEQ ID NO: 470 - DNA, 101 bp, synthetic construct
  source 1..101: mol_type = other DNA
ccagtgggccgggctggtggagaaggtgcaggctgccgtgggcaccagcgccgcccctgtgcccagcgacaatcactgaggaaatgtcagagccagcgtct

SEQ ID NO: 471 - DNA, 102 bp, synthetic construct
  source 1..102: mol_type = other DNA
aggtcctcaatcgcactggatgaaggttctgtgggctgcgttgctggtcacattcctggcaggatgccaggccaaggtggagcaagcggtggagacagagcc

SEQ ID NO: 472 - DNA, 83 bp, synthetic construct
  source 1..83: mol_type = other DNA
ggagcccgagctgcgccagcagaccgagtggcagagcggccagcgctgggaactggcactgggtcgcttttgggattacctgc

SEQ ID NO: 473 - DNA, 80 bp, synthetic construct
  source 1..80: mol_type = other DNA
gctgggtgcagacactgtctgagcaggtgcaggaggagctgctcagctcccaggtcacccaggaactgagggcgctgatg

SEQ ID NO: 474 - DNA, 79 bp, synthetic construct
  source 1..79: mol_type = other DNA
gacgagaccatgaaggagttgaaggcctacaaatcggaactggaggaacaactgaccccggtggcggaggagacgcggg

SEQ ID NO: 475 - DNA, 82 bp, synthetic construct
  source 1..82: mol_type = other DNA
cacggctgtccaaggagctgcaggcggcgcaggcccggctgggcgcggacatggaggacgtgtgcggccgcctggtgcagta

SEQ ID NO: 476 - DNA, 83 bp, synthetic construct
  source 1..83: mol_type = other DNA
ccgcggcgaggtgcaggccatgctcggccagagcaccgaggagctgcgggtgcgcctcgcctcccacctgcgcaagctgcgta

SEQ ID NO: 477 - DNA, 77 bp, synthetic construct
  source 1..77: mol_type = other DNA
agcggctcctccgcgatgccgatgacctgcagaagcgcctggcagtgtaccaggccggggcccgcgagggcgccgag

SEQ ID NO: 478 - DNA, 77 bp, synthetic construct
  source 1..77: mol_type = other DNA
cgcggcctcagcgccatccgcgagcgcctggggcccctggtggaacagggccgcgtgcgggccgccactgtgggctc

SEQ ID NO: 479 - DNA, 76 bp, synthetic construct
  source 1..76: mol_type = other DNA
cctggccggccagccgctacaggagcgggcccaggcctggggcgagcggctgcgcgcgcggatggaggagatgggc

SEQ ID NO: 480 - DNA, 78 bp, synthetic construct
  source 1..78: mol_type = other DNA
agccggacccgcgaccgcctggacgaggtgaaggagcaggtggcggaggtgcgcgccaagctggaggagcaggcccag

SEQ ID NO: 481 - DNA, 83 bp, synthetic construct
  source 1..83: mol_type = other DNA
cagatacgcctgcaggccgaggccttccaggcccgcctcaagagctggttcgagcccctggtggaagacatgcagcgccagtg

SEQ ID NO: 482 - DNA, 95 bp, synthetic construct
  source 1..95: mol_type = other DNA
ggccgggctggtggagaaggtgcaggctgccgtgggcaccagcgccgcccctgtgcccagcgacaatcactgaggaaatgtcagagccagcgtct

SEQ ID NO: 483 - DNA, 102 bp, synthetic construct; identical to SEQ ID NO: 471
  source 1..102: mol_type = other DNA

SEQ ID NO: 484 - DNA, 103 bp, synthetic construct
  source 1..103: mol_type = other DNA
gcaggtaatcccaaaagcgacccagtgccagttcccagcgctggccgctctgccactcggtctgctggcgcagctcgggctccggctctgtctccaccgcttg

SEQ ID NO: 485 - DNA, 100 bp, synthetic construct
  source 1..100: mol_type = other DNA
tcgcttttgggattacctgcgctgggtgcagacactgtctgagcaggtgcaggaggagctgctcagctcccaggtcacccaggaactgagggcgctgatg

SEQ ID NO: 486 - DNA, 99 bp, synthetic construct
  source 1..99: mol_type = other DNA
cccgcgtctcctccgccaccggggtcagttgttcctccagttccgatttgtaggccttcaactccttcatggtctcgtccatcagcgccctcagttcct

SEQ ID NO: 487 - DNA, 102 bp, synthetic construct
  source 1..102: mol_type = other DNA
ggtggcggaggagacgcgggcacggctgtccaaggagctgcaggcggcgcaggcccggctgggcgcggacatggaggacgtgtgcggccgcctggtgcagta

SEQ ID NO: 488 - DNA, 103 bp, synthetic construct
  source 1..103: mol_type = other DNA
tacgcagcttgcgcaggtgggaggcgaggcgcacccgcagctcctcggtgctctggccgagcatggcctgcacctcgccgcggtactgcaccaggcggccgca

SEQ ID NO: 489 - DNA, 97 bp, synthetic construct
  source 1..97: mol_type = other DNA
ccacctgcgcaagctgcgtaagcggctcctccgcgatgccgatgacctgcagaagcgcctggcagtgtaccaggccggggcccgcgagggcgccgag

SEQ ID NO: 490 - DNA, 97 bp, synthetic construct
  source 1..97: mol_type = other DNA
gagcccacagtggcggcccgcacgcggccctgttccaccaggggccccaggcgctcgcggatggcgctgaggccgcgctcggcgccctcgcgggccc

SEQ ID NO: 491 - DNA, 96 bp, synthetic construct
  source 1..96: mol_type = other DNA
cgggccgccactgtgggctccctggccggccagccgctacaggagcgggcccaggcctggggcgagcggctgcgcgcgcggatggaggagatgggc

SEQ ID NO: 492 - DNA, 98 bp, synthetic construct
  source 1..98: mol_type = other DNA
ctgggcctgctcctccagcttggcgcgcacctccgccacctgctccttcacctcgtccaggcggtcgcgggtccggctgcccatctcctccatccgcg

SEQ ID NO: 493 - DNA, 103 bp, synthetic construct
  source 1..103: mol_type = other DNA
agctggaggagcaggcccagcagatacgcctgcaggccgaggccttccaggcccgcctcaagagctggttcgagcccctggtggaagacatgcagcgccagtg

SEQ ID NO: 494 - DNA, 115 bp, synthetic construct
  source 1..115: mol_type = other DNA
agacgctggctctgacatttcctcagtgattgtcgctgggcacaggggcggcgctggtgcccacggcagcctgcaccttctccaccagcccggcccactggcgctgcatgtcttc

SEQ ID NO: 495 - DNA, 102 bp, synthetic construct; identical to SEQ ID NO: 471
  source 1..102: mol_type = other DNA

SEQ ID NO: 496 - DNA, 102 bp, synthetic construct
  source 1..102: mol_type = other DNA
ggctctgtctccaccgcttgctccaccttggcctggcatcctgccaggaatgtgaccagcaacgcagcccacagaaccttcatccagtgcgattgaggacct

SEQ ID NO: 497 - DNA, 103 bp, synthetic construct
  source 1..103: mol_type = other DNA
caagcggtggagacagagccggagcccgagctgcgccagcagaccgagtggcagagcggccagcgctgggaactggcactgggtcgcttttgggattacctgc

SEQ ID NO: 498 - DNA, 103 bp, synthetic construct; identical to SEQ ID NO: 484
  source 1..103: mol_type = other DNA

SEQ ID NO: 499 - DNA, 100 bp, synthetic construct; identical to SEQ ID NO: 485
  source 1..100: mol_type = other DNA

SEQ ID NO: 500 - DNA, 100 bp, synthetic construct
  source 1..100: mol_type = other DNA
catcagcgccctcagttcctgggtgacctgggagctgagcagctcctcctgcacctgctcagacagtgtctgcacccagcgcaggtaatcccaaaagcga

SEQ ID NO: 501 - DNA, 99 bp, synthetic construct
  source 1..99: mol_type = other DNA
aggaactgagggcgctgatggacgagaccatgaaggagttgaaggcctacaaatcggaactggaggaacaactgaccccggtggcggaggagacgcggg

SEQ ID NO: 502 - DNA, 99 bp, synthetic construct; identical to SEQ ID NO: 486
  source 1..99: mol_type = other DNA

SEQ ID NO: 503 - DNA, 102 bp, synthetic construct; identical to SEQ ID NO: 487
  source 1..102: mol_type = other DNA

SEQ ID NO: 504 - DNA, 102 bp, synthetic construct
  source 1..102: mol_type = other DNA
tactgcaccaggcggccgcacacgtcctccatgtccgcgcccagccgggcctgcgccgcctgcagctccttggacagccgtgcccgcgtctcctccgccacc

SEQ ID NO: 505 - DNA, 103 bp, synthetic construct
  source 1..103: mol_type = other DNA
tgcggccgcctggtgcagtaccgcggcgaggtgcaggccatgctcggccagagcaccgaggagctgcgggtgcgcctcgcctcccacctgcgcaagctgcgta

SEQ ID NO: 506 - DNA, 103 bp, synthetic construct; identical to SEQ ID NO: 488
  source 1..103: mol_type = other DNA

SEQ ID NO: 507 - DNA, 97 bp, synthetic construct; identical to SEQ ID NO: 489
  source 1..97: mol_type = other DNA

SEQ ID NO: 508 - DNA, 97 bp, synthetic construct
  source 1..97: mol_type = other DNA
ctcggcgccctcgcgggccccggcctggtacactgccaggcgcttctgcaggtcatcggcatcgcggaggagccgcttacgcagcttgcgcaggtgg

SEQ ID NO: 509 - DNA, 97 bp, synthetic construct
  source 1..97: mol_type = other DNA
gggcccgcgagggcgccgagcgcggcctcagcgccatccgcgagcgcctggggcccctggtggaacagggccgcgtgcgggccgccactgtgggctc

SEQ ID NO: 510 - DNA, 97 bp, synthetic construct; identical to SEQ ID NO: 490
  source 1..97: mol_type = other DNA

SEQ ID NO: 511 - DNA, 96 bp, synthetic construct; identical to SEQ ID NO: 491
  source 1..96: mol_type = other DNA

SEQ ID NO: 512 - DNA, 96 bp, synthetic construct
  source 1..96: mol_type = other DNA
gcccatctcctccatccgcgcgcgcagccgctcgccccaggcctgggcccgctcctgtagcggctggccggccagggagcccacagtggcggcccg

SEQ ID NO: 513 - DNA, 98 bp, synthetic construct
  source 1..98: mol_type = other DNA
cgcggatggaggagatgggcagccggacccgcgaccgcctggacgaggtgaaggagcaggtggcggaggtgcgcgccaagctggaggagcaggcccag

SEQ ID NO: 514 - DNA, 98 bp, synthetic construct; identical to SEQ ID NO: 492
  source 1..98: mol_type = other DNA

SEQ ID NO: 515 - DNA, 103 bp, synthetic construct; identical to SEQ ID NO: 493
  source 1..103: mol_type = other DNA

SEQ ID NO: 516 - DNA, 103 bp, synthetic construct
  source 1..103: mol_type = other DNA
cactggcgctgcatgtcttccaccaggggctcgaaccagctcttgaggcgggcctggaaggcctcggcctgcaggcgtatctgctgggcctgctcctccagct

SEQ ID NO: 517 - DNA, 115 bp, synthetic construct
  source 1..115: mol_type = other DNA
gaagacatgcagcgccagtgggccgggctggtggagaaggtgcaggctgccgtgggcaccagcgccgcccctgtgcccagcgacaatcactgaggaaatgtcagagccagcgtct

SEQ ID NO: 518 - DNA, 115 bp, synthetic construct; identical to SEQ ID NO: 494
  source 1..115: mol_type = other DNA

SEQ ID NO: 519 - DNA, 102 bp, synthetic construct; identical to SEQ ID NO: 233
  source 1..102: mol_type = other DNA

SEQ ID NO: 520 - DNA, 72 bp, synthetic construct; identical to SEQ ID NO: 235
  source 1..72: mol_type = other DNA

SEQ ID NO: 521 - DNA, 75 bp, synthetic construct; identical to SEQ ID NO: 237
  source 1..75: mol_type = other DNA

SEQ ID NO: 522 - DNA, 129 bp, synthetic construct; identical to SEQ ID NO: 239
  source 1..129: mol_type = other DNA

SEQ ID NO: 523 - DNA, 92 bp, synthetic construct; identical to SEQ ID NO: 241
  source 1..92: mol_type = other DNA

SEQ ID NO: 524 - DNA, 98 bp, synthetic construct
  source 1..98: mol_type = other DNA
gcactgaaggtcctcaatcgcactggaaacatcaaggtcggtctacaccaagatctctcgatcatcatcagtctccgttgaaggaggacgacgacgag

SEQ ID NO: 525 - DNA, 78 bp, synthetic construct
  source 1..78: mol_type = other DNA
gacgaggaccatggggaggacgaggaattggaggactcagtggctcaggagatctcgacgtacttggcggtcgacgag

SEQ ID NO: 526 - DNA, 75 bp, synthetic construct
  source 1..75: mol_type = other DNA
gaccatggggtcgaggttatggaccttctggctctgtatcagtctcagtctctgtcgaacgaggaatccgacgag

SEQ ID NO: 527 - DNA, 129 bp, synthetic construct
  source 1..129: mol_type = other DNA
gaccatggggacgacgaggaaaggtttcaatcagccgatccgtttccattgaacgaattgtcaccccaggcgtctacaccaagatctctcgatcatcatcagtctccgttgaaggaggacgacgacgag

SEQ ID NO: 528 - DNA, 96 bp, synthetic construct
  source 1..96: mol_type = other DNA
gaccatggggaggacgaggaattggaggactcagtggctcaggagatctcgacgtacttggcggtcgcttggattctgcgtttgtttccgtctacg

SEQ ID NO: 529 - DNA, 92 bp, synthetic construct
  source 1..92: mol_type = other DNA
gcactgaaggtcctcaatcgcactggaaacatcaaggtcggtctacaccaagatctctcgatcatcatcagtctccgttgaaggaggacgac

SEQ ID NO: 530 - DNA, 72 bp, synthetic construct
  source 1..72: mol_type = other DNA
gacgaggaccatggggaggacgaggaattggaggactcagtggctcaggagatctcgacgtacttggcggtc

SEQ ID NO: 531 - DNA, 75 bp, synthetic construct
  source 1..75: mol_type = other DNA
gacgaggaccatggggtcgaggttatggaccttctggctctgtatcagtctcagtctctgtcgaacgaggaatcc

SEQ ID NO: 532 - DNA, 129 bp, synthetic construct
  source 1..129: mol_type = other DNA
gacgaggaccatggggacgacgaggaaaggtttcaatcagccgatccgtttccattgaacgaattgtcaccccaggcgtctacaccaagatctctcgatcatcatcagtctccgttgaaggaggacgac

SEQ ID NO: 533 - DNA, 102 bp, synthetic construct
  source 1..102: mol_type = other DNA
gacgaggaccatggggaggacgaggaattggaggactcagtggctcaggagatctcgacgtacttggcggtcgcttggattctgcgtttgtttccgtctacg

SEQ ID NO: 534 - DNA, 102 bp, synthetic construct
  source 1..102: mol_type = other DNA
gcactgaaggtcctcaatcgcactggaaacatcaaggtcggtctacaccaagatctctcgatcatcatcagtctccgttgaaggaggacgacgacgaggacc

SEQ ID NO: 535 - DNA, 92 bp, synthetic construct
  source 1..92: mol_type = other DNA
ggtcctcgtcgaccgccaagtacgtcgagatctcctgagccactgagtcctccaattcctcgtcctccccatggtcctcgtcgtcgtcctcc

SEQ ID NO: 536 - DNA, 95 bp, synthetic construct
  source 1..95: mol_type = other DNA
cttggcggtcgacgaggaccatggggtcgaggttatggaccttctggctctgtatcagtctcagtctctgtcgaacgaggaatccgacgaggacc

SEQ ID NO: 537 - DNA, 149 bp, synthetic construct
  source 1..149: mol_type = other DNA
ggtcctcgtcgtcgtcctccttcaacggagactgatgatgatcgagagatcttggtgtagacgcctggggtgacaattcgttcaatggaaacggatcggctgattgaaacctttcctcgtcgtccccatggtcctcgtcggattcctcg

SEQ ID NO: 538 - DNA, 112 bp, synthetic construct
  source 1..112: mol_type = other DNA
ggaggacgacgacgaggaccatggggaggacgaggaattggaggactcagtggctcaggagatctcgacgtacttggcggtcgcttggattctgcgtttgtttccgtctacg

SEQ ID NO: 539 - DNA, 112 bp, synthetic construct
  source 1..112: mol_type = other DNA
gcactgaaggtcctcaatcgcactggaaacatcaaggtcggtctacaccaagatctctcgatcatcatcagtctccgttgaaggaggacgacgacgaggaccatggggagga

SEQ ID NO: 540 - DNA, 112 bp, synthetic construct
  source 1..112: mol_type = other DNA
tcctccccatggtcctcgtcgtcgtcctccttcaacggagactgatgatgatcgagagatcttggtgtagaccgaccttgatgtttccagtgcgattgaggaccttcagtgc

SEQ ID NO: 541 - DNA, 92 bp, synthetic construct
  source 1..92: mol_type = other DNA
gacgaggaccatggggaggacgaggaattggaggactcagtggctcaggagatctcgacgtacttggcggtcgacgaggaccatggggtcga

SEQ ID NO: 542 - DNA, 92 bp, synthetic construct
  source 1..92: mol_type = other DNA
tcgaccccatggtcctcgtcgaccgccaagtacgtcgagatctcctgagccactgagtcctccaattcctcgtcctccccatggtcctcgtc

SEQ ID NO: 543 - DNA, 95 bp, synthetic construct
  source 1..95: mol_type = other DNA
gacgaggaccatggggtcgaggttatggaccttctggctctgtatcagtctcagtctctgtcgaacgaggaatccgacgaggaccatggggacga

SEQ ID NO: 544 - DNA, 95 bp, synthetic construct
  source 1..95: mol_type = other DNA
tcgtccccatggtcctcgtcggattcctcgttcgacagagactgagactgatacagagccagaaggtccataacctcgaccccatggtcctcgtc

SEQ ID NO: 545 - DNA, 149 bp, synthetic construct
  source 1..149: mol_type = other DNA
gacgaggaccatggggacgacgaggaaaggtttcaatcagccgatccgtttccattgaacgaattgtcaccccaggcgtctacaccaagatctctcgatcatcatcagtctccgttgaaggaggacgacgacgaggaccatggggagga

SEQ ID NO: 546 - DNA, 149 bp, synthetic construct
  source 1..149: mol_type = other DNA
tcctccccatggtcctcgtcgtcgtcctccttcaacggagactgatgatgatcgagagatcttggtgtagacgcctggggtgacaattcgttcaatggaaacggatcggctgattgaaacctttcctcgtcgtccccatggtcctcgtc

SEQ ID NO: 547 - DNA, 102 bp, synthetic construct; identical to SEQ ID NO: 533
  source 1..102: mol_type = other DNA

SEQ ID NO: 548 - DNA, 102 bp, synthetic construct
  source 1..102: mol_type = other DNA
cgtagacggaaacaaacgcagaatccaagcgaccgccaagtacgtcgagatctcctgagccactgagtcctccaattcctcgtcctccccatggtcctcgtc

SEQ ID NO: 549 - DNA, 3235 bp, synthetic construct
  source 1..3235: mol_type = other DNA
atccatgcctgaattcgcggccgcttctagaggaaatcataaaaaatttatttgctttgtgagcggataacaattataatagattcaattgtgagcggataacaattactagaggagtcacacaggaaagtacatgaaatttggaaactttttgcttacataccaacctccccaattttctcaaacagaggtaatgaaacgtttggttaaattaggtcgcatctctgaggagtgtggttttgataccgtatggttactggagcatcatttcacggagtttggtttgcttggtaacccttatgtcgctgctgcatatttacttggcgcgactaaaaaattgaatgtaggaactgccgctattgttcttcccacagcccatccagtacgccaacttgaagatgtgaatttattggatcaaatgtcaaaaggacgatttcggtttggtatttgccgagggctttacaacaaggactttcgcgtattcggcacagatatgaataacagtcgcgccttagcggaatgctggtacgggctgataaagaatggcatgacagagggatatatggaagctgataatgaacatatcaagttccataaggtaaaagtaaaccccgcggcgtatagcagaggtggcgcaccggtttatgtggtggctgaatcagcttcgacgactgagtgggctgctcaatttggcctaccgatgatattaagttggattataaatactaacgaaaagaaagcacaacttgagctttataatgaagtggctcaagaatatgggcacgatattcataatatcgaccattgcttatcatatataacatctgtagatcatgactcaattaaagcgaaagagatttgccggaaatttctggggcattggtatgattcttatgtgaatgctacgactatttttgatgattcagaccaaacaagaggttatgatttcaataaagggcagtggcgtgactttgtattaaaaggacataaagatactaatcgccgtattgattacagttacgaaatcaatcccgtgggaacgccgcaggaatgtattgacataattcaaaaagacattgatgctacaggaatatcaaatatttgttgtggatttgaagctaatggaacagtagacgaaattattgcttccatgaagctcttccagtctgatgtcatgccatttcttaaagaaaaacaacgttcgctattatattaataatactagagagagaaagacaggacccactatgaaatttggattgttcttccttaacttcatcaattcaacaactgttcaagaacaaagtatagttcgcatgcaggaaataacggagtatgttgataagttgaattttgaacagattttagtgtatgaaaatcatttttcagataatggtgttgtcggcgctcctctgactgtttctggttttctgctcggtttaacagagaaaattaaaattggttcattaaatcacatcattacaactcatcatcctgtccgcatagcggaggaagcttgcttattggatcagttaagtgaagggagatttattttagggtttagtgattgcgaaaaaaaagatgaaatgcatttttttaatcgcccggttgaatatcaacagcaactatttgaagagtgttatgaaatcattaacgatgctttaacaacaggctattgtaatccagataacgatttttatagcttccctaaaatatctgtaaatccccatgcttatacgccaggcggacctcggaaatatgtaacagcaaccagtcatcatattgttgagtgggcggccaaaaaaggtattcctctcatctttaagtgggatgattctaatgatgttagatatgaatatgctgaaagatataaagccgttgcggataaatatgacgttgacctatcagagatagaccatcagttaatgatattagttaactataacgaagatagtaataaagctaaacaagagacgcgtgcatttattagtgattatgttcttgaaatgcaccctaatgaaaatttcgaaaataaacttgaagaaataattgcagaaaacgctgtcggaaattatacggagtgtataactgcggctaagttggcaattgaaaagtgtggtgcgaaaagtgtattgctgtcctttgaaccaatgaatgatttgatgagccaaaaaaatgtaatcaatattgttgatgataatattaagaagtaccacatggaatatacctaataatactagagaaagaggagaaatactagatgactaaaaaaatttcattcattattaacggccaggttgaaatctttcccgaaagtgatgatttagtgcaatccattaattttggtgataatagtgtttacctgccaatattgaatgactctcatgtaaaaaacattattgattgtaatggaaataacgaattacggttgcataacattgtcaattttctctatacggtagggcaaagatggaaaaatgaagaatactcaagacgcaggacatacattcgtgacttaaaaaaatatatgggatattcagaagaaatggctaagctagaggccaattggatatctatgattttatgttctaaaggcggcctttatgatgttgtagaaaatgaacttggttctcgccatatcatggatgaatggctacctcaggatgaaagttatgttcgggcttttccgaaaggtaaatctgtacatctgttggcaggtaatgttccattatctgggatcatgtctatattacgcgcaattttaactaagaatcagtgtattataaaaacatcgtcaaccgatccttttaccgctaatgcattagcgttaagttttattgatgtagaccctaatcatccgataacgcgctctttatctgttatatattggccccaccaaggtgatacatcactcgcaaaagaaattatgcgacatgcggatgttattgtcgcttggggagggccagatgcgattaattgggcggtagagcatgcgccatcttatgctgatgtgattaaatttggttctaaaaagagtctttgcattatcgataatcctgttgatttgacgtccgcagcgacaggtgcggctcatgatgtttgtttttacgatcagcgagcttgtttttctgcccaaaacatatattacatgggaaatcattatgaggaatttaagttagcgttgatagaaaaacttaatctatatgcgcatatattaccgaatgccaaaaaagat

SEQ ID NO: 550 - DNA, 1773 bp, synthetic construct
  source 1..1773: mol_type = other DNA
gcactgaaggtcctcaatcgcactggaaacatcaaggtcgctgacctcctgccagcaatagtaagacaacacgcaaagtcgttgacaattaatcatcggcatagtatatcggcatagtataatacgacaaggtgaggaactaaaccatggtgtcgaaaggtgaggagcttttcacgggggtggttccaatcttagtcgagcttgatggtgacgtgaatggccataagtttagcgtgcgcggcgagggtgagggcgatgcgacgaacggtaaacttactttaaagtttatttgtactacaggcaaattaccggtcccgtggccgaccctggtcactacgcttgggtatggggtagcatgttttgcccgttaccctgaccacatgaaacagcacgatttctttaaatctgccatgcccgaagggtatgttcaggagcgcactatcagttttaaagacgacggaacgtacaaaactcgtgctgaagtaaagttcgaaggtgatacccttgtgaaccgtatcgtccttaaaggcatcgacttcaaagaggacggtaacattttaggccacaaattagagtacaacttcaattcacataaggtttatatcacagcagataagcaaaaaaatggcatcaaggcaaattttaagacccgccacaacgtagaagatggtggagtccagttggcggaccactaccagcaaaatacaccgattggcgatggtccggttttgcttcccgacaatcactatcttagccaccagagtaaactgagtaaggacccaaatgaaaagcgcgaccacatggtattgaaagagcgcgttacagcagctggaattacacacgatatggacgaactttataagtaaatctatttgacagctagctcagtcctaggtataatgctagcaaagaggagaaatatggtcagtaaaggcgaagcagttatcaaagagttcatgcgcttcaaagttcatatggaagggtcgatgaacgggcacgaatttgaaattgaaggcgaaggcgaaggccgcccatatgaagggacccaaaccgcaaagcttaaggttactaaaggcggtccattacccttttcgtgggacattttaagcccacagtttatgtacgggagtcgcgctttcatcaagcaccctgcggacatcccagattactacaaacagtctttccccgaggggttcaagtgggagcgcgtgatgaacttcgaggatggcggagccgtgacggtcacccaagatacctctttggaggacggtacgttgatctacaaagtgaaattgcgtggcacgaattttccacctgatgggcctgtcatgcagaaaaagacaatgggatgggaagcttccacggagcgcctttacccagaggacggtgttcttaaaggggatatcaaaatggcgctgcgtcttaaagatggaggccgctacctggcggacttcaagactacttacaaggccaaaaaaccagtgcagatgccgggtgcgtacaatgtagatcgtaaattagatattacaagtcacaatgaagattacacggtcgtagagcagtatgagcgcagtgaggggcgtcactctacgggcggtatggacgagttatacaagtaaggtaccctcgagtttgtttatttttctaaatacattcaaatatgtatccgctcatgagacaataaccctgataaatgcttcaataatattgaaaaaggaagagtatgagccatattcaacgggaaacgtcttgctctaggccgcgattaaattc

SEQ ID NO: 551 - DNA, 995 bp, synthetic construct
  source 1..995: mol_type = other DNA
aggtcctcaatcgcactggatgaaggttctgtgggctgcgttgctggtcacattcctggcaggatgccaggccaaggtggagcaagcggtggagacagagccggagcccgagctgcgccagcagaccgagtggcagagcggccagcgctgggaactggcactgggtcgcttttgggattacctgcgctgggtgcagacactgtctgagcaggtgcaggaggagctgctcagctcccaggtcacccaggaactgagggcgctgatggacgagaccatgaaggagttgaaggcctacaaatcggaactggaggaacaactgaccccggtggcggaggagacgcgggcacggctgtccaaggagctgcaggcggcgcaggcccggctgggcgcggacatggaggacgtgtgcggccgcctggtgcagtaccgcggcgaggtgcaggccatgctcggccagagcaccgaggagctgcgggtgcgcctcgcctcccacctgcgcaagctgcgtaagcggctcctccgcgatgccgatgacctgcagaagcgcctggcagtgtaccaggccggggcccgcgagggcgccgagcgcggcctcagcgccatccgcgagcgcctggggcccctggtggaacagggccgcgtgcgggccgccactgtgggctccctggccggccagccgctacaggagcgggcccaggcctggggcgagcggctgcgcgcgcggatggaggagatgggcagccggacccgcgaccgcctggacgaggtgaaggagcaggtggcggaggtgcgcgccaagctggaggagcaggcccagcagatacgcctgcaggccgaggccttccaggcccgcctcaagagctggttcgagcccctggtggaagacatgcagcgccagtgggccgggctggtggagaaggtgcaggctgccgtgggcaccagcgccgcccctgtgcccagcgacaatcactgaggaaatgtcagagccagcgtct

SEQ ID NO: 552 - DNA, 470 bp, synthetic construct
  source 1..470: mol_type = other DNA
gcactgaaggtcctcaatcgcactggaaacatcaaggtcggtctacaccaagatctctcgatcatcatcagtctccgttgaaggaggacgacgacgaggaccatggggaggacgaggaattggaggactcagtggctcaggagatctcgacgtacttggcggtcgacgaggaccatggggtcgaggttatggaccttctggctctgtatcagtctcagtctctgtcgaacgaggaatccgacgaggaccatggggacgacgaggaaaggtttcaatcagccgatccgtttccattgaacgaattgtcaccccaggcgtctacaccaagatctctcgatcatcatcagtctccgttgaaggaggacgacgacgaggaccatggggaggacgaggaattggaggactcagtggctcaggagatctcgacgtacttggcggtcgcttggattctgcgtttgtttccgtctacg

SEQ ID NO: 553 - DNA, 864 bp, synthetic construct
  source 1..864: mol_type = other DNA
cattactcgcatccattctcaggctgtctcgtctcgtctcctcgtctcaaccaaagcaatcaacccatcaaccacctggtctatttgacagctagctcagtcctaggtataatgctagcaaagaggagaaaggatctatggcttcactggttaaaaaagacatgtgcatcaaaatgacgatggaaggaacagtaaacggtcaccatttcaagtgtgtaggagaaggcgaaggcaaaccatttgaagggacccaggtggaaaagatacgcatcactgaaggtgggcccttaccatttgcgtatgatattttggccccttgttgcatgtatggcagtaaaaccttcattaagcatgtgtcgggtattccggattactttaaggagtcttttcctgagggctttacctgggaaagaacacaaatcttcgaggatggcggctatctcaccatacaccaggacacgagccttcagggtaataattttattttcaaagttaatgtcatcggtgccaacttccctgcaaacggtcccgtgatgcagaaaaaaacagctggatgggaaccgtgcgttgagatgctttatccgcgggacggcgtcctgtgtggtcagagcctgatggccctgaaatgcactgatggcaatcatctgacgtcccacctgcgcactacctatcgttctcgcaagccatccaatgcagttaacatgccggaatttcattttggggatcatcgcattgagattttgaaagctgaacaaggtaaattttatgaacaatacgagtcagcggtggcccgttactgtgaggcggcaccgagtaaattagggcatcactaataacgaccttgatgtttccagtgcgattgag

SEQ ID NO: 554 - DNA, 876 bp, synthetic construct
  source 1..876: mol_type = other DNA
cattactcgcatccattctcaggctgtctcgtctcgtctctgacctcctgccagcaatagtaagacaacacgcaaagtcttgacagctagctcagtcctaggtataatgctagcaaagaggagaaaggatctatggtgtcgaaaggtgaggagcttttcacgggggtggttccaatcttagtcgagcttgatggtgacgtgaatggccataagtttagcgtgcgcggcgagggtgagggcgatgcgacgaacggtaaacttactttaaagtttatttgtactacaggcaaattaccggtcccgtggccgaccctggtcactacgcttgggtatggggtagcatgttttgcccgttaccctgaccacatgaaacagcacgatttctttaaatctgccatgcccgaagggtatgttcaggagcgcactatcagttttaaagacgacggaacgtacaaaactcgtgctgaagtaaagttcgaaggtgatacccttgtgaaccgtatcgtccttaaaggcatcgacttcaaagaggacggtaacattttaggccacaaattagagtacaacttcaattcacataaggtttatatcacagcagataagcaaaaaaatggcatcaaggcaaattttaagacccgccacaacgtagaagatggtggagtccagttggcggaccactaccagcaaaatacaccgattggcgatggtccggttttgcttcccgacaatcactatcttagccaccagagtaaactgagtaaggacccaaatgaaaagcgcgaccacatggtattgaaagagcgcgttacagcagctggaattacacacgatatggacgaactttataagtaacgaccttgatgtttccagtgcgat

SEQ ID NO: 555 - DNA, 864 bp, synthetic construct
  source 1..864: mol_type = other DNA
cattactcgcatccattctcaggctgtctcgtctcgtctgctgggagttcgtagacggaaacaaacgcagaatccaagctctatttgacagctagctcagtcctaggtataatgctagcaaagaggagaaaggatctatggtcagtaaaggcgaagcagttatcaaagagttcatgcgcttcaaagttcatatggaagggtcgatgaacgggcacgaatttgaaattgaaggcgaaggcgaaggccgcccatatgaagggacccaaaccgcaaagcttaaggttactaaaggcggtccattacccttttcgtgggacattttaagcccacagtttatgtacgggagtcgcgctttcatcaagcaccctgcggacatcccagattactacaaacagtctttccccgaggggttcaagtgggagcgcgtgatgaacttcgaggatggcggagccgtgacggtcacccaagatacctctttggaggacggtacgttgatctacaaagtgaaattgcgtggcacgaattttccacctgatgggcctgtcatgcagaaaaagacaatgggatgggaagcttccacggagcgcctttacccagaggacggtgttcttaaaggggatatcaaaatggcgctgcgtcttaaagatggaggccgctacctggcggacttcaagactacttacaaggccaaaaaaccagtgcagatgccgggtgcgtacaatgtagatcgtaaattagatattacaagtcacaatgaagattacacggtcgtagagcagtatgagcgcagtgaggggcgtcactctacgggcggtatggacgagttatacaagtaacgaccttgatgtttccagtgcgattgag

SEQ ID NO: 556 - DNA, 843 bp, synthetic construct
  source 1..843: mol_type = other DNA
gcactgaaggtcctcaatcgcactggaaacatcaaggtcgnnnnnnnnnnnttgacagctagctcagtcctaggtataatgctagcaaagaggagaaaggatctatggtcagtaaaggagaagaacttttcactggagttgtcccaattcttgttgaattagatggtgatgttaatgggcacaaattctctgtcagtggagagggtgaaggtgatgcaacatacggaaaacttacccttaaatttatttgcactactggaaagctacctgttccatggccaacacttgtcactactttgacttatggtgttcaatgcttttcaagatacccagatcatatgaaacagcatgactttttcaagagtgccatgcccgaaggttatgtacaggaaagaactatattttacaaagatgacgggaactacaaatcacgtgctgaagtcaagtttgaaggtgataccctcgttaatagaattgagttaaaaggtattgattttaaagaagatggaaacattcttggacacaaaatggaatacaactataactcacacaatgtatacatcatggcagacaaacaaaagaatggaatcaaagttaacttcaaaattagacacaacattgaagatggaagcgttcaactagcagaccattatcaacaaaatactccaattggcgatggccctgtccttttaccagacaaccattacctgtccacacaatctgccctttccaaagatcccaacgaaaagagagatcacatgatccttcttgagtttgtaacagctgctgggattacacatggcatggatgaactatacaaataagtttccgtctacgaactcc

SEQ ID NO: 557 - DNA, 1738 bp, synthetic construct
  source 1..1738: mol_type = other DNA
aaatctatcttgatcggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaatttcgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaatttgatatcgagctcgcttggactcctgttgatagatccagtaatgacctcagaactccatctggatttgttcagaacgctcggttgccgccgggcgttttttattggtgagaatccaagcactagtaacaacttatatcgtatggggctgacttcaggtgctacatttgaagagataaattgcactgaaatctagaaatattttatctgattaataagatgatcttcttgagatcgttttggtctgcgcgtaatctcttgctctgaaaacgaaaaaaccgccttgcagggcggtttttcgaaggttctctgagctaccaactctttgaaccgaggtaactggcttggaggagcgcagtcaccaaaacttgtcctttcagtttagccttaaccggcgcatgacttcaagactaactcctctaaatcaattaccagtggctgctgccagtggtgcttttgcatgtctttccgggttggactcaagacgatagttaccggataaggcgcagcggtcggactgaacggggggttcgtgcatacagtccagcttggagcgaactgcctacccggaactgagtgtcaggcgtggaatgagacaaacgcggccataacagcggaatgacaccggtaaaccgaaaggcaggaacaggagagcgcacgagggagccgccagggggaaacgcctggtatctttatagtcctgtcgggtttcgccaccactgatttgagcgtcagatttcgtgatgcttgtcaggggggcggagcctatggaaaaacggctttgccgcggccctctcacttccctgttaagtatcttcctggcatcttccaggaaatctccgccccgttcgtaagccatttccgctcgccgcagtcgaacgaccgagcgtagcgagtcagtgagcgaggaagcggaatatatccctaggtataaacgcagaaaggcccacccgaaggtgagccag

SEQ ID NO: 558 - DNA, 3915 bp, synthetic construct
  source 1..3915: mol_type = other DNA
ttgacaattaatcatcggctcgtataatgtgtggaattgtgagcggataacaagctactagagtcatttatgaaagtactagatgaatacgattaacatcgctaagaacgacttctctgacatcgaactggctgctatcccgttcaacactctggctgaccattacggtgagcgtttagctcgcgaacagttggcccttgagcatgagtcttacgagatgggtgaagcacgcttccgcaagatgtttgagcgtcaacttaaagctggtgaggttgcggataacgctgccgccaagcctctcatcactaccctactccctaagatgattgcacgcatcaacgactggtttgaggaagtgaaagctaagcgcggcaagcgcccgacagccttccagttcctgcaagaaatcaagccggaagccgtagcgtacatcaccattaagaccactctggcttgcctaaccagtgctgacaatacaaccgttcaggctgtagcaagcgcaatcggtcgggccattgaggacgaggctcgcttcggtcgtatccgtgaccttgaagctaagcacttcaagaaaaacgttgaggaacaactcaacaagcgcgtagggcacgtctacaagaaagcatttatgcaagttgtcgaggctgacatgctctctaagggtctactcggtggcgaggcgtggtcttcgtggcataaggaagactctattcatgtaggagtacgctgcatcgagatgctcattgagtcaaccggaatggttagcttacaccgccaaaatgctggcgtagtaggtcaagactctgagactatcgaactcgcacctgaatacgctgaggctatcgcaacccgtgcaggtgcgctggctggcatctctccgatgttccaaccttgcgtagttcctcctaagccgtggactggcattactggtggtggctattgggctaacggtcgtcgtcctctggcgctggtgcgtactcacagtaagaaagcactgatgcgctacgaagacgtttacatgcctgaggtgtacaaagcgattaacattgcgcaaaacaccgcatggaaaatcaacaagaaagtcctagcggtcgccaacgtaatcaccaagtggaagcattgtccggtcgaggacatccctgcgattgagcgtgaagaactcccgatgaaaccggaagacatcgacatgaatcctgaggctctcaccgcgtggaaacgtgctgccgctgctgtgtaccgcaaggacaaggctcgcaagtctcgccgtatcagccttgagttcatgcttgagcaagccaataagtttgctaaccataaggccatctggttcccttacaacatggactggcgcggtcgtgtttacgctgtgtcaatgttcaacccgcaaggtaacgatatgaccaaaggactgcttacgctggcgaaaggtaaaccaatcggtaaggaaggttactactggctgaaaatccacggtgcaaactgtgcgggtgtcgataaggttccgttccctgagcgcatcaagttcattgaggaaaaccacgagaacatcatggcttgcgctaagtctccactggagaacacttggtgggctgagcaagattctccgttctgcttccttgcgttctgctttgagtacgctggggtacagcaccacggcctgagctataactgctcccttccgctggcgtttgacgggtcttgctctggcatccagcacttctccgcgatgctccgagatgaggtaggtggtcgcgcggttaacttgcttcctagtgaaaccgttcaggacatctacgggattgttgctaagaaagtcaacgagattctacaagcagacgcaatcaatgggaccgataacgaagtagttaccgtgaccgatgagaacactggtgaaatctctgagaaagtcaagctgggcactaaggcactggctggtcaatggctggcttacggtgttactcgcagtgtgactaagcgttcagtcatgacgctggcttacgggtccaaagagttcggcttccgtcaacaagtgctggaagataccattcagccagctattgattccggcaagggtctgatgttcactcagccgaatcaggctgctggatacatggctaagctgatttgggaatctgtgagcgtgacggtggtagctgcggttgaagcaatgaactggcttaagtctgctgctaagctgctggctgctgaggtcaaagataagaagactggagagattcttcgcaagcgttgcgctgtgcattgggtaactcctgatggtttccctgtgtggcaggaatacaagaagcctattcagacgcgcttgaacctgatgttcctcggtcagttccgcttacagcctaccattaacaccaacaaagatagcgagattgatgcacacaaacaggagtctggtatcgctcctaactttgtacacagccaagacggtagccaccttcgtaagactgtagtgtgggcacacgagaagtacggaatcgaatcttttgcactgattcacgactccttcggtaccattccggctgacgctgcgaacctgttcaaagcagtgcgcgaaactatggttgacacatatgagtcttgtgatgtactggctgatttctacgaccagttcgctgaccagttgcacgagtctcaattggacaaaatgccagcacttccggctaaaggtaacttgaacctccgtgacatcttagagtcggacttcgcgttcgcgtaaaggctgtctcgtctcgtctcgacaccatcgaatggtgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtga

(19) United States
(12) Patent Application Publication
(10) Pub. No.: US 2026/0193813 A1
(43) Pub. Date: Jul. 9, 2026

(54) SIDEWINDER THREE-WAY JUNCTION DNA ASSEMBLY

(71) Applicant: California Institute of Technology, Pasadena, CA (US)

(72) Inventors: Kaihang Wang, Pasadena, CA (US); Noah E. Robinson, Pasadena, CA (US)

(21) Appl. No.: 19/441,759

(22) Filed: Jan. 6, 2026

Related U.S. Application Data
(60) Provisional application No. 63/742,744, filed on Jan. 7, 2025.

(51) Int. Cl. C40B 40/06; C12N 9/00; C12N 9/12; C12N 15/113; C12N 15/63; C12P 19/34; C40B 30/06; C40B 70/00

(52) U.S. Cl. CPC C40B 40/06; C12N 9/1241; C12N 9/93; C12N 15/113; C12N 15/63; C12P 19/34; C40B 30/06; C40B 70/00

(57) ABSTRACT

Disclosed herein include methods, compositions, and kits suitable for use in polynucleotide assembly. Methods, compositions, systems, and kits provided herein can employ a strategy which implements highly specific external barcodes that are not incorporated into the final assembled product. In some embodiments, a highly specific DNA barcode pair forms an external third helix to hold synthetic fragments together at a temperature prohibiting interactions of short complementary toehold sequences alone before enzymatically ligating nicks in the lower strand to covalently fix the connection between fragments. The method can comprise removal of the external third helix either enzymatically, or by PCR amplification of the lower strand without the external third helix, to form a seamless connection.

### RELATED APPLICATIONS

This application claims the benefit under 35 U.S.C. § 119 (e) of U.S. Provisional Patent Application Ser. No. 63/742,744, filed Jan. 7, 2025, the content of this related application is incorporated herein by reference in its entirety for all purposes.

### STATEMENT REGARDING FEDERALLY SPONSORED R&D

This invention was made with government support under Grant No. GM140937 awarded by the National Institutes of Health. The government has certain rights in the invention.

### REFERENCE TO SEQUENCE LISTING

The present application is being filed along with a Sequence Listing in electronic format. The Sequence Listing is provided as a file entitled 30KJ-810006-US, created Jan. 6, 2026, which is 558,958 bytes in size. The information in the electronic format of the Sequence Listing is incorporated herein by reference in its entirety.

### BACKGROUND

### Field

The present disclosure relates generally to the field of polynucleotide assembly.

### Description of the Related Art

DNA encodes the information required for biological systems to carry out a broad range of functions. The understanding of this relationship has sparked inquiries across vast fields of biology and biological engineering as investigators read, edit, and write the genetic information of organisms. Great advancements have been made toward these pursuits, from revolutions in DNA reading through long read sequencing and the ability to generate terabytes of data from a single run, to the breakthroughs in DNA editing with the major advancements in CRISPR/Cas technologies over the last decade. However, writing DNA, as the ability to construct DNA of any length, complexity, or diversity, lags behind since DNA oligo synthesis can only reach short lengths and DNA assembly of oligos and short DNA fragments is fundamentally limited. While the need for affordable, large, and complex synthetic DNA has grown exponentially, advancements in DNA construction have not sufficiently improved to meet the scale and efficiency which is required for the age of synthetic genomes, biomaterials, massively multiplexed machine-learning Protein Language Models, and directed protein evolution. There is a need for compositions, methods, systems, and kits for polynucleotide assembly.

### SUMMARY

Disclosed herein include compositions. The composition can comprise: n fragments, wherein n is an integer greater than 2. In some embodiments, each fragment comprises a first polynucleotide strand and a second polynucleotide strand. In some embodiments, each (i)th fragment comprises a first barcode, a first toehold, a second barcode, and a second toehold, wherein 1<i<n. In some embodiments, the first fragment comprises a first terminal region, a second barcode, and a first toehold, optionally the first terminal region is a 5′ first terminal region. In some embodiments, the (n)th fragment comprises a first barcode, a second toehold, and a second terminal region, optionally the second terminal region is a 3′ second terminal region. In some embodiments, for each (i)th fragment, wherein 1<i<n: the first polynucleotide strand comprises a 5′ overhang and a 3′ overhang; the 5′ overhang of the first polynucleotide strand comprises the first barcode; the 3′ overhang of the first polynucleotide strand comprises the second barcode; the first barcode of the (i)th fragment is complementary to the second barcode of the (i−1)th fragment; the first toehold of the (i)th fragment is complementary to the second toehold of the (i+1)th fragment; the second barcode of the (i)th fragment is complementary to the first barcode of the (i+1)th fragment; and the second toehold of the (i)th fragment is complementary to the first toehold of the (i−1)th fragment. The methods, compositions, systems, and kits provided herein can comprise the generation of a linear product (See FIG. 2B).

Disclosed herein include compositions. The composition can comprise: n fragments, wherein n is an integer greater than 2. In some embodiments, each fragment comprises a first barcode, a first toehold, a second barcode, and a second toehold. In some embodiments, each fragment comprises a first polynucleotide strand and a second polynucleotide strand. In some embodiments, the first polynucleotide strand comprises a 5′ overhang and a 3′ overhang. In some embodiments, the 5′ overhang of the first polynucleotide strand comprises the first barcode. In some embodiments, the 3′ overhang of the first polynucleotide strand comprises the second barcode. In some embodiments, for each (i)th fragment, wherein 1<i<n: the first barcode of the (i)th fragment is complementary to the second barcode of the (i−1)th fragment; the first toehold of the (i)th fragment is complementary to the second toehold of the (i+1)th fragment; the second barcode of the (i)th fragment is complementary to the first barcode of the (i+1)th fragment; and the second toehold of the (i)th fragment is complementary to the first toehold of the (i−1)th fragment. In some embodiments, the first barcode of the first fragment is complementary to the second barcode of the (n)th fragment. In some embodiments, the second toehold of the first fragment is complementary to the first toehold of the (n)th fragment. The methods, compositions, systems, and kits provided herein can comprise the generation of a circular product (See FIG. 18).

In some embodiments, for each (i)th fragment, wherein 1<i<n: the first barcode of the (i)th fragment is not complementary to the first barcode of any of the n fragments; and the first barcode of the (i)th fragment is not complementary to the second barcode of any (k)th fragment, wherein k is an integer not equal to (i-1). In some embodiments, the 3′ overhang of the first polynucleotide strand comprises the first toehold, the first toehold is 5′ of the second barcode, the second polynucleotide strand comprises a 3′ overhang, and the 3′ overhang of the second polynucleotide strand comprises the second toehold (See FIG. 2F). In some embodiments, the 5′ overhang of the first polynucleotide strand comprises the second toehold, the second toehold is 3′ of the first barcode, the second polynucleotide strand comprises a 5′ overhang, and the 5′ overhang of the second polynucleotide strand comprises the first toehold (See FIG. 11D).

In some embodiments, said complementarity is or comprises: at least 80%, 85%, 90%, 95%, 99%, or 100% complementarity; less than five, four, three, two, or one, base pair mismatches; reverse complementarity; canonical Watson-Crick base pairing; wobble base pairing, optionally G-U wobble; and/or DNA nanotechnology interactions, optionally Hoogsteen base pairing, G-quadruplex(es), DNA origami, aptamer-ligand interactions, or any combination thereof. In some embodiments, at least 80%, 85%, 90%, 95%, 99%, or 100% of the fragments comprise a payload segment. In some embodiments, at least 80%, 85%, 90%, 95%, 99%, or 100% of the fragments comprise a toehold-flanked internal payload segment. In some embodiments, the payload segment comprises the sequence of the first toehold and/or the second toehold. In some embodiments, the payload segment does not comprise the sequence of the first barcode or the second barcode.

In some embodiments, the first fragment, the (i)th fragment, the (n)th fragment, one or more of the n fragments, the first toehold, the second toehold, the first barcode, the second barcode, the payload segment, terminal region, and/or the internal payload segment: is at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 110, 120, 130, 140, 150, 160, 170, 180, 190, 200, 210, 220, 230, 240, 250, 1-5, 1-10, 10-100, 10-250, 25-50, 25-100, 25-250, 50-100, 50-200, 50-250, 75-100, 75-200, 75-250, 100-150, 100-200, 100-250, 150-200, 150-250, 200-250, or a number or a range between any two of these values, nucleotides in length; comprises a GC content of about 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, 20%-50%, 20%-75%, 20%-100%, 30%-60%, 30%-75%, 30%-100%, 40%-60%, 40%-75%, 40%-100%, 50%-75%, 50%-100%, 60%-75%, 60%-100%, 75%-100%, or a number or a range between any two of these values; comprises a melting temperature (Tₘ) of about 35° C., 36° C., 37° C., 38° C., 39° C., 40° C., 41° C., 42° C., 43° C., 44° C., 45° C., 46° C., 47° C., 48° C., 49° C., 50° C., 51° C., 52° C., 53° C., 54° C., 55° C., 56° C., 57° C., 58° C., 59° C., 60° C., 61° C., 62° C., 63° C., 64° C., 65° C., 66° C., 67° C., 68° C., 69° C., 70° C., 71° C., 72° C., 73° C., 74° C., 75° C., 35° C.-55° C., 35° C.-75° C., 35° C.-100° C., 45° C.-55° C., 45° C.-75° C., 45° C.-100° C., 55° C.-75° C., 55° C.-100° C., 65° C.-75° C., 65° C.-100° C., 75° C.-100° C., or a number or a range between any two of these values; comprises DNA; comprises RNA; and/or comprises one or more nucleic acid analogs, optionally selected from the group consisting of RNA, 2′-O-methyl RNA, locked nucleic acid (LNA), peptide nucleic acid (PNA), morpholino, phosphorodiamidate morpholino oligomer (PMO), HNA, FANA, TNA, ANA, GNA, CeNA, UNA, L-DNA, or any combination thereof.

In some embodiments, the melting temperature (Tₘ) of the first barcode and the second barcode is at least about 5° C., 6° C., 7° C., 8° C., 9° C., 10° C., 11° C., 12° C., 13° C., 14° C., 15° C., 16° C., 17° C., 18° C., 19° C., 20° C., 21° C., 22° C., 23° C., 24° C., 25° C., or a number or a range between any two of these values, higher than the Tₘ of the first toehold and the second toehold. In some embodiments, a first barcode comprises the sequence of the first 1, 2, 3, 4, 5, 6, 7, 8, 9, 10, 11, 12, 13, 14, 15, 16, 17, 18, 19, or 20, nucleotides, of any one of SEQ ID Nos: 1-548 or SEQ ID Nos: 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, and 300. In some embodiments, a second barcode comprises the sequence of the final 1, 2, 3, 4, 5, 6, 7, 8, 9, 10, 11, 12, 13, 14, 15, 16, 17, 18, 19, or 20, nucleotides, of any one of SEQ ID Nos: 1-548 or SEQ ID Nos: 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, and 300.

In some embodiments, the fragments, the first polynucleotide strand, and/or the second polynucleotide strand: comprise or are derived from synthetic oligonucleotides; and/or comprise or are derived from rolling circle amplification products, restriction enzyme digestion products, reverse transcription products, CRISPR-excised products, PCR amplification products, template-independent polymerase products, recombinase-generated products, phage-derived products, or any combination thereof. In some embodiments, the first barcode of the (i)th fragment forms a pair with the second barcode of the (i−1)th fragment. In some embodiments, the second barcode of the (i)th fragment forms a pair with the first barcode of the (i+1)th fragment. In some embodiments, each pair is optimized for maximum mutual specificity within a pair and absolute exclusivity across different pairs. In some embodiments, n is at least 3, 4, 5, 6, 7, 8, 9, 10, 11, 12, 13, 14, 15, 16, 17, 18, 19, 20, 22, 24, 26, 28, 30, 32, 34, 36, 38, 40, 42, 44, 46, 48, 50, 55, 60, 65, 70, 75, 80, 85, 90, 95, 100, 125, 150, 175, 200, 225, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, 1000, 10-25, 10-50, 10-75, 10-100, 10-500, 10-1000, 25-50, 25-75, 25-100, 25-500, 25-1000, 50-75, 50-100, 50-500, 50-1000, 75-100, 75-500, 75-1000, 100-500, 100-1000, 500-1000, or a number or a range between any two of these values.

In some embodiments, upon incubation in a reaction mixture, the n fragments are capable of joining together via at least one three-way junction (3WJ) intermediate to generate an intermediate product. In some embodiments, a ligase is capable of ligating nicks on the second polynucleotide strands of said intermediate product to generate an assembled product. In some embodiments, a ligase and/or a chemical coupling agent is capable of forming a covalent linkage between adjacent second polynucleotide strands of said intermediate product to generate an assembled product. In some embodiments, the covalent linkage is formed by a click ligation between complementary reactive handles on adjacent second polynucleotide strands, optionally copper (I)-catalyzed azide-alkyne cycloaddition (CuAAC), strain promoted azide-alkyne cycloaddition (SPAAC), or inverse electron demand Diels-Alder (iEDDA) reaction between a trans cyclooctene and a tetrazine oxime formation, hydrazone formation, Michael addition, disulfide formation, carbodiimide-mediated coupling, native chemical ligation, or any combination thereof. In some embodiments, the second polynucleotide strands comprise synthetic modifications and/or modified synthetic nucleotides, optionally selected a 5′ alkyne, a 3′ azide, a trans-cyclooctene, a tetrazine, a 5′ amine, an aldehyde, an aminooxy group, a thiol, a maleimide, or a phosphorothioate, or any combination thereof. In some embodiments, the chemical coupling agent comprises a click chemistry reagent, a copper (I) source, a copper (I)-stabilizing ligand, a strain-promoted cycloaddition reagent, a tetrazine, an EDC or other carbodiimide, an aniline or p-phenylenediamine catalyst, or any combination thereof.

In some embodiments, the assembled product comprises a final synthetic sequence, wherein the final synthetic sequence does not comprise the sequence of the first barcode or the second barcode of any of the n fragments, and wherein the final synthetic sequence comprises the scarless assembly of the payload segments of the n fragments. In some embodiments, the lengths of the first toehold and the second toehold are configured to ensure effective ligase docking and ligation of nicks on the second polynucleotide strands of said intermediate product, optionally at least 6 nucleotides in length. In some embodiments, the final synthetic sequence is at least about 500 bases, 750 bases, 1 kb, 2 kb, 3 kb, 4 kb, 5 kb, 6 kb, 7 kb, 8 kb, 9 kb, 10 kb, 15 kb, 20 kb, 25 kb, 50 kb, 75 kb, 100 kb, 250 kb, 500 kb, 750 kb, 1 MB, or a number or a range between any two of these values, in length.

In some embodiments, the final synthetic sequence, the first toehold, the second toehold, the payload segment, and/or the internal payload segment: comprises an elevated GC content of at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values; comprises a reduced GC content of less about 40%, 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, 1%, 40%-30%, 40%-20%, 40%-10%, 40%-5%, 40%-1%, 30%-20%, 30%-10%, 30%-5%, 30%-1%, 20%-10%, 20%-5%, 20%-1%, 10%-5%, 10%-1%, 5%-1%, or a number or a range between any two of these values; comprises two or more repeats, optionally tandem repeats, optionally at least 4 nt in length, optionally occurring at least 3, 4, 5, 6, 7, 8, 9, 10, 11, 12, 13, 14, 15, 16, 17, 18, 19, 20 times, or a number or a range between any two of these values, within the final synthetic sequence; and/or comprises two or more mononucleotide stretches, optionally at least 4 nt in length, optionally occurring at least 3, 4, 5, 6, 7, 8, 9, 10, 11, 12, 13, 14, 15, 16, 17, 18, 19, 20 times, or a number or a range between any two of these values, within the final synthetic sequence. In some embodiments, for each (i)th fragment, the first toehold of the (i)th fragment is not complementary to the second toehold of any (k)th fragment, wherein k is an integer not equal to (i+1). In some embodiments, for at least one (i)th fragment, the first toehold of the (i)th fragment is complementary to the second toehold of one or more (k)th fragments, wherein k is an integer not equal to (i+1).

In some embodiments, the first fragment: is an invariant fragment, wherein all instances of the invariant first fragment in the composition are identical; or is a variant fragment, wherein two or more instances of the variant first fragment in the composition differ with respect to the sequence of the internal payload segment. In some embodiments, at least one (i)th fragment is an invariant fragment, wherein all instances of the invariant (i)th fragment in the composition are identical. In some embodiments, at least one (i)th fragment is a variant fragment, wherein two or more instances of the variant (i)th fragment in the composition differ with respect to the sequence of the internal payload segment. In some embodiments, the (n)th fragment: is an invariant fragment, wherein all instances of the invariant (n)th fragment in the composition are identical; or is a variant fragment, wherein two or more instances of the variant (n)th fragment in the composition differ with respect to the sequence of the internal payload segment. In some embodiments, variant fragments comprise predefined codon variations, optionally codons variations configured to achieve modified and/or improved protein function(s).

In some embodiments, the composition comprises y sets of n fragments. In some embodiments, the value of n is the same between at least two of the y sets. In some embodiments, the value of n is the different between at least two of the y sets. In some embodiments, the first barcode and the second barcode of each set are not complementary to the first barcode and the second barcode of any other set. In some embodiments, upon incubation of the y sets together in a single reaction mixture, each set of n fragments is capable of, in parallel, joining together via three-way junction (3WJ) intermediates to generate y intermediate products. In some embodiments, the y intermediate products are candidate design variants. In some embodiments, the y intermediate products, or products thereof, are capable of being individually amplified or universally amplified. In some embodiments, y is an integer greater than 1, optionally at least 2, 3, 4, 5, 6, 7, 8, 9, 10, 11, 12, 13, 14, 15, 16, 17, 18, 19, 20, 21, 22, 23, 24, 25, 26, 27, 28, 29, 30, or a number or a range between any two of these values.

In some embodiments, the final synthetic sequence comprises one or more payload genes, optionally the one or more payload genes encode one or more RNA payload(s) and/or one or more payload protein(s). In some embodiments, the one or more RNA payload(s) are selected from the group comprising a CRISPR single-guide RNA (sgRNA), a small interfering RNA (siRNA), a CRISPR RNA (crRNA), a small hairpin RNA (shRNA), a microRNA (miRNA), a piwi-interacting RNA (piRNA), an antisense oligonucleotide, an antagomir, an aptamer, a ribozyme, or any combination thereof. In some embodiments, a payload protein comprises: fluorescence activity, polymerase activity, protease activity, phosphatase activity, kinase activity, SUMOylating activity, deSUMOylating activity, ribosylation activity, deribosylation activity, myristoylation activity demyristoylation activity, or any combination thereof; nuclease activity, methyltransferase activity, demethylase activity, DNA repair activity, DNA damage activity, deamination activity, dismutase activity, alkylation activity, depurination activity, oxidation activity, pyrimidine dimer forming activity, integrase activity, transposase activity, recombinase activity, polymerase activity, ligase activity, helicase activity, photolyase activity, glycosylase activity, acetyltransferase activity, deacetylase activity, adenylation activity, deadenylation activity, or any combination thereof; a biomaterials payload, optionally a structural polypeptide, further optionally silk fibroin, spider silk spidroin, a resilin, a resilin-like polypeptide, an elastin, an elastin-like polypeptide, a collagen, or a collagen-like polypeptide; a cellular reprogramming factor capable of differentiating a given cell into a desired differentiated state, optionally nerve growth factor (NGF), fibroblast growth factor (FGF), interleukin-6 (IL-6), bone morphogenic protein (BMP), neurogenin3 (Ngn3), pancreatic and duodenal homeobox 1 (Pdx1), Mafa, or any combination thereof; an agonistic or antagonistic antibody or antigen-binding fragment thereof specific to a checkpoint inhibitor or checkpoint stimulator molecule, optionally PD1, PD-L1, PD-L2, CD27, CD28, CD40, CD137, OX40, GITR, ICOS, A2AR, B7-H3, B7-H4, BTLA, CTLA4, IDO, KIR, LAG3, PD-1, and/or TIM-3; a secretion tag, optionally the secretion tag is selected from the group comprising AbnA, AmyE, AprE, BgIC, BgIS, Bpr, Csn, Epr, Ggt, GlpQ, HtrA, LipA, LytD, MntA, Mpr, NprE, OppA, PbpA, PbpX, Pel, PelB, PenP, PhoA, PhoB, PhoD, PstS, TasA, Vpr, WapA, WprA, XynA, XynD, YbdN, Ybxl, YcdH, YclQ, YdhF, YdhT, YfkN, YflE, YfmC, Yfnl, YhcR, YlqB, YncM, YnfF, YoaW, YocH, YolA, YqiX, Yqxl, YrpD, YrpE, YuaB, Yurl, YvcE, YvgO, YvpA, YwaD, YweA, YwoF, YwtD, YwtF, YxaLk, YxiA, and YxkC; a constitutive signal peptide for protein degradation, optionally PEST; a nuclear localization signal (NLS) or a nuclear export signal (NES); a dosage indicator protein, optionally the dosage indicator protein is detectable, optionally the dosage indicator protein comprises green fluorescent protein (GFP), enhanced green fluorescent protein (EGFP), yellow fluorescent protein (YFP), enhanced yellow fluorescent protein (EYFP), blue fluorescent protein (BFP), red fluorescent protein (RFP), TagRFP, Dronpa, Padron, mApple, mCherry, mruby3, rsCherry, rsCherryRev, derivatives thereof, or any combination thereof; a cellular reprogramming factor capable of converting an at least partially differentiated cell to a less differentiated cell, optionally Oct-3, Oct-4, Sox2, c-Myc, Klf4, Nanog, Lin28, ASCL1, MYTIL, TBX3b, SV40 large T, hTERT, miR-291, miR-294, miR-295, or any combinations thereof; a programmable nuclease, optionally the programmable nuclease is selected from the group comprising: SpCas9 or a derivative thereof; VRER, VQR, EQR SpCas9; xCas9-3.7; eSpCas9; Cas9-HF1; HypaCas9; evoCas9; HiFi Cas9; ScCas9; StCas9; NmCas9; SaCas9; CjCas9; CasX; Cas9 H940A nickase; Cas12 and derivatives thereof; dcas9-APOBEC1 fusion, BE3, and dcas9-deaminase fusions; dcas9-Krab, dCas9-VP64, dCas9-Tet1, and dcas9-transcriptional regulator fusions; Dcas9-fluorescent protein fusions; Cas13-fluorescent protein fusions; RCas9-fluorescent protein fusions; Cas13-adenosine deaminase fusions, or any combination thereof; a CRE recombinase, GCaMP, a cell therapy component, a knock-down gene therapy component, a cell-surface exposed epitope, or any combination thereof; a bispecific T cell engager (BiTE); a synthetic receptor, optionally a Synthetic Notch (SynNotch) receptor, a Modular Extracellular Sensor Architecture (MESA) receptor, Tango, dCas9-synR, or any combination thereof; a cytokine, optionally the cytokine is selected from the group consisting of interleukin-1 (IL-1), IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33, IL-34, IL-35, interleukin-1 (IL-1), IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33, IL-34, IL-35, granulocyte macrophage colony stimulating factor (GM-CSF), M-CSF, SCF, TSLP, oncostatin M, leukemia-inhibitory factor (LIF), CNTF, Cardiotropin-1, NNT-1/BSF-3, growth hormone, Prolactin, Erythropoietin, Thrombopoietin, Leptin, G-CSF, or receptor or ligand thereof; a member of the TGF-β/BMP family selected from the group consisting of TGF-β1, TGF-β2, TGF-β3, BMP-2, BMP-3a, BMP-3b, BMP-4, BMP-5, BMP-6, BMP-7, BMP-8a, BMP-8b, BMP-9, BMP-10, BMP-11, BMP-15, BMP-16, endometrial bleeding associated factor (EBAF), growth differentiation factor-1 (GDF-1), GDF-2, GDF-3, GDF-5, GDF-6, GDF-7, GDF-8, GDF-9, GDF-12, GDF-14, mullerian inhibiting substance (MIS), activin-1, activin-2, activin-3, activin-4, and activin-5; a member of the TNF family of cytokines selected from the group consisting of TNF-alpha, TNF-beta, LT-beta, CD40 ligand, Fas ligand, CD 27 ligand, CD 30 ligand, and 4-1 BBL; a member of the immunoglobulin superfamily of cytokines selected from the group consisting of B7.1 (CD80) and B7.2 (B70); an interferon, optionally the interferon is selected from interferon alpha, interferon beta, or interferon gamma; a chemokine, optionally the chemokine is selected from CCL1, CCL2, CCL3, CCR4, CCL5, CCL7, CCL8/MCP-2, CCL11, CCL13/MCP-4, HCC-1/CCL14, CTAC/CCL17, CCL19, CCL22, CCL23, CCL24, CCL26, CCL27, VEGF, PDGF, lymphotactin (XCL1), Eotaxin, FGF, EGF, IP-10, TRAIL, GCP-2/CXCL6, NAP-2/CXCL7, CXCL8, CXCL10, ITAC/CXCL11, CXCL12, CXCL13, or CXCL15; an interleukin, optionally the interleukin is selected from IL-10 IL-12, IL-1, IL-6, IL-7, IL-15, IL-2, IL-18 or IL-21; a tumor necrosis factor (TNF), optionally the TNF is selected from TNF-alpha, TNF-beta, TNF-gamma, CD252, CD154, CD178, CD70, CD153, or 4-1BBL; a factor locally down-regulating the activity of endogenous immune cells; a factor capable of remodeling a tumor microenvironment and/or reducing immunosuppression at a target site of a subject; a chimeric antigen receptor (CAR) or T-cell receptor (TCR), optionally the CAR and/or TCR comprises one or more of an antigen binding domain, a transmembrane domain, and an intracellular signaling domain, optionally wherein the intracellular signaling domain comprises a primary signaling domain, a costimulatory domain, or both of a primary signaling domain and a costimulatory domain; and/or an activity regulator, optionally the activity regulator is capable of reducing T cell activity.

In some embodiments, a payload protein is associated with an agricultural trait of interest selected from the group consisting of increased yield, increased abiotic stress tolerance, increased drought tolerance, increased flood tolerance, increased heat tolerance, increased cold and frost tolerance, increased salt tolerance, increased heavy metal tolerance, increased low-nitrogen tolerance, increased disease resistance, increased pest resistance, increased herbicide resistance, increased biomass production, male sterility, or any combination thereof. In some embodiments, a payload protein is associated with a biological manufacturing process selected from the group comprising fermentation, distillation, biofuel production, production of a compound, production of a polypeptide, or any combination thereof.

In some embodiments, the one or more payload genes are selected from the group comprising a nitrogen fixation gene, a plant stress-induced gene, a nutrient utilization gene, a gene that affects plant pigmentation, a gene that encodes an antisense or ribozyme molecule, a gene encoding an antigen capable of being secreted, a toxin gene, a receptor gene, a ligand gene, a seed storage gene, a hormone gene, an enzyme gene, an interleukin gene, a cytokine gene, a growth factor gene, a transcription factor gene, a transcriptional repressor gene, a DNA-binding protein gene, a recombination gene, a DNA replication gene, a programmed cell death gene, a kinase gene, a phosphatase gene, a G protein gene, a cyclin gene, a cell cycle control gene, a gene involved in transcription, a gene involved in translation, a gene involved in RNA processing, a gene involved in RNAi, an organellar gene, a intracellular trafficking gene, an integral membrane protein gene, a transporter gene, a membrane channel protein gene, a cell wall gene, a gene involved in protein processing, a gene involved in protein modification, a gene involved in protein degradation, a gene involved in metabolism, a gene involved in biosynthesis, a gene involved in assimilation of nitrogen or other elements or nutrients, a gene involved in controlling carbon flux, gene involved in respiration, a gene involved in photosynthesis, a gene involved in light sensing, a gene involved in organogenesis, a gene involved in embryogenesis, a gene involved in differentiation, a gene involved in meiotic drive, a gene involved in self incompatibility, a gene involved in development, a gene involved in nutrient, metabolite or mineral transport, a gene involved in nutrient, metabolite or mineral storage, a calcium-binding protein gene, a lipid-binding protein gene, or any combination thereof.

In some embodiments, the one or more payload genes are selected from the group comprising a gene encoding an enzyme involved in metabolizing biochemical wastes for use in bioremediation, a gene that encodes an enzyme for modifying pathways that produce secondary plant metabolites, a gene that encodes an enzyme that produces a pharmaceutical, a gene that encodes an enzyme that improves or changes the nutritional content of a plant, a gene that encodes an enzyme involved in vitamin synthesis, a gene that encodes an enzyme involved in carbohydrate, polysaccharide or starch synthesis, a gene that encodes an enzyme involved in mineral accumulation or availability, a gene that encodes a phytase, a gene that encodes an enzyme involved in fatty acid, fat or oil synthesis, a gene that encodes an enzyme involved in synthesis of chemicals or plastics, a gene that encodes an enzyme involved in synthesis of a fuel, a gene that encodes an enzyme involved in synthesis of a fragrance, a gene that encodes an enzyme involved in synthesis of a flavor, a gene that encodes an enzyme involved in synthesis of a pigment or dye, a gene that encodes an enzyme involved in synthesis of a hydrocarbon, a gene that encodes an enzyme involved in synthesis of a structural or fibrous compound, a gene that encodes an enzyme involved in synthesis of a food additive, a gene that encodes an enzyme involved in synthesis of a chemical insecticide, a gene that encodes an enzyme involved in synthesis of an insect repellent, a gene controlling carbon flux in a plant, or any combination thereof.

In some embodiments, the one or more payload proteins comprise components of a synthetic protein circuit, optionally payload proteins configured to form one or more logic gates selected from the group comprising an OR logic gate, AND logic gate, NOR logic gate, NAND logic gate, IMPLY logic gate, NIMPLY logic gate, XOR logic gate, and an XNOR logic gate. In some embodiments, a payload protein is capable of modulating the expression, concentration, localization, stability, and/or activity of the one or more endogenous proteins of a cell. In some embodiments, the payload protein is a therapeutic protein or a variant thereof, optionally a therapeutic protein configured to prevent or treat a disease or disorder of a subject, further optionally the subject suffers from a deficiency of said therapeutic protein.

In some embodiments, one or more of the payload gene(s) comprise: a 5′UTR and/or a 3′UTR; a tandem gene expression element selected from the group an internal ribosomal entry site (IRES), foot-and-mouth disease virus 2A peptide (F2A), equine rhinitis A virus 2A peptide (E2A), porcine teschovirus 2A peptide (P2A) or Thosea asigna virus 2A peptide (T2A), or any combination thereof; and/or a transcript stabilization element, optionally the transcript stabilization element comprises woodchuck hepatitis post-translational regulatory element (WPRE), bovine growth hormone polyadenylation (bGH-polyA) signal sequence, human growth hormone polyadenylation (hGH-polyA) signal sequence, or any combination thereof. In some embodiments, at least one of the payload genes is operably connected to a promoter selected from the group comprising: an RNA pol I promoter; a pol II promoter, optionally CMV, SV40 early region or adenovirus major late promoter; or pol III promoter, optionally a U6 or H1 promoter; a minimal promoter, optionally TATA, miniCMV, and/or miniPromo; a bacteriophage promoter, optionally a bacteriophage T3 promoter, a bacteriophage T7 promoter, a bacteriophage SP6 promoter, or a combination thereof; a tissue-specific promoter and/or a lineage-specific promoter; an inducible promoter, optionally a T7 RNA polymerase promoter, a T3 RNA polymerase promoter, an Isopropyl-beta-D-thiogalactopyranoside (IPTG)-regulated promoter, a lactose induced promoter, a heat shock promoter, or a Tetracycline-regulated promoter, a tetracycline-dependent promoter, a lac-dependent promoter, a pB ad-dependent promoter, an AlcA-dependent promoter, a LexA-dependent promoter, or a heat-shock promoter; a ubiquitous promoter, optionally a cytomegalovirus (CMV) immediate early promoter, a CMV promoter, a viral simian virus 40 (SV40) (e.g., early or late), a Moloney murine leukemia virus (MoMLV) LTR promoter, a Rous sarcoma virus (RSV) LTR, an RSV promoter, a herpes simplex virus (HSV) (thymidine kinase) promoter, H5, P7.5, and P11 promoters from vaccinia virus, an elongation factor 1-alpha (EF1a) promoter, early growth response 1 (EGR1), ferritin H (FerH), ferritin L (FerL), Glyceraldehyde 3-phosphate dehydrogenase (GAPDH), eukaryotic translation initiation factor 4A1 (EIF4A1), heat shock 70 kDa protein 5 (HSPA5), heat shock protein 90 kDa beta, member 1 (HSP90B1), heat shock protein 70 kDa (HSP70), β-kinesin (β-KIN), the human ROSA 26 locus, a Ubiquitin C promoter (UBC), a phosphoglycerate kinase-1 (PGK) promoter, 3-phosphoglycerate kinase promoter, a cytomegalovirus enhancer, human β-actin (HBA) promoter, chicken β-actin (CBA) promoter, a CAG promoter, a CASI promoter, a CBH promoter; or any combination thereof.

In some embodiments, the final synthetic sequence is or comprises all or a portion of a vector. In some embodiments, a viral vector, a plasmid, a transposable element, a naked DNA vector, or any combination thereof. In some embodiments, an AAV vector, a lentivirus vector, a retrovirus vector, an adenovirus vector, a herpesvirus vector, a herpes simplex virus vector, a cytomegalovirus vector, a vaccinia virus vector, a MVA vector, a baculovirus vector, a vesicular stomatitis virus vector, a human papillomavirus vector, an avipox virus vector, a Sindbis virus vector, a VEE vector, a Measles virus vector, an influenza virus vector, a hepatitis B virus vector, an integration-deficient lentivirus (IDLV) vector, or any combination thereof. In some embodiments, the transposable element is piggybac transposon or sleeping beauty transposon. In some embodiments, the final synthetic sequence is configured for propagation in a eukaryotic or a prokaryotic cell. In some embodiments, the final synthetic sequence comprises: a bacterial origin of replication, optionally ColE1, p15A, pSC101, and RK2; an origin of transfer (oriT) and one or more mobilization genes configured to enable conjugative transfer; an autonomously replicating sequence (ARS), a centromeric sequence (CEN), and/or 2u elements; a rolling-circle replication origin, optionally derived from pC194, pE194, and pUB110; a mammalian origin of replication, optionally oriP/EBNA1 and/or SV40 ori; a selection marker, optionally an antibiotic resistance marker and/or a fluorescence marker; and/or a counter-selection marker, optionally sacB, rpsL, galK, CYH2, and/or URA3. In some embodiments, the final synthetic sequence is configured for insertion into a genome. In some embodiments, the final synthetic sequence comprises: recognition sites for an RNA-guided DNA binding complex, wherein the RNA-guided DNA binding complex comprises one or more Cas proteins, a transposase, one or more crRNAs, or any combination thereof; recognition sites for a transposition complex comprising one or more transposases; homology arms, optionally targeting a safe-harbor locus selected from AAVSI, ROSA26, CCR5, and H11; one or more recombination sites, optionally loxP, FRT, attB, attP, attL, and attR; and/or a reporter cassette. In some embodiments, the final synthetic sequence comprises a digital data storage payload encoded in nucleic acid sequence.

In some embodiments, each of the n fragments is housed in a separate vessel, optionally a tube, a well, or a microfluidic chamber. In some embodiments, the first polynucleotide strand and the second polynucleotide strand that constitute each of the n fragments is housed in a separate vessel, optionally a tube, a well, or a microfluidic chamber. In some embodiments, the composition further comprises: a non-thermostable ligase, a thermostable ligase, a chemical coupling agent, a polymerase, a primer capable of binding the first terminal region (or a complement thereof), a primer capable of binding the second terminal region (or a complement thereof), or any combination thereof. In some embodiments, the composition further comprises a ligation buffer. The ligation buffer can comprise: HiFi Taq buffer; one or more of Tris HCl at about 10 mM to about 200 mM, at a pH of about 7.0 to about 9.5 at the incubation temperature, Mg2+ at about 0.5 mM to about 20 mM, monovalent cation(s) at about 10 mM to about 300 mM, and a reducing agent at about 0.1 mM to about 20 mM; a ligase cofactor, optionally selected from ATP at about 0.05 mM to about 5 mM or NAD+ at about 0.01 mM to about 2 mM; a buffering species selected from Tris, HEPES, Bis Tris, MOPS, and PIPES, optionally configured to maintain pH between 8.3-8.8 at 25° C.; and/or one or more additives, optionally selected from bovine serum albumin at about 0.01 mg/mL to about 1 mg/mL, polyethylene glycol at about 1% to about 20% (w/v), betaine at about 0.1 M to about 2.0 M, dimethyl sulfoxide at about 1% to about 20% (v/v), formamide at about 0.5% to about 10% (v/v), glycerol at about 1% to about 20% (v/v), and/or a non-ionic detergent at about 0.001% to about 0.1% (v/v). In some embodiments, the composition does not comprise one or more reagents employed with Polymerase Cycling Assembly (PCA), Gibson assembly, USER, Yeast Assembly, Homologous Recombination, and/or Golden Gate assembly, optionally an exonuclease, an endonuclease, a single stranded DNA binding protein, a restriction endonuclease, a recombinase, or any combination thereof.

Disclosed herein include compositions. The composition can comprise: a pre-assembly reaction mixture comprising the n fragments disclosed herein at equimolar concentrations, optionally the temperature of the pre-assembly reaction mixture is above the melting temperature of the first and second toeholds and below the melting temperature of the first and second barcodes, and optionally the pre-assembly reaction mixture comprises a ligase or a chemical coupling agent. The composition can comprise: an intermediate reaction mixture comprising the n fragments disclosed herein joined together via three-way junction (3WJ) intermediates to generate an intermediate product, optionally said 3WJ intermediates each comprise a helix, and optionally the intermediate reaction mixture comprises a ligase or a chemical coupling agent. The composition can comprise: a post-ligation reaction mixture comprising an assembled product wherein the second polynucleotide strand does not comprise nicks, optionally the assembled product comprises three-way junction (3WJ) intermediates, optionally said 3WJ intermediates each comprise a helix.

Disclosed herein include methods. The method can comprise: providing n fragments, wherein n is an integer greater than 2. In some embodiments, each fragment comprises a first polynucleotide strand and a second polynucleotide strand. In some embodiments, each (i)th fragment comprises a first barcode and a second barcode on the first polynucleotide strand, wherein 1<i<n. In some embodiments, the first barcode of the (i)th fragment forms a pair with the second barcode of the (i−1)th fragment. In some embodiments, the second barcode of the (i)th fragment forms a pair with the first barcode of the (i+1)th fragment. The method can comprise: incubating the n fragments in a reaction mixture under reaction conditions such that: the first barcode of the (i)th fragment hybridizes to the second barcode of the (i−1)th fragment; and the second barcode of the (i)th fragment hybridizes to the first barcode of the (i+1)th fragment, thereby joining together the n fragments via three-way junction (3WJ) intermediates to generate an intermediate product. The method can comprise: ligating nicks on the second polynucleotide strands to generate an assembled product.

Disclosed herein include methods. The method can comprise: providing n fragments disclosed herein. The method can comprise: incubating the n fragments in a reaction mixture under reaction conditions such that: the first barcode of the (i)th fragment hybridizes to the second barcode of the (i−1)th fragment; and the second barcode of the (i)th fragment hybridizes to the first barcode of the (i+1)th fragment, thereby joining together the n fragments via three-way junction (3WJ) intermediates to generate an intermediate product. The method can comprise: ligating nicks on the second polynucleotide strands to generate an assembled product.

In some embodiments, hybridization of a first barcode and a second barcode of adjacent fragments forms a helix, wherein said 3WJ intermediates each comprise a helix. In some embodiments, (i) the hybridization of the first toehold and the second toehold of adjacent fragments further stabilizes the 3WJ intermediates; and/or (ii) one or more fragments do not comprise a toehold and the intermediate product is sufficiently stabilized by hybridization between first and second barcodes. In some embodiments, the formation of the helix holds adjacent fragments together at a temperature prohibiting interactions of the first toehold and second toehold of adjacent fragments alone. In some embodiments, the helix orthogonally winds up on the side of the final assembled sequence, thereby joining adjacent fragments together via the 3WJ intermediate. In some embodiments, the association of the first toehold and second toehold of adjacent fragments is unstable at the temperature(s) of the incubation step in the absence of the formation of the helix.

In some embodiments, the incubation step comprises: temperature(s) above the melting temperature (Tₘ) of the first toehold and second toehold. In some embodiments, the incubation step comprises: temperature(s) below the melting temperature (Tₘ) of the first barcode and second barcode. In some embodiments, the incubation comprises: incubation at a first incubation temperature for a first period of time. In some embodiments, the incubation comprises: addition of a ligase to the reaction mixture. In some embodiments, the incubation comprises: z assembly cycles, where z is an integer greater than 1. In some embodiments, each assembly cycle comprises: at least about 5 sec, 6 sec, 7 sec, 8 sec, 9 sec, 10 sec, 20 sec, 30 sec, 40 sec, 50 sec, 60 sec, 1 min, 2 min, 3 min, 4 min, 5 min, 6 min, 7 min, 8 min, 9 min, 10 min, or a number or a range between any two of these values, at a first incubation temperature, optionally 85° C. for 1 min; and at least about 10 sec, 20 sec, 30 sec, 40 sec, 50 sec, 60 sec, 1 min, 2 min, 3 min, 4 min, 5 min, 6 min, 7 min, 8 min, 9 min, 10 min, or a number or a range between any two of these values, at a second incubation temperature, optionally 50° C. for 2 min. In some embodiments, the incubation comprises: incubation at a second incubation temperature for a second period of time. In some embodiments, the incubation comprises: incubation at a first incubation temperature for a first period of time; cooling the reaction mixture from the first incubation temperature to the second incubation temperature at a predetermined cooling rate (optionally the predetermined cooling rate comprises a reduction of 0.1° C. per 1 sec, 2 sec, 3 sec, 4 sec, 5 sec, 6 sec, 7 sec, 8 sec, 9 sec, or 10 sec); addition of a ligase to the reaction mixture; and incubation at the second incubation temperature for a second period of time. In some embodiments, the first incubation temperature is about 80° C., 81° C., 82° C., 83° C., 84° C., 85° C., 86° C., 87° C., 88° C., 89° C., 90° C., or a number or a range between any two of these values, optionally 85° C. In some embodiments, the second incubation temperature is about 35° C., 36° C., 37° C., 38° C., 39° C., 40° C., 41° C., 42° C., 43° C., 44° C., 45° C., 46° C., 47° C., 48° C., 49° C., 50° C., 51° C., 52° C., 53° C., 54° C., 55° C., or a number or a range between any two of these values, optionally 50° C. In some embodiments, the first period of time is about 10 sec, 20 sec, 30 sec, 40 sec, 50 sec, 60 sec, 2 min, 3 min, 4 min, 5 min, 6 min, 7 min, 8 min, 9 min, 10 min, or a number or a range between any two of these values, optionally five min. In some embodiments, the second period of time is about 10 min, 20 min, 30 min, 40 min, 50 min, 60 min, 2 hr, 4 hr, 6 hr, 8 hr, 10 hr, 12 hr, or a number or a range between any two of these values, optionally at least one hour.

In some embodiments, the assembly of the n fragments occurs independently of the sequence of the internal payload segments, and the assembly of the n fragments is directed by the formation of the helices between adjacent fragments, and thereby the molecular information which directs assembly of the n fragments is decoupled from the final synthetic sequence. In some embodiments, the assembled product comprises a final synthetic sequence, wherein the final synthetic sequence does not comprise the sequence of the first barcode or the second barcode of any of the n fragments. In some embodiments, the final synthetic sequence comprises a linear polynucleotide comprising the structure 5′-[first payload segment]-[second payload segment] . . . [(n)th payload segment]-3′. In some embodiments, the final synthetic sequence is a linear polynucleotide. In some embodiments, the final synthetic sequence is a circular polynucleotide wherein the 3′ end of the [(n)th payload segment] is linked to the 5′ end of [first payload segment] by a phosphodiester bond. In some embodiments, the final synthetic sequence is at least about 500 bases, 750 bases, 1 kb, 2 kb, 3 kb, 4 kb, 5 kb, 6 kb, 7 kb, 8 kb, 9 kb, 10 kb, 15 kb, 20 kb, 25 kb, 50 kb, 75 kb, 100 kb, 250 kb, 500 kb, 750 kb, 1 MB, or a number or a range between any two of these values, in length. In some embodiments, the ligating step is performed with a ligase, optionally a thermostable ligase, optionally said ligase is selected from the group comprising T3 ligase, T4 ligase, T7 ligase, SplintR, E. coli DNA ligase, Hi-T4 ligase, HiFi Taq ligase, Taq ligase, 9°N, or any combination thereof. In some embodiments, the ligating step comprises contacting the intermediate product with a chemical coupling agent effective to form a covalent linkage between adjacent second polynucleotide strands, optionally one or more click chemistry reagents, optionally CuAAC, SPAAC, iEDDA, oxime formation, hydrazone formation, Michael addition, disulfide formation, carbodiimide mediated coupling, native chemical ligation, or any combination thereof.

In some embodiments, the method further comprises removing the helices to generate a scarless assembly. In some embodiments, the method comprises hybridizing a primer to the first terminal region and extending with a DNA polymerase, optionally a strand-displacing DNA polymerase. In some embodiments, the method comprises PCR amplification of the assembled product, or a product thereof, optionally using a primer capable of binding the first terminal region (or a complement thereof) and/or a primer capable of binding the second terminal region (or a complement thereof). In some embodiments, the base of the 3WJ comprise non-canonical nucleotide(s), and the method comprises: (i) contacting the assembled product with cleavage agent(s) to remove the 3WJ; and (ii) ligating nicks on the first polynucleotide strand, optionally: the non-canonical nucleotide(s) comprises deoxyuridine, deoxyinosine, deoxy-7-methylguanosine, deoxy-5,6-dihydroxythymidine, deoxy-3-methyladenosine, 5-methyl-deoxycytidine, O-6-methyl-deoxyguanosine, 5-iodo-deoxyuridine, 8-oxy-deoxyguanine, 1,N6-ethenoadenine, 8-oxo-guanine (80x0G), or any combination thereof; and/or the cleavage agent(s) comprise USER Enzyme, a DNA glycosylase, an AP cleaving agent, APE 1 (AP Endonuclease 1), Endo III (Endonuclease III), Endo IV (Endonuclease IV), Endo V (Endonuclease V), Endo VIII (Endonuclease VIII), Fpg (formamido-pyrimidine-DNA glycosylase), OGG1 (8-oxoguanine DNA glycosylase 1), NEIL1 (Endonuclease VIII-like 1), T7 Endo I (T7 Endonuclease I), T4 PDG (T4 pyrimidine dimer DNA glycosylase), UDG (uracil DNA glycosylase), SMUG1 (Single-strand selective monofunctional uracil DNA glycosylase), AAG (methylpurine DNA glycosylase), or any combination thereof. In some embodiments, the incubating step comprises combining the n fragments in a single reaction mix at equimolar concentrations, optionally at about 0.1 nM, 0.5 nM, 0.75 nM, 0.9 nM, 1.0 nM, 1.1 nM, 1.25 nM, 1.5 nM, 1.75 nM, 2 nM, 5 nM, 10 nM, or a number or a range between any two of these values.

In some embodiments, the providing step comprises: generating the n fragments. In some embodiments, said generating step comprises annealing the first polynucleotide strand and the second polynucleotide strand components of each of the n fragments to generate heteroduplexes. In some embodiments, the n fragments are each generated in separate reactions. In some embodiments, the generating step comprises phosphorylation of the second polynucleotide strands, further optionally via T4 polynucleotide kinase. In some embodiments, said annealing step comprises an initial denaturation step followed by a gradual decrease in temperature. In some embodiments, the heteroduplexes undergo one or more purification steps, such as: gel electrophoresis, including pulsed-field gel electrophoresis (PFGE); solid or solution phase hybridization/capture; precipitation; dialysis; solid phase reversible immobilization (SPRI) cleanup, optionally performing size selection using SPRI beads, further optionally single-sided or double-sided; and/or column purification.

In some embodiments, the method further comprises PCR amplification of the assembled product, or a product thereof, to generate an amplified product. In some embodiments, PCR amplification comprises amplifying the assembled product, or a product thereof, using a primer capable of hybridizing to the first terminal region or a complement thereof, and a primer capable of hybridizing the second terminal region or a complement thereof. In some embodiments, the method comprises purification of the assembled product, the amplified product, or products thereof. In some embodiments, said purification step compromises: gel electrophoresis of the assembled product, the amplified product, or products thereof; solid phase reversible immobilization (SPRI) cleanup, optionally performing size selection using SPRI beads, further optionally single-sided or double-sided; and/or column purification. In some embodiments, the method comprises replication of the assembled product, the amplified product, or products thereof, in a cell.

In some embodiments, the providing step comprises providing y sets of n fragments. In some embodiments, the incubating step comprises incubating the y sets of n fragments in a single reaction mixture, wherein the n fragments of each set are joined together in parallel via three-way junction (3WJ) intermediates to generate y intermediate products. In some embodiments, the ligating step comprises ligating nicks on the second polynucleotide strands of each of the y intermediate products to generate y assembled products. In some embodiments, the value of n is the same between at least two of the y sets. In some embodiments, the value of n is the same different between at least two of the y sets. In some embodiments, the first barcode and the second barcode of each set are not complementary to the first barcode and the second barcode of any other set. In some embodiments, the y intermediate products are candidate design variants. In some embodiments, the method comprises the y intermediate products, or products thereof, being individually amplified or universally amplified. In some embodiments, y is an integer greater than 1, optionally at least 2, 3, 4, 5, 6, 7, 8, 9, 10, 11, 12, 13, 14, 15, or a number or a range between any two of these values. In some embodiments, at least one of the n fragments is a variant fragment, and wherein the assembled products comprise a combinatorial library of at least p variants, wherein p is an integer greater than 1. In some embodiments, p is at least about 10, 50, 100, 250, 500, 750, 1000, 10000, 50000, 100000, 250000, 500000, 750000, 1000000, 5000000, 10000000, or a number or a range between any two of these values. In some embodiments, the combinatorial library achieves a variant coverage of at least 80%, 81%, 82%, 83%, 84%, 85%, 86%, 87%, 88%, 89%, 90%, 91%, 92%, 93%, 94%, 95%, 96%, 97%, 98%, 99%, 99.99%, or a number or a range between any two of these values, of the theoretical variant library. In some embodiments, every codon mutation profile is represented in the library with an average absolute deviation of less than about 10%, 9%, 8%, 7%, 6%, 5%, 4%, 3%, 2%, 1%, 0.1%, 0.01%, or a number or a range between any two of these values, from the theoretical proportion of occurrence for that codon.

In some embodiments, at least 95%, 96%, 97%, 98%, 99%, 99.9%, 99.99%, 99.999%, 9.9999%, or a number or a range between any two of these values, of the assembled products, or products thereof, comprise all of the intended payload segments in the intended order. In some embodiments, less than 10%, 9%, 8%, 7%, 6%, 5%, 4%, 3%, 2%, 1%, 0.1%, 0.01%, or a number or a range between any two of these values, of the assembled products, or products thereof, are a partial assembly missing one or more payload segments. In some embodiments, less than 1 in 1000, 1 in 10000, 1 in 100000, 1 in 1000000, 1 in 10000000, 1 in 100000000, or a number or a range between any two of these values, of the assembled products are missing one or more payload segments or comprise a mis-assembled junction. In some embodiments, the mis-ligation rate at the 3WJ is less than 1 in 1000, 1 in 10000, 1 in 100000, 1 in 1000000, 1 in 10000000, 1 in 100000000, or a number or a range between any two of these values. In some embodiments, n is at least 3, 4, 5, 6, 7, 8, 9, 10, 11, 12, 13, 14, 15, 16, 17, 18, 19, 20, 22, 24, 26, 28, 30, 32, 34, 36, 38, 40, 42, 44, 46, 48, 50, 55, 60, 65, 70, 75, 80, 85, 90, 95, 100, 125, 150, 175, 200, 225, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, 1000, 10-25, 10-50, 10-75, 10-100, 10-500, 10-1000, 25-50, 25-75, 25-100, 25-500, 25-1000, 50-75, 50-100, 50-500, 50-1000, 75-100, 75-500, 75-1000, 100-500, 100-1000, 500-1000, or a number or a range between any two of these values. In some embodiments, the yield of correctly assembled products is at least 1-fold, 2-fold, 4-fold, 8-fold, 10-fold, 20-fold, 50-fold, 100-fold, 500-fold, or 1000-fold, greater than the yield of a polynucleotide assembly method not comprising 3WJ, optionally Polymerase Cycling Assembly (PCA), Gibson assembly, USER, Yeast Assembly, Homologous Recombination, and/or Golden Gate assembly. In some embodiments, at least about 10%, 15%, 20%, 25%, 30%, 35%, 40%, 45%, 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or a number or a range between any two of these values, of the incubated fragments become a component of an assembled product.

Provided herein include compositions comprising assembled products, or products thereof, generated by a method disclosed herein. In some embodiments, the composition comprises a plurality of cells comprising the assembled products, or products thereof. Disclosed herein include methods. The method can comprise: providing a combinatorial library disclosed herein, or a product thereof; expressing the one or more payload genes in cell(s); and screening for a property of interest. In some embodiments, screening comprises fluorescence-activated cell sorting (FACS), cell viability assay, ELISA, co-immunoprecipitation, a bead-based immunoassay, or any combination thereof. In some embodiments, the property of interest comprises modified enzymatic activity, improved enzymatic activity, modified binding activity, improved binding activity, modified stability, improved stability, modified localization, improved localization, modified solubility, improved solubility, modified expression, improved expression, modified inhibitor resistance, improved inhibitor resistance, modified substrate specificity, improved substrate specificity, or any combination thereof. In some embodiments, the method exposing the cell(s) to one or more agents. In some embodiments, the one or more agents comprise: one or more of a chemical agent, a pharmaceutical, small molecule, a biologic, a CRISPR single-guide RNA (sgRNA), a small interfering RNA (siRNA), CRISPR RNA (crRNA), a small hairpin RNA (shRNA), a microRNA (miRNA), a piwi-interacting RNA (piRNA), an antisense oligonucleotide, a peptide or peptidomimetic inhibitor, an aptamer, an antibody, an intrabody, or any combination thereof; an expression vector, wherein the expression vector encodes one or more of the following: an mRNA, an antisense nucleic acid molecule, a RNAi molecule, a shRNA, a mature miRNA, a pre-miRNA, a pri-miRNA, an anti-miRNA, a ribozyme, any combination thereof; an infectious agent, an anti-infectious agent, or a mixture thereof; a cytotoxic agent, optionally a chemotherapeutic agent, a biologic agent, a toxin, a radioactive isotope, or any combination thereof; and/or one or more of an epigenetic modifying agent, epigenetic enzyme, a bicyclic peptide, a transcription factor, a DNA or protein modification enzyme, a DNA-intercalating agent, an efflux pump inhibitor, a nuclear receptor activator or inhibitor, a proteasome inhibitor, a competitive inhibitor for an enzyme, a protein synthesis inhibitor, a nuclease, a protein fragment or domain, a tag or marker, an antigen, an antibody or antibody fragment, a ligand or a receptor, a synthetic or analog peptide from a naturally-bioactive peptide, an anti-microbial peptide, a pore-forming peptide, a targeting or cytotoxic peptide, a degradation or self-destruction peptide, a CRISPR component system or component thereof, DNA, RNA, artificial nucleic acids, a nanoparticle, an oligonucleotide aptamer, a peptide aptamer, or any combination thereof. In some embodiments, the property of interest comprises a property of the cell, such as improved drug resistance, altered drug sensitivity, improved or modified growth rate under selective pressure, modified or improved cell viability or survival, modified or improved stress tolerance, modified or improved secretion of a compound, altered signaling pathway activation, or any combination thereof. In some embodiments, the method comprises cloning the assembled products, or products thereof, into expression vector(s), optionally prior to an expressing step. In some embodiments, the expression vector is selected from a plasmid, a viral vector, a transposable element, a bacterial artificial chromosome, a yeast artificial chromosome, or any combination thereof. In some embodiments, the cloning step operably connects the final synthetic sequence with one or more regulatory elements selected from a promoter, an enhancer, a polyadenylation signal, a 5′UTR, a 3′ UTR, and a selection marker. In some embodiments, the method comprises transforming or transfecting host cells with the cloned expression vector, optionally bacterial cells for propagation and/or sequence verification and subsequently eukaryotic cells for expression, optionally mammalian, yeast, insect, plant, or fungal cells.

Provided herein include systems and kits for synthesizing nucleic acids. The system or kit can comprise: the n fragments provided herein, optionally: (i) y sets of n fragments; (ii) each of the n fragments is housed in a separate vessel, optionally a tube, a well, or a microfluidic chamber, and/or (iii) the first polynucleotide strand and the second polynucleotide strand that constitute each of the n fragments is housed in a separate vessel, optionally a tube, a well, or a microfluidic chamber. The system or kit can comprise: a non-thermostable ligase, a thermostable ligase, a chemical coupling agent, a polymerase, a primer capable of binding the first terminal region (or a complement thereof), a primer capable of binding the second terminal region (or a complement thereof), or any combination thereof. The system or kit can comprise: a ligation buffer. The ligation buffer can comprise: HiFi Taq buffer; one or more of Tris HCl at about 10 mM to about 200 mM, at a pH of about 7.0 to about 9.5 at the incubation temperature, Mg2+ at about 0.5 mM to about 20 mM, monovalent cation(s) at about 10 mM to about 300 mM, and a reducing agent at about 0.1 mM to about 20 mM; a ligase cofactor, optionally selected from ATP at about 0.05 mM to about 5 mM or NAD+ at about 0.01 mM to about 2 mM; a buffering species selected from Tris, HEPES, Bis Tris, MOPS, and PIPES, optionally configured to maintain pH between 8.3-8.8 at 25° C.; one or more additives, optionally selected from bovine serum albumin at about 0.01 mg/mL to about 1 mg/mL, polyethylene glycol at about 1% to about 20% (w/v), betaine at about 0.1 M to about 2.0 M, dimethyl sulfoxide at about 1% to about 20% (v/v), formamide at about 0.5% to about 10% (v/v), glycerol at about 1% to about 20% (v/v), and/or a non-ionic detergent at about 0.001% to about 0.1% (v/v). The system or kit can comprise; and/or one or more purification reagent(s), such as: gel electrophoresis reagent(s), optionally pulsed-field gel electrophoresis (PFGE); solid or solution phase hybridization/capture reagent(s); precipitation reagent(s); dialysis reagent(s); solid phase reversible immobilization (SPRI) cleanup reagent(s), optionally performing size selection using SPRI beads, further optionally single-sided or double-sided; and/or column purification reagent(s). In some embodiments, the system or kit does not comprise one or more reagents employed with Polymerase Cycling Assembly (PCA), Gibson assembly, USER, Yeast Assembly, Homologous Recombination, and/or Golden Gate assembly, optionally an exonuclease, an endonuclease, a single stranded DNA binding protein, a restriction endonuclease, a recombinase, or any combination thereof.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1C depict non-limiting exemplary schematics and data related to how sidewinder uses 3-Way Junctions (3WJs) to direct DNA assembly in an entirely sequence independent manner. FIG. 1A. Sidewinder directs DNA assembly via the formation of the 3WJ. (i) Sidewinder fragments contain short “toehold” pairs t/t*, and long, unique “barcode” pairs b/b *. (ii) Sidewinder DNA assembly is directed by b/b *. (iii) The association of b/b* brings together the two fragments which is further stabilized by t/t* to form the 3WJ. (iv) Tochold t* is ligated to the neighboring fragment X, irreversibly connecting the two fragments. (v) The barcode helix b/b* is removed to restore the 2-Way Junction (2WJ), resulting in a scarless assembly. FIG. 1B. A 2-fragment ligation requires complementary t/t* and b/b *. Fragment Y heteroduplex, tagged with fluorophore Cy3 (right), undergoes an assembly with one of four possible fragment X heteroduplexes (left) with either matching or mismatching toehold and barcode. Only the fragment X with both a complementary barcode and complementary toehold (i) can be successfully ligated to the fluorophore tagged complex. Mismatched toehold x or/and mismatched barcode y result in no ligation (ii-iv). FIG. 1C. All four reactions from panel b (i-iv) and control (C, fragment Y alone) were run on an unstained TBE-Urea denature gel which allows tracking of the migration of fluorophore containing molecules only. The gel depicts ligation efficiency through the difference in migration of un-ligated product (lower band, control lane C) compared to the ligated product (upper band) which is present only in lane (i).

FIGS. 2A-2F depict non-limiting exemplary schematics and data related to how sidewinder scales to both ends of an arbitrary number of DNA fragments simultaneously. FIG. 2A. Sidewinder fragment i heteroduplexes are generated by annealing ssDNA barcode and coding oligos together to form a stable heteroduplex. FIG. 2B. (i) Sidewinder fragments are individually processed as in panel a prior to being mixed together in the assembly reaction. (ii) The fragments associate with their proper assembly partner through the direction of their high-fidelity barcodes bⁱ/bⁱ*and are ligated subsequent to the formation of the 3WJs, resulting in the 3WJ assembly. (iii) All barcode oligos are either displaced or destroyed through DNA polymerase extension of primer p^{F}, restoring the 2WJ throughout the assembly. This conversion step can be integrated as a part of the selective Polymerase Chain Reaction (PCR) with primers p^{F} and p^{R} to further amplify the assembled Sidewinder product. FIG. 2C. DNA agarose gel depicting the PCR product comparing the industry standard DNA assembly technique from oligos (PCA) to Sidewinder with increasing assembly size and number of fragments. A segment of the LuxABCDE cassette was assembled with 5, 10, and 20-piece assemblies for both techniques and a 40-piece assembly for Sidewinder only. FIG. 2D. Analysis of the 40-piece Sidewinder assembly Nanopore sequencing reads depicted as a pie chart colored by proportion accurate assemblies (blue) and proportion of artifacts (grey). FIG. 2E. Analysis of all possible combinations of ligated junctions in the 40-piece Sidewinder assembly Nanopore sequencing comparing the number of correctly and incorrectly ligated junctions. FIG. 2F. Annotation of Fragment 2 of FIG. 2B.

FIGS. 3A-3H depict non-limiting exemplary schematics and data related to how Sidewinder reliably assembles complex DNA sequences with high GC content and high repeats. FIG. 3A. Graphical representation of the local GC content of a 20-nucleotide sliding window in the coding sequence of the human ApoE gene (teal) compared to the GC content of the 10-piece assembly of the Lux cassette (grey). FIG. 3B. DNA agarose gel depicting the high GC assembly post PCR with a single strong target band. FIG. 3C. Nanopore sequencing analysis of the high GC Sidewinder assembly depicted as a pie chart colored by proportion accurate assemblies (teal) and proportion of artifacts (grey). FIG. 3D. Analysis of all possible combinations of ligated junctions in the high GC assembly Nanopore sequencing comparing the number of correctly and incorrectly ligated junctions. FIG. 3E. Self alignment of a segment of LuxA (i) contrasted to the assembled highly repetitive segment of the h-fibroin protein (ii). Places where at least 8 bases repeat are plotted according to the position in the sequence (x axis) and where it repeats (y axis). (iii) The location of the identical toeholds t/t* is highlighted in dark purple corresponding to their position in the assembly. FIG. 3F. DNA agarose gel depicting the identical toehold assembly post PCR (upper band), as well as minor byproducts (lower band) resulting from mis-priming between fragments F1 and F5 during the PCR step and not from mis-assembly during the Sidewinder reaction. FIG. 3G. Nanopore sequencing analysis of the gel extracted identical toehold Sidewinder assembly depicted as a pie chart colored by proportion of Sidewinder products (purple) and proportion of PCR and sequencing artifacts (grey). FIG. 3H. Analysis of all possible combinations of ligated junctions in the identical toehold assembly Nanopore sequencing comparing the number of correctly and incorrectly ligated junctions.

FIGS. 4A-4E depict non-limiting exemplary schematics and data related to how Sidewinder independently assembles multiple distinct constructs in one pot with high fidelity. FIG. 4A. Sidewinder fragments for three 10-piece assemblies corresponding to phenotypic markers mScarlet, mGL, and aeBlue are mixed together in the same reaction tube where they are assembled in parallel. This reaction mix is then used as the template for a PCR reaction that can individually amplify target constructs or universally amplify the pool of all constructs simultaneously. FIG. 4B. DNA agarose gel depicting the final PCR for each of the individual constructs as well as all three simultaneously (Pool) with a single strong target band. FIG. 4C. Nanopore sequencing analysis of the individual and Pool assemblies depicted as pie charts colored by proportion Sidewinder Products corresponding to mScarlet (red), mGL (green), and aeBlue (blue) as well as proportion of PCR and sequencing artifacts and incorrect assemblies (grey). FIG. 4D. Analysis of all possible combinations of ligated junctions for the individually amplified construct's Nanopore sequencing comparing the number of correctly and incorrectly ligated junctions. FIG. 4E. Assemblies were cloned and transformed. The pool plate is colored using superimposed images of the plate under ambient light and a blue light.

FIGS. 5A-5L depict non-limiting exemplary schematics and data related to how Sidewinder generates large combinatorial libraries with high coverage. FIG. 5A. Sidewinder library fragments are generated by annealing a barcode oligo to an arbitrary number of library specific coding oligos containing predefined mutations (colored diamonds). FIG. 5B. Schematic of the 10-piece assembly conducted to generate the fluorescent protein library (position not to scale). FIG. 5C. DNA agarose gel depicts PCR product of library assembly with a single strong target band. FIG. 5D. PacBio sequencing analysis of the pre-clonal Sidewinder library depicted as a pie chart with proportion Sidewinder Products (pale orange), proportion of partial aligned (subset of fragments 1-10 in the correct order) and PCR artifacts and barcode artifacts (grey). FIG. 5E. Junction analysis of the library PacBio sequencing comparing the number of correctly and incorrectly ligated junctions. FIG. 5F. Violin plot depicts the distribution of per-base accuracies for the oligos used in the assembly, excluding intended library mutation positions and the flanking bases. FIG. 5G. Mutation diversity at the codon level showing pre-cloning experimental distribution (saturated, left) and the corresponding theoretical codon distribution (desaturated, right). FIG. 5H. Mutation diversity at the gene level showing the proportion of PacBio reads assigned to each of the possible mutation combinations pre-cloning (pale orange) and post-cloning (orange). FIG. 5I. Venn diagram depicting the sequence space of all possible mutation combinations (grey) and the mutation combinations represented from the pre-clonal sequencing (pale orange), post-clonal sequencing (orange) and those combinations seen in both (dark orange).

FIG. 5J. Proportion of all observed variants in the pre-clonal and post-clonal sequencing plotted relative to one another. FIG. 5K. Percentage of diversity achieved considering every combination of N mutation positions across the 17 diversity positions of the Sidewinder library. FIG. 5L. Fluorescence area vs. height plots showing populations positive for blue, green, yellow, and red fluorescence, respectively. Proportion of hits identified over the threshold is labeled for each color.

FIGS. 6A-6C depict non-limiting exemplary schematics related to how conventional DNA assembly techniques rely on the formation of a 2WJ using information within the synthetic construct to direct assembly. FIG. 6A. DNA assembly via single stranded overhangs generated using enzymes. Enzymes which nick, cleave, or digest a portion of the dsDNA strand are used to expose complementary sequences on assembly fragments which are part of the final synthetic sequence and direct assembly between two fragments. FIG. 6B. DNA assembly via single stranded overhangs generated by denaturation and annealing. Complementary ends of DNA on assembly fragments which are part of the final synthetic sequence come together after denaturation and annealing and direct assembly between two fragments. FIG. 6C. Sub-optimal overhangs relying on information found within the final synthetic construct generated from all techniques inevitably lead to unintended byproducts which decreases the overall efficiency and fidelity of assembly.

FIGS. 7A-7C depict non-limiting exemplary data related to how Sidewinder 3WJ ligation efficiency is dependent on the position of the nick relative to 3WJ and ligase used. FIG. 7A. Comparison of 15 different 2-piece assemblies with varying toehold lengths on the left (negative numbers) and on the right (positive numbers) of the 3WJ. Ligations run on an unstained TBE-Urea denature gel allows tracking of the migration of fluorophore containing molecules only, free from hydrogen-bond interactions as in FIG. 1. The gel depicts ligation efficiency through the difference in migration of un-ligated product (lower band) compared to the ligated product (upper band). FIG. 7B. Comparison of 6 different 2-piece assemblies with varying toehold lengths using Taq ligase. FIG. 7C. DNA agarose gel of the PCR product of a 10-piece assembly of mScarlet with a toehold length of −10 using different ligases during assembly: T3 ligase (i), T4 ligase (ii), T7 ligase (iii), SplintR (iv), E. coli DNA ligase (v), Hi-T4 ligase (vi), HiFi Taq ligase (vii), Taq ligase (viii), 9°N (ix) all acquired from New England Biolabs. Ligation efficiency determined by strength and purity of the correct size band post amplification.

FIGS. 8A-8F depict non-limiting exemplary schematics and data related to how Sidewinder outperforms conventional DNA assembly technologies. FIG. 8A. Analogous assembly fragments to the Sidewinder fragments in FIG. 2 were established with 4 alternative assembly techniques. Oligos ordered to cover the same regions as Sidewinder assembly fragments are phosphorylated and annealed. FIG. 8B. Assembly fragments are assembled according to their respective protocol to accurately reflect the conventional technique. FIG. 8C. Post assembly products are used as the template for a PCR reaction to amplify the final product as is done with Sidewinder. FIG. 8D. DNA agarose gel depicting the PCR product (50 ng product for PCA and 1 μL product for other techniques) of a segment of the LuxABCDE cassette assembled with 5, 10, and 20-piece assemblies for conventional assembly techniques. FIG. 8E. DNA agarose gel depicts PCR product comparing the industry standard DNA assembly technique from oligos (PCA) to Sidewinder with increasing assembly size. An mGL and mScarlet fusion cassette was assembled with 5, 10, and 20-piece assemblies for both techniques. Only Sidewinder is successful after 5 pieces for all assembly sizes. FIG. 8F. DNA agarose gel depicts PCR product of the 40-piece Sidewinder assembly for LuxABC using low-purity standard desalt oligos. The minus depicts a negative control for the PCR with the assembly buffer as template.

FIGS. 9A-9B depict non-limiting exemplary schematics related to a proposed mechanism for mis-priming of unreacted Sidewinder oligos with both a left and right toehold design. FIG. 9A. Un-ligated DNA fragments are present in the PCR reaction after the Sidewinder assembly. During normal PCR cycling, unreacted fragment oligos are denatured and oligos with a sufficiently complementary toehold to elsewhere in the sequence (internal or matching toeholds) can act as primers, anneal together and extend by the polymerase. The truncated products can then act as the template for PCR and are amplified. FIG. 9B. Due to antiparallel rules and the 3′ extension by polymerases, un-ligated right toehold 3WJ assemblies can participate in mis-priming but require one additional cycle before exponential amplification of the mis-amplified target product.

FIGS. 10A-10C depict non-limiting exemplary schematics and data related to processing and fidelity of the Sidewinder helix from the Sidewinder 3WJ assembly. FIG. 10A. A 3WJ analogous to those formed in Sidewinder is annealed using three 120mer oligos, two of which contain either a thymine or a deoxyuracil at the base of the Sidewinder helix. Treatment with the USER enzyme overnight at 37° C. excises out the deoxyuracil, releasing that strand, removing the Sidewinder helix in condition (ii). FIG. 10B. DNA agarose gel depicting conditions T-T (i), U-U (ii), T-U (iii), U-T (iv) showing the 180 nucleotide 3WJ (upper band), partially digested or completely digested 3WJ coding helix (middle band), and the partially or completely digested Sidewinder helix (lower band). FIG. 10C. Heatmap depicting all observed ligated junctions in the Sidewinder 40-piece sequencing dataset after PCR amplification to remove the 3WJ.

FIGS. 11A-11D depict non-limiting exemplary schematics and data related to how Sidewinder assembles complex sequences where other techniques fail. FIG. 11A. The region of the highly repetitive h-fibroin gene was constructed using a deliberately extreme reaction conditions of a 5 fragment, identical toehold assembly. Sidewinder fragments are designed deliberately to contain identical sequence right-side toeholds. Fragments are annealed individually and all heteroduplexes are mixed in the assembly reaction simultaneously and they associate with their proper assembly partner through the direction of their high-fidelity Sidewinder barcodes. The fragments are ligated together, irreversibly connecting them and forming the 3WJ assembly. The 3WJ assembly is used as the template for a PCR reaction which amplifies the coding strand only, simultaneously removing the 3WJs and amplifying a scarless PCR amplicon. FIG. 11B. DNA agarose gel depicting the PCR product (50 ng product for PCA and 1 μL product for other techniques) for the 12-piece assembly of the high GC content ApoE gene using conventional assembly techniques. Correct size should be 1.0 kb. FIG. 11C. DNA agarose gel depicting the PCR product (50 ng product for PCA and 1 μL product for other techniques) for the 5-piece identical toehold assembly of the segment of h-fibroin using conventional assembly techniques. Correct size should be 0.5 kb. FIG. 11D. Annotation of Fragment 2 of FIG. 11A.

FIGS. 12A-12B depict data related to how in vivo transformation data of the parallel assembly pool is consistent with in vitro Nanopore analysis. FIG. 12A. DNA agarose gels depicting PCR amplicons across the Sidewinder assembly of 56 non-colored colonies post transformation. All truncated amplicons and 5 amplicons of the correct size were sequenced for identifying the source of the loss of phenotype. FIG. 12B. Pie chart depicting the accuracy of the Sidewinder parallel assembly transformants, colored by proportion of accurate assemblies corresponding to mScarlet (red), mGL (green), and aeBlue (blue). The remaining proportions, composed of non-colored clones, are labeled based on an extrapolation of the genotyping data from Panel a. The * on the labels indicates an extrapolated value.

FIG. 13 depicts data related to how optimized Sidewinder design has a junction misconnection rate of almost 1 in 1,000,000. The dot plot depicts the per-junction error rate of each of the Sidewinder assemblies conducted in this study. Sidewinder barcodes are either designed by hand using a pre-generated (pre-gen) set or bespoke using NUPACK. Toeholds were either designed to be identical in sequence or distinct from one another. The final amplification to remove the Sidewinder helixes either amplifies an individual construct or a pool. The High GC and 40-piece assembly had no observed mis-ligated junctions at this sequencing depth.

FIGS. 14A-14C depict non-limiting exemplary schematics and data related to sidewinder library per-base accuracy and fragment level mutation profile characterization. FIG. 14A. Accuracy and identity of each nucleotide for each position in the open reading frame of the Sidewinder library, not including intended mutation positions and the next flanking base/homopolymeric run. Per-base accuracy is highest surrounding fragment junctions (dashed line) and decreases from 3′ to 5′ prime of the coding oligo. FIG. 14B. Box and whisker plots depicting the designed mutation diversity by fragment of the pre-clonal Sidewinder library. Each intended fragment mutation profile (dots) are distributed according to their fold change from the theoretical proportion of occurrences for that fragment mutation profile. Fragments 1, 3, and 5 did not contain any designed mutated codons and Fragments 4 and 7 had the highest number of possible mutation profiles. Refer to FIG. 5B for fragment design. FIG. 14C. Box and whisker plots containing the same data points as in Panel b but now replotted according to number of nucleotide mismatches between barcode and coding oligo. This hamming distance is characterized by the number of mismatched bases between the universal barcode oligo and variable coding oligo for any given mutation profile due to the intended design of the Sidewinder library. Each mutation profile (dots) are distributed according to their fold change from the theoretical proportion of occurrences for that mutation profile in its respective fragment. Low sampling of large hamming distances may contribute to data bias.

FIGS. 15A-15D depict non-limiting exemplary schematics and data related to screening and characterization of the combinatorial library of fluorescent protein variants using Fluorescence-Activated Cell Sorting (FACS). FIG. 15A. Schematic of the FACS workflow, showing encapsulation of transformed clones into hydrogel microparticles, fluorescence excitation by lasers, and sorting into multi-well plates based on detected fluorescence signals.

FIG. 15B. Forward scatter vs. side scatter plot, with gating for single colonies. FIG. 15C. Positive hits were then re-cloned into a secondary backbone (clones i-vi) and screened for excitation (dashed) and emission (solid) spectra. FIG. 15D. Fluorescence microscopy images of clones i-vi as patched on induced LB-agar plates.

FIG. 16 depicts non-limiting exemplary schematics related to how all current methods use part of synthetic sequences to form sticky ends a and a* with limited specificity to guide DNA assembly.

FIG. 17 depicts non-limiting exemplary schematics related to how “Sidewinder” uses highly specific external barcode pairs b and b* to guide assembly. “Sidewinder” helix b-b* winds up to the side to join fragments X and Y together via the 3WJ intermediate but is not a part of the final synthetic sequence.

FIG. 18 depicts non-limiting exemplary schematics related to how a high number of unique, highly specific, mutually exclusive “sidewinder” barcode pairs bⁱ and bⁱ*can be readily designed, and the “sidewinder” strategy can be scaled to assemble many DNA building blocks into a defined order with high specificity and yield. After the assembly, the “sidewinder” helices bⁱ/bⁱ*can be enzymatically removed.

FIG. 19 depicts non-limiting exemplary schematics related to how through the “sidewinder” reaction, the top stand is held together with the “sidewinder” helices bⁱ/bⁱ*, while the bottom strand is joint as a single continuous DNA strand with all the fragments linked together. The bottom strand can then be readily PCR amplified with a unique pair of primers primer^{L} and primer^{R} binding to unique regions (p^{L} and p^{R}) of the lower strand of the two terminal fragments to yield the final seamlessly assembled product.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein and made part of the disclosure herein.

All patents, published patent applications, other publications, and sequences from GenBank, and other databases referred to herein are incorporated by reference in their entirety with respect to the related technology.

### Definitions

Unless defined otherwise, technical and scientific terms used herein have the same meaning as commonly understood by one of ordinary skill in the art to which the present disclosure belongs. See, e.g. Singleton et al., Dictionary of Microbiology and Molecular Biology 2nd ed., J. Wiley & Sons (New York, NY 1994); Sambrook et al., Molecular Cloning, A Laboratory Manual, Cold Spring Harbor Press (Cold Spring Harbor, NY 1989). For purposes of the present disclosure, the following terms are defined below.

As used herein, the term “about” shall be being its ordinary meaning, and shall also refer to plus or minus 5% of the provided value.

The terms “polynucleotide” and “nucleic acid” are used interchangeably herein and refer to a polymeric form of nucleotides of any length, either ribonucleotides or deoxyribonucleotides. A polynucleotide can be single-, double-, or multi-stranded DNA or RNA, genomic DNA, cDNA, DNA-RNA hybrids/triple helices, or a polymer including purine and pyrimidine bases or other natural, chemically or biochemically modified, non-natural, or derivatized nucleotide bases. In some embodiments, a polynucleotide comprises a nucleotide sequence encoding a gene product operably linked to one or more expression control elements (e.g., a promoter), as an expression cassette. Any of the RNA sequences disclosed herein may also be DNA (either single-stranded or double-stranded), e.g., wherein “U” is converted to “T.” Any of the DNA sequences disclosed herein may also be RNA, e.g., wherein “T” is converted to “U.”

As used herein, the term “binding” refers to a non-covalent interaction between macromolecules (e.g., between a protein and a nucleic acid). While in a state of non-covalent interaction, the macromolecules are said to be “associated” or “interacting” or “binding” (e.g., when a molecule X is said to interact with a molecule Y, it means that the molecule X binds to molecule Y in a non-covalent manner). Binding interactions can be characterized by a dissociation constant (Kd), for example a Kd of, or a Kd less than, 10⁻⁶ M, 10⁻⁷ M, 10⁻⁸ M, 10⁻⁹ M, 10⁻¹⁰ M, 10⁻¹¹ M, 10⁻¹² M, 10⁻¹³ M, 10⁻¹⁴ M, 10⁻¹⁵ M, or a number or a range between any two of these values. Kd can be dependent on environmental conditions, e.g., pH and temperature. “Affinity” refers to the strength of binding, and increased binding affinity is correlated with a lower Kd.

The terms “complementarity” and “complementary” can mean that a nucleic acid can form hydrogen bond(s) with another nucleic acid based on traditional Watson-Crick base paring rule, that is, adenine (A) pairs with thymine (U) and guanine (G) pairs with cytosine (C). Complementarity can be perfect (e.g. complete complementarity) or imperfect (e.g. partial complementarity). Perfect or complete complementarity indicates that each and every nucleic acid base of one strand is capable of forming hydrogen bonds according to Watson-Crick canonical base pairing with a corresponding base in another, antiparallel nucleic acid sequence. Partial complementarity indicates that only a percentage of the contiguous residues of a nucleic acid sequence can form Watson-Crick base pairing with the same number of contiguous residues in another, antiparallel nucleic acid sequence. In some embodiments, the complementarity can be at least 70%, 80%, 90%, 100% or a number or a range between any two of these values. In some embodiments, the complementarity is perfect, i.e. 100%. For example, the complementary candidate sequence segment is perfectly complementary to the candidate sequence segment, whose sequence can be deducted from the candidate sequence segment using the Watson-Crick base pairing rules.

As used herein, the term “unstable,” when referring to the association of the first toehold and the second toehold of adjacent sidewinder fragments in the absence of the sidewinder helix, can mean that under the stated incubation conditions the toehold-to-toehold interaction does not form or does not persist to an extent sufficient to maintain productive association of the adjacent fragments. Unless otherwise specified, “unstable” is assessed in the reaction buffer and at the strand concentrations used in the assembly reaction immediately prior to ligation, and at the relevant incubation temperature(s) described herein. In some embodiments, “unstable” is defined operationally by one or more of the following criteria, any one of which can be sufficient to meet the requirement. For example, at the incubation temperature, and at a strand concentration of about 0.1-20 nM per strand in an assembly buffer comprising 10-200 mM Tris-HCl, pH 7.0-9.5 at temperature, 25-250 mM monovalent cation(s), and 0.5-10 mM Mg2+, the fraction of toehold-only assembled molecules is less than about 10% or about 5%, as determined by UV-absorbance melting, native gel shift, microscale thermophoresis, fluorescence anisotropy, or another standard hybridization assay. Alternatively, at the incubation temperature and under the assembly buffer and strand concentration conditions, the dissociation of the toehold-only complex exhibits a dissociation rate constant k_{off} of at least about 0.1 s⁻¹ or about 0.5 s⁻¹, corresponding to a mean residence time of no more than about 10 s or about 2 s, respectively, as determined by a standard kinetic assay (e.g., stopped-flow FRET). Alternately, under the incubation conditions, in the absence of barcode hybridization that forms the helix, the toehold-only association does not support detectable ligation of the nicked second polynucleotide strands within the assay time (e.g., less than about 1% ligated product after 1 hour), as assessed by denaturing PAGE or capillary electrophoresis. In contrast, when the sidewinder helix is present under the same buffer, concentration, and temperature, ligated product is detected at least at a level of about 5-10% or higher in the same time frame.

As used herein, “melting temperature” or “Tₘ” of, e.g., a nucleic acid region (such as a toehold or barcode), refers to the temperature at which 50% of the molecules are in the duplexed state and 50% are single stranded under a defined set of conditions. Unless otherwise specified, Tₘ values reported herein are predicted or measured under standard salt and strand conditions and can be adjusted depending on the context. Tₘ can determined using a nearest neighbor thermodynamic model with salt correction and strand concentration adjustment. In some embodiments, Tₘ can calculated using a web based calculator provided by Integrated DNA Technologies (IDT OligoAnalyzer), with default parameters for DNA/DNA duplexes (50 mM Na+, no Mg2+, 25° C. reference), and a strand concentration of 0.5 μM per strand; in other embodiments, Tₘ is calculated using IDT OligoAnalyzer with user specified monovalent and divalent ion concentrations and strand concentrations that match the intended reaction conditions. Equivalent calculations can be performed using NUPACK, MELTING, DINAMelt, Primer3, or other software implementing nearest neighbor parameters. For RNA or nucleic acid analogs, the corresponding DNA/RNA or RNA/RNA parameter sets and applicable ion corrections are used. In some embodiments, Tₘ is measured experimentally by UV absorbance (A260) thermal denaturation using a spectrophotometer with temperature control. Measurements are performed in a buffer comprising, e.g., 10 mM sodium phosphate (pH 7.0) and 100 mM NaCl with an oligonucleotide duplex concentration of 1 μM (strand concentration defined as total single stranded equivalents), using a heating/cooling rate of 0.5-1.0° C./min. Tₘ is determined as the midpoint of the first derivative of the melting curve. Equivalent buffer systems (e.g., 10 mM Tris HCl, pH 7.5-8.0, with 50-150 mM NaCl and 0-2 mM MgCl2) may be used provided that the composition is reported and the Tₘ is adjusted or recalculated for the intended reaction conditions.

The term “vector” as used herein, can refer to a vehicle for carrying or transferring a nucleic acid. Non-limiting examples of vectors include plasmids, bacteria, and viruses. The term “construct,” as used herein, can refer to a recombinant nucleic acid that has been generated for the purpose of the expression of a specific nucleotide sequence(s), or that is to be used in the construction of other recombinant nucleotide sequences. As used herein, the term “plasmid” can refer to a nucleic acid that can be used to replicate recombinant DNA sequences within a host organism. The sequence can be a double stranded DNA.

As used herein, the term “promoter” is a nucleotide sequence that permits binding of RNA polymerase and directs the transcription of a gene. Typically, a promoter is located in the 5′ non-coding region of a gene, proximal to the transcriptional start site of the gene. Sequence elements within promoters that function in the initiation of transcription are often characterized by consensus nucleotide sequences. Examples of promoters include, but are not limited to, promoters from bacteria, yeast, plants, viruses, and mammals (including humans). A promoter can be inducible, repressible, and/or constitutive. Inducible promoters initiate increased levels of transcription from DNA under their control in response to some change in culture conditions, such as a change in temperature.

As used herein, the term “operably linked” is used to describe the connection between regulatory elements and a gene or its coding region. Typically, gene expression is placed under the control of one or more regulatory elements, for example, without limitation, constitutive or inducible promoters, tissue-specific regulatory elements, and enhancers. A gene or coding region is said to be “operably linked to” or “operatively linked to” or “operably associated with” the regulatory elements, meaning that the gene or coding region is controlled or influenced by the regulatory element. For instance, a promoter is operably linked to a coding sequence if the promoter effects transcription or expression of the coding sequence.

As used herein, “sequence identity” or “identity” in the context of two nucleic acid or polypeptide sequences makes reference to the nucleotide bases or amino acid residues in the two sequences that are the same when aligned for maximum correspondence over a specified comparison window. When percentage of sequence identity or similarity is used in reference to proteins, it is recognized that residue positions which are not identical often differ by conservative amino acid substitutions, where amino acid residues are substituted with a functionally equivalent residue of the amino acid residues with similar physiochemical properties and therefore do not change the functional properties of the molecule.

As used herein in the term “derived from”, in the context of an amino acid sequence or polynucleotide sequence (e.g., an amino acid sequence “derived from” a conjugation system or a transposase system), is meant to indicate that the polypeptide or nucleic acid has a sequence that is based on that of a reference polypeptide or nucleic acid, and is not meant to be limiting as to the source or method in which the protein or nucleic acid is made. By way of example, the term “derived from” includes homologs or variants of reference amino acid or DNA sequences. As used herein, the term “derived from” can also refer to a specified nucleotide sequence that may be obtained from a particular specified source or species, albeit not necessarily directly from that specified source or species.

Standard techniques can be used for recombinant DNA, oligonucleotide synthesis, and cell culture and transformation (e.g., electroporation, lipofection). Enzymatic reactions and purification techniques can be performed according to manufacturer's specifications or as commonly accomplished in the art or as described herein. The foregoing techniques and procedures can be generally performed according to conventional methods well known in the art and as described in various general and more specific references that are cited and discussed throughout the present specification. See, e.g., Sambrook et al., Molecular Cloning: A Laboratory Manual (2d ed., Cold Spring Harbor Laboratory Press, Cold Spring Harbor, N.Y. (1989)), which is incorporated herein by reference for any purpose. Unless specific definitions are provided, the nomenclatures utilized in connection with, and the laboratory procedures and techniques of, analytical chemistry, synthetic organic chemistry, and medicinal and pharmaceutical chemistry described herein are those commonly known and used in the art. Standard techniques can be used for chemical syntheses, chemical analyses, pharmaceutical preparation, formulation, and delivery, and treatment of patients.

### “Sidewinder” Three-Way Junction as a Novel Method of DNA Assembly

To generate longer DNA strands, multiple smaller DNA pieces need to be assembled in the correct order. Provided herein are methods, compositions, systems, and kits for DNA assembly using DNA three-way junctions (3WJ) to assemble a number of DNA fragments to a bigger size.

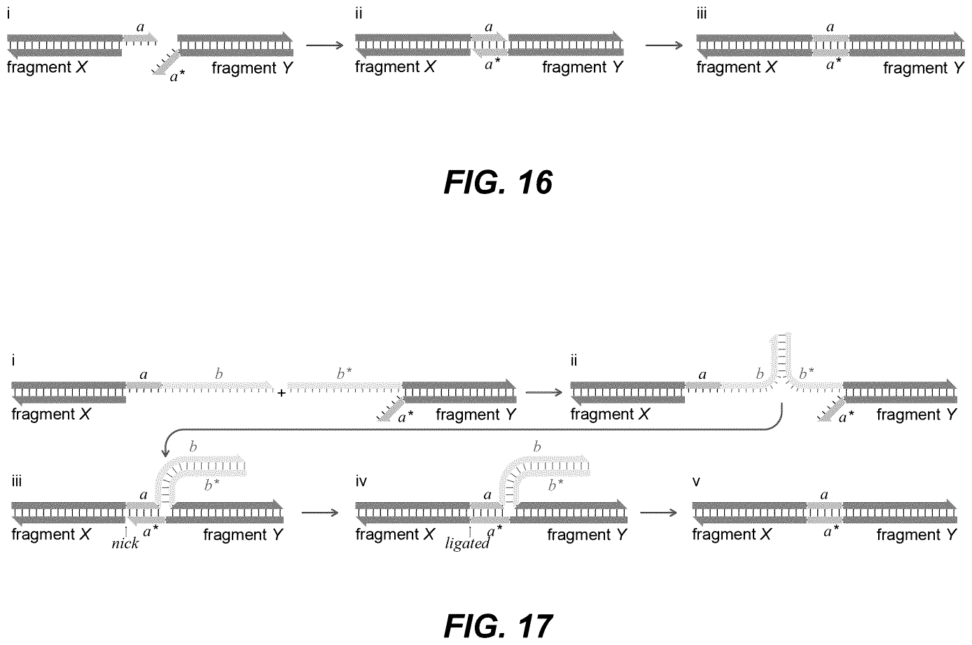

Current assembly methods share the same intrinsic limitation. The pairing of complementary top and bottom single-stranded “sticky ends” (a and a* in FIG. 16) catalyzes the assembly between fragments (X and Y in FIG. 16). In all current methods, the sticky ends ala* become a part of the final synthetic coding sequence connecting fragments X and Y (FIG. 16), thus they cannot be extensively optimized to prevent partial annealing among incorrect ends, leading to mis-assembled final products. This intrinsically limits the number of pieces, and therefore DNA sizes, that can be assembled with high accuracy, yield, throughput, and sequence complexity.

To overcome this ubiquitous limitation, provided herein are methods, compositions, systems, and kits employing a “Sidewinder” strategy which implements highly specific external barcodes that are not incorporated into the final assembled product (FIG. 17). In some embodiments “Sidewinder” uses a highly specific DNA barcode pair (b and b* in FIG. 17, i) to form an external 3^{rd} helix (FIG. 17, ii & iii) to hold synthetic fragments together (FIG. 17, iii) at a temperature prohibiting interactions of very short complementary sequences a and a* alone before enzymatically ligating the nick in the lower strand (FIG. 17, iv) to covalently fix the connection between fragments. A user can then remove the b/b* “sidewinder” helix either enzymatically, or by PCR amplification of the lower strand without the b/b* “sidewinder” helix, to form a seamless connection (FIG. 17, v).

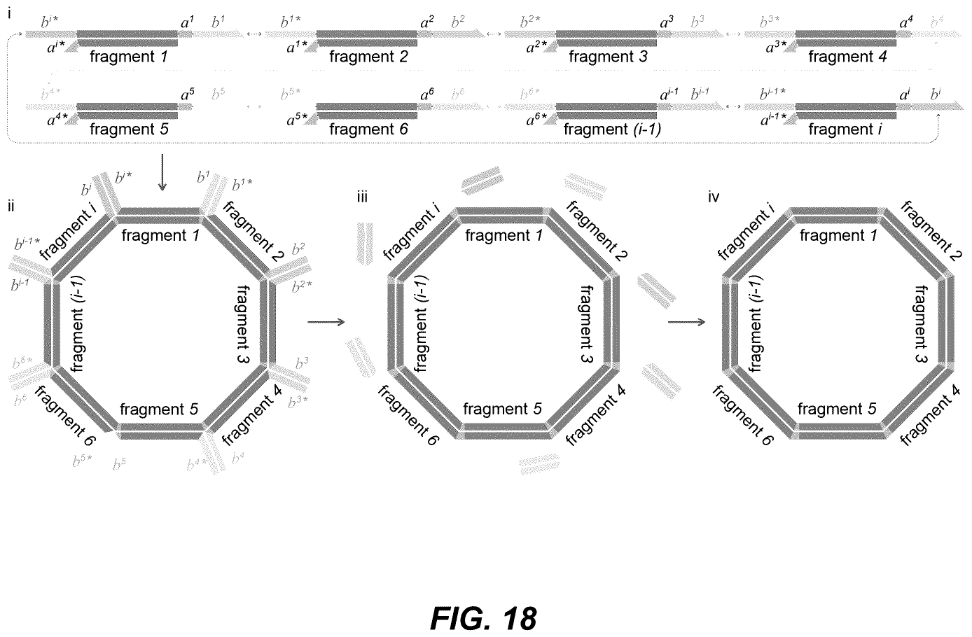

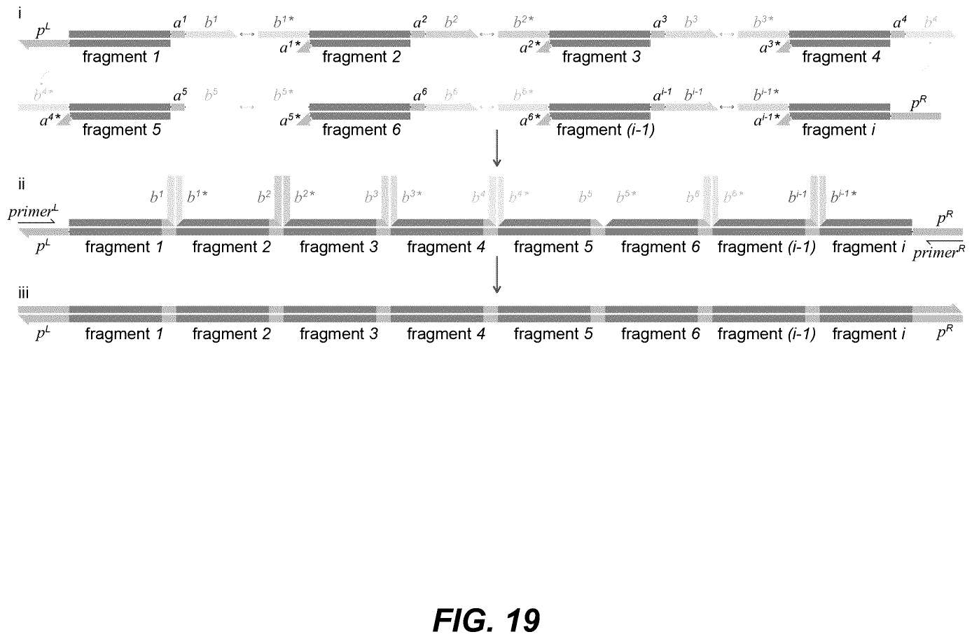

By relying on non-coding, highly mutually exclusive external barcodes bⁱ and bⁱ*to dictate assembly, this principle can be easily scaled up to both ends of a very large number of DNA fragments (FIG. 18; FIG. 19) in some embodiments. The many “Sidewinder” helices can be removed either enzymatically (FIG. 18), or by PCR amplification of the “sidewinder-free” lower strand (FIG. 19) in some embodiments.

Provided herein are methods, compositions, systems, and kits employing “Sidewinder” that offer intrinsic advantages over all current DNA assembly methods. Not restricted to being a part of the final assembly sequences, the “sidewinder” barcodes bⁱ and bⁱ* (FIGS. 17-19) can be extensively optimized for maximum mutual specificity within a pair and absolute exclusivity across different pairs. These designed barcodes can be readily adapted for “sidewinder” strategy, enabling assembly of a high number of building blocks with high specificity and yield to a bigger size.

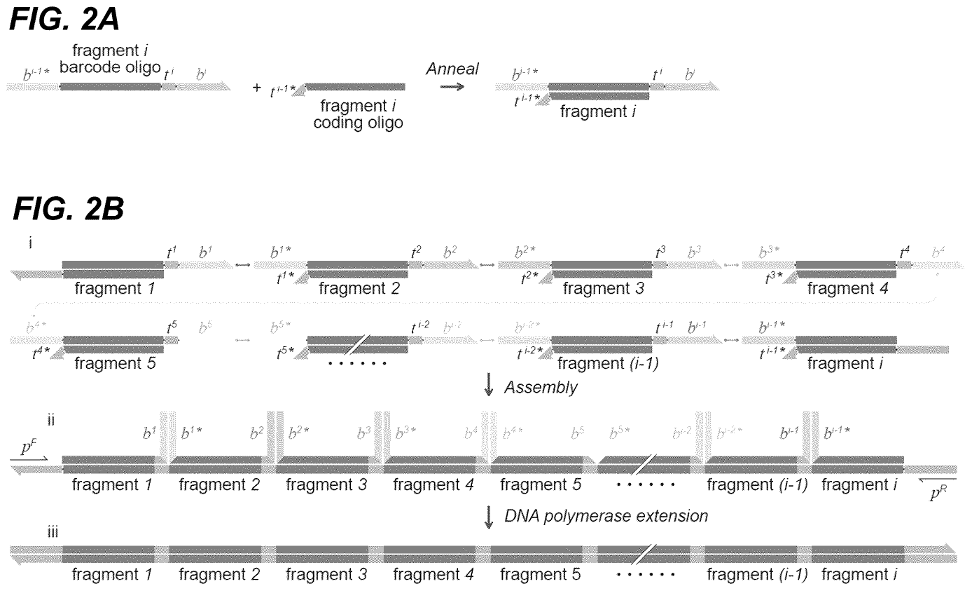

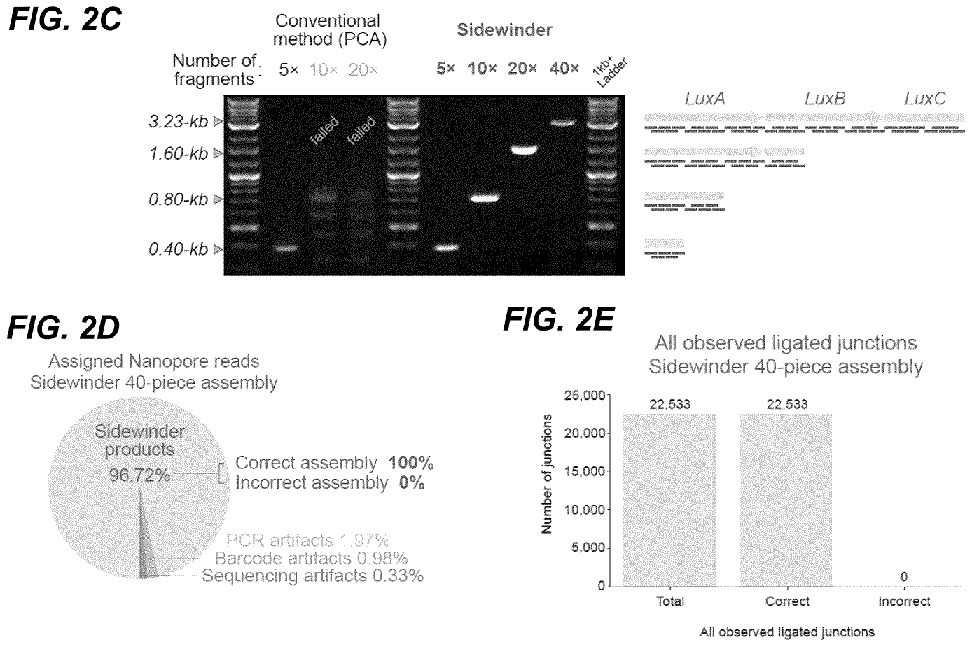

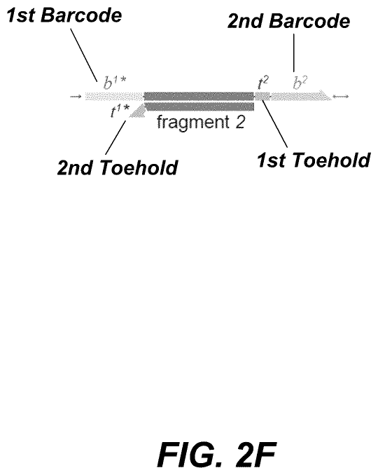

Disclosed herein include compositions. The composition can comprise: n fragments, wherein n is an integer greater than 2. Each fragment can comprise a first polynucleotide strand and a second polynucleotide strand. Each (i)th fragment can comprise a first barcode, a first toehold, a second barcode, and a second toehold, wherein 1<i<n. The first fragment can comprise a first terminal region, a second barcode, and a first toehold, optionally the first terminal region is a 5′ first terminal region. The (n)th fragment can comprise a first barcode, a second toehold, and a second terminal region, optionally the second terminal region is a 3′ second terminal region. In some embodiments, for each (i)th fragment, wherein 1<i<n: the first polynucleotide strand comprises a 5′ overhang and a 3′ overhang; the 5′ overhang of the first polynucleotide strand comprises the first barcode; the 3′ overhang of the first polynucleotide strand comprises the second barcode; the first barcode of the (i)th fragment is complementary to the second barcode of the (i−1)th fragment; the first toehold of the (i)th fragment is complementary to the second toehold of the (i+1)th fragment; the second barcode of the (i)th fragment is complementary to the first barcode of the (i+1)th fragment; and the second toehold of the (i)th fragment is complementary to the first toehold of the (i−1)th fragment. The methods, compositions, systems, and kits provided herein can comprise the generation of a linear product (See FIG. 2B).

Disclosed herein include compositions. The composition can comprise: n fragments, wherein n is an integer greater than 2. Each fragment can comprise a first barcode, a first toehold, a second barcode, and a second toehold. Each fragment can comprise a first polynucleotide strand and a second polynucleotide strand. The first polynucleotide strand can comprise a 5′ overhang and a 3′ overhang. The 5′ overhang of the first polynucleotide strand can comprise the first barcode. The 3′ overhang of the first polynucleotide strand can comprise the second barcode. In some embodiments, for each (i)th fragment, wherein 1<i<n: the first barcode of the (i)th fragment is complementary to the second barcode of the (i−1)th fragment; the first toehold of the (i)th fragment is complementary to the second toehold of the (i+1)th fragment; the second barcode of the (i)th fragment is complementary to the first barcode of the (i+1)th fragment; and the second toehold of the (i)th fragment is complementary to the first toehold of the (i−1)th fragment. The first barcode of the first fragment can be complementary to the second barcode of the (n)th fragment. The second toehold of the first fragment can be complementary to the first toehold of the (n)th fragment. The methods, compositions, systems, and kits provided herein can comprise the generation of a circular product (See FIG. 18).

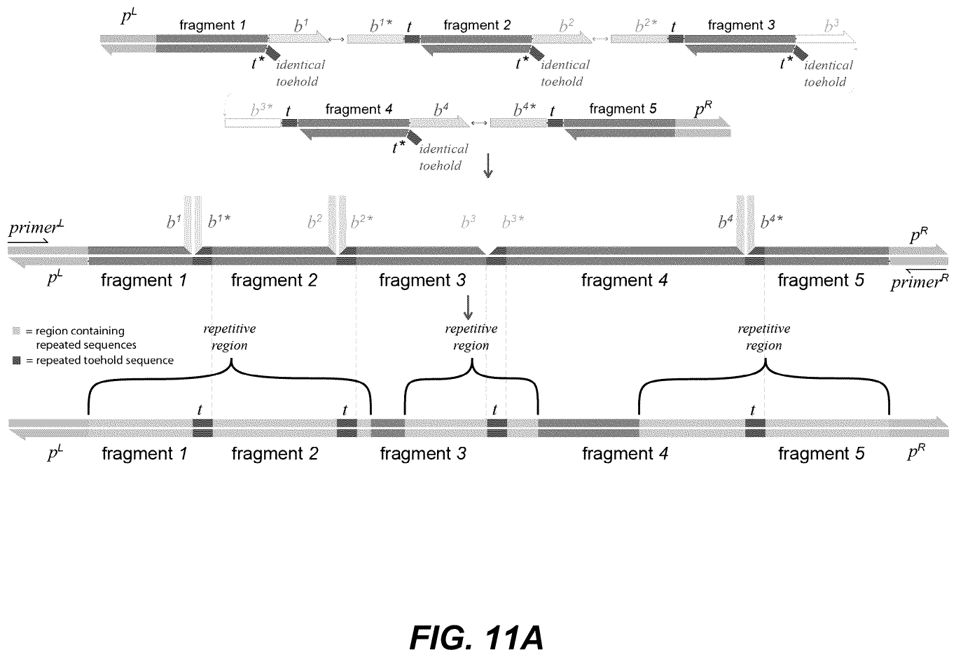

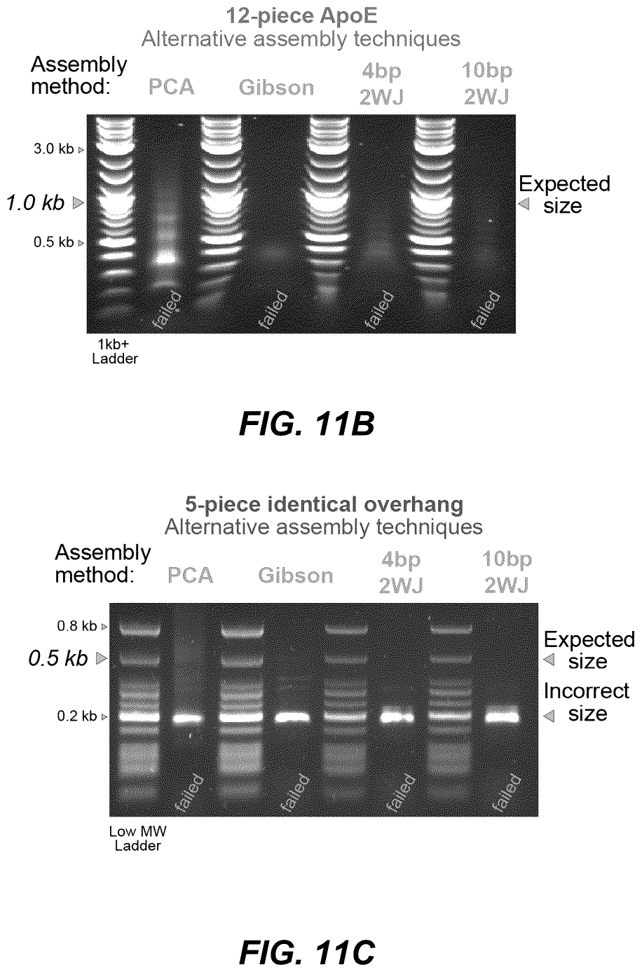

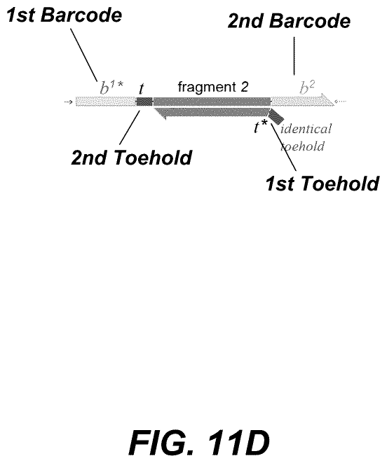

In some embodiments, for each (i)th fragment, wherein 1<i<n: the first barcode of the (i)th fragment is not complementary to the first barcode of any of the n fragments; and the first barcode of the (i)th fragment is not complementary to the second barcode of any (k)th fragment, wherein k is an integer not equal to (i-1). In some embodiments, the 3′ overhang of the first polynucleotide strand comprises the first toehold, the first toehold is 5′ of the second barcode, the second polynucleotide strand comprises a 3′ overhang, and the 3′ overhang of the second polynucleotide strand comprises the second toehold (See FIG. 2F). In some embodiments, the 5′ overhang of the first polynucleotide strand comprises the second toehold, the second toehold is 3′ of the first barcode, the second polynucleotide strand comprises a 5′ overhang, and the 5′ overhang of the second polynucleotide strand comprises the first toehold (See FIG. 11D).

Said complementarity can be or can comprise: at least 80%, 85%, 90%, 95%, 99%, or 100% complementarity; less than five, four, three, two, or one, base pair mismatches; reverse complementarity; canonical Watson-Crick base pairing; wobble base pairing, optionally G-U wobble; and/or DNA nanotechnology interactions, optionally Hoogsteen base pairing, G-quadruplex(es), DNA origami, aptamer-ligand interactions, or any combination thereof. At least 80%, 85%, 90%, 95%, 99%, or 100% of the fragments can comprise a payload segment. At least 80%, 85%, 90%, 95%, 99%, or 100% of the fragments can comprise a toehold-flanked internal payload segment. The payload segment can comprise the sequence of the first toehold and/or the second toehold. In some embodiments, the payload segment does not comprise the sequence of the first barcode or the second barcode.

In some embodiments, the first fragment, the (i)th fragment, the (n)th fragment, one or more of the n fragments, the first toehold, the second toehold, the first barcode, the second barcode, the payload segment, terminal region, and/or the internal payload segment: is at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 110, 120, 130, 140, 150, 160, 170, 180, 190, 200, 210, 220, 230, 240, 250, 1-5, 1-10, 10-100, 10-250, 25-50, 25-100, 25-250, 50-100, 50-200, 50-250, 75-100, 75-200, 75-250, 100-150, 100-200, 100-250, 150-200, 150-250, 200-250, or a number or a range between any two of these values, nucleotides in length; comprises a GC content of about 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, 20%-50%, 20%-75%, 20%-100%, 30%-60%, 30%-75%, 30%-100%, 40%-60%, 40%-75%, 40%-100%, 50%-75%, 50%-100%, 60%-75%, 60%-100%, 75%-100%, or a number or a range between any two of these values; comprises a melting temperature (Tₘ) of about 35° C., 36° C., 37° C., 38° C., 39° C., 40° C., 41° C., 42° C., 43° C., 44° C., 45° C., 46° C., 47° C., 48° C., 49° C., 50° C., 51° C., 52° C., 53° C., 54° C., 55° C., 56° C., 57° C., 58° C., 59° C., 60° C., 61° C., 62° C., 63° C., 64° C., 65° C., 66° C., 67° C., 68° C., 69° C., 70° C., 71° C., 72° C., 73° C., 74° C., 75° C., 35° C.-55° C., 35° C.-75° C., 35° C.-100° C., 45° C.-55° C., 45° C.-75° C., 45° C.-100° C., 55° C.-75° C., 55° C.-100° C., 65° C.-75° C., 65° C.-100° C., 75° C.-100° C., or a number or a range between any two of these values; comprises DNA; comprises RNA; and/or comprises one or more nucleic acid analogs, optionally selected from the group consisting of RNA, 2′-O-methyl RNA, locked nucleic acid (LNA), peptide nucleic acid (PNA), morpholino, phosphorodiamidate morpholino oligomer (PMO), HNA, FANA, TNA, ANA, GNA, CeNA, UNA, L-DNA, or any combination thereof.

The melting temperature (Tₘ) of the first barcode and the second barcode can be at least about 5° C., 6° C., 7° C., 8° C., 9° C., 10° C., 11° C., 12° C., 13° C., 14° C., 15° C., 16° C., 17° C., 18° C., 19° C., 20° C., 21° C., 22° C., 23° C., 24° C., 25° C., or a number or a range between any two of these values, higher than the Tₘ of the first toehold and the second toehold. A first barcode can comprise the sequence of the first 1, 2, 3, 4, 5, 6, 7, 8, 9, 10, 11, 12, 13, 14, 15, 16, 17, 18, 19, or 20, nucleotides, of any one of SEQ ID Nos: 1-548 or SEQ ID Nos: 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, and 300. A second barcode can comprise the sequence of the final 1, 2, 3, 4, 5, 6, 7, 8, 9, 10, 11, 12, 13, 14, 15, 16, 17, 18, 19, or 20, nucleotides, of any one of SEQ ID Nos: 1-548 or SEQ ID Nos: 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, and 300. In some embodiments, the fragments, the first polynucleotide strand, and/or the second polynucleotide strand: comprise or are derived from synthetic oligonucleotides; and/or comprise or are derived from rolling circle amplification products, restriction enzyme digestion products, reverse transcription products, CRISPR-excised products, PCR amplification products, template-independent polymerase products, recombinase-generated products, phage-derived products, or any combination thereof.

In some embodiments, the first barcode of the (i)th fragment forms a pair with the second barcode of the (i−1)th fragment. In some embodiments, the second barcode of the (i)th fragment forms a pair with the first barcode of the (i+1)th fragment. Each pair can be optimized for maximum mutual specificity within a pair and absolute exclusivity across different pairs. The integer n can be at least 3, 4, 5, 6, 7, 8, 9, 10, 11, 12, 13, 14, 15, 16, 17, 18, 19, 20, 22, 24, 26, 28, 30, 32, 34, 36, 38, 40, 42, 44, 46, 48, 50, 55, 60, 65, 70, 75, 80, 85, 90, 95, 100, 125, 150, 175, 200, 225, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, 1000, 10-25, 10-50, 10-75, 10-100, 10-500, 10-1000, 25-50, 25-75, 25-100, 25-500, 25-1000, 50-75, 50-100, 50-500, 50-1000, 75-100, 75-500, 75-1000, 100-500, 100-1000, 500-1000, or a number or a range between any two of these values. Upon incubation in a reaction mixture, the n fragments can be capable of joining together via at least one three-way junction (3WJ) intermediate to generate an intermediate product. A ligase can be capable of ligating nicks on the second polynucleotide strands of said intermediate product to generate an assembled product. A ligase and/or a chemical coupling agent can be capable of forming a covalent linkage between adjacent second polynucleotide strands of said intermediate product to generate an assembled product. The covalent linkage can be formed by a click ligation between complementary reactive handles on adjacent second polynucleotide strands, optionally copper (I)-catalyzed azide-alkyne cycloaddition (CuAAC), strain promoted azide-alkyne cycloaddition (SPAAC), or inverse electron demand Diels-Alder (iEDDA) reaction between a trans cyclooctene and a tetrazine oxime formation, hydrazone formation, Michael addition, disulfide formation, carbodiimide-mediated coupling, native chemical ligation, or any combination thereof. The second polynucleotide strands can comprise synthetic modifications and/or modified synthetic nucleotides, optionally selected a 5′ alkyne, a 3′ azide, a trans-cyclooctene, a tetrazine, a 5′ amine, an aldehyde, an aminooxy group, a thiol, a maleimide, or a phosphorothioate, or any combination thereof. The chemical coupling agent can comprise a click chemistry reagent, a copper (I) source, a copper (I)-stabilizing ligand, a strain-promoted cycloaddition reagent, a tetrazine, an EDC or other carbodiimide, an aniline or p-phenylenediamine catalyst, or any combination thereof.

In some embodiments, the assembled product comprises a final synthetic sequence, wherein the final synthetic sequence does not comprise the sequence of the first barcode or the second barcode of any of the n fragments, and wherein the final synthetic sequence comprises the scarless assembly of the payload segments of the n fragments. The lengths of the first toehold and the second toehold can be configured to ensure effective ligase docking and ligation of nicks on the second polynucleotide strands of said intermediate product, optionally at least 6 nucleotides in length. The final synthetic sequence can be at least about 500 bases, 750 bases, 1 kb, 2 kb, 3 kb, 4 kb, 5 kb, 6 kb, 7 kb, 8 kb, 9 kb, 10 kb, 15 kb, 20 kb, 25 kb, 50 kb, 75 kb, 100 kb, 250 kb, 500 kb, 750 kb, 1 MB, or a number or a range between any two of these values, in length.

In some embodiments, the final synthetic sequence, the first toehold, the second toehold, the payload segment, and/or the internal payload segment: comprises an elevated GC content of at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values; comprises a reduced GC content of less about 40%, 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, 1%, 40%-30%, 40%-20%, 40%-10%, 40%-5%, 40%-1%, 30%-20%, 30%-10%, 30%-5%, 30%-1%, 20%-10%, 20%-5%, 20%-1%, 10%-5%, 10%-1%, 5%-1%, or a number or a range between any two of these values; comprises two or more repeats, optionally tandem repeats, optionally at least 4 nt in length, optionally occurring at least 3, 4, 5, 6, 7, 8, 9, 10, 11, 12, 13, 14, 15, 16, 17, 18, 19, 20 times, or a number or a range between any two of these values, within the final synthetic sequence; and/or comprises two or more mononucleotide stretches, optionally at least 4 nt in length, optionally occurring at least 3, 4, 5, 6, 7, 8, 9, 10, 11, 12, 13, 14, 15, 16, 17, 18, 19, 20 times, or a number or a range between any two of these values, within the final synthetic sequence. In some embodiments, for each (i)th fragment, the first toehold of the (i)th fragment is not complementary to the second toehold of any (k)th fragment, wherein k is an integer not equal to (i+1). In some embodiments, for at least one (i)th fragment, the first toehold of the (i)th fragment is complementary to the second toehold of one or more (k)th fragments, wherein k is an integer not equal to (i+1).

In some embodiments, the first fragment: is an invariant fragment, wherein all instances of the invariant first fragment in the composition are identical; or is a variant fragment, wherein two or more instances of the variant first fragment in the composition differ with respect to the sequence of the internal payload segment. At least one (i)th fragment can be an invariant fragment, wherein all instances of the invariant (i)th fragment in the composition are identical. At least one (i)th fragment can be a variant fragment, wherein two or more instances of the variant (i)th fragment in the composition differ with respect to the sequence of the internal payload segment. The (n)th fragment: can be an invariant fragment, wherein all instances of the invariant (n)th fragment in the composition are identical; or can be a variant fragment, wherein two or more instances of the variant (n)th fragment in the composition differ with respect to the sequence of the internal payload segment. Accordingly, as described herein, the methods, compositions, systems, and kits provided herein can comprise re-use of barcodes among the composition (e.g., for building libraries). Variant fragments can comprise predefined codon variations, optionally codons variations configured to achieve modified and/or improved protein function(s).

The composition can comprise y sets of n fragments. The value of n can be the same between at least two of the y sets. The value of n can be the different between at least two of the y sets. In some embodiments, the first barcode and the second barcode of each set are not complementary to the first barcode and the second barcode of any other set. Upon incubation of the y sets together in a single reaction mixture, each set of n fragments can be capable of, in parallel, joining together via three-way junction (3WJ) intermediates to generate y intermediate products. The y intermediate products can be candidate design variants. The y intermediate products, or products thereof, can be capable of being individually amplified or universally amplified. The integer y can be an integer greater than 1, optionally at least 2, 3, 4, 5, 6, 7, 8, 9, 10, 11, 12, 13, 14, 15, 16, 17, 18, 19, 20, 21, 22, 23, 24, 25, 26, 27, 28, 29, 30, or a number or a range between any two of these values. The final synthetic sequence can comprise one or more payload genes, optionally the one or more payload genes encode one or more RNA payload(s) and/or one or more payload protein(s). The one or more RNA payload(s) can be selected from the group comprising a CRISPR single-guide RNA (sgRNA), a small interfering RNA (siRNA), a CRISPR RNA (crRNA), a small hairpin RNA (shRNA), a microRNA (miRNA), a piwi-interacting RNA (piRNA), an antisense oligonucleotide, an antagomir, an aptamer, a ribozyme, or any combination thereof.

In some embodiments, a payload protein comprises: fluorescence activity, polymerase activity, protease activity, phosphatase activity, kinase activity, SUMOylating activity, deSUMOylating activity, ribosylation activity, deribosylation activity, myristoylation activity demyristoylation activity, or any combination thereof; nuclease activity, methyltransferase activity, demethylase activity, DNA repair activity, DNA damage activity, deamination activity, dismutase activity, alkylation activity, depurination activity, oxidation activity, pyrimidine dimer forming activity, integrase activity, transposase activity, recombinase activity, polymerase activity, ligase activity, helicase activity, photolyase activity, glycosylase activity, acetyltransferase activity, deacetylase activity, adenylation activity, deadenylation activity, or any combination thereof; a biomaterials payload, optionally a structural polypeptide, further optionally silk fibroin, spider silk spidroin, a resilin, a resilin-like polypeptide, an elastin, an elastin-like polypeptide, a collagen, or a collagen-like polypeptide; a cellular reprogramming factor capable of differentiating a given cell into a desired differentiated state, optionally nerve growth factor (NGF), fibroblast growth factor (FGF), interleukin-6 (IL-6), bone morphogenic protein (BMP), neurogenin3 (Ngn3), pancreatic and duodenal homeobox 1 (Pdx1), Mafa, or any combination thereof; an agonistic or antagonistic antibody or antigen-binding fragment thereof specific to a checkpoint inhibitor or checkpoint stimulator molecule, optionally PD1, PD-L1, PD-L2, CD27, CD28, CD40, CD137, OX40, GITR, ICOS, A2AR, B7-H3, B7-H4, BTLA, CTLA4, IDO, KIR, LAG3, PD-1, and/or TIM-3; a secretion tag, optionally the secretion tag is selected from the group comprising AbnA, AmyE, AprE, BglC, BglS, Bpr, Csn, Epr, Ggt, GlpQ, HtrA, LipA, LytD, MntA, Mpr, NprE, OppA, PbpA, PbpX, Pel, PelB, PenP, PhoA, PhoB, PhoD, PstS, TasA, Vpr, WapA, WprA, XynA, XynD, YbdN, Ybxl, YcdH, YclQ, YdhF, YdhT, YfkN, YflE, YfmC, Yfnl, YhcR, YlqB, YncM, YnfF, YoaW, YocH, YolA, YqiX, Yqxl, YrpD, YrpE, YuaB, Yurl, YvcE, YvgO, YvpA, YwaD, YweA, YwoF, YwtD, YwtF, YxaLk, YxiA, and YxkC; a constitutive signal peptide for protein degradation, optionally PEST; a nuclear localization signal (NLS) or a nuclear export signal (NES); a dosage indicator protein, optionally the dosage indicator protein is detectable, optionally the dosage indicator protein comprises green fluorescent protein (GFP), enhanced green fluorescent protein (EGFP), yellow fluorescent protein (YFP), enhanced yellow fluorescent protein (EYFP), blue fluorescent protein (BFP), red fluorescent protein (RFP), TagRFP, Dronpa, Padron, mApple, mCherry, mruby3, rsCherry, rsCherryRev, derivatives thereof, or any combination thereof; a cellular reprogramming factor capable of converting an at least partially differentiated cell to a less differentiated cell, optionally Oct-3, Oct-4, Sox2, c-Myc, Klf4, Nanog, Lin28, ASCL1, MYTIL, TBX3b, SV40 large T, hTERT, miR-291, miR-294, miR-295, or any combinations thereof; a programmable nuclease, optionally the programmable nuclease is selected from the group comprising: SpCas9 or a derivative thereof; VRER, VQR, EQR SpCas9; xCas9-3.7; eSpCas9; Cas9-HF1; HypaCas9; evoCas9; HiFi Cas9; ScCas9; StCas9; NmCas9; SaCas9; CjCas9; CasX; Cas9 H940A nickase; Cas12 and derivatives thereof; dcas9-APOBEC1 fusion, BE3, and dcas9-deaminase fusions; dcas9-Krab, dCas9-VP64, dCas9-Tet1, and dcas9-transcriptional regulator fusions; Dcas9-fluorescent protein fusions; Cas13-fluorescent protein fusions; RCas9-fluorescent protein fusions; Cas13-adenosine deaminase fusions, or any combination thereof; a CRE recombinase, GCaMP, a cell therapy component, a knock-down gene therapy component, a cell-surface exposed epitope, or any combination thereof; a bispecific T cell engager (BiTE); a synthetic receptor, optionally a Synthetic Notch (SynNotch) receptor, a Modular Extracellular Sensor Architecture (MESA) receptor, Tango, dCas9-synR, or any combination thereof; a cytokine, optionally the cytokine is selected from the group consisting of interleukin-1 (IL-1), IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33, IL-34, IL-35, interleukin-1 (IL-1), IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33, IL-34, IL-35, granulocyte macrophage colony stimulating factor (GM-CSF), M-CSF, SCF, TSLP, oncostatin M, leukemia-inhibitory factor (LIF), CNTF, Cardiotropin-1, NNT-1/BSF-3, growth hormone, Prolactin, Erythropoietin, Thrombopoietin, Leptin, G-CSF, or receptor or ligand thereof; a member of the TGF-β/BMP family selected from the group consisting of TGF-β1, TGF-β2, TGF-β3, BMP-2, BMP-3a, BMP-3b, BMP-4, BMP-5, BMP-6, BMP-7, BMP-8a, BMP-8b, BMP-9, BMP-10, BMP-11, BMP-15, BMP-16, endometrial bleeding associated factor (EBAF), growth differentiation factor-1 (GDF-1), GDF-2, GDF-3, GDF-5, GDF-6, GDF-7, GDF-8, GDF-9, GDF-12, GDF-14, mullerian inhibiting substance (MIS), activin-1, activin-2, activin-3, activin-4, and activin-5; a member of the TNF family of cytokines selected from the group consisting of TNF-alpha, TNF-beta, LT-beta, CD40 ligand, Fas ligand, CD 27 ligand, CD 30 ligand, and 4-1 BBL; a member of the immunoglobulin superfamily of cytokines selected from the group consisting of B7.1 (CD80) and B7.2 (B70); an interferon, optionally the interferon is selected from interferon alpha, interferon beta, or interferon gamma; a chemokine, optionally the chemokine is selected from CCL1, CCL2, CCL3, CCR4, CCL5, CCL7, CCL8/MCP-2, CCL11, CCL13/MCP-4, HCC-1/CCL14, CTAC/CCL17, CCL19, CCL22, CCL23, CCL24, CCL26, CCL27, VEGF, PDGF, lymphotactin (XCL1), Eotaxin, FGF, EGF, IP-10, TRAIL, GCP-2/CXCL6, NAP-2/CXCL7, CXCL8, CXCL10, ITAC/CXCL11, CXCL12, CXCL13, or CXCL15; an interleukin, optionally the interleukin is selected from IL-10 IL-12, IL-1, IL-6, IL-7, IL-15, IL-2, IL-18 or IL-21; a tumor necrosis factor (TNF), optionally the TNF is selected from TNF-alpha, TNF-beta, TNF-gamma, CD252, CD154, CD178, CD70, CD153, or 4-1BBL; a factor locally down-regulating the activity of endogenous immune cells; a factor capable of remodeling a tumor microenvironment and/or reducing immunosuppression at a target site of a subject; a chimeric antigen receptor (CAR) or T-cell receptor (TCR), optionally the CAR and/or TCR comprises one or more of an antigen binding domain, a transmembrane domain, and an intracellular signaling domain, optionally wherein the intracellular signaling domain comprises a primary signaling domain, a costimulatory domain, or both of a primary signaling domain and a costimulatory domain; and/or an activity regulator, optionally the activity regulator is capable of reducing T cell activity.

A payload protein can be associated with an agricultural trait of interest selected from the group consisting of increased yield, increased abiotic stress tolerance, increased drought tolerance, increased flood tolerance, increased heat tolerance, increased cold and frost tolerance, increased salt tolerance, increased heavy metal tolerance, increased low-nitrogen tolerance, increased disease resistance, increased pest resistance, increased herbicide resistance, increased biomass production, male sterility, or any combination thereof. A payload protein can be associated with a biological manufacturing process selected from the group comprising fermentation, distillation, biofuel production, production of a compound, production of a polypeptide, or any combination thereof.

The one or more payload genes can be selected from the group comprising a nitrogen fixation gene, a plant stress-induced gene, a nutrient utilization gene, a gene that affects plant pigmentation, a gene that encodes an antisense or ribozyme molecule, a gene encoding an antigen capable of being secreted, a toxin gene, a receptor gene, a ligand gene, a seed storage gene, a hormone gene, an enzyme gene, an interleukin gene, a cytokine gene, a growth factor gene, a transcription factor gene, a transcriptional repressor gene, a DNA-binding protein gene, a recombination gene, a DNA replication gene, a programmed cell death gene, a kinase gene, a phosphatase gene, a G protein gene, a cyclin gene, a cell cycle control gene, a gene involved in transcription, a gene involved in translation, a gene involved in RNA processing, a gene involved in RNAi, an organellar gene, a intracellular trafficking gene, an integral membrane protein gene, a transporter gene, a membrane channel protein gene, a cell wall gene, a gene involved in protein processing, a gene involved in protein modification, a gene involved in protein degradation, a gene involved in metabolism, a gene involved in biosynthesis, a gene involved in assimilation of nitrogen or other elements or nutrients, a gene involved in controlling carbon flux, gene involved in respiration, a gene involved in photosynthesis, a gene involved in light sensing, a gene involved in organogenesis, a gene involved in embryogenesis, a gene involved in differentiation, a gene involved in meiotic drive, a gene involved in self incompatibility, a gene involved in development, a gene involved in nutrient, metabolite or mineral transport, a gene involved in nutrient, metabolite or mineral storage, a calcium-binding protein gene, a lipid-binding protein gene, or any combination thereof.

The one or more payload genes can be selected from the group comprising a gene encoding an enzyme involved in metabolizing biochemical wastes for use in bioremediation, a gene that encodes an enzyme for modifying pathways that produce secondary plant metabolites, a gene that encodes an enzyme that produces a pharmaceutical, a gene that encodes an enzyme that improves or changes the nutritional content of a plant, a gene that encodes an enzyme involved in vitamin synthesis, a gene that encodes an enzyme involved in carbohydrate, polysaccharide or starch synthesis, a gene that encodes an enzyme involved in mineral accumulation or availability, a gene that encodes a phytase, a gene that encodes an enzyme involved in fatty acid, fat or oil synthesis, a gene that encodes an enzyme involved in synthesis of chemicals or plastics, a gene that encodes an enzyme involved in synthesis of a fuel, a gene that encodes an enzyme involved in synthesis of a fragrance, a gene that encodes an enzyme involved in synthesis of a flavor, a gene that encodes an enzyme involved in synthesis of a pigment or dye, a gene that encodes an enzyme involved in synthesis of a hydrocarbon, a gene that encodes an enzyme involved in synthesis of a structural or fibrous compound, a gene that encodes an enzyme involved in synthesis of a food additive, a gene that encodes an enzyme involved in synthesis of a chemical insecticide, a gene that encodes an enzyme involved in synthesis of an insect repellent, a gene controlling carbon flux in a plant, or any combination thereof.

The one or more payload proteins can comprise components of a synthetic protein circuit, optionally payload proteins configured to form one or more logic gates selected from the group comprising an OR logic gate, AND logic gate, NOR logic gate, NAND logic gate, IMPLY logic gate, NIMPLY logic gate, XOR logic gate, and an XNOR logic gate. A payload protein can be capable of modulating the expression, concentration, localization, stability, and/or activity of the one or more endogenous proteins of a cell. The payload protein can be a therapeutic protein or a variant thereof, optionally a therapeutic protein configured to prevent or treat a disease or disorder of a subject, further optionally the subject suffers from a deficiency of said therapeutic protein.

In some embodiments, one or more of the payload gene(s) comprise: a 5′UTR and/or a 3′UTR; a tandem gene expression element selected from the group an internal ribosomal entry site (IRES), foot-and-mouth disease virus 2A peptide (F2A), equine rhinitis A virus 2A peptide (E2A), porcine teschovirus 2A peptide (P2A) or Thosea asigna virus 2A peptide (T2A), or any combination thereof; and/or a transcript stabilization element, optionally the transcript stabilization element comprises woodchuck hepatitis post-translational regulatory element (WPRE), bovine growth hormone polyadenylation (bGH-polyA) signal sequence, human growth hormone polyadenylation (hGH-polyA) signal sequence, or any combination thereof. At least one of the payload genes can be operably connected to a promoter selected from the group comprising: an RNA pol I promoter; a pol II promoter, optionally CMV, SV40 early region or adenovirus major late promoter; or pol III promoter, optionally a U6 or H1 promoter; a minimal promoter, optionally TATA, miniCMV, and/or miniPromo; a bacteriophage promoter, optionally a bacteriophage T3 promoter, a bacteriophage T7 promoter, a bacteriophage SP6 promoter, or a combination thereof; a tissue-specific promoter and/or a lineage-specific promoter; an inducible promoter, optionally a T7 RNA polymerase promoter, a T3 RNA polymerase promoter, an Isopropyl-beta-D-thiogalactopyranoside (IPTG)-regulated promoter, a lactose induced promoter, a heat shock promoter, or a Tetracycline-regulated promoter, a tetracycline-dependent promoter, a lac-dependent promoter, a pB ad-dependent promoter, an AlcA-dependent promoter, a LexA-dependent promoter, or a heat-shock promoter; a ubiquitous promoter, optionally a cytomegalovirus (CMV) immediate early promoter, a CMV promoter, a viral simian virus 40 (SV40) (e.g., early or late), a Moloney murine leukemia virus (MoMLV) LTR promoter, a Rous sarcoma virus (RSV) LTR, an RSV promoter, a herpes simplex virus (HSV) (thymidine kinase) promoter, H5, P7.5, and P11 promoters from vaccinia virus, an elongation factor 1-alpha (EF1a) promoter, early growth response 1 (EGR1), ferritin H (FerH), ferritin L (FerL), Glyceraldehyde 3-phosphate dehydrogenase (GAPDH), eukaryotic translation initiation factor 4A1 (EIF4A1), heat shock 70 kDa protein 5 (HSPA5), heat shock protein 90 kDa beta, member 1 (HSP90B1), heat shock protein 70 kDa (HSP70), β-kinesin (β-KIN), the human ROSA 26 locus, a Ubiquitin C promoter (UBC), a phosphoglycerate kinase-1 (PGK) promoter, 3-phosphoglycerate kinase promoter, a cytomegalovirus enhancer, human β-actin (HBA) promoter, chicken β-actin (CBA) promoter, a CAG promoter, a CASI promoter, a CBH promoter; or any combination thereof.

The final synthetic sequence can be or can comprise all or a portion of a vector. In some embodiments, a viral vector, a plasmid, a transposable element, a naked DNA vector, or any combination thereof. In some embodiments, an AAV vector, a lentivirus vector, a retrovirus vector, an adenovirus vector, a herpesvirus vector, a herpes simplex virus vector, a cytomegalovirus vector, a vaccinia virus vector, a MVA vector, a baculovirus vector, a vesicular stomatitis virus vector, a human papillomavirus vector, an avipox virus vector, a Sindbis virus vector, a VEE vector, a Measles virus vector, an influenza virus vector, a hepatitis B virus vector, an integration-deficient lentivirus (IDLV) vector, or any combination thereof. The transposable element can be piggybac transposon or sleeping beauty transposon. The final synthetic sequence can be configured for propagation in a eukaryotic or a prokaryotic cell. In some embodiments, the final synthetic sequence comprises: a bacterial origin of replication, optionally ColEl, p15A, pSC101, and RK2; an origin of transfer (oriT) and one or more mobilization genes configured to enable conjugative transfer; an autonomously replicating sequence (ARS), a centromeric sequence (CEN), and/or 2u elements; a rolling-circle replication origin, optionally derived from pC194, pE194, and pUB110; a mammalian origin of replication, optionally oriP/EBNA1 and/or SV40 ori; a selection marker, optionally an antibiotic resistance marker and/or a fluorescence marker; and/or a counter-selection marker, optionally sacB, rpsL, galK, CYH2, and/or URA3.

The final synthetic sequence can be configured for insertion into a genome. In some embodiments, the final synthetic sequence comprises: recognition sites for an RNA-guided DNA binding complex, wherein the RNA-guided DNA binding complex comprises one or more Cas proteins, a transposase, one or more crRNAs, or any combination thereof; recognition sites for a transposition complex comprising one or more transposases; homology arms, optionally targeting a safe-harbor locus selected from AAVS1, ROSA26, CCR5, and H11; one or more recombination sites, optionally loxP, FRT, attB, attP, attL, and attR; and/or a reporter cassette. The final synthetic sequence can comprise a digital data storage payload encoded in nucleic acid sequence.

Each of the n fragments can be housed in a separate vessel, optionally a tube, a well, or a microfluidic chamber. The first polynucleotide strand and the second polynucleotide strand that constitute each of the n fragments can be housed in a separate vessel, optionally a tube, a well, or a microfluidic chamber. In some embodiments, the composition further comprises: a non-thermostable ligase, a thermostable ligase, a chemical coupling agent, a polymerase, a primer capable of binding the first terminal region (or a complement thereof), a primer capable of binding the second terminal region (or a complement thereof), or any combination thereof. In some embodiments, the composition further comprises a ligation buffer. The ligation buffer can comprise: HiFi Taq buffer; one or more of Tris HCl at about 10 mM to about 200 mM, at a pH of about 7.0 to about 9.5 at the incubation temperature, Mg2+ at about 0.5 mM to about 20 mM, monovalent cation(s) at about 10 mM to about 300 mM, and a reducing agent at about 0.1 mM to about 20 mM; a ligase cofactor, optionally selected from ATP at about 0.05 mM to about 5 mM or NAD+ at about 0.01 mM to about 2 mM; a buffering species selected from Tris, HEPES, Bis Tris, MOPS, and PIPES, optionally configured to maintain pH between 8.3-8.8 at 25° C.; and/or one or more additives, optionally selected from bovine serum albumin at about 0.01 mg/mL to about 1 mg/mL, polyethylene glycol at about 1% to about 20% (w/v), betaine at about 0.1 M to about 2.0 M, dimethyl sulfoxide at about 1% to about 20% (v/v), formamide at about 0.5% to about 10% (v/v), glycerol at about 1% to about 20% (v/v), and/or a non-ionic detergent at about 0.001% to about 0.1% (v/v). In some embodiments, the composition does not comprise one or more reagents employed with Polymerase Cycling Assembly (PCA), Gibson assembly, USER, Yeast Assembly, Homologous Recombination, and/or Golden Gate assembly, optionally an exonuclease, an endonuclease, a single stranded DNA binding protein, a restriction endonuclease, a recombinase, or any combination thereof.

Disclosed herein include compositions. The composition can comprise: a pre-assembly reaction mixture comprising the n fragments disclosed herein at equimolar concentrations, optionally the temperature of the pre-assembly reaction mixture is above the melting temperature of the first and second toeholds and below the melting temperature of the first and second barcodes, and optionally the pre-assembly reaction mixture comprises a ligase or a chemical coupling agent. The composition can comprise: an intermediate reaction mixture comprising the n fragments disclosed herein joined together via three-way junction (3WJ) intermediates to generate an intermediate product, optionally said 3WJ intermediates each comprise a helix, and optionally the intermediate reaction mixture comprises a ligase or a chemical coupling agent. The composition can comprise: a post-ligation reaction mixture comprising an assembled product wherein the second polynucleotide strand does not comprise nicks, optionally the assembled product comprises three-way junction (3WJ) intermediates, optionally said 3WJ intermediates each comprise a helix.

Disclosed herein include methods. The method can comprise: providing n fragments, wherein n is an integer greater than 2. Each fragment can comprise a first polynucleotide strand and a second polynucleotide strand. Each (i)th fragment can comprise a first barcode and a second barcode on the first polynucleotide strand, wherein 1<i<n. In some embodiments, the first barcode of the (i)th fragment forms a pair with the second barcode of the (i-1)th fragment. In some embodiments, the second barcode of the (i)th fragment forms a pair with the first barcode of the (i+1)th fragment. The method can comprise: incubating the n fragments in a reaction mixture under reaction conditions such that: the first barcode of the (i)th fragment hybridizes to the second barcode of the (i−1)th fragment; and the second barcode of the (i)th fragment hybridizes to the first barcode of the (i+1)th fragment, thereby joining together the n fragments via three-way junction (3WJ) intermediates to generate an intermediate product. The method can comprise: ligating nicks on the second polynucleotide strands to generate an assembled product.

Disclosed herein include methods. The method can comprise: providing n fragments disclosed herein. The method can comprise: incubating the n fragments in a reaction mixture under reaction conditions such that: the first barcode of the (i)th fragment hybridizes to the second barcode of the (i−1)th fragment; and the second barcode of the (i)th fragment hybridizes to the first barcode of the (i+1)th fragment, thereby joining together the n fragments via three-way junction (3WJ) intermediates to generate an intermediate product. The method can comprise: ligating nicks on the second polynucleotide strands to generate an assembled product.

In some embodiments, hybridization of a first barcode and a second barcode of adjacent fragments forms a helix, wherein said 3WJ intermediates each comprise a helix. In some embodiments, (i) the hybridization of the first toehold and the second toehold of adjacent fragments further stabilizes the 3WJ intermediates; and/or (ii) one or more fragments do not comprise a toehold and the intermediate product is sufficiently stabilized by hybridization between first and second barcodes. In some embodiments of the methods, compositions, systems, and kits provided herein, some or all of the fragments do not comprise a first toehold and/or a second toehold, and the hybridization of first and second barcodes to form 3WJs are sufficient to generate an intermediate product suitable for ligation to generate an assembled product. In some embodiments, the formation of the helix holds adjacent fragments together at a temperature prohibiting interactions of the first toehold and second toehold of adjacent fragments alone. In some embodiments, the helix orthogonally winds up on the side of the final assembled sequence, thereby joining adjacent fragments together via the 3WJ intermediate. The association of the first toehold and second toehold of adjacent fragments can be unstable at the temperature(s) of the incubation step in the absence of the formation of the helix.

In some embodiments, the incubation step comprises: temperature(s) above the melting temperature (Tₘ) of the first toehold and second toehold. In some embodiments, the incubation step comprises: temperature(s) below the melting temperature (Tₘ) of the first barcode and second barcode. In some embodiments, the incubation comprises: incubation at a first incubation temperature for a first period of time. In some embodiments, the incubation comprises: addition of a ligase to the reaction mixture. In some embodiments, the incubation comprises: z assembly cycles, where z is an integer greater than 1. In some embodiments, each assembly cycle comprises: at least about 5 sec, 6 sec, 7 sec, 8 sec, 9 sec, 10 sec, 20 sec, 30 sec, 40 sec, 50 sec, 60 sec, 1 min, 2 min, 3 min, 4 min, 5 min, 6 min, 7 min, 8 min, 9 min, 10 min, or a number or a range between any two of these values, at a first incubation temperature, optionally 85° C. for 1 min; and at least about 10 sec, 20 sec, 30 sec, 40 sec, 50 sec, 60 sec, 1 min, 2 min, 3 min, 4 min, 5 min, 6 min, 7 min, 8 min, 9 min, 10 min, or a number or a range between any two of these values, at a second incubation temperature, optionally 50° C. for 2 min. In some embodiments, the incubation comprises: incubation at a second incubation temperature for a second period of time.

In some embodiments, the incubation comprises: incubation at a first incubation temperature for a first period of time; cooling the reaction mixture from the first incubation temperature to the second incubation temperature at a predetermined cooling rate (optionally the predetermined cooling rate comprises a reduction of 0.1° C. per 1 sec, 2 sec, 3 sec, 4 sec, 5 sec, 6 sec, 7 sec, 8 sec, 9 sec, or 10 sec); addition of a ligase to the reaction mixture; and incubation at the second incubation temperature for a second period of time. The first incubation temperature can be about 80° C., 81° C., 82° C., 83° C., 84° C., 85° C., 86° C., 87° C., 88° C., 89° C., 90° C., or a number or a range between any two of these values, optionally 85° C. The second incubation temperature can be about 35° C., 36° C., 37° C., 38° C., 39° C., 40° C., 41° C., 42° C., 43° C., 44° C., 45° C., 46° C., 47° C., 48° C., 49° C., 50° C., 51° C., 52° C., 53° C., 54° C., 55° C., or a number or a range between any two of these values, optionally 50° C. The first period of time can be about 10 sec, 20 sec, 30 sec, 40 sec, 50 sec, 60 sec, 2 min, 3 min, 4 min, 5 min, 6 min, 7 min, 8 min, 9 min, 10 min, or a number or a range between any two of these values, optionally five min. The second period of time can be about 10 min, 20 min, 30 min, 40 min, 50 min, 60 min, 2 hr, 4 hr, 6 hr, 8 hr, 10 hr, 12 hr, or a number or a range between any two of these values, optionally at least one hour.

In some embodiments, the assembly of the n fragments occurs independently of the sequence of the internal payload segments, and the assembly of the n fragments is directed by the formation of the helices between adjacent fragments, and thereby the molecular information which directs assembly of the n fragments is decoupled from the final synthetic sequence. In some embodiments, the assembled product comprises a final synthetic sequence, wherein the final synthetic sequence does not comprise the sequence of the first barcode or the second barcode of any of the n fragments. The final synthetic sequence can comprise a linear polynucleotide comprising the structure 5′-[first payload segment]-[second payload segment] . . . [(n)th payload segment]-3′. The final synthetic sequence can be a linear polynucleotide. The final synthetic sequence can be a circular polynucleotide wherein the 3′ end of the [(n)th payload segment] is linked to the 5′ end of [first payload segment] by a phosphodiester bond.

The final synthetic sequence can be at least about 500 bases, 750 bases, 1 kb, 2 kb, 3 kb, 4 kb, 5 kb, 6 kb, 7 kb, 8 kb, 9 kb, 10 kb, 15 kb, 20 kb, 25 kb, 50 kb, 75 kb, 100 kb, 250 kb, 500 kb, 750 kb, 1 MB, or a number or a range between any two of these values, in length. The ligating step can be performed with a ligase, optionally a thermostable ligase, optionally said ligase is selected from the group comprising T3 ligase, T4 ligase, T7 ligase, SplintR, E. coli DNA ligase, Hi-T4 ligase, HiFi Taq ligase, Taq ligase, 9°N, or any combination thereof. The ligating step can comprise contacting the intermediate product with a chemical coupling agent effective to form a covalent linkage between adjacent second polynucleotide strands, optionally one or more click chemistry reagents, optionally CuAAC, SPAAC, iEDDA, oxime formation, hydrazone formation, Michael addition, disulfide formation, carbodiimide mediated coupling, native chemical ligation, or any combination thereof.

The method further can comprise removing the helices to generate a scarless assembly. The method can comprise hybridizing a primer to the first terminal region and extending with a DNA polymerase, optionally a strand-displacing DNA polymerase. The method can comprise PCR amplification of the assembled product, or a product thereof, optionally using a primer capable of binding the first terminal region (or a complement thereof) and/or a primer capable of binding the second terminal region (or a complement thereof). In some embodiments, the base of the 3WJ comprise non-canonical nucleotide(s), and the method comprises: (i) contacting the assembled product with cleavage agent(s) to remove the 3WJ; and (ii) ligating nicks on the first polynucleotide strand, optionally: the non-canonical nucleotide(s) comprises deoxyuridine, deoxyinosine, deoxy-7-methylguanosine, deoxy-5,6-dihydroxythymidine, deoxy-3-methyladenosine, 5-methyl-deoxycytidine, O-6-methyl-deoxyguanosine, 5-iodo-deoxyuridine, 8-oxy-deoxyguanine, 1,N6-ethenoadenine, 8-oxo-guanine (8ox0G), or any combination thereof; and/or the cleavage agent(s) comprise USER Enzyme, a DNA glycosylase, an AP cleaving agent, APE 1 (AP Endonuclease 1), Endo III (Endonuclease III), Endo IV (Endonuclease IV), Endo V (Endonuclease V), Endo VIII (Endonuclease VIII), Fpg (formamido-pyrimidine-DNA glycosylase), OGG1 (8-oxoguanine DNA glycosylase 1), NEIL1 (Endonuclease VIII-like 1), T7 Endo I (T7 Endonuclease I), T4 PDG (T4 pyrimidine dimer DNA glycosylase), UDG (uracil DNA glycosylase), SMUG1 (Single-strand selective monofunctional uracil DNA glycosylase), AAG (methylpurine DNA glycosylase), or any combination thereof.

The incubating step can comprise combining the n fragments in a single reaction mix at equimolar concentrations, optionally at about 0.1 nM, 0.5 nM, 0.75 nM, 0.9 nM, 1.0 nM, 1.1 nM, 1.25 nM, 1.5 nM, 1.75 nM, 2 nM, 5 nM, 10 nM, or a number or a range between any two of these values. In some embodiments, the providing step comprises: generating the n fragments. Said generating step can comprise annealing the first polynucleotide strand and the second polynucleotide strand components of each of the n fragments to generate heteroduplexes. The n fragments can be each generated in separate reactions. The generating step can comprise phosphorylation of the second polynucleotide strands, further optionally via T4 polynucleotide kinase. Said annealing step can comprise an initial denaturation step followed by a gradual decrease in temperature. In some embodiments, the heteroduplexes undergo one or more purification steps, such as: gel electrophoresis, including pulsed-field gel electrophoresis (PFGE); solid or solution phase hybridization/capture; precipitation; dialysis; solid phase reversible immobilization (SPRI) cleanup, optionally performing size selection using SPRI beads, further optionally single-sided or double-sided; and/or column purification.

The method further can comprise PCR amplification of the assembled product, or a product thereof, to generate an amplified product. In some embodiments, PCR amplification can comprise amplifying the assembled product, or a product thereof, using a primer capable of hybridizing to the first terminal region or a complement thereof, and a primer capable of hybridizing the second terminal region or a complement thereof. The method can comprise purification of the assembled product, the amplified product, or products thereof. In some embodiments, said purification step compromises: gel electrophoresis of the assembled product, the amplified product, or products thereof; solid phase reversible immobilization (SPRI) cleanup, optionally performing size selection using SPRI beads, further optionally single-sided or double-sided; and/or column purification. The method can comprise replication of the assembled product, the amplified product, or products thereof, in a cell.

The providing step can comprise providing y sets of n fragments. The incubating step can comprise incubating the y sets of n fragments in a single reaction mixture, and the n fragments of each set can be joined together in parallel via three-way junction (3WJ) intermediates to generate y intermediate products. The ligating step can comprise ligating nicks on the second polynucleotide strands of each of the y intermediate products to generate y assembled products. The value of n can be the same between at least two of the y sets. The value of n can be the same different between at least two of the y sets. In some embodiments, the first barcode and the second barcode of each set are not complementary to the first barcode and the second barcode of any other set. The y intermediate products can be candidate design variants. The method can comprise the y intermediate products, or products thereof, being individually amplified or universally amplified. The integer y can be an integer greater than 1, optionally at least 2, 3, 4, 5, 6, 7, 8, 9, 10, 11, 12, 13, 14, 15, or a number or a range between any two of these values.

At least one of the n fragments can be a variant fragment, the assembled products can comprise a combinatorial library of at least p variants, and p can be an integer greater than 1. The integer p can be at least about 10, 50, 100, 250, 500, 750, 1000, 10000, 50000, 100000, 250000, 500000, 750000, 1000000, 5000000, 10000000, or a number or a range between any two of these values. In some embodiments, the combinatorial library achieves a variant coverage of at least 80%, 81%, 82%, 83%, 84%, 85%, 86%, 87%, 88%, 89%, 90%, 91%, 92%, 93%, 94%, 95%, 96%, 97%, 98%, 99%, 99.99%, or a number or a range between any two of these values, of the theoretical variant library. Every codon mutation profile can be represented in the library with an average absolute deviation of less than about 10%, 9%, 8%, 7%, 6%, 5%, 4%, 3%, 2%, 1%, 0.1%, 0.01%, or a number or a range between any two of these values, from the theoretical proportion of occurrence for that codon.

At least 95%, 96%, 97%, 98%, 99%, 99.9%, 99.99%, 99.999%, 9.9999%, or a number or a range between any two of these values, of the assembled products, or products thereof, can comprise all of the intended payload segments in the intended order. In some embodiments, less than 10%, 9%, 8%, 7%, 6%, 5%, 4%, 3%, 2%, 1%, 0.1%, 0.01%, or a number or a range between any two of these values, of the assembled products, or products thereof, are a partial assembly missing one or more payload segments. Less than 1 in 1000, 1 in 10000, 1 in 100000, 1 in 1000000, 1 in 10000000, 1 in 100000000, or a number or a range between any two of these values, of the assembled products can be missing one or more payload segments or can comprise a mis-assembled junction. The mis-ligation rate at the 3WJ can be less than 1 in 1000, 1 in 10000, 1 in 100000, 1 in 1000000, 1 in 10000000, 1 in 100000000, or a number or a range between any two of these values.

The integer n can be at least 3, 4, 5, 6, 7, 8, 9, 10, 11, 12, 13, 14, 15, 16, 17, 18, 19, 20, 22, 24, 26, 28, 30, 32, 34, 36, 38, 40, 42, 44, 46, 48, 50, 55, 60, 65, 70, 75, 80, 85, 90, 95, 100, 125, 150, 175, 200, 225, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, 1000, 10-25, 10-50, 10-75, 10-100, 10-500, 10-1000, 25-50, 25-75, 25-100, 25-500, 25-1000, 50-75, 50-100, 50-500, 50-1000, 75-100, 75-500, 75-1000, 100-500, 100-1000, 500-1000, or a number or a range between any two of these values. In some embodiments, the yield of correctly assembled products is at least 1-fold, 2-fold, 4-fold, 8-fold, 10-fold, 20-fold, 50-fold, 100-fold, 500-fold, or 1000-fold, greater than the yield of a polynucleotide assembly method not comprising 3WJ, optionally Polymerase Cycling Assembly (PCA), Gibson assembly, USER, Yeast Assembly, Homologous Recombination, and/or Golden Gate assembly. In some embodiments, at least about 10%, 15%, 20%, 25%, 30%, 35%, 40%, 45%, 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or a number or a range between any two of these values, of the incubated fragments become a component of an assembled product. Provided herein include compositions comprising assembled products, or products thereof, generated by a method disclosed herein. In some embodiments, the composition comprises a plurality of cells comprising the assembled products, or products thereof.

Disclosed herein include methods. The method can comprise: providing a combinatorial library disclosed herein, or a product thereof; expressing the one or more payload genes in cell(s); and screening for a property of interest. Screening can comprise fluorescence-activated cell sorting (FACS), cell viability assay, ELISA, co-immunoprecipitation, a bead-based immunoassay, or any combination thereof. The property of interest can comprise modified enzymatic activity, improved enzymatic activity, modified binding activity, improved binding activity, modified stability, improved stability, modified localization, improved localization, modified solubility, improved solubility, modified expression, improved expression, modified inhibitor resistance, improved inhibitor resistance, modified substrate specificity, improved substrate specificity, or any combination thereof. In some embodiments, the method comprises exposing the cell(s) to one or more agents. In some embodiments, the one or more agents comprise: one or more of a chemical agent, a pharmaceutical, small molecule, a biologic, a CRISPR single-guide RNA (sgRNA), a small interfering RNA (siRNA), CRISPR RNA (crRNA), a small hairpin RNA (shRNA), a microRNA (miRNA), a piwi-interacting RNA (piRNA), an antisense oligonucleotide, a peptide or peptidomimetic inhibitor, an aptamer, an antibody, an intrabody, or any combination thereof; an expression vector, wherein the expression vector encodes one or more of the following: an mRNA, an antisense nucleic acid molecule, a RNAi molecule, a shRNA, a mature miRNA, a pre-miRNA, a pri-miRNA, an anti-miRNA, a ribozyme, any combination thereof; an infectious agent, an anti-infectious agent, or a mixture thereof; a cytotoxic agent, optionally a chemotherapeutic agent, a biologic agent, a toxin, a radioactive isotope, or any combination thereof; and/or one or more of an epigenetic modifying agent, epigenetic enzyme, a bicyclic peptide, a transcription factor, a DNA or protein modification enzyme, a DNA-intercalating agent, an efflux pump inhibitor, a nuclear receptor activator or inhibitor, a proteasome inhibitor, a competitive inhibitor for an enzyme, a protein synthesis inhibitor, a nuclease, a protein fragment or domain, a tag or marker, an antigen, an antibody or antibody fragment, a ligand or a receptor, a synthetic or analog peptide from a naturally-bioactive peptide, an anti-microbial peptide, a pore-forming peptide, a targeting or cytotoxic peptide, a degradation or self-destruction peptide, a CRISPR component system or component thereof, DNA, RNA, artificial nucleic acids, a nanoparticle, an oligonucleotide aptamer, a peptide aptamer, or any combination thereof. The property of interest can comprise a property of the cell, such as improved drug resistance, altered drug sensitivity, improved or modified growth rate under selective pressure, modified or improved cell viability or survival, modified or improved stress tolerance, modified or improved secretion of a compound, altered signaling pathway activation, or any combination thereof. The method can comprise cloning the assembled products, or products thereof, into expression vector(s), optionally prior to an expressing step. The expression vector can be selected from a plasmid, a viral vector, a transposable element, a bacterial artificial chromosome, a yeast artificial chromosome, or any combination thereof. In some embodiments, the cloning step operably connects the final synthetic sequence with one or more regulatory elements selected from a promoter, an enhancer, a polyadenylation signal, a 5′UTR, a 3′ UTR, and a selection marker. The method can comprise transforming or transfecting host cells with the cloned expression vector, optionally bacterial cells for propagation and/or sequence verification and subsequently eukaryotic cells for expression, optionally mammalian, yeast, insect, plant, or fungal cells.

Provided herein include systems and kits for synthesizing nucleic acids. The system or kit can comprise: the n fragments provided herein, optionally: (i) y sets of n fragments; (ii) each of the n fragments is housed in a separate vessel, optionally a tube, a well, or a microfluidic chamber, and/or (iii) the first polynucleotide strand and the second polynucleotide strand that constitute each of the n fragments is housed in a separate vessel, optionally a tube, a well, or a microfluidic chamber. The system or kit can comprise: a non-thermostable ligase, a thermostable ligase, a chemical coupling agent, a polymerase, a primer capable of binding the first terminal region (or a complement thereof), a primer capable of binding the second terminal region (or a complement thereof), or any combination thereof. The system or kit can comprise: a ligation buffer. The ligation buffer can comprise: HiFi Taq buffer; one or more of Tris HCl at about 10 mM to about 200 mM, at a pH of about 7.0 to about 9.5 at the incubation temperature, Mg2+ at about 0.5 mM to about 20 mM, monovalent cation(s) at about 10 mM to about 300 mM, and a reducing agent at about 0.1 mM to about 20 mM; a ligase cofactor, optionally selected from ATP at about 0.05 mM to about 5 mM or NAD+ at about 0.01 mM to about 2 mM; a buffering species selected from Tris, HEPES, Bis Tris, MOPS, and PIPES, optionally configured to maintain pH between 8.3-8.8 at 25° C.; one or more additives, optionally selected from bovine serum albumin at about 0.01 mg/mL to about 1 mg/mL, polyethylene glycol at about 1% to about 20% (w/v), betaine at about 0.1 M to about 2.0 M, dimethyl sulfoxide at about 1% to about 20% (v/v), formamide at about 0.5% to about 10% (v/v), glycerol at about 1% to about 20% (v/v), and/or a non-ionic detergent at about 0.001% to about 0.1% (v/v). The system or kit can comprise; and/or one or more purification reagent(s), such as: gel electrophoresis reagent(s), optionally pulsed-field gel electrophoresis (PFGE); solid or solution phase hybridization/capture reagent(s); precipitation reagent(s); dialysis reagent(s); solid phase reversible immobilization (SPRI) cleanup reagent(s), optionally performing size selection using SPRI beads, further optionally single-sided or double-sided; and/or column purification reagent(s). In some embodiments, the system or kit does not comprise one or more reagents employed with Polymerase Cycling Assembly (PCA), Gibson assembly, USER, Yeast Assembly, Homologous Recombination, and/or Golden Gate assembly, optionally an exonuclease, an endonuclease, a single stranded DNA binding protein, a restriction endonuclease, a recombinase, or any combination thereof.

### EXAMPLES

Some aspects of the embodiments discussed above are disclosed in further detail in the following examples, which are not in any way intended to limit the scope of the present disclosure.

### Example 1

### Construction of Complex and Diverse DNA Sequences Using DNA 3-Way Junctions

The ability to construct entirely new synthetic DNA sequences de novo is essential to engineering and studying biology. The capacity to produce long complex synthetic DNA sequences and libraries currently lags behind the capacity to sequence and edit DNA. All existing DNA assembly technologies rely on DNA sequence information found within the final construct to direct assembly between DNA molecules. As a result of this paradigm, these sequences cannot be extensively optimized specifically for assembly without affecting the final sequence. To fundamentally address this challenge, herein the development of a new DNA assembly technique named Sidewinder is provided that separates the information that guides assembly from the final assembled sequence using DNA 3-Way Junctions. The transformative nature of the Sidewinder technique is demonstrated herein with highly robust and accurate construction of a 40-piece multi-fragment assembly, complex DNA sequences of both high GC content and high repeats, parallel assembly of multiple distinct genes in the same reaction, and a combinatorial library with a large number of diversified positions across the entire length of the gene for high coverage of a library of 442,368 variants. This technology enables high-fidelity DNA assembly with a misconnection rate at the 3WJ of approximately 1 in 1,000,000 in some embodiments.

### INTRODUCTION

De novo construction of DNA relies on synthetic single-stranded DNA (ssDNA) oligos as input produced either with phosphoramidite synthesis or enzymatically using Terminal deoxynucleotidyl transferase (TdT). Due to the cyclical nature of the synthesis process and the limited coupling efficiency at each step, the accuracy and yield of synthesized oligos decreases exponentially with increasing length. Consequently, de novo production of DNA larger than just a few hundred bases requires accurate DNA assembly of these short oligos together in the correct order.

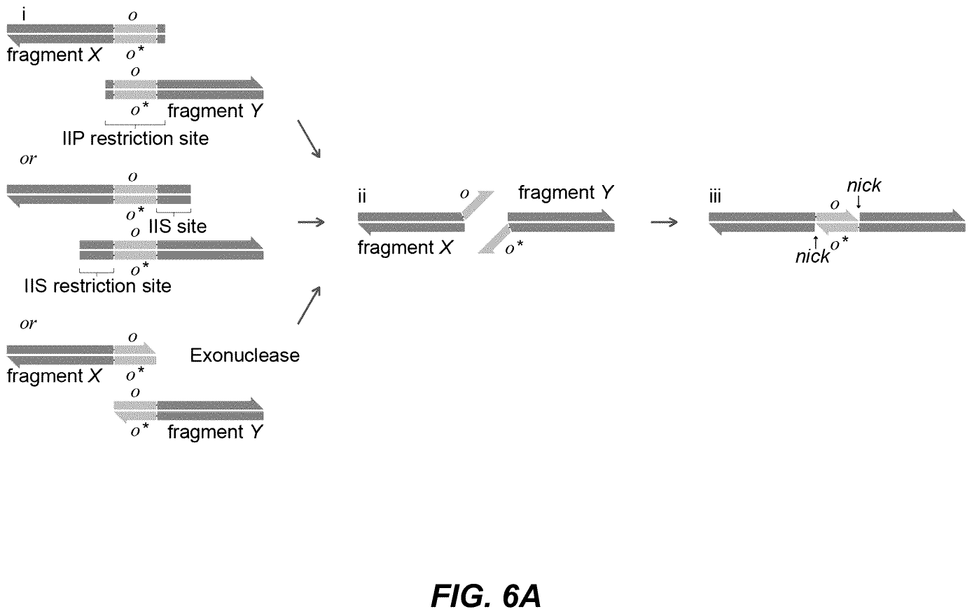

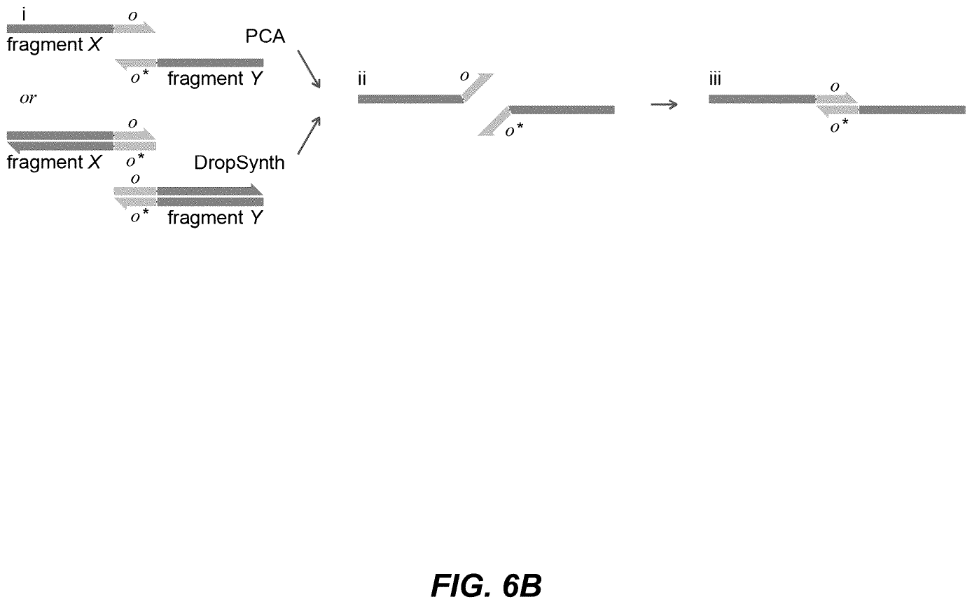

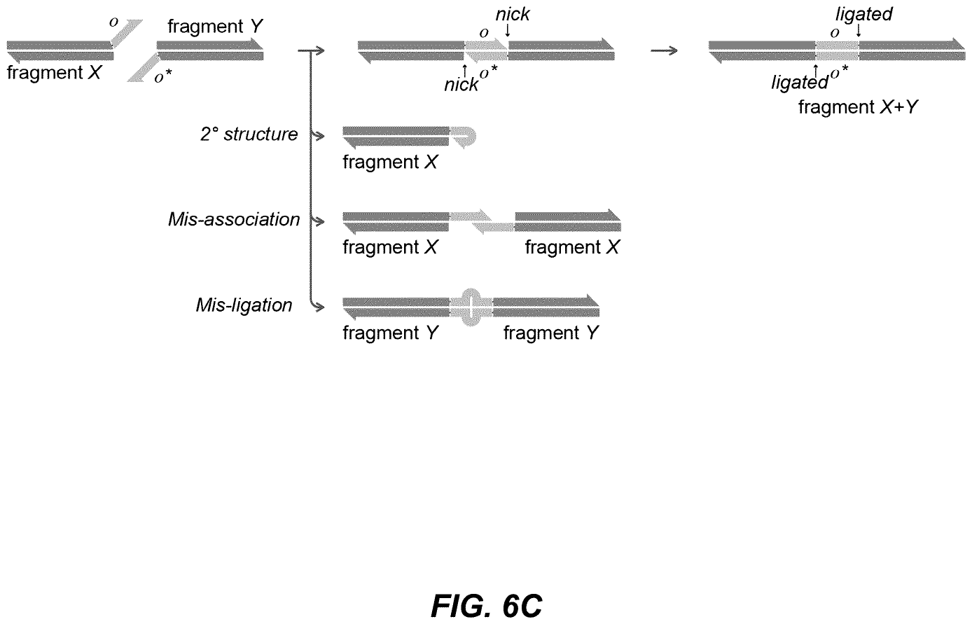

All prior DNA assembly techniques, either utilized by nature or invented by humankind, rely on the native 2-Way Junction (2WJ) between two complementary ssDNA overhangs (o and complementing o*) to guide the assembly, only differing in method of generating o/o* overhangs and whether to use double-stranded DNA (dsDNA) or ssDNA as input (FIG. 6). With this design, the complementation of olo* directs the assembly and consequently, olo* are incorporated as part of the final assembled sequence. Due to the duality in the function of olo*, overhang sequences cannot be extensively optimized for mutual exclusivity to maximize assembly efficiency without changing the final synthetic sequence. This intrinsic paradox unavoidably results in mis-assemblies that compoundingly limit the efficiency, size, and complexity of synthetic constructs.

To address this fundamental problem, provided herein are compositions, methods, systems, and kits employing the new “Sidewinder” approach, the first DNA assembly technique based on the novel DNA 3-Way Junction (3WJ) that can be reliably applied towards the construction of any DNA sequence without limitation. The 3WJ design enables the assembly to be directed by highly optimized sequences which are not present in the final product, facilitating robust assembly independent of the context of the assembled sequence. This example demonstrates and characterizes Sidewinder construction of synthetic DNA in a variety of contexts such as large multi-fragment assemblies, highly complex DNA sequences, parallel assembly of distinct constructs, and combinatorial library construction with high diversity coverage across the length of a gene.

### Results

### Establishing Sidewinder

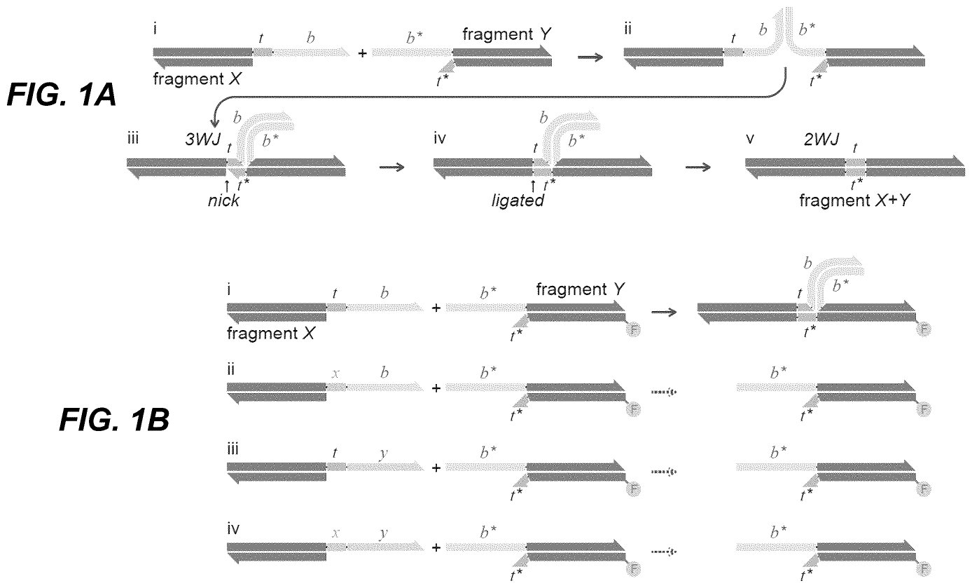

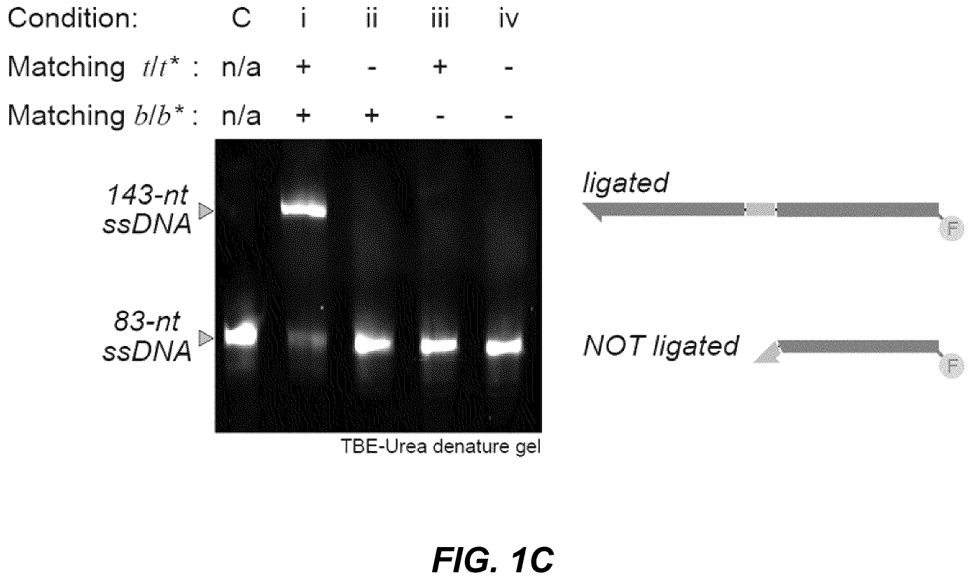

Sidewinder is fundamentally different from all previous techniques as it relies on information encoded within a third distinct helix to direct assembly between DNA fragments via the formation of a 3WJ (FIG. 1A). The 3WJ is one of the many unique DNA confirmations found among a wide variety of non-canonical or artificial DNA interactions employed in DNA Nanotechnology but has not been previously utilized in DNA assembly. The third helix, here after referred to as the Sidewinder helix, orthogonally winds up on the side of the final assembled sequence, hence the name “Sidewinder”. The Sidewinder helix is not part of the final assembled sequence and thus remove constraints on where assembly occurs, what sequences are being assembled, and how many DNA fragments can be assembled at once.

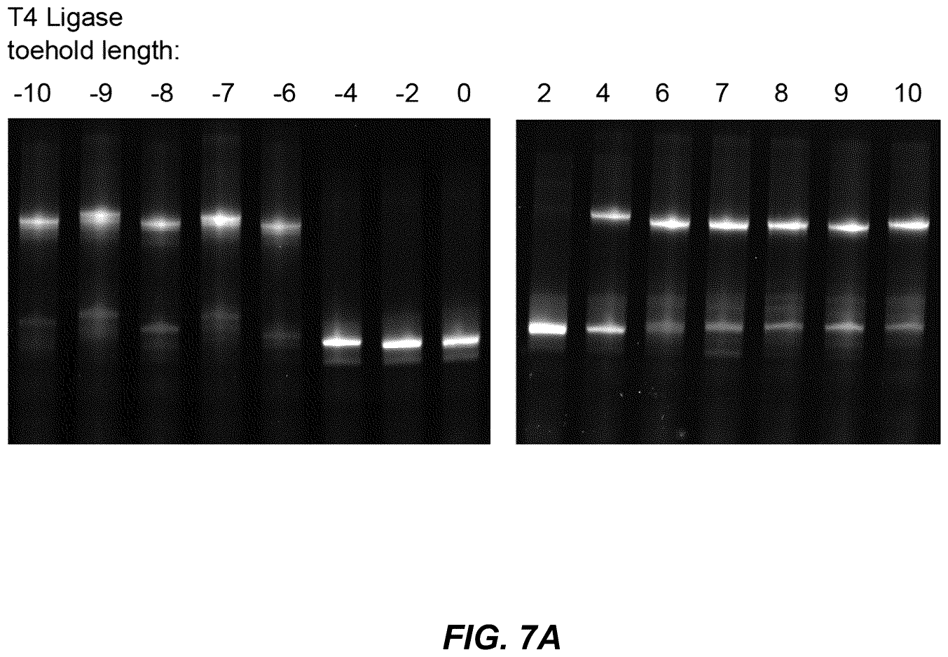

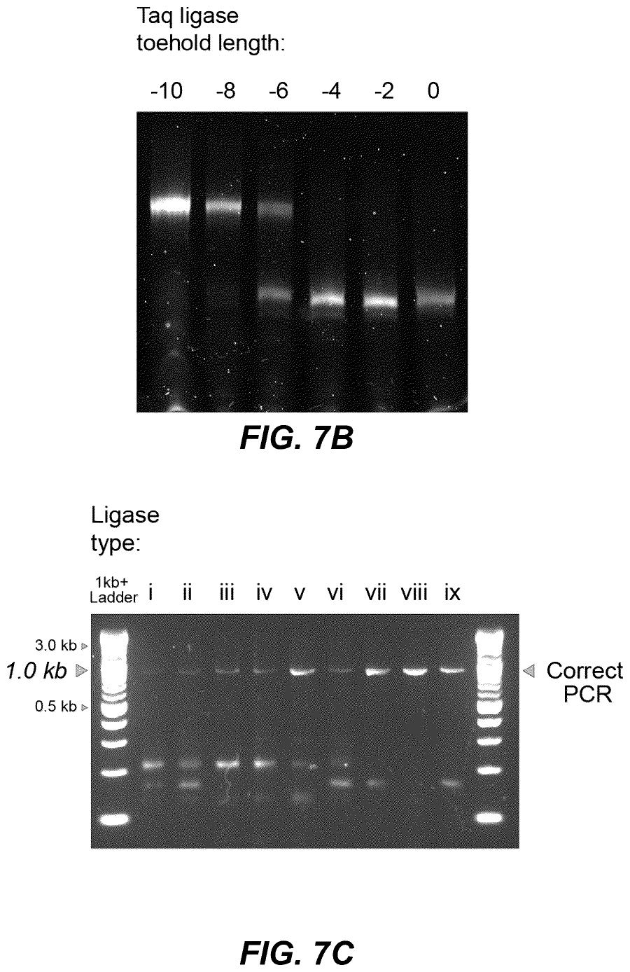

Sidewinder assembly fragments can contain unique terminal secondary structures referred to hereout as “toeholds” (t/t*) and Sidewinder “barcodes” (b/b*) (FIG. 1A). Toeholds t/t* are shortened exposed single-stranded sequences which will be found in the final synthetic product—these can be thought of as analogous to 2WJ olo* overhangs in other techniques (FIG. 6). Sidewinder barcodes b/b* are exposed single-stranded sequences which constitute the Sidewinder helix in the 3WJ, are not found in the final product, and can be highly optimized for each specific assembly (FIG. 1A, i). When Sidewinder fragments are mixed at a temperature higher than the melting temperature (Tₘ) of t/t*, the increased Tₘ of the longer Sidewinder barcodes b/b* can direct Sidewinder fragments to associate specifically (FIG. 1a, ii). The Sidewinder barcodes b/b* can wind up to form the Sidewinder helix, bringing together the complementary, but otherwise unstable at the reaction Tₘ, toeholds t/t* to further stabilize the 3WJ, leaving only a nick between the two Sidewinder fragments (FIG. 1A, iii). The nick can then ligated, irreversibly connecting the two fragments together (FIG. 1A, iv, FIG. 7). This “3WJ assembly” can then be further processed to remove the Sidewinder helix and scarlessly restore the conventional 2WJ, completing the construction of the synthetic DNA product (FIG. 1A, v).

With this Sidewinder 3WJ assembly scheme, both complementary toeholds t/t* and complementary Sidewinder barcodes b/b* are required for successful assembly in some embodiments. To demonstrate the feasibility of Sidewinder, four separate 2-fragment assemblies were set up with each possible combination of either matching or mismatching toeholds or barcodes (FIG. 1B). Successful conversion of un-ligated fragments to ligated product was determined by tracking the migration of a fluorophore-tagged oligo in fragment Y on a TBE-Urea denature gel (FIG. 1C). An upper band, which is indicative of ligation at the 3WJ, was only seen for the condition where the untagged fragment X had both a complementary toehold and complementary barcode to fragment Y. All other combinations showed only the lower band, indicative of an un-ligated fragments. Using Sidewinder barcodes b/b*, Sidewinder physically decouples the final assembled sequence from the instructions for assembly. This new paradigm allows assembly conditions to be established which provide unprecedented exclusivity and specificity for assembly between DNA fragments.

### Scaling Sidewinder

Sidewinder can be robustly scaled up to both ends of a large number of DNA fragments, allowing for large multi-fragment assemblies without limitations of conventional methods such as, but not limited to, restriction enzyme recognition sequences or the need to shift junctions to accommodate for orthogonal overhangs for assembly. Despite Sidewinder being theoretically compatible with DNA fragments from any source, this example focuses on assembly of synthetic oligos (FIG. 2). To construct an entirely synthetic sequence using Sidewinder, the length of each Sidewinder fragment can be determined by the max length of the input oligos, in this study 120mers were used due to quality and cost. To design the assembly oligos, the target sequence to be constructed is first bioinformatically split by choosing the positions of the toeholds (Methods). Then barcodes either pre-validated for exclusivity (See Plesa C., et al. Multiplexed gene synthesis in emulsions for exploring protein functional landscapes. Science 359, 343-347 (2018), the content of which is incorporated herein by reference in its entirety) or bespoke designed in-house using NUPACK (See Fornace, M. E., et al. NUPACK: analysis and design of nucleic acid structures, devices, and systems. ChemRxiv (2022), See Zadeh, J. N., et al. NUPACK: analysis and design of nucleic acid systems. J Comput Chem, 32,170-173 (2011), the contents of which are incorporated herein by reference in their entireties) are chosen for that particular toehold and combination of toeholds. (Methods).

Assembly fragments can be composed of two synthetic oligos. The top ssDNA oligo can be deemed the “barcode oligo” and contain Sidewinder barcodes on both ends. The bottom ssDNA oligo can be deemed the “coding oligo” and can be complementary to the majority of the barcode oligo but shifted slightly to expose the toeholds at both ends of the fragment. The coding oligo can then phosphorylated individually and annealed to the barcode oligo using standard conditions customary to DNA Nanotechnology, resulting in the dsDNA Sidewinder fragment heteroduplex with the unique secondary structures desired at both ends of the assembly fragment (FIG. 2A).

The oligo annealing process can be conducted for an arbitrary number of pairs of oligos to generate the Sidewinder fragments (FIG. 2B, i). PAGE extraction can be performed on the Sidewinder fragments in order to purify away any unannealed oligos. The individually processed Sidewinder fragments can then mixed, self-assembled, and ligated to compose the “3WJ assembly” (FIG. 2B, ii). Upon the completion of the 3WJ assembly, each of the coding oligos from each of the fragments has been ligated together to compose the uninterrupted synthetic sequence of the final target construct. In a single step, all barcode oligos can be either displaced or destroyed through DNA polymerase extension of a primer using the now connected coding strand as template, restoring the 2WJ throughout the assembly, thereby completing the construction (FIG. 2B, iii). This DNA polymerase extension can be integrated as a part of the selective Polymerase Chain Reaction (PCR) to further amplify the assembled Sidewinder product.

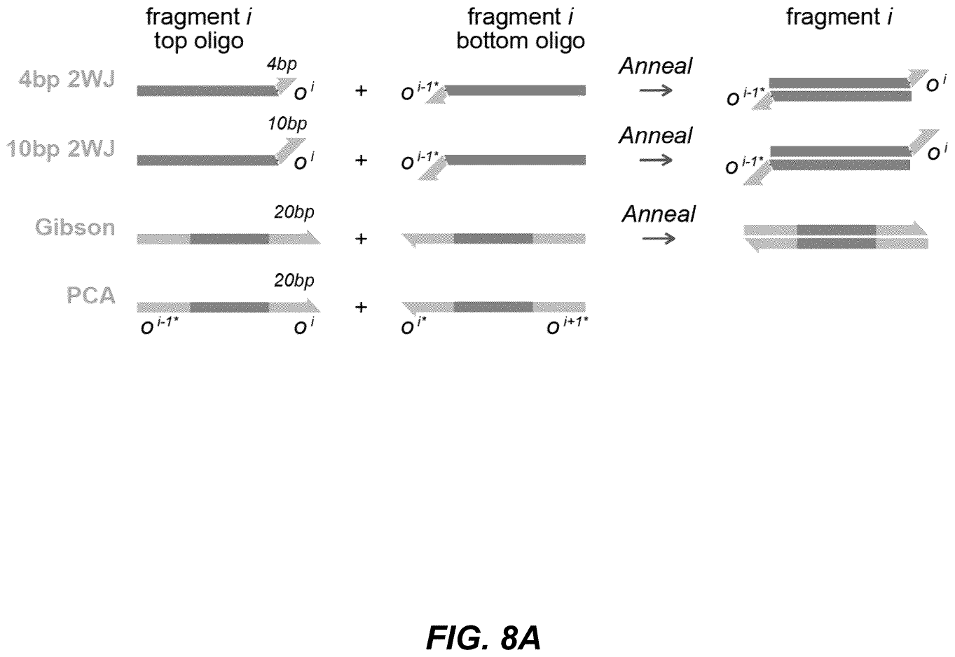

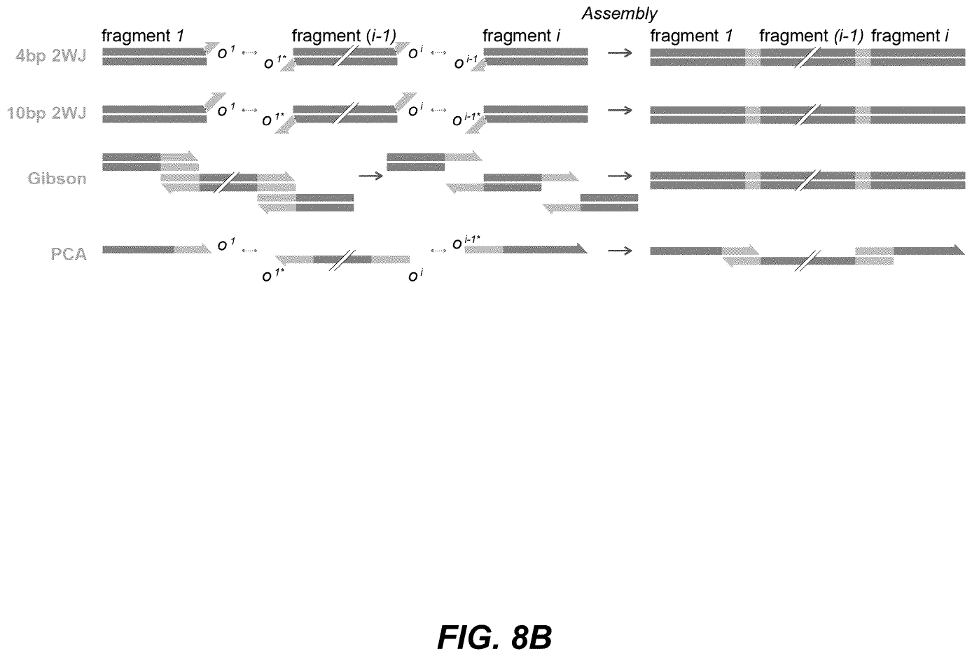

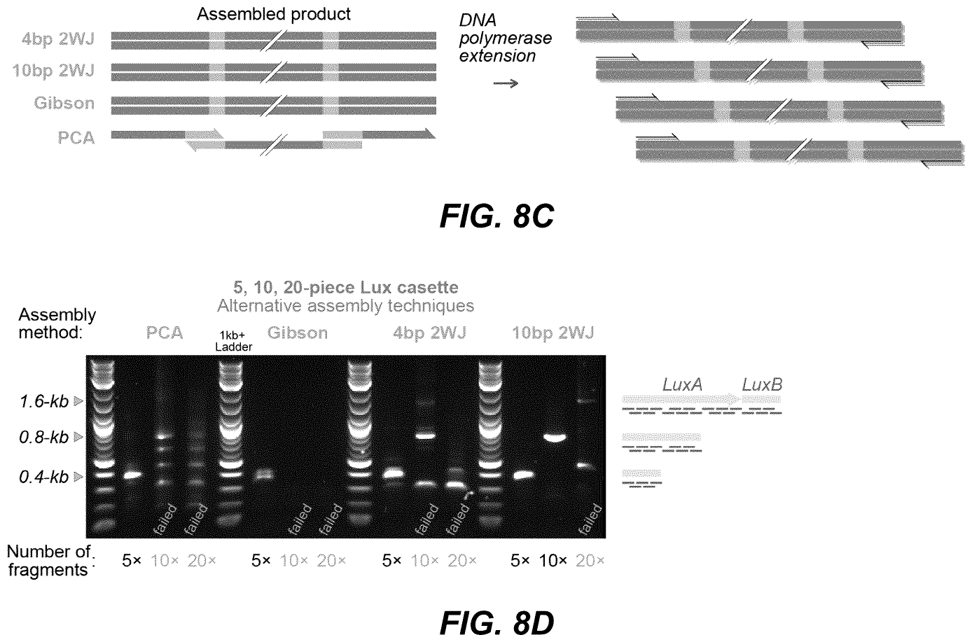

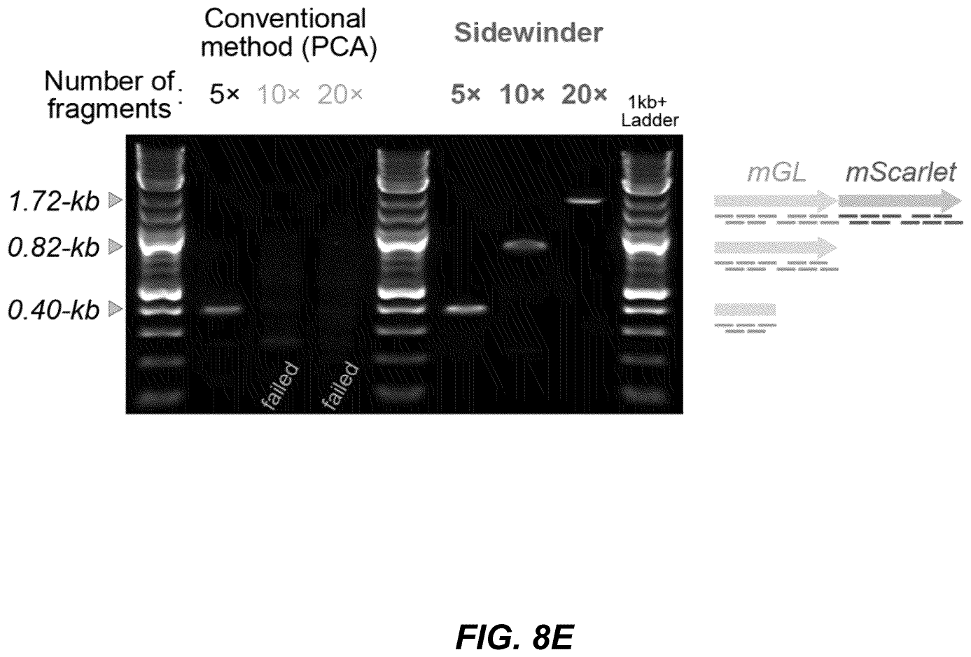

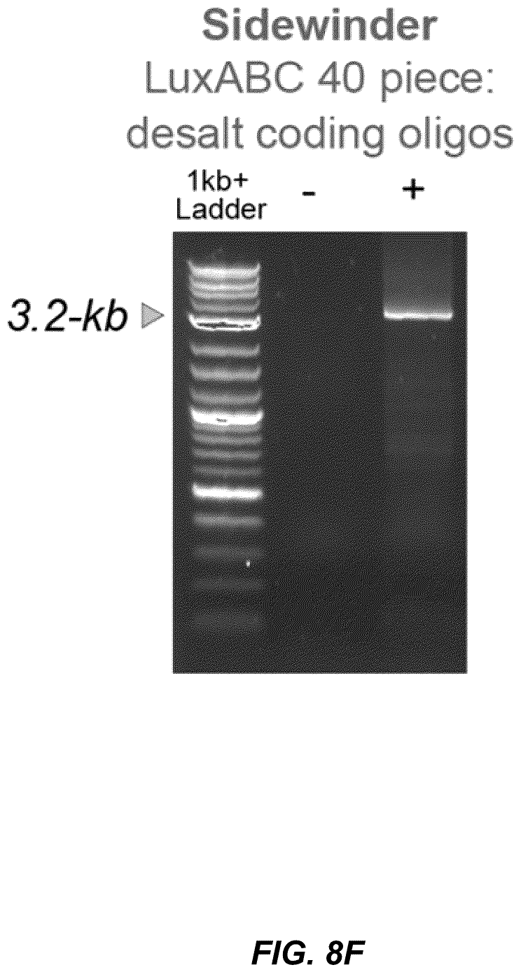

Using Sidewinder, it was first demonstrated robust assemblies of increasing size from 5, 10, 20, & 40 fragments to construct a segment of the LuxABCDE operon. Sidewinder produced a single, strong, target amplicon of the expected size in all reactions with no sign of mis-assemblies (FIG. 2C). In order to provide a reference for the impact of this feat, analogous assemblies of the Lux operon with Polymerase Cycling Assembly (PCA), Gibson assembly, a 4 bp overhang ligation (analogous to Golden Gate assembly), and a 10 bp overhang ligation (analogous to Sidewinder without the barcodes) (FIG. 8A) were set up for 5, 10, and 20-piece assemblies. The fragments were then processed to mirror Sidewinder, including the final PCR amplification step (FIGS. 8B-8C). All prior techniques tested fail beyond 5-piece assembly except for the 10 bp overhang assembly which produces a clean 10-piece product but fails at 20-pieces (FIG. 2C, FIG. 8D). Sidewinder was further applied to construct a distinct series of assemblies of an mGL and mScarlet fusion cassette where PCA failed again beyond a 5-piece assembly while Sidewinder has success for all reaction sizes (FIG. 8E).

While the gels demonstrate a qualitative performance of the assembly, Nanopore sequencing was conducted on the amplicon to quantitatively confirm robust assembly. All Nanopore reads were analyzed and assigned to categories based on the characteristics of the read (Methods). A fragment level analysis was first conducted by compiling and manually analyzing Nanopore sequencing reads from the 40-fragment Sidewinder assembly of LuxABC. Out of 609 reads, 12 reads were identified as primer mis-priming, constituting the 1.97% PCR artifacts; 2 reads (0.33%) were identified as sequencing artifacts; 6 reads (0.98%) were identified as barcode artifacts. Sidewinder products constitute all remaining reads, composing 589 out of 609 (96.72%) of the total reads. Notably, 100% of those Sidewinder products were correctly assembled 40-piece constructs with all fragments in the correct order (FIG. 2D). In contrast, out of over 5,000 reads for the corresponding 20-piece PCA reaction, not a single read was a correct assembly with the largest partial assembly only having 12 fragments (NCBI accession number PRJNA1201800).

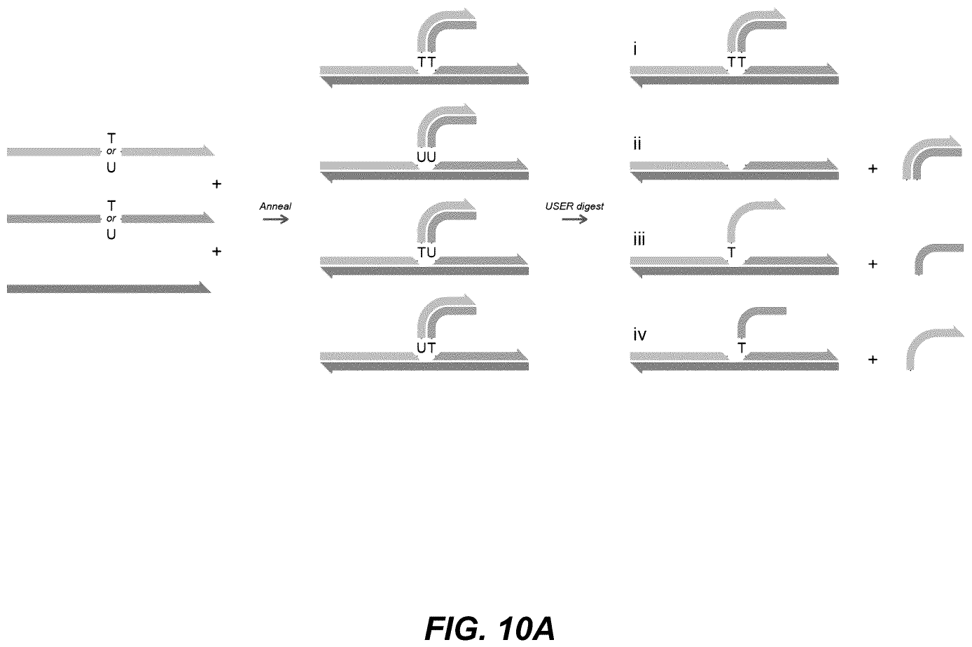

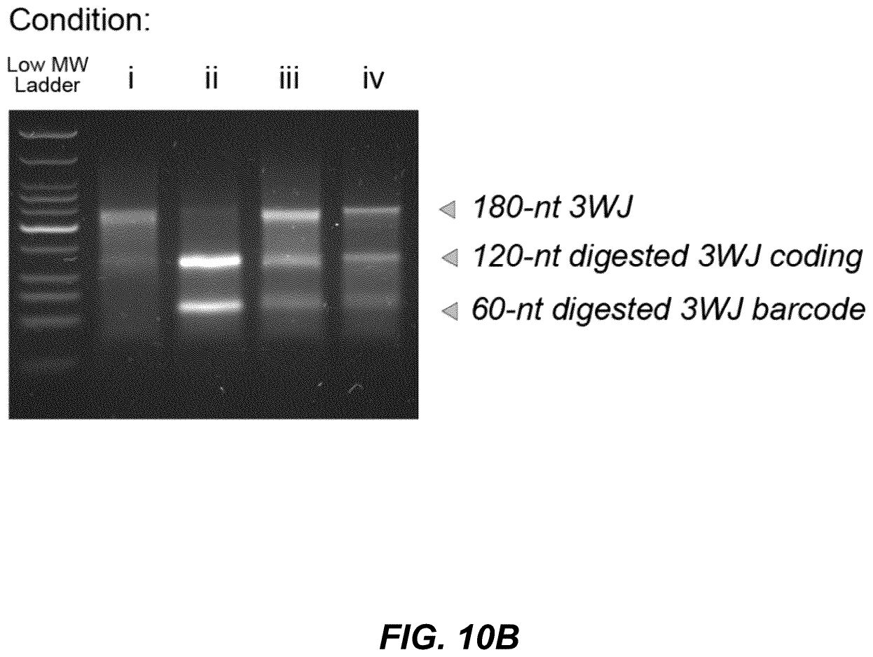

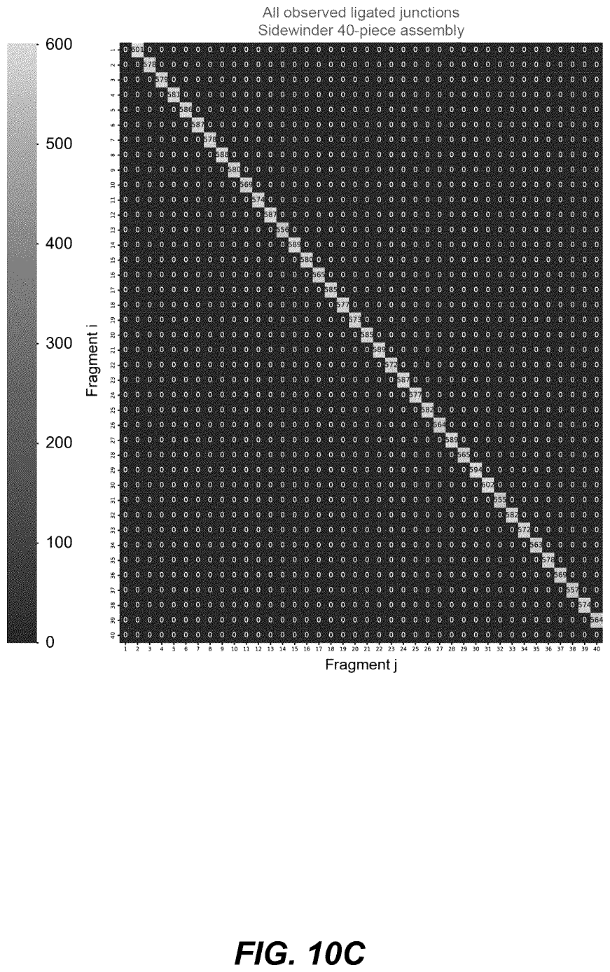

A separate analysis pipeline was further applied to the dataset in order to reduce bias from assigning reads at the fragment level. The entirety of the unfiltered raw sequencing data was searched to identify all instances of ligated 3WJs that could result from either correct or incorrect ligation. All together 22,533 ligated junctions were identified in this sequencing data. All 22,533 were correctly assembled junctions with 0 observed mis-ligations (FIG. 2E, FIG. 10).

Sidewinder's large multi-fragment assemblies enabled by the exclusivity and fidelity of the 3WJ interactions lift the current limitations on the number of long oligos that can be assembled in a single reaction such that the main limitation to the construct size is shifted to errors in oligo synthesis and the likelihood of finding a single mutation-free clone.

### Sidewinder Constructs Complex DNA

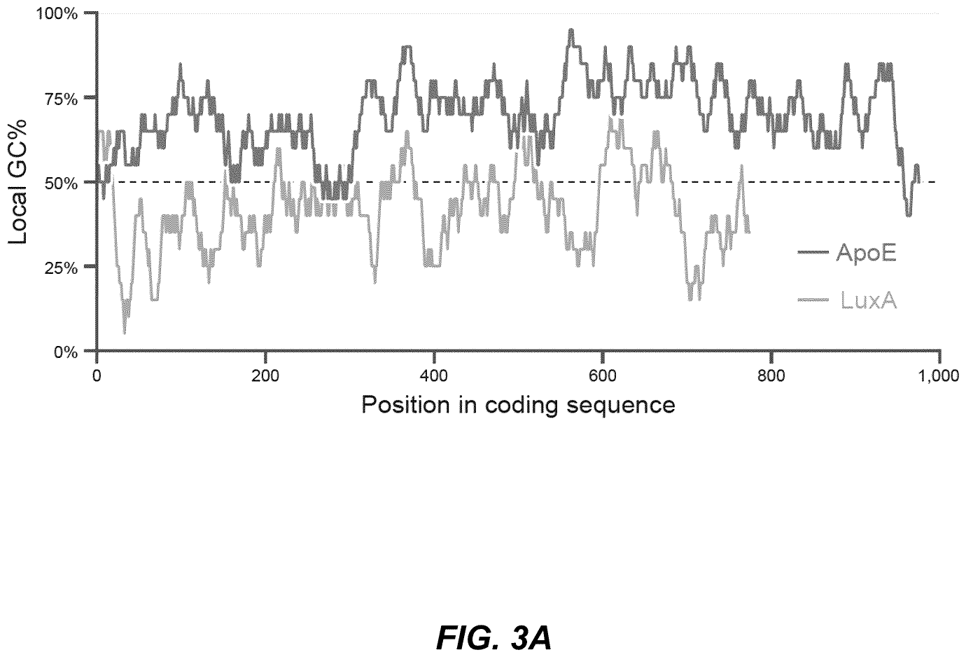

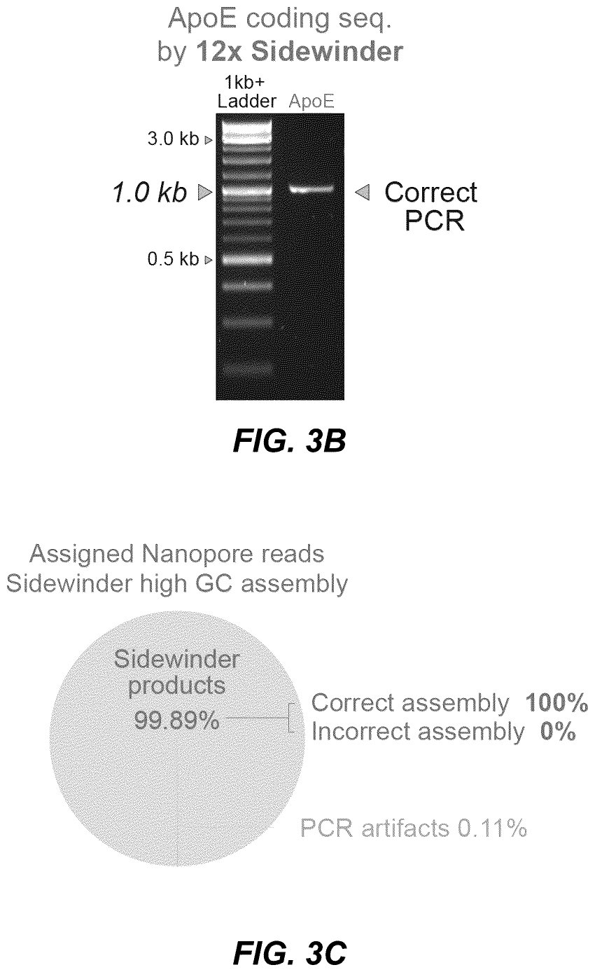

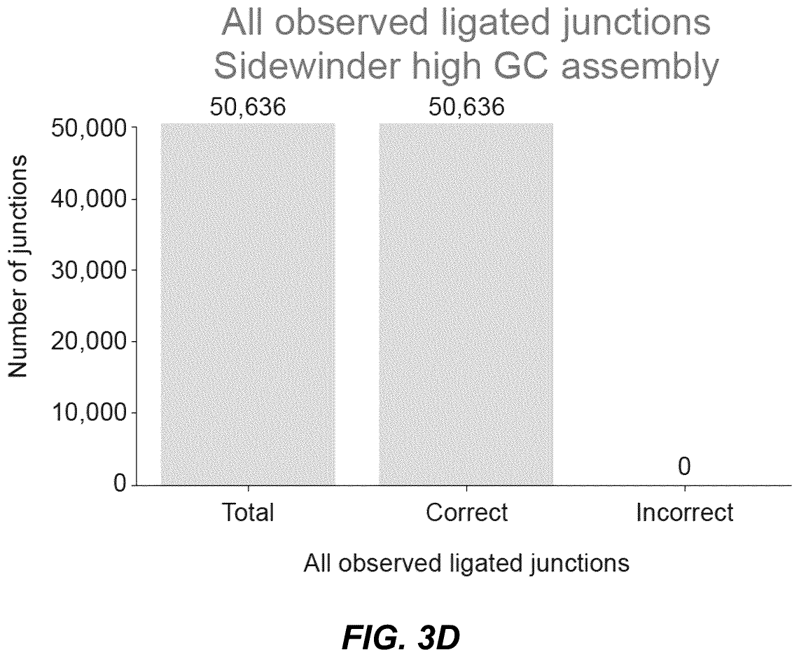

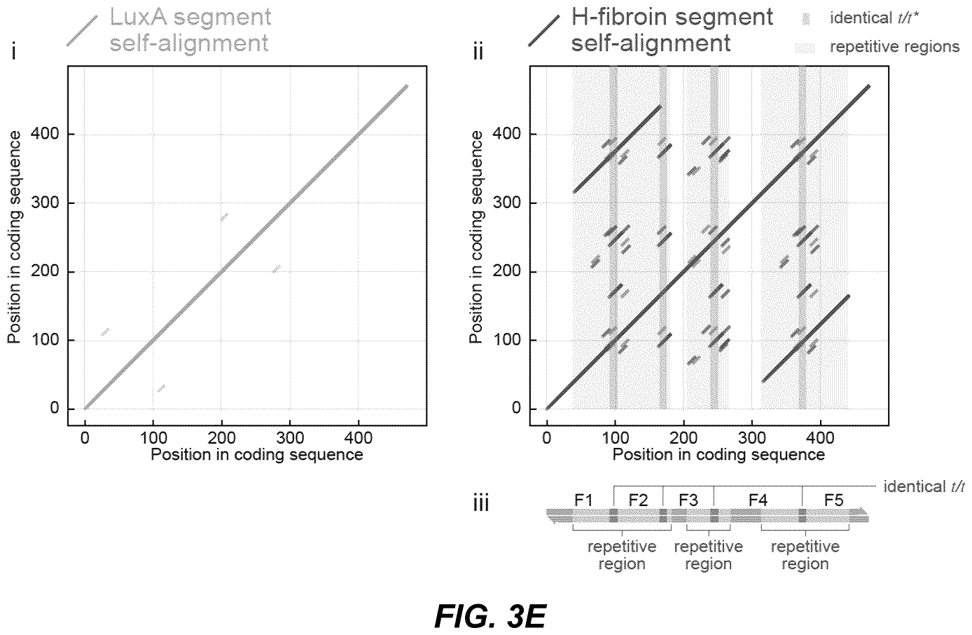

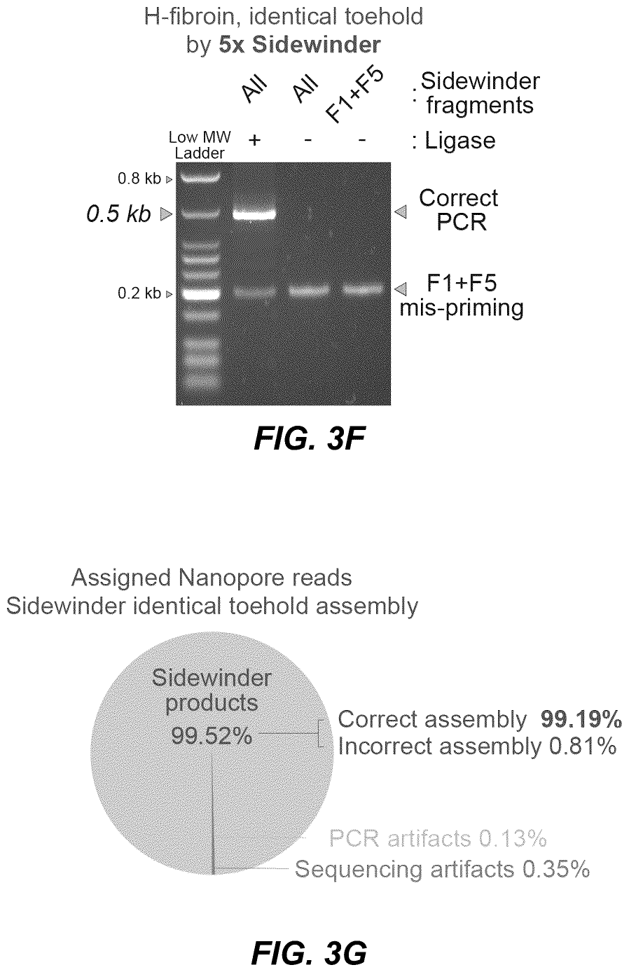

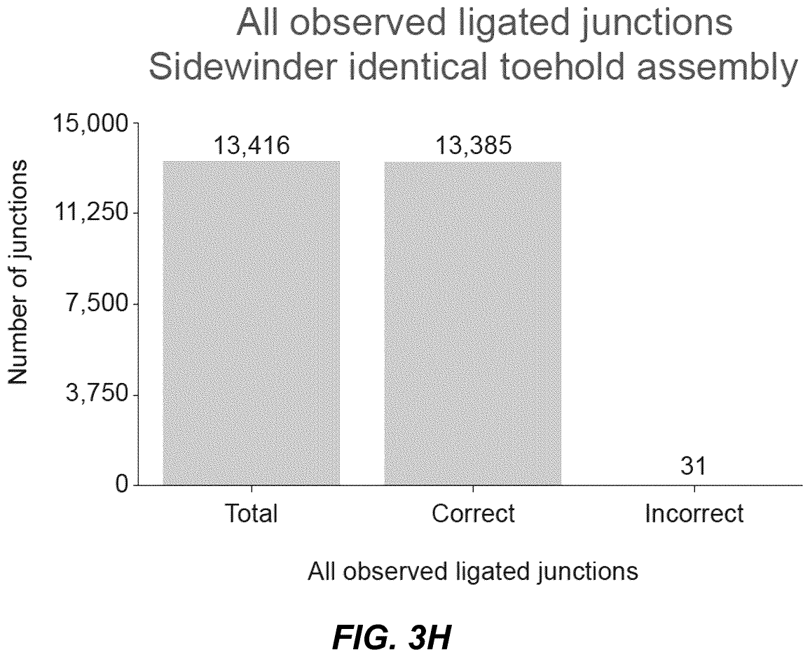

In addition to reliably assembling a larger number of fragments far beyond prior methods, Sidewinder enables the construction of complex DNA sequences which are otherwise difficult to assemble. First, the native coding sequence of the human protein Apolipoprotein E (ApoE) was assembled, which has a high proportion of guanine and cytosine (GC) bases across the gene. ApoE regulates cholesterol transport and maintains lipid homeostasis in the brain, and has allelic polymorphisms associated with increased risks of Alzheimer's disease and cerebral amyloid angiopathy. Its coding sequence is 70% GC with segments of the gene having as high as a 95% GC content (FIG. 3A). With a 12-piece Sidewinder assembly, a single clean product was produced (FIG. 3B) which when sequenced has 99.89% Sidewinder products across over 4,500 Nanopore reads with again 100% of these being correct assemblies (FIG. 3C). Applying the junction analysis pipeline identified a total of 50,636 ligated junctions with all 50,636 being correctly ligated (FIG. 3D) demonstrating a robust capacity for assembling high GC content sequences via the 3WJ.

In addition to GC rich sequences, a segment of the highly repetitive silk protein h-fibroin from Glyphotaelius pellucidus was constructed. Silk proteins are of interest because of their biodegradability and their highly repetitive sequences which give rise to their unique mechanical properties and potential applications as a biomaterial. This segment of h-fibroin was selected to demonstrate Sidewinder's ability to handle extremely repetitive DNA sequences which are notoriously difficult to reliably assemble. To further push the limits of this assembly, the Sidewinder fragments used in this construction were designed to use identical t/t* toehold sequences which are nested within regions of the construct which are dense with repeats (FIG. 3E, FIG. 11A). Without Sidewinder's signature 3WJ b/b* barcodes, this would be analogous to a multi-fragment assembly where the olo* overhangs are completely identical to each other which would be intrinsically impossible to accurately direct assembly with any prior method.

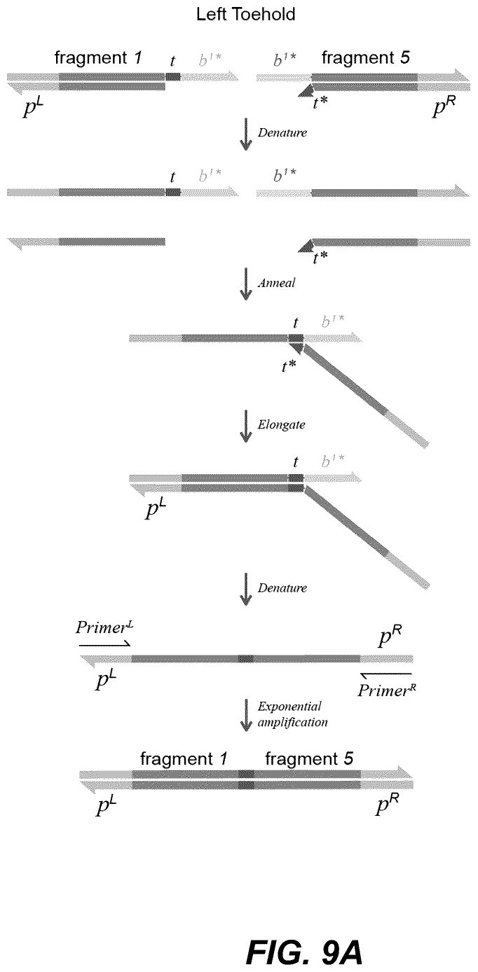

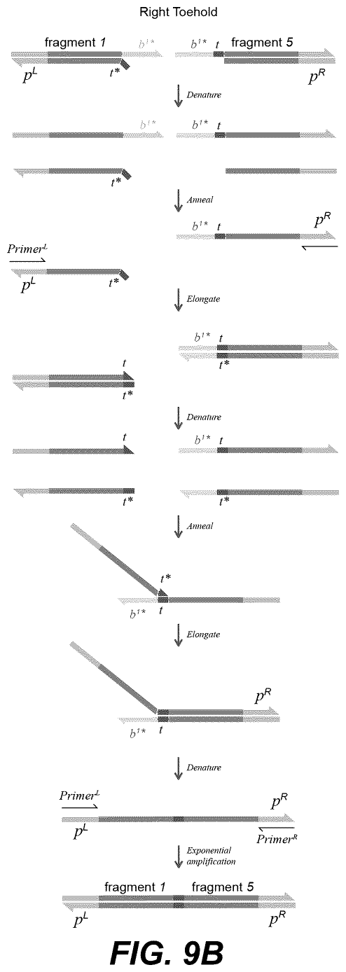

Sidewinder's high specificity for proper ligation at the 3WJ enables this 5-piece identical toehold assembly to be possible with a strong assembly product successfully constructed despite the extreme reaction conditions (FIG. 3F). The DNA agarose gel depicting a strong target band for the 5-piece identical toehold construct shows only a single minor byproduct which appears as a result of a PCR artifact as demonstrated by the same 200 bp byproduct appearing even when the 3WJ is not ligated or only Fragment 1 (F1) and Fragment 5 (F5) are provided as PCR template without ligation. Sanger sequencing of this byproduct indicates that it is the mis-priming of the F1 toehold to the F5 toehold and getting preferentially amplified by PCR's bias towards amplification of shorter products (FIG. 9).

Gel extraction of the correct size band was used for Nanopore sequencing. The results indicate a highly specific assembly and amplification of the proper 5-piece product with extremely high fidelity with 99.52% of Nanopore reads being Sidewinder products with 99.19% of those being correct assemblies and only 0.81% having evidence of mis-ligation during assembly (FIG. 3G). The junction analysis identifies just 31 mis-assembled junctions out of a total of 13,416 sequenced junctions, or 99.77% accuracy per junction for Sidewinder assembly in this deliberately extreme reaction (FIG. 3H).

To provide a reference point for the difficulty of these assemblies, the four prior assembly methods (PCA, Gibson assembly, 4 bp overhang analogue to Golden Gate, and 10 bp overhang) were also applied to both of these complex sequences. Analogous fragments were designed using oligos which are compatible with each assembly method as previously described (FIG. 8). Again, it was seen each prior assembly method fail to produce a clean band of the target size in all instances for both assemblies (FIGS. 11B-11C). These comparison experiments demonstrate the advantage of Sidewinder's 3WJ paradigm in assembling complex sequences in addition to large multi-fragment assemblies.

### Sidewinder One-Pot Parallel Assemblies

Sidewinder's fidelity allows multiple assemblies of distinct constructs simultaneously in the same reaction tube. This can be particularly applicable in the field of AI facilitated DNA and protein design where in silico methods generate multiple competing designs which can be difficult and costly to synthesize and evaluate simultaneously in the physical world.

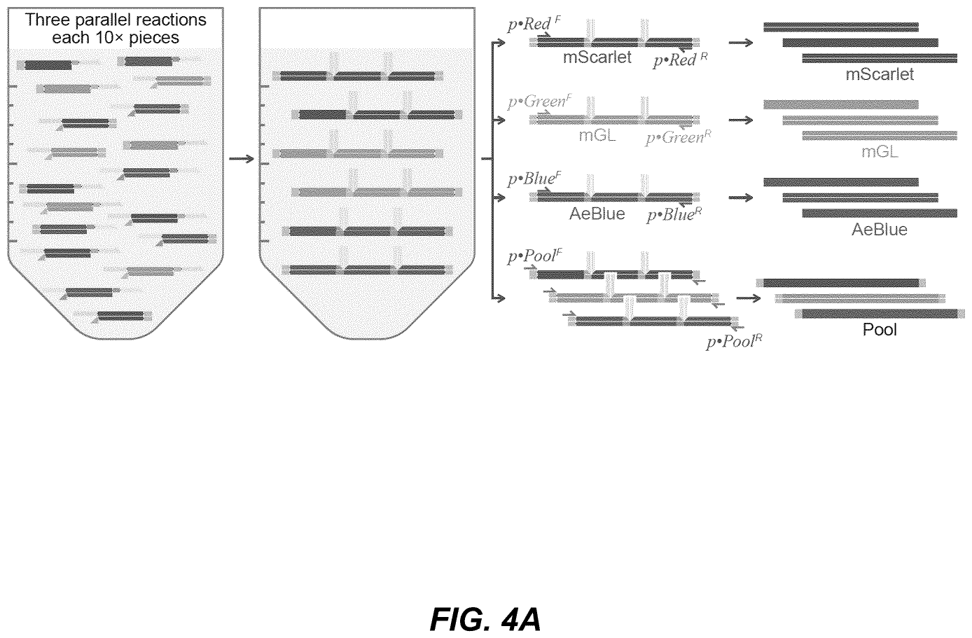

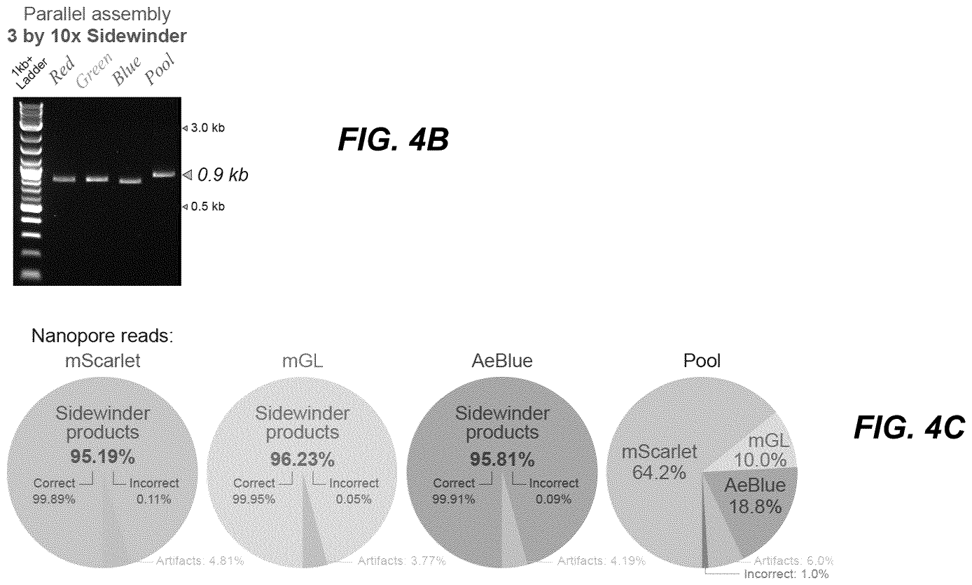

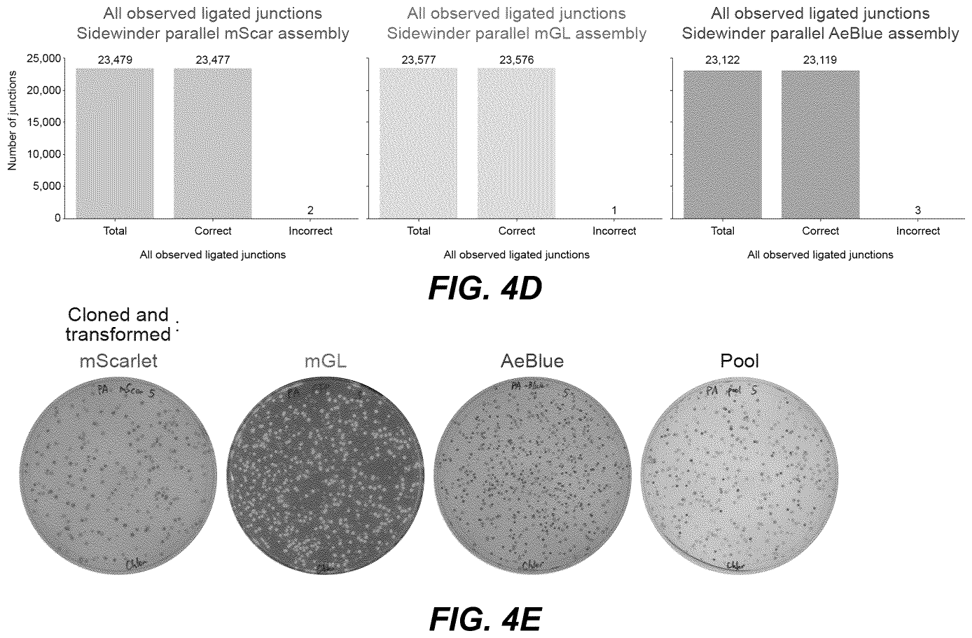

The Sidewinder fragments were combined for three distinct 10-piece assemblies each encoding for different colorimetric phenotypic markers: mScarlet, mGL, and the chromoprotein aeBlue. Sidewinder assemblies were conducted for each of the constructs simultaneously in the same reaction tube (FIG. 4A). Through selective amplification using either specific primer pairs for each construct or a universal primer pair for all three constructs, any of the 3 individual constructs or the pool of all 3 constructs can be dialed out producing a single, clean target band in all cases (FIG. 4B).

Quantitative assessment of the assemblies using Nanopore sequencing analysis of the pre-clonal Sidewinder PCR products indicates a high fidelity for the proper assembly product across each of the different constructs with 95.19%, 96.23%, and 95.81% Sidewinder products for mScarlet, mGL and aeBlue respectively with again 99.9% of these being correct, exclusive assemblies for each construct (FIG. 4C). For the combined pool of all three constructs, a distribution of reads assigned to each of the three constructs was seen with again low rates of mis-priming and mis-ligation (FIG. 4C). In order to better simulate pooled conditions, the parallel assembly Sidewinder fragments were not PAGE extracted prior to assembly which is the likely cause for the observed increase in mis-priming. Junction analysis shows very low rates of mis-ligation at the 3WJ for each of the individually amplified samples with just 2, 1, and 3 mis-ligated junctions out of over 23,000 junctions for each construct (FIG. 4D).

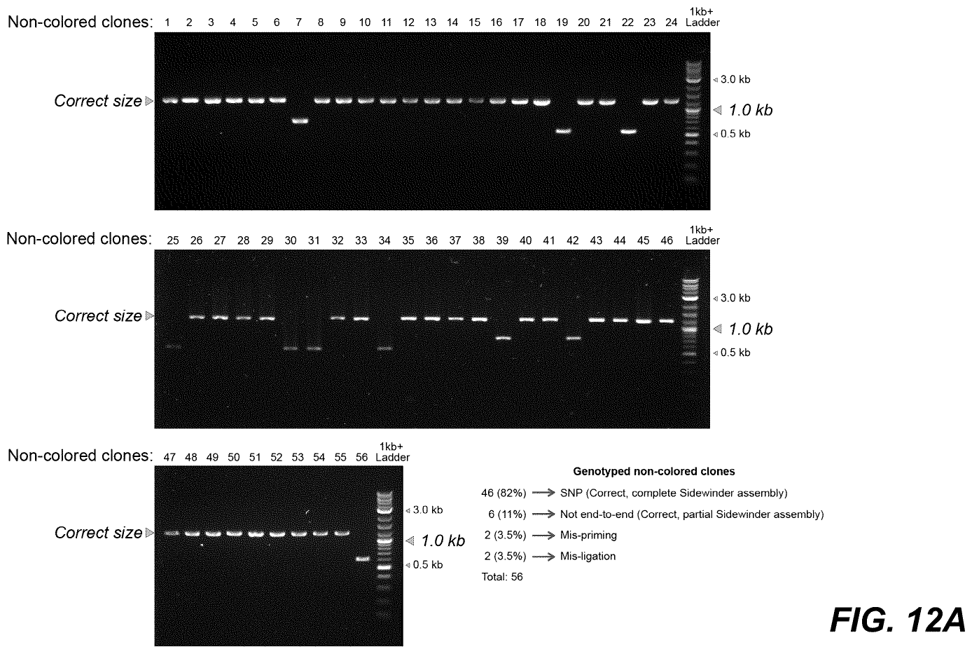

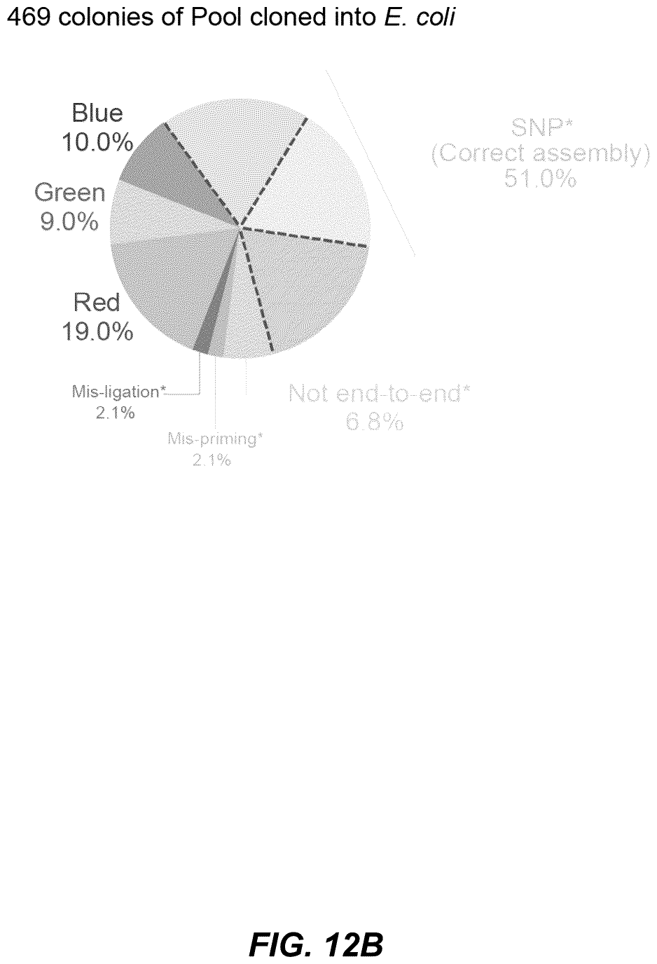

E. coli transformation of each dialed-out parallelly-assembled construct only yielded clones of the expected color (FIG. 4E). Transformation of the pool yielded distribution of all three expected phenotypes. A set of 56 non-colored clones were further characterized by PCR and sequencing to extrapolate the assembly accuracy for the pooled constructs post-transformation (FIG. 12). The in vivo characterization supports the quantitative assessments of the Nanopore sequencing data which further aligns with the qualitative clean gel bands, all together demonstrating the robustness of the Sidewinder assembly.

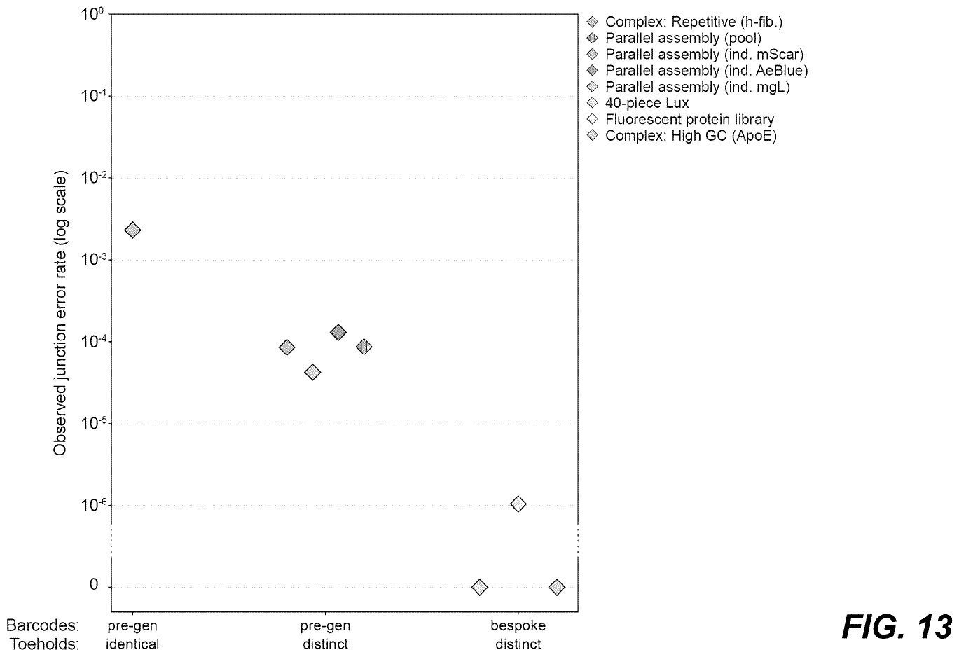

Higher rates of mis-ligation at the 3WJ were seen for the parallel and h-fibroin assembly, designed using a set of pre-generated orthogonal barcode sequences, compared to the high GC assembly and 40-piece assembly which had bespoke barcode designs using the NUPACK python package (FIG. 13). Thus, the minor crosstalk observed between fragments of the different genes in the parallel assembly is likely due to the sub-optimal design of the hand-picked barcode pairs and can be easily rectified in future experiments.

### Sidewinder Constructs DNA Libraries

Sidewinder can also assemble defined diversities across a large number of positions along the entire length of a DNA sequence to construct combinatorial libraries. In a combinatorial library, each variable position can diversified and assembled into a synthetic sequence with other diversified positions through DNA assembly. These libraries can then sorted, selected or screened for desired functions. This approach can be particularly useful in protein engineering where specific codons are varied at known or predicted residues to achieve a modified or improved protein function. Current methods for constructing combinatorial libraries using existing DNA assembly technologies can be limited in various aspects such as the theoretical library size, coverage, number of positions diversified simultaneously, and accuracy of assembly during construction.

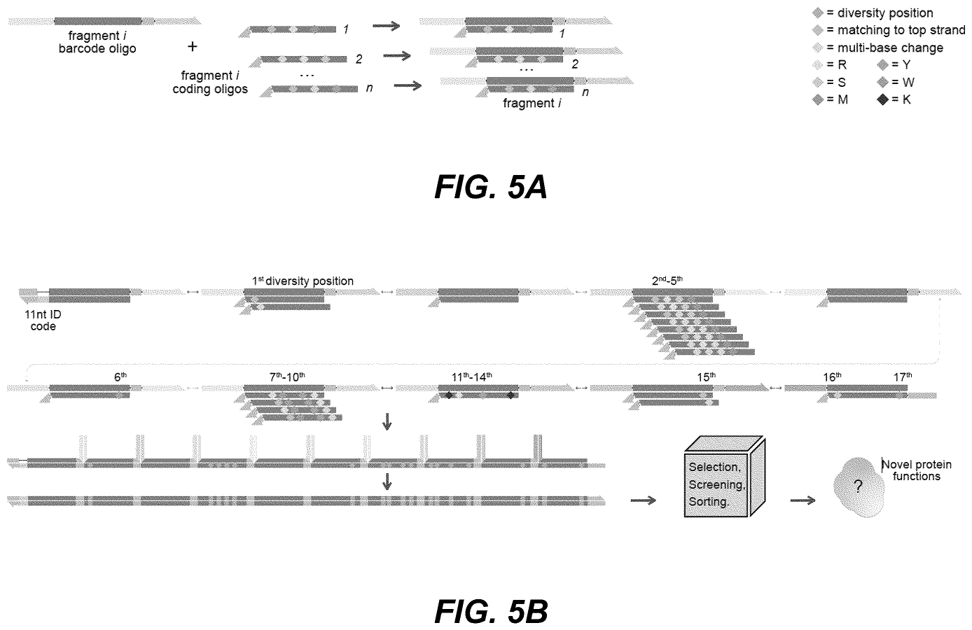

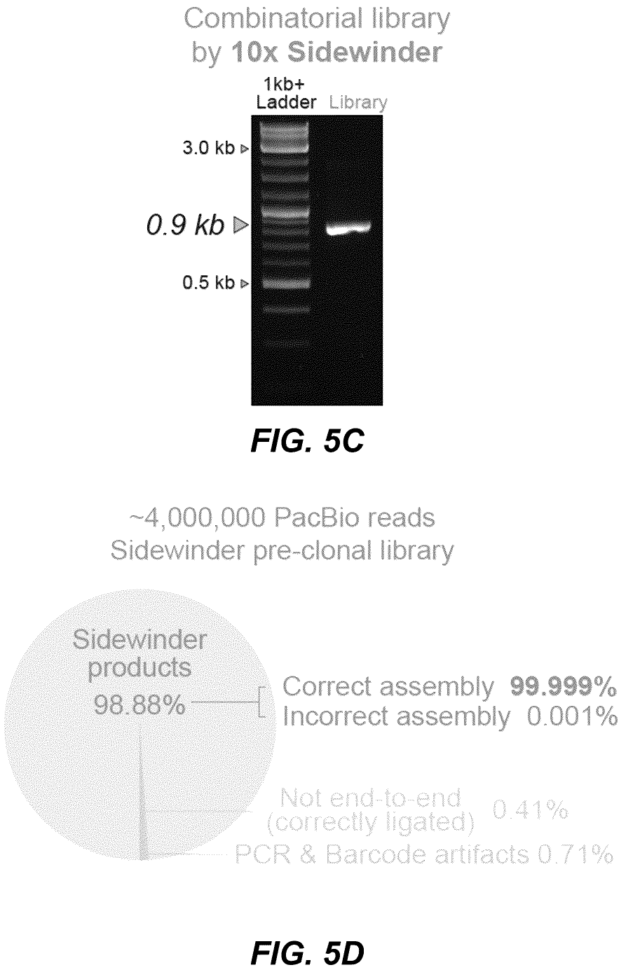

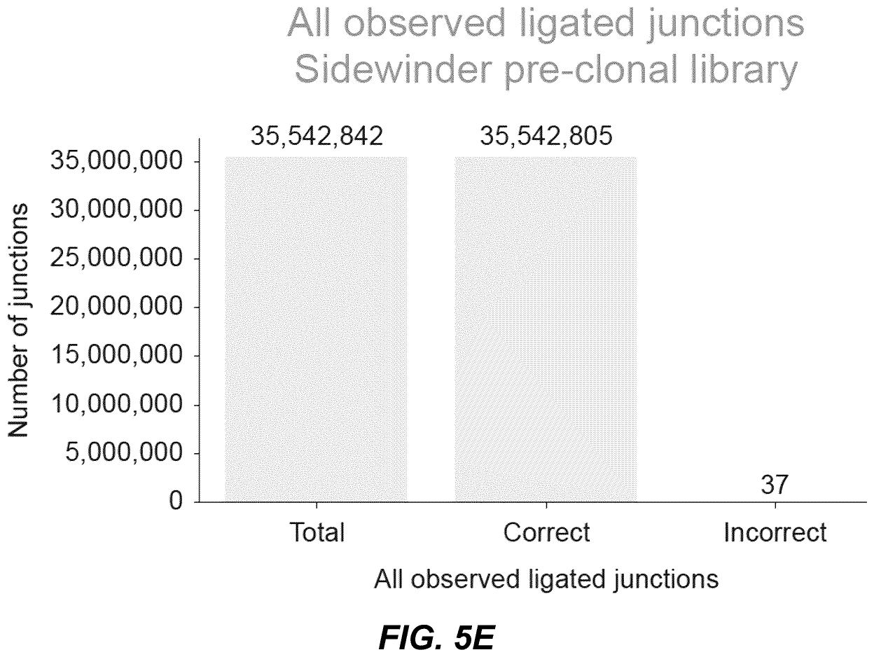

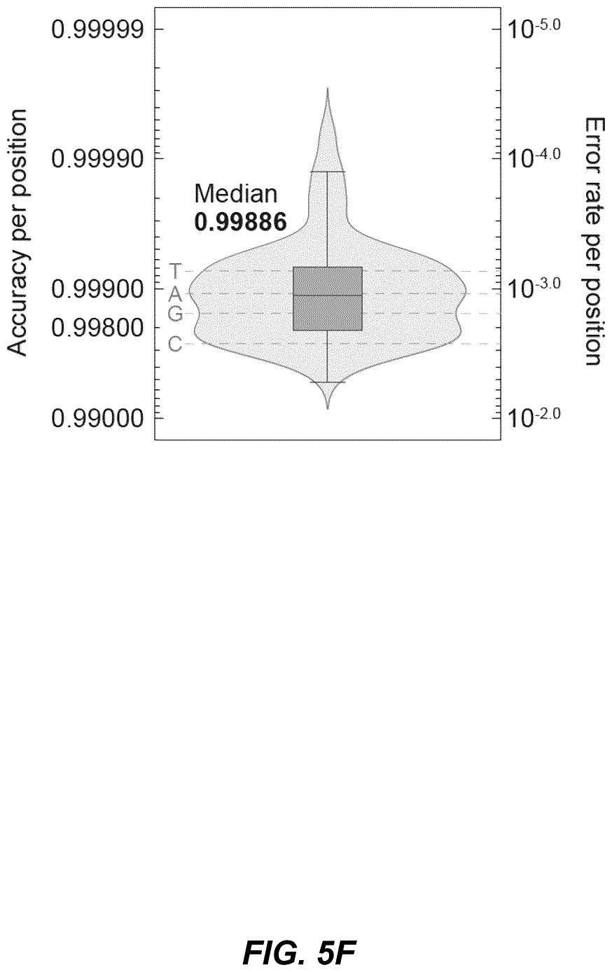

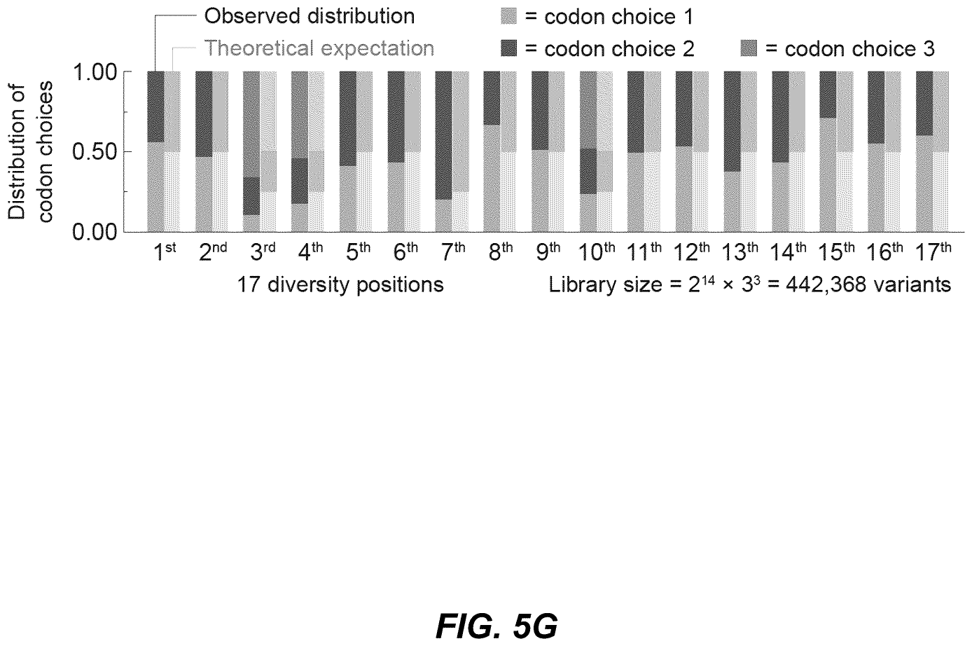

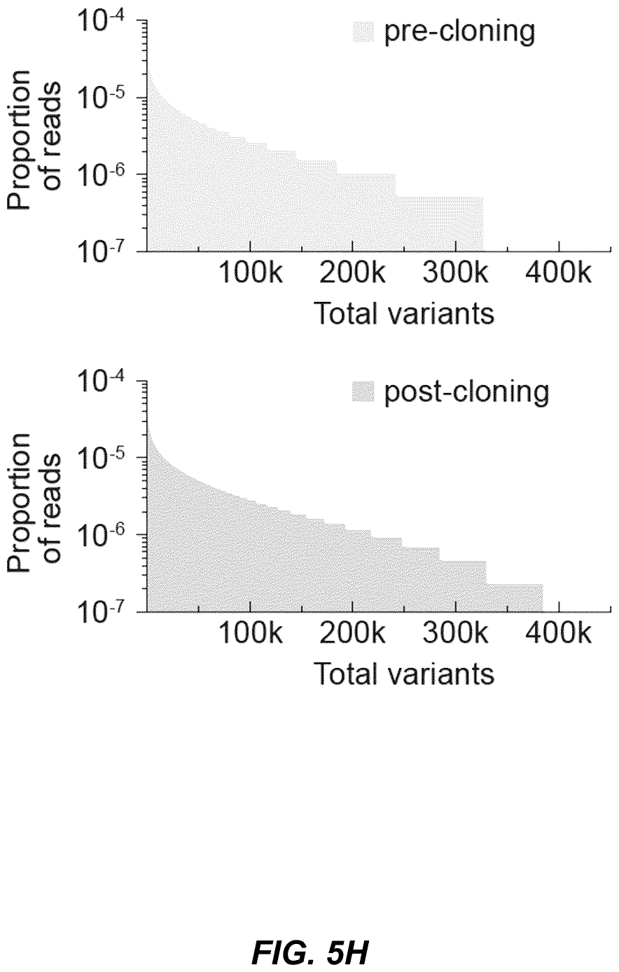

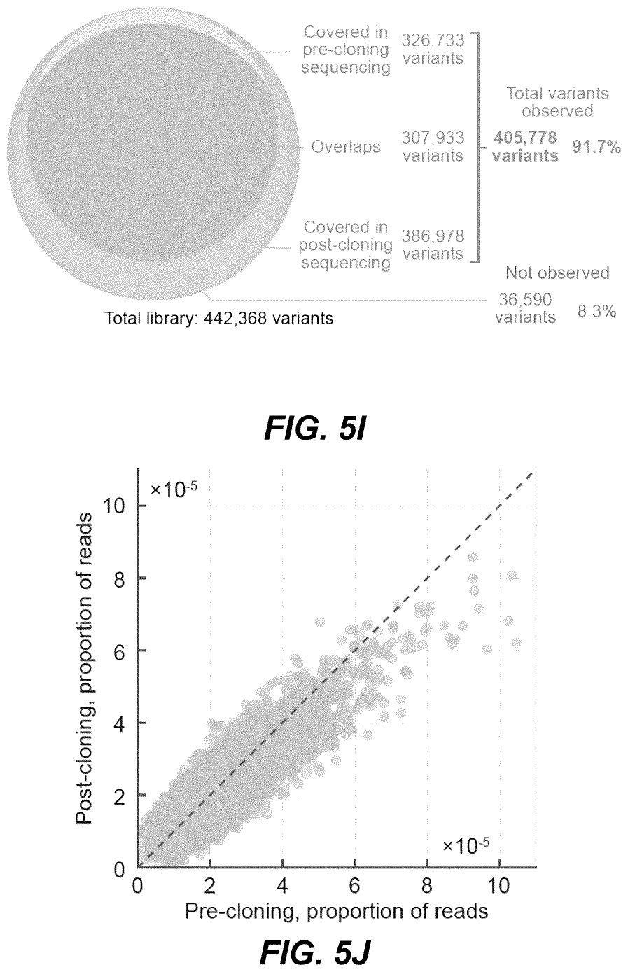

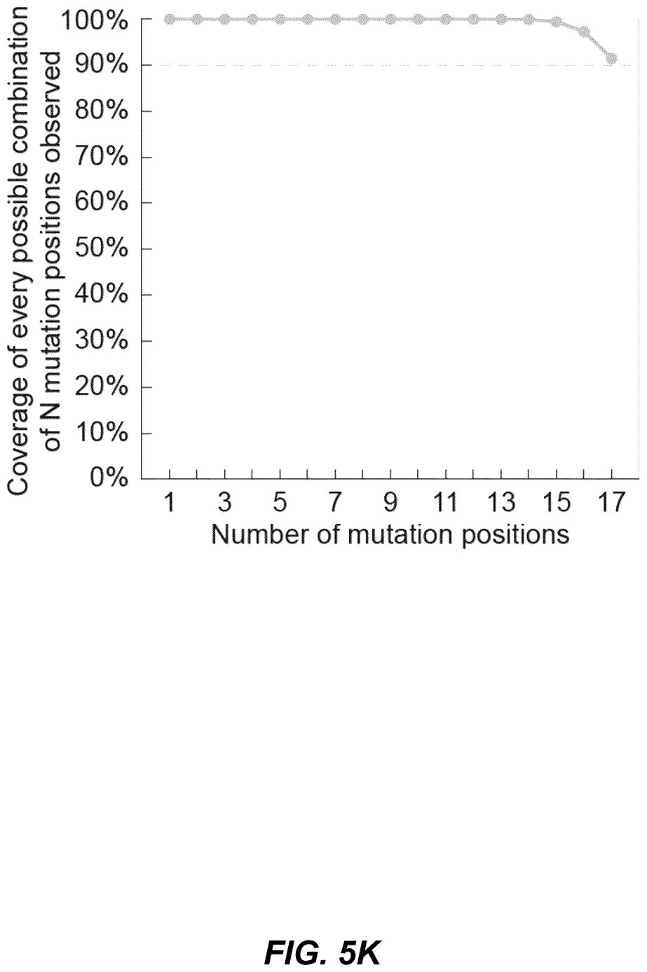

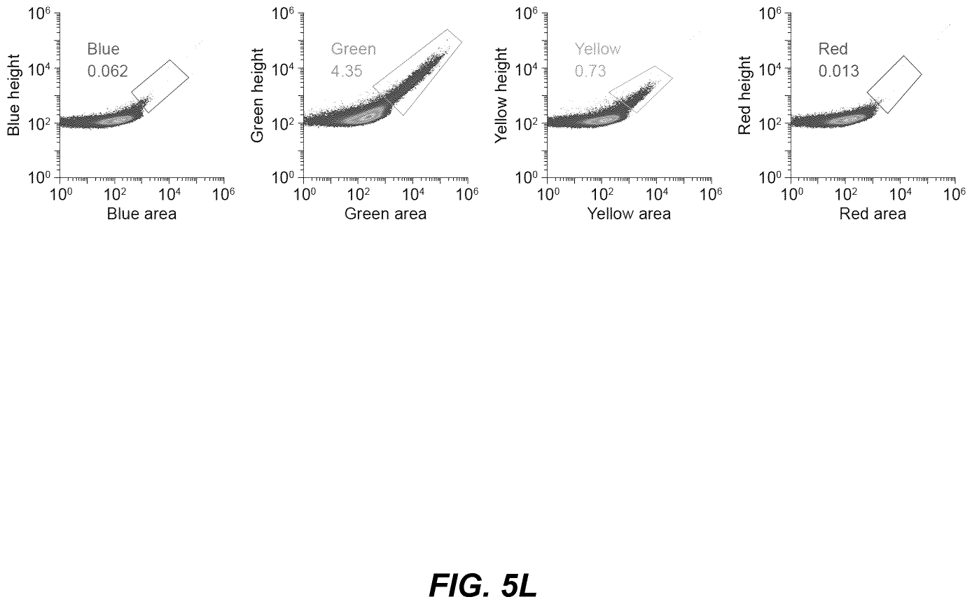

Sidewinder was applied to generate a combinatorial library by designing the assembly fragments to divide the gene for the fluorescent protein EGFP into a 10-piece Sidewinder assembly, where predefined codon variations were combinatorially diversified across 17 positions across the entire gene, yielding a theoretical library size of 442,368 possible mutation profiles (FIGS. 5A-5B). The Sidewinder library assembly resulted in a single strong target band (FIG. 5C) which was then cloned into a plasmid and transformed into E. coli cells. Fractions of the library both pre- and post-cloning were analyzed using PacBio sequencing for high-fidelity, single molecule long read sequencing.

For the pre-clonal Sidewinder assembly, 98.88% (3,832,803 reads) were correct 10-piece assemblies, 0.41% were partially assembled with the correct connection of a subset of the 10 pieces, and 0.71% were composed of PCR and barcode artifacts (FIG. 5D). Reassuringly, the high-fidelity PacBio data is consistent with previous Nanopore data, further supporting the robustness of the Sidewinder assembly in all demonstrated circumstances. Further analyzing the PacBio dataset for all instances of mis-ligated junctions observes only 37 mis-ligated junctions out of 35,542,842 total observed junctions (FIG. 5E). This corresponds to a misconnection rate at the 3WJ of just 1 in 960,616 (FIG. 13).

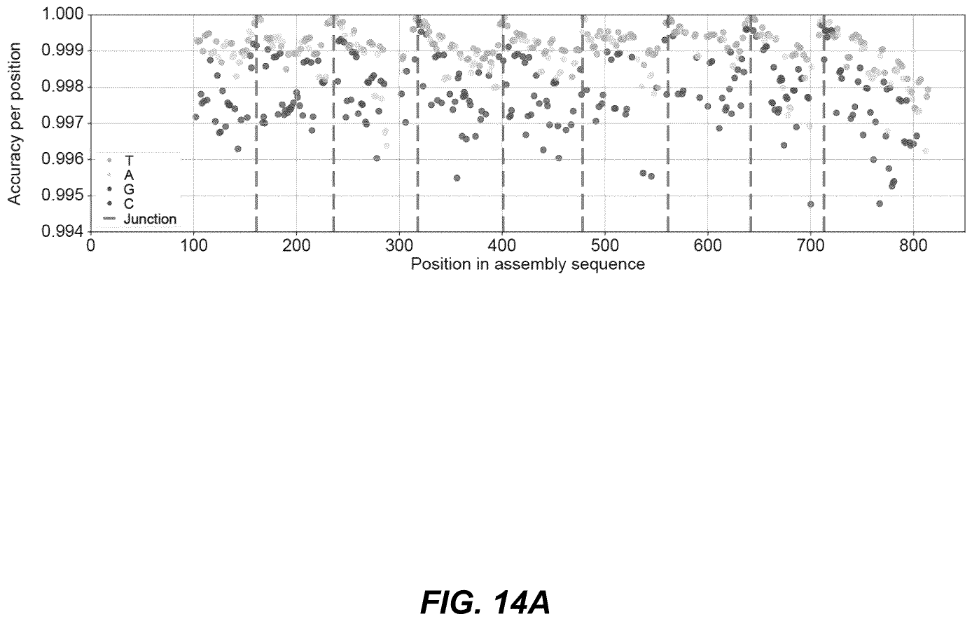

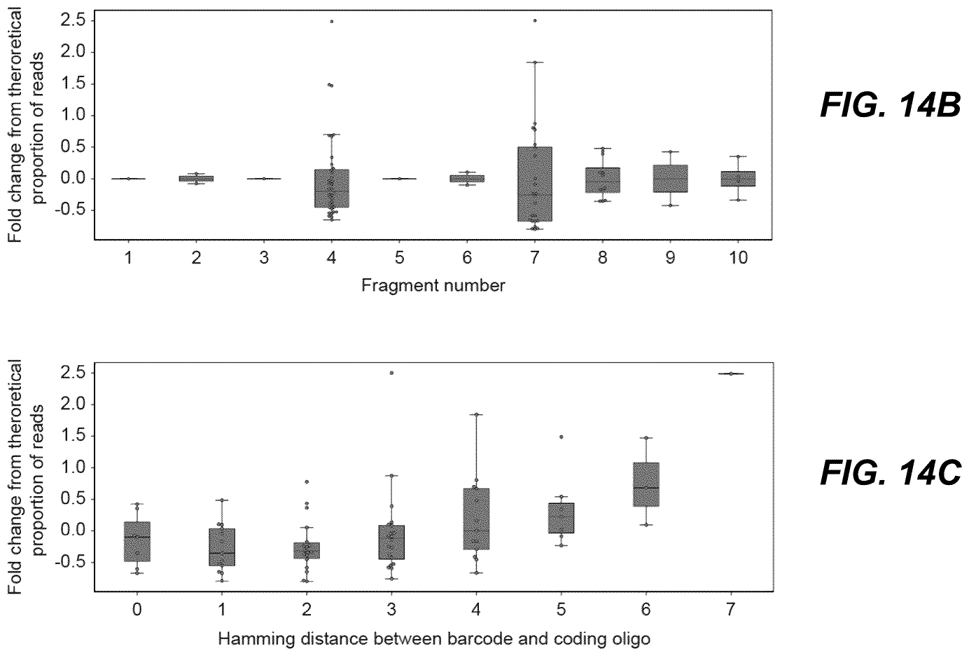

The median error rate for the oligos used for the assembly was calculated to be 10^{-2.943} (1 error in 877 bases, or 99.886% chance of a base being correct) (FIG. 5F). It was seen that, for these oligos, the per-base accuracy decreases with increased oligo length but due to the required ligation at the 3WJ, accuracy increases across assembly junctions (FIG. 14A). These observations suggest that Sidewinder does not introduce additional errors during the assembly and may subtly improve oligo fidelity. Due to Sidewinder's high-fidelity for multi-fragment assemblies, shorter DNA oligos composing a higher number of Sidewinder fragments may provide an advantage in synthesizing nucleotide-perfect genes. Based on the observed per-base error rate, an estimated 44.14% of the EGFP variants constructed are expected to be nucleotide perfect genes. This theoretical value is compared to a true value of 40.88% nucleotide perfect post-clonal genes in the PacBio sequencing data. This is contrasted to just 8.2% nucleotide perfect clones reported for a library of a 1 kb gene using PCA.

The diversity of the combinatorial library can be assessed by analyzing the mutation profiles (identity of the deliberately encoded mutations) at the codon level, fragment level, and gene level to compare the theoretical and experimental distribution of mutations at each level of library. At the codon level every codon mutation profile is represented in the library with an average absolute deviation of just 8.23 percentage points from the theoretical proportion of occurrence for that codon (FIG. 5G). At the fragment level, all 82 fragment mutation profiles are represented in the final library where generally the distribution of mutation profiles seems to have higher variance for fragments which had a higher number of possible mutation profiles such as fragment 4 (N=36) when compared to fragments with less possible diversity like fragment 2 (N=2) (FIG. 14B). Further, there does not appear to be a decrease in the likelihood of incorporation of a coding oligo when there are more mismatches to the fragment's barcode oligo (FIG. 14C) except for when those diversity positions appear in closer proximity to the junction as with diversity position 15 (FIG. 5G).

At the gene level, out of the 442,368 possible mutation profiles, a nearly identical distribution of occurrences in the mutation profiles of the pre- and post-clonal sequencing was observed and achieved a library coverage of 326,733 and 386,978 variants respectively for a combined total of 405,778 variants (307,933 overlap) (FIGS. 5H-51). By plotting the proportion of occurrences of the mutation profiles which are represented in both the pre- and post-clonal sequencing, a general trend was seen where the more highly represented clones pre-cloning remain highly represented post-cloning for this gene (FIG. 5J). The 405,778 variants observed correspond to a total library coverage of >91.7% of the 442,368 possible combinations of the 17 mutation positions. Within these mutation profiles, nearly every possible combination of as many as 15 mutation positions (>99.4%) in the library was observed with continued high representation all the way through every possible combination of 17 positions (FIG. 5K), which is an improvement over a recent comparable construction with Golden Gate.

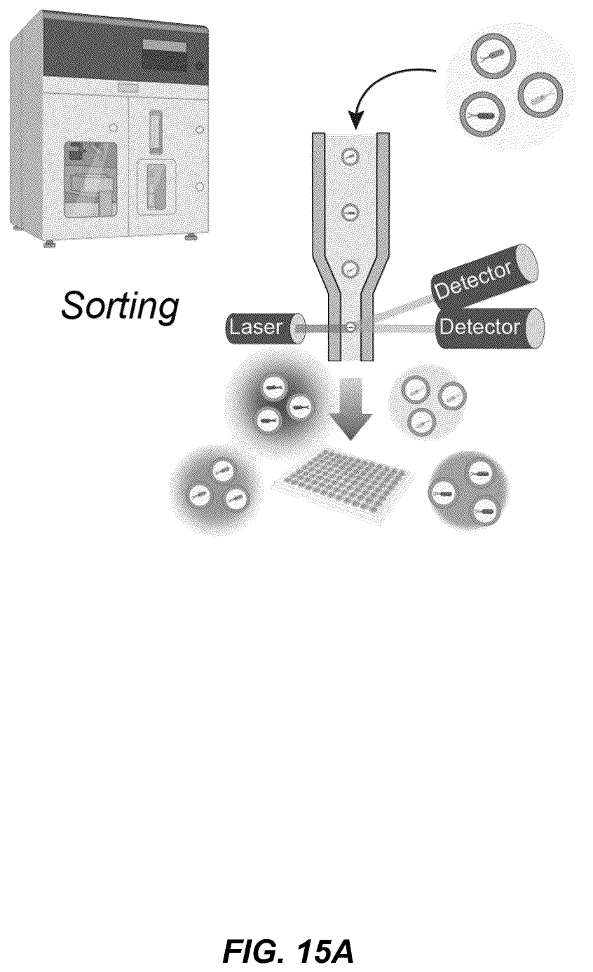

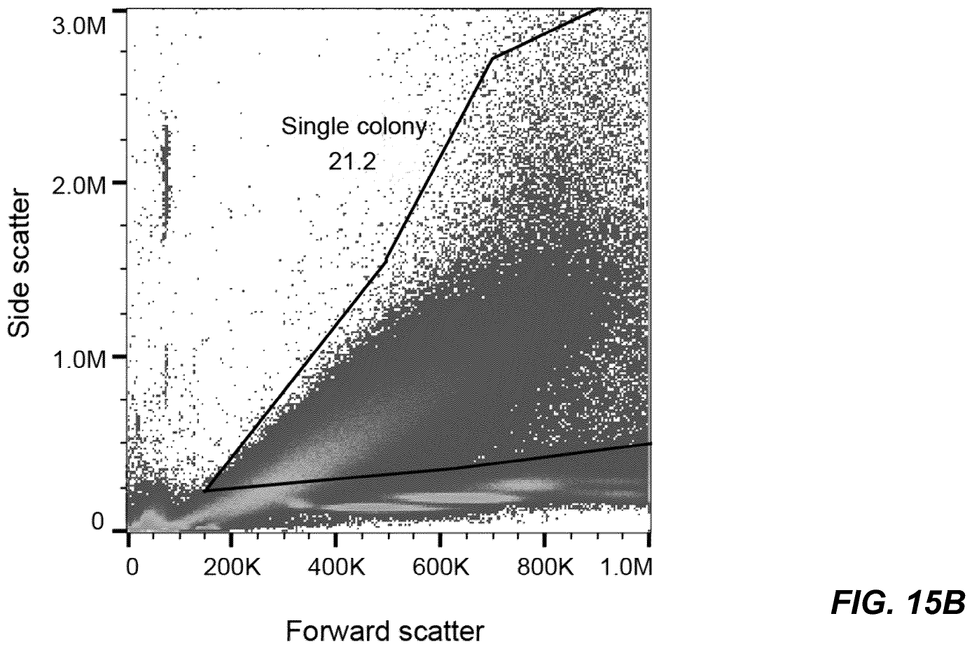

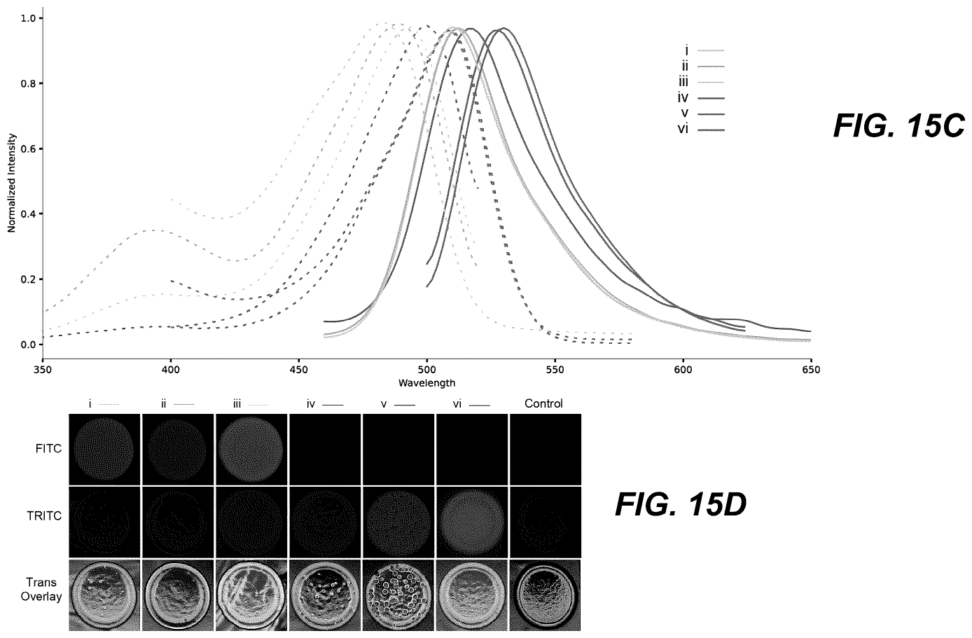

Sidewinder can be suitable for libraries of exceedingly large sizes, primarily limited by the fidelity of the oligos used and the ability to select/screen/sort post-clonal products. This library was designed by combining mutations that produce known phenotypes in fluorescent proteins with diverse excitation and emission spectra. The fluorescence signals for each member of the library was amplified by growing fluorescent protein-expressing clonal populations in hydrogel microparticles which were then screened by fluorescence-activated cell sorting (FACS). This approach enabled the rapid visualization and identification of distinct protein fluorescence expressed within the diverse library. Approximately 5,000,000 clones from the starting library were encapsulated into individual hydrogel microparticles and of those, 500,000 individual clones were screened using FACS and sorted to isolate mutations that resulted in different fluorescence emission characteristics from 400 nm to 700 nm when excited with 405 nm, 488 nm, 561 nm, and 638 nm lasers (FIGS. 15A-15B). Among the 500,000 screened colonies, variants with fluorescence signal corresponding to blue (0.06%), green (4.35%), yellow (0.73%) and red (0.01%) fluorescence proteins (FIG. 5L) were observed.

A subset of these sorted clones was further analyzed and a diversity of excitation and emission peaks was seen (FIG. 15C), confirmed through fluorescence microscopy (FIG. 15D). This demonstrates that Sidewinder not only enables the assembly of functional DNA fragments but also allows the simultaneous introduction of combinatorial mutations, resulting in highly diverse and functional molecular libraries.

### DISCUSSION

Sidewinder is the first DNA assembly method to decouple the DNA sequence information from the assembly information using the 3WJ, enabling true sequence-independent assembly and allowing construction of complex and diverse DNA sequences which were previously difficult to assemble. The reaction-specific barcode pairs b/b* allow the molecular information which directs assembly to be outsourced to DNA sequences not present in the final synthetic construct, permitting greater flexibility in exploring the entire DNA sequence landscape.

The potential of Sidewinder to conduct large assemblies is only fundamentally limited by the quality and size of the input DNA in some embodiments, as this Example demonstrates that Sidewinder does not introduce additional undesigned mutations. While the technology is in principle compatible with PCR and clonal DNA, herein is shown Sidewinder assembly of large numbers of oligos—the basis for all de novo synthetic DNA constructs. This Example also demonstrates Sidewinder's advancements to remove limits on the complexity of a constructed sequence by conducting assemblies which would be difficult or inaccessible with any other assembly technique.

Sidewinder can robustly assemble multiple distinct sequences simultaneously in a one-pot reaction. This capacity can be adapted to utilize large oligo pools to substantially reduce the cost per construct but requires further engineering to account for the formation of the unintended Sidewinder heteroduplexes prior to assembly and the higher truncation rate of pooled oligos. This can be important for the future of AI-facilitated design where Sidewinder will enable fast, scalable, and robust generation of the physical molecule which corresponds to the in silico prediction to facilitate connection between DNA sequence and expressed function.

Sidewinder can also assemble constructs with many defined diversities across the entire gene to generate large combinatorial libraries. Sidewinder can improve library accuracy, reliability, and coverage, overcoming prior limitations in constructing DNA libraries. Sidewinder libraries can be applied to different downstream pipelines for screening, selecting, or sorting for unique protein functions. The example demonstrated here shows how the reliability of Sidewinder paired with high-throughput FACS can enable the generation and screening of potentially millions of diverse DNA sequences encoding unique protein functions, all while maintaining exceptional throughput.

These results suggest that Sidewinder can be an important tool in the bioengineering toolbox as the technique can be interfaced with other genetic engineering techniques to better study and engineer biology. This technology can also impact diverse fields such as synthetic genomics, medicine, agriculture, material science, data storage and other bioengineering applications.

### Methods

### Fragment Design

When starting from DNA oligos, the number of junctions needed to assemble a construct can depend on the length of the construct and the length of the starting oligos being used. All component oligos which compose Sidewinder fragments used in these experiments are listed in Table 1. For an oligo of length L, the maximum bases of coding information for a fragment composed from these oligos (L_{c}) is L−2 L_{b} where L_{b} is the length of the barcode. Toeholds are then chosen starting maximally from L_{c} bases away from the previous junction. Hand-designed assemblies standardly use the maximal length fragments and use toeholds from position L_{c}-10 to L_{c} but can be shifted to avoid unintended toehold secondary structure. NUPACK designed assemblies choose a 10-base toehold within the range of position L_{c}-25 to L_{c} with an ensemble defect <0.1 from a secondary structure free toehold.

A range of toehold lengths and designs was tested, varying the ligation site from −10 bases to +10 bases on either side of the Sidewinder helix. Effective ligation was found occurring equal to or further than +6 bases from the Sidewinder helix (FIGS. 7A-7B). This led us to standardize the toehold length to 10 bases for the experiments described in this Example as to ensure sufficient distance of the nick from the Sidewinder helix to accommodate ligase docking and effective ligation.

Barcodes were designed to be compatible with their respective toehold after the location of the junction is chosen. Barcode sequences were chosen or generated based on the predicted secondary structure and cross talk between other toehold-barcode sequences at the assembly's ligation temperature. The h-fibroin and parallel assembly barcodes were designed using a “guess-check” method choosing from a set list of pre-generated orthogonal barcodes. Starting with the first toehold, a barcode sequence was arbitrarily chosen (“guess”) and appended to the 3′ end of the toehold and checked for secondary structure at 50° C. with complex size 2 using NUPACK web-browser (“check”). The subsequent barcodes were then chosen from the pre-generated list, checked individually in the same manner, then checked for cross reactivity against all previously chosen toehold-barcode sense and antisense sequences at 50° C. and complex size 2. All barcodes in this study use natural bases but it is anticipated that the specificity and diversity of Sidewinder barcodes can be expanded to include unnatural bases and other DNA nanotechnology interactions.

The 5 to 40-piece Lux assemblies, the ApoE assembly, and the library assembly had bespoke barcode sequences generated for the specific assembly using NUPACK python package. Target strand (NUPACK variable) secondary structure was defined to be fully unpaired for each single stranded barcode/toehold pair combination. Complexes (NUPACK variable) were defined to take on the desired 3WJ structure for barcode/toeholds. Step tubes (NUPACK variable) are defined such that in Step 0 individual barcode/toehold sequences take on the desired unpaired secondary structure prior to assembly, and in Step 1, barcode/toeholds sequences pair with the intended assembly partner during assembly at 50° C. with an ensemble defect <0.1. After barcode generation, all secondary structure and cross reactivity of chosen barcodes were checked using NUPACK web-browser.

For the length of the Sidewinder barcodes, barcode lengths were tested from 15 to 21 bases, both with and without a T-T or U-U mismatch at the base of the 3WJ for added stability and neither seem to have bearing on ligation efficiency (Table 1). A variety of commercially available ligases were also tested and found high variability in ligation efficiency at −10 bases from the Sidewinder helix across the ligases tested (FIG. 7C). Of the various ligases tested, Taq ligase and HiFi Taq ligase were picked as the preferred ligases due to their efficiency of ligation at the 3WJ and stability at extremely high temperatures.

### Oligo Purchasing

All assembly oligos were purchased from Millipore-Sigma with Standard DNA Synthesis for DNA Oligos in Tubes which has a max oligo length of 120 bases. The only exception was fragment 4 of the identical toehold assembly which was ordered as a Long Oligo in order to enable 4 identical toeholds (Table 1). Barcode oligos were ordered with standard desalt purification and coding oligos were ordered PAGE purified but has since been seen to be superfluous (FIG. 8F). Both barcode and coding oligos for the fluorescent protein library were ordered with cartridge purification. Both barcode and coding oligos for the Sidewinder characterization in FIG. 1 were ordered PAGE purified. PCR amplification primers were ordered from Integrated DNA Technologies with standard desalt purity. All oligos are listed in Table 1. All Sidewinder component oligos were shipped dry.

TABLE 1
Synthetic DNA oligos utilized in experiments

| | SEQ | | | | | |
|---|---|---|---|---|---|---|
| | ID | | FIG. | | | |
| Oligo_ID | NO: | Purity | # | Method | Construct | Use |
| SP_1 | 1 | PAGE | 1 | Sidewinder | 1+1_Fluor | Assembly |
| SP_2 | 2 | PAGE | 1 | Sidewinder | 1+1_Fluor | Assembly |
| SP_3 | 3 | PAGE | 1 | Sidewinder | 1+1_Fluor | Assembly |
| SP_4 | 4 | PAGE | 1 | Sidewinder | 1+1_Fluor | Assembly |
| SP_5 | 5 | PAGE | 1 | Sidewinder | 1+1_Fluor | Assembly |
| SP_6 | 6 | PAGE | 1 | Sidewinder | 1+1_Fluor | Assembly |
| SP_7 | 7 | PAGE | 1 | Sidewinder | 1+1_Fluor | Assembly |
| SP_8 | 8 | Desalt | 2 | Sidewinder | LUX ABC | Assembly |
| SP_9 | 9 | PAGE | 2 | Sidewinder | LUX ABC | Assembly |
| SP_10 | 10 | Desalt | 2 | Sidewinder | LUX ABC | Assembly |
| SP_11 | 11 | PAGE | 2 | Sidewinder | LUX ABC | Assembly |
| SP_12 | 12 | Desalt | 2 | Sidewinder | LUX ABC | Assembly |
| SP_13 | 13 | PAGE | 2 | Sidewinder | LUX ABC | Assembly |
| SP_14 | 14 | Desalt | 2 | Sidewinder | LUX ABC | Assembly |
| SP_15 | 15 | PAGE | 2 | Sidewinder | LUX ABC | Assembly |
| SP_16 | 16 | Desalt | 2 | Sidewinder | LUX ABC | Assembly |
| SP_17 | 17 | PAGE | 2 | Sidewinder | LUX ABC | Assembly |
| SP_18 | 18 | Desalt | 2 | Sidewinder | LUX ABC | Assembly |
| SP_19 | 19 | PAGE | 2 | Sidewinder | LUX ABC | Assembly |
| SP_20 | 20 | Desalt | 2 | Sidewinder | LUX ABC | Assembly |
| SP_21 | 21 | PAGE | 2 | Sidewinder | LUX ABC | Assembly |
| SP_22 | 22 | Desalt | 2 | Sidewinder | LUX ABC | Assembly |
| SP_23 | 23 | PAGE | 2 | Sidewinder | LUX ABC | Assembly |
| SP_24 | 24 | Desalt | 2 | Sidewinder | LUX ABC | Assembly |
| SP_25 | 25 | PAGE | 2 | Sidewinder | LUX ABC | Assembly |
| SP_26 | 26 | Desalt | 2 | Sidewinder | LUX ABC | Assembly |
| SP_27 | 27 | PAGE | 2 | Sidewinder | LUX ABC | Assembly |
| SP_28 | 28 | Desalt | 2 | Sidewinder | LUX ABC | Assembly |
| SP_29 | 29 | PAGE | 2 | Sidewinder | LUX ABC | Assembly |
| SP_30 | 30 | Desalt | 2 | Sidewinder | LUX ABC | Assembly |
| SP_31 | 31 | PAGE | 2 | Sidewinder | LUX ABC | Assembly |
| SP_32 | 32 | Desalt | 2 | Sidewinder | LUX ABC | Assembly |
| SP_33 | 33 | PAGE | 2 | Sidewinder | LUX ABC | Assembly |
| SP_34 | 34 | Desalt | 2 | Sidewinder | LUX ABC | Assembly |
| SP_35 | 35 | PAGE | 2 | Sidewinder | LUX ABC | Assembly |
| SP_36 | 36 | Desalt | 2 | Sidewinder | LUX ABC | Assembly |
| SP_37 | 37 | PAGE | 2 | Sidewinder | LUX ABC | Assembly |
| SP_38 | 38 | Desalt | 2 | Sidewinder | LUX ABC | Assembly |
| SP_39 | 39 | PAGE | 2 | Sidewinder | LUX ABC | Assembly |
| SP_40 | 40 | Desalt | 2 | Sidewinder | LUX ABC | Assembly |
| SP_41 | 41 | PAGE | 2 | Sidewinder | LUX ABC | Assembly |
| SP_42 | 42 | Desalt | 2 | Sidewinder | LUX ABC | Assembly |
| SP_43 | 43 | PAGE | 2 | Sidewinder | LUX ABC | Assembly |
| SP_44 | 44 | Desalt | 2 | Sidewinder | LUX ABC | Assembly |
| SP_45 | 45 | PAGE | 2 | Sidewinder | LUX ABC | Assembly |
| SP_46 | 46 | Desalt | 2 | Sidewinder | LUX ABC | Assembly |
| SP_47 | 47 | PAGE | 2 | Sidewinder | LUX ABC | Assembly |
| SP_48 | 48 | Desalt | 2 | Sidewinder | LUX ABC | Assembly |
| SP_49 | 49 | PAGE | 2 | Sidewinder | LUX ABC | Assembly |
| SP_50 | 50 | Desalt | 2 | Sidewinder | LUX ABC | Assembly |
| SP_51 | 51 | PAGE | 2 | Sidewinder | LUX ABC | Assembly |
| SP_52 | 52 | Desalt | 2 | Sidewinder | LUX ABC | Assembly |
| SP_53 | 53 | PAGE | 2 | Sidewinder | LUX ABC | Assembly |
| SP_54 | 54 | Desalt | 2 | Sidewinder | LUX ABC | Assembly |
| SP_55 | 55 | PAGE | 2 | Sidewinder | LUX ABC | Assembly |
| SP_56 | 56 | Desalt | 2 | Sidewinder | LUX ABC | Assembly |
| SP_57 | 57 | PAGE | 2 | Sidewinder | LUX ABC | Assembly |
| SP_58 | 58 | Desalt | 2 | Sidewinder | LUX ABC | Assembly |
| SP_59 | 59 | PAGE | 2 | Sidewinder | LUX ABC | Assembly |
| SP_60 | 50 | Desalt | 2 | Sidewinder | LUX ABC | Assembly |
| SP_61 | 61 | PAGE | 2 | Sidewinder | LUX ABC | Assembly |
| SP_62 | 62 | Desalt | 2 | Sidewinder | LUX ABC | Assembly |
| SP_63 | 63 | PAGE | 2 | Sidewinder | LUX ABC | Assembly |
| SP_64 | 64 | Desalt | 2 | Sidewinder | LUX ABC | Assembly |
| SP_65 | 65 | PAGE | 2 | Sidewinder | LUX ABC | Assembly |
| SP_66 | 66 | Desalt | 2 | Sidewinder | LUX ABC | Assembly |
| SP_67 | 67 | PAGE | 2 | Sidewinder | LUX ABC | Assembly |
| SP_68 | 68 | Desalt | 2 | Sidewinder | LUX ABC | Assembly |
| SP_69 | 69 | PAGE | 2 | Sidewinder | LUX ABC | Assembly |
| SP_70 | 70 | Desalt | 2 | Sidewinder | LUX ABC | Assembly |
| SP_71 | 71 | PAGE | 2 | Sidewinder | LUX ABC | Assembly |
| SP_72 | 72 | Desalt | 2 | Sidewinder | LUX ABC | Assembly |
| SP_73 | 73 | PAGE | 2 | Sidewinder | LUX ABC | Assembly |
| SP_74 | 74 | Desalt | 2 | Sidewinder | LUX ABC | Assembly |
| SP_75 | 75 | PAGE | 2 | Sidewinder | LUX ABC | Assembly |
| SP_76 | 76 | Desalt | 2 | Sidewinder | LUX ABC | Assembly |
| SP_77 | 77 | PAGE | 2 | Sidewinder | LUX ABC | Assembly |
| SP_78 | 78 | Desalt | 2 | Sidewinder | LUX ABC | Assembly |
| SP_79 | 79 | PAGE | 2 | Sidewinder | LUX ABC | Assembly |
| SP_80 | 80 | Desalt | 2 | Sidewinder | LUX ABC | Assembly |
| SP_81 | 81 | PAGE | 2 | Sidewinder | LUX ABC | Assembly |
| SP_82 | 82 | Desalt | 2 | Sidewinder | LUX ABC | Assembly |
| SP_83 | 83 | PAGE | 2 | Sidewinder | LUX ABC | Assembly |
| SP_84 | 84 | Desalt | 2 | Sidewinder | LUX ABC | Assembly |
| SP_85 | 85 | PAGE | 2 | Sidewinder | LUX ABC | Assembly |
| SP_86 | 86 | Desalt | 2 | Sidewinder | LUX ABC | Assembly |
| SP_87 | 87 | PAGE | 2 | Sidewinder | LUX ABC | Assembly |
| SP_88 | 88 | Desalt | 2 | PCA | LUX ABC | Assembly |
| SP_89 | 89 | Desalt | 2 | PCA | LUX ABC | Assembly |
| SP_90 | 90 | Desalt | 2 | PCA | LUX ABC | Assembly |
| SP_91 | 91 | Desalt | 2 | PCA | LUX ABC | Assembly |
| SP_92 | 92 | Desalt | 2 | PCA | LUX ABC | Assembly |
| SP_93 | 93 | Desalt | 2 | PCA | LUX ABC | Assembly |
| SP_94 | 94 | Desalt | 2 | PCA | LUX ABC | Assembly |
| SP_95 | 95 | Desalt | 2 | PCA | LUX ABC | Assembly |
| SP_96 | 96 | Desalt | 2 | PCA | LUX ABC | Assembly |
| SP_97 | 97 | Desalt | 2 | PCA | LUX ABC | Assembly |
| SP_98 | 98 | Desalt | 2 | PCA | LUX ABC | Assembly |
| SP_99 | 99 | Desalt | 2 | PCA | LUX ABC | Assembly |
| SP_100 | 100 | Desalt | 2 | PCA | LUX ABC | Assembly |
| SP_101 | 101 | Desalt | 2 | PCA | LUX ABC | Assembly |
| SP_102 | 102 | Desalt | 2 | PCA | LUX ABC | Assembly |
| SP_103 | 103 | Desalt | 2 | PCA | LUX ABC | Assembly |
| SP_104 | 104 | Desalt | 2 | PCA | LUX ABC | Assembly |
| SP_105 | 105 | Desalt | 2 | PCA | LUX ABC | Assembly |
| SP_106 | 106 | Desalt | 2 | PCA | LUX ABC | Assembly |
| SP_107 | 107 | Desalt | 2 | PCA | LUX ABC | Assembly |
| SP_108 | 108 | PAGE | 8 | Sidewinder | mGL + mScar | Assembly |
| SP_109 | 109 | PAGE | 8 | Sidewinder | mGL + mScar | Assembly |
| SP_110 | 110 | PAGE | 8 | Sidewinder | mGL + mScar | Assembly |
| SP_111 | 111 | PAGE | 8 | Sidewinder | mGL + mScar | Assembly |
| SP_112 | 112 | PAGE | 8 | Sidewinder | mGL + mScar | Assembly |
| SP_113 | 113 | PAGE | 8 | Sidewinder | mGL + mScar | Assembly |
| SP_114 | 114 | PAGE | 8 | Sidewinder | mGL + mScar | Assembly |
| SP_115 | 115 | PAGE | 8 | Sidewinder | mGL + mScar | Assembly |
| SP_116 | 116 | PAGE | 8 | Sidewinder | mGL + mScar | Assembly |
| SP_117 | 117 | PAGE | 8 | Sidewinder | mGL + mScar | Assembly |
| SP_118 | 118 | PAGE | 8 | Sidewinder | mGL + mScar | Assembly |
| SP_119 | 119 | PAGE | 8 | Sidewinder | mGL + mScar | Assembly |
| SP_120 | 120 | PAGE | 8 | Sidewinder | mGL + mScar | Assembly |
| SP_121 | 121 | PAGE | 8 | Sidewinder | mGL + mScar | Assembly |
| SP_122 | 122 | PAGE | 8 | Sidewinder | mGL + mScar | Assembly |
| SP_123 | 123 | PAGE | 8 | Sidewinder | mGL + mScar | Assembly |
| SP_124 | 124 | PAGE | 8 | Sidewinder | mGL + mScar | Assembly |
| SP_125 | 125 | PAGE | 8 | Sidewinder | mGL + mScar | Assembly |
| SP_126 | 126 | PAGE | 8 | Sidewinder | mGL + mScar | Assembly |
| SP_127 | 127 | Desalt | 8 | Sidewinder | mGL + mScar | Assembly |
| SP_128 | 128 | Desalt | 8 | Sidewinder | mGL + mScar | Assembly |
| SP_129 | 129 | Desalt | 8 | Sidewinder | mGL + mScar | Assembly |
| SP_130 | 130 | Desalt | 8 | Sidewinder | mGL + mScar | Assembly |
| SP_131 | 131 | Desalt | 8 | Sidewinder | mGL + mScar | Assembly |
| SP_132 | 132 | Desalt | 8 | Sidewinder | mGL + mScar | Assembly |
| SP_133 | 133 | Desalt | 8 | Sidewinder | mGL + mScar | Assembly |
| SP_134 | 134 | Desalt | 8 | Sidewinder | mGL + mScar | Assembly |
| SP_135 | 135 | Desalt | 8 | Sidewinder | mGL + mScar | Assembly |
| SP_136 | 136 | Desalt | 8 | Sidewinder | mGL + mScar | Assembly |
| SP_137 | 137 | Desalt | 8 | Sidewinder | mGL + mScar | Assembly |
| SP_138 | 138 | Desalt | 8 | Sidewinder | mGL + mScar | Assembly |
| SP_139 | 139 | Desalt | 8 | Sidewinder | mGL + mScar | Assembly |
| SP_140 | 140 | Desalt | 8 | Sidewinder | mGL + mScar | Assembly |
| SP_141 | 141 | Desalt | 8 | Sidewinder | mGL + mScar | Assembly |
| SP_142 | 142 | Desalt | 8 | Sidewinder | mGL + mScar | Assembly |
| SP_143 | 143 | Desalt | 8 | Sidewinder | mGL + mScar | Assembly |
| SP_144 | 144 | Desalt | 8 | Sidewinder | mGL + mScar | Assembly |
| SP_145 | 145 | Desalt | 8 | Sidewinder | mGL + mScar | Assembly |
| SP_146 | 146 | Desalt | 8 | Sidewinder | mGL + mScar | Assembly |
| SP_147 | 147 | Desalt | 9 | Sidewinder | mGL + mScar | Assembly |
| SP_148 | 148 | Desalt | 8 | PCA | mGL + mScar | Assembly |
| SP_149 | 149 | Desalt | 8 | PCA | mGL + mScar | Assembly |
| SP_150 | 150 | Desalt | 8 | PCA | mGL + mScar | Assembly |
| SP_151 | 151 | Desalt | 8 | PCA | mGL + mScar | Assembly |
| SP_152 | 152 | Desalt | 8 | PCA | mGL + mScar | Assembly |
| SP_153 | 153 | Desalt | 8 | PCA | mGL + mScar | Assembly |
| SP_154 | 154 | Desalt | 8 | PCA | mGL + mScar | Assembly |
| SP_155 | 155 | Desalt | 8 | PCA | mGL + mScar | Assembly |
| SP_156 | 156 | Desalt | 8 | PCA | mGL + mScar | Assembly |
| SP_157 | 157 | Desalt | 8 | PCA | mGL + mScar | Assembly |
| SP_158 | 158 | Desalt | 8 | PCA | mGL + mScar | Assembly |
| SP_159 | 159 | Desalt | 8 | PCA | mGL + mScar | Assembly |
| SP_160 | 160 | Desalt | 8 | PCA | mGL + mScar | Assembly |
| SP_161 | 161 | Desalt | 8 | PCA | mGL + mScar | Assembly |
| SP_162 | 162 | Desalt | 8 | PCA | mGL + mScar | Assembly |
| SP_163 | 163 | Desalt | 8 | PCA | mGL + mScar | Assembly |
| SP_164 | 164 | Desalt | 8 | PCA | mGL + mScar | Assembly |
| SP_165 | 165 | Desalt | 8 | PCA | mGL + mScar | Assembly |
| SP_166 | 166 | Desalt | 8 | PCA | mGL + mScar | Assembly |
| SP_167 | 167 | Desalt | 8 | PCA | mGL + mScar | Assembly |
| SP_168 | 168 | Desalt | 8 | Sidewinder, 4bp overhang, | LUX ABC | Assembly |
| | | | | 10bp overhang | | |
| SP_169 | 169 | Desalt | 8 | Sidewinder, 4bp overhang, | LUX ABC | Assembly |
| | | | | 10bp overhang | | |
| SP_170 | 170 | Desalt | 8 | Sidewinder, 4bp overhang, | LUX ABC | Assembly |
| | | | | 10bp overhang | | |
| SP_171 | 171 | Desalt | 8 | Sidewinder, 4bp overhang, | LUX ABC | Assembly |
| | | | | 10bp overhang | | |
| SP_172 | 172 | Desalt | 8 | Sidewinder, 4bp overhang, | LUX ABC | Assembly |
| | | | | 10bp overhang | | |
| SP_173 | 173 | Desalt | 8 | Sidewinder, 4bp overhang, | LUX ABC | Assembly |
| | | | | 10bp overhang | | |
| SP_174 | 174 | Desalt | 8 | Sidewinder, 4bp overhang, | LUX ABC | Assembly |
| | | | | 10bp overhang | | |
| SP_175 | 175 | Desalt | 8 | Sidewinder, 4bp overhang, | LUX ABC | Assembly |
| | | | | 10bp overhang | | |
| SP_176 | 176 | Desalt | 8 | Sidewinder, 4bp overhang, | LUX ABC | Assembly |
| | | | | 10bp overhang | | |
| SP_177 | 177 | Desalt | 8 | Sidewinder, 4bp overhang, | LUX ABC | Assembly |
| | | | | 10bp overhang | | |
| SP_178 | 178 | Desalt | 8 | Sidewinder, 4bp overhang, | LUX ABC | Assembly |
| | | | | 10bp overhang | | |
| SP_179 | 179 | Desalt | 8 | Sidewinder, 4bp overhang, | LUX ABC | Assembly |
| | | | | 10bp overhang | | |
| SP_180 | 180 | Desalt | 8 | Sidewinder, 4bp overhang, | LUX ABC | Assembly |
| | | | | 10bp overhang | | |
| SP_181 | 181 | Desalt | 8 | Sidewinder, 4bp overhang, | LUX ABC | Assembly |
| | | | | 10bp overhang | | |
| SP_182 | 182 | Desalt | 8 | Sidewinder, 4bp overhang, | LUX ABC | Assembly |
| | | | | 10bp overhang | | |
| SP_183 | 183 | Desalt | 8 | Sidewinder, 4bp overhang, | LUX ABC | Assembly |
| | | | | 10bp overhang | | |
| SP_184 | 184 | Desalt | 8 | Sidewinder, 4bp overhang, | LUX ABC | Assembly |
| | | | | 10bp overhang | | |
| SP_185 | 185 | Desalt | 8 | Sidewinder, 4bp overhang, | LUX ABC | Assembly |
| | | | | 10bp overhang | | |
| SP_186 | 186 | Desalt | 8 | Sidewinder, 4bp overhang, | LUX ABC | Assembly |
| | | | | 10bp overhang | | |
| SP_187 | 187 | Desalt | 8 | Sidewinder, 4bp overhang, | LUX ABC | Assembly |
| | | | | 10bp overhang | | |
| SP_188 | 188 | Desalt | 8 | Sidewinder, 4bp overhang, | LUX ABC | Assembly |
| | | | | 10bp overhang | | |
| SP_189 | 189 | Desalt | 8 | Sidewinder, 4bp overhang, | LUX ABC | Assembly |
| | | | | 10bp overhang | | |
| SP_190 | 190 | Desalt | 8 | Sidewinder, 4bp overhang, | LUX ABC | Assembly |
| | | | | 10bp overhang | | |
| SP_191 | 191 | Desalt | 8 | Sidewinder, 4bp overhang, | LUX ABC | Assembly |
| | | | | 10bp overhang | | |
| SP_192 | 192 | Desalt | 8 | Sidewinder, 4bp overhang, | LUX ABC | Assembly |
| | | | | 10bp overhang | | |
| SP_193 | 193 | Desalt | 8 | Sidewinder, 4bp overhang, | LUX ABC | Assembly |
| | | | | 10bp overhang | | |
| SP_194 | 194 | Desalt | 8 | Sidewinder, 4bp overhang, | LUX ABC | Assembly |
| | | | | 10bp overhang | | |
| SP_195 | 195 | Desalt | 8 | Sidewinder, 4bp overhang, | LUX ABC | Assembly |
| | | | | 10bp overhang | | |
| SP_196 | 196 | Desalt | 8 | Sidewinder, 4bp overhang, | LUX ABC | Assembly |
| | | | | 10bp overhang | | |
| SP_197 | 197 | Desalt | 8 | Sidewinder, 4bp overhang, | LUX ABC | Assembly |
| | | | | 10bp overhang | | |
| SP_198 | 198 | Desalt | 8 | Sidewinder, 4bp overhang, | LUX ABC | Assembly |
| | | | | 10bp overhang | | |
| SP_199 | 199 | Desalt | 8 | Sidewinder, 4bp overhang, | LUX ABC | Assembly |
| | | | | 10bp overhang | | |
| SP_200 | 200 | Desalt | 8 | Sidewinder, 4bp overhang, | LUX ABC | Assembly |
| | | | | 10bp overhang | | |
| SP_201 | 201 | Desalt | 8 | Sidewinder, 4bp overhang, | LUX ABC | Assembly |
| | | | | 10bp overhang | | |
| SP_202 | 202 | Desalt | 8 | Sidewinder, 4bp overhang, | LUX ABC | Assembly |
| | | | | 10bp overhang | | |
| SP_203 | 203 | Desalt | 8 | Sidewinder, 4bp overhang, | LUX ABC | Assembly |
| | | | | 10bp overhang | | |
| SP_204 | 204 | Desalt | 8 | Sidewinder, 4bp overhang, | LUX ABC | Assembly |
| | | | | 10bp overhang | | |
| SP_205 | 205 | Desalt | 8 | Sidewinder, 4bp overhang, | LUX ABC | Assembly |
| | | | | 10bp overhang | | |
| SP_206 | 206 | Desalt | 8 | Sidewinder, 4bp overhang, | LUX ABC | Assembly |
| | | | | 10bp overhang | | |
| SP_207 | 207 | Desalt | 8 | Sidewinder, 4bp overhang, | LUX ABC | Assembly |
| | | | | 10bp overhang | | |
| SP_208 | 208 | Desalt | 3 | Sidewinder | ApoE | Assembly |
| SP_209 | 209 | PAGE | 3 | Sidewinder | ApoE | Assembly |
| SP_210 | 210 | Desalt | 3 | Sidewinder | ApoE | Assembly |
| SP_211 | 211 | PAGE | 3 | Sidewinder | ApoE | Assembly |
| SP_212 | 212 | Desalt | 3 | Sidewinder | ApoE | Assembly |
| SP_213 | 213 | PAGE | 3 | Sidewinder | ApoE | Assembly |
| SP_214 | 214 | Desalt | 3 | Sidewinder | ApoE | Assembly |
| SP_215 | 215 | PAGE | 3 | Sidewinder | ApoE | Assembly |
| SP_216 | 216 | Desalt | 3 | Sidewinder | ApoE | Assembly |
| SP_217 | 217 | PAGE | 3 | Sidewinder | ApoE | Assembly |
| SP_218 | 218 | Desalt | 3 | Sidewinder | ApoE | Assembly |
| SP_219 | 219 | PAGE | 3 | Sidewinder | ApoE | Assembly |
| SP_220 | 220 | Desalt | 3 | Sidewinder | ApoE | Assembly |
| SP_221 | 221 | PAGE | 3 | Sidewinder | ApoE | Assembly |
| SP_222 | 222 | Desalt | 3 | Sidewinder | ApoE | Assembly |
| SP_223 | 223 | PAGE | 3 | Sidewinder | ApoE | Assembly |
| SP_224 | 224 | Desalt | 3 | Sidewinder | ApoE | Assembly |
| SP_225 | 225 | PAGE | 3 | Sidewinder | ApoE | Assembly |
| SP_226 | 226 | Desalt | 3 | Sidewinder | ApoE | Assembly |
| SP_227 | 227 | PAGE | 3 | Sidewinder | ApoE | Assembly |
| SP_228 | 228 | Desalt | 3 | Sidewinder | ApoE | Assembly |
| SP_229 | 229 | PAGE | 3 | Sidewinder | ApoE | Assembly |
| SP_230 | 230 | Desalt | 3 | Sidewinder | ApoE | Assembly |
| SP_231 | 231 | PAGE | 3 | Sidewinder | ApoE | Assembly |
| SP_232 | 232 | Desalt | 3 | Sidewinder | H-Fibroin | Assembly |
| SP_233 | 233 | PAGE | 3 | Sidewinder | H-Fibroin | Assembly |
| SP_234 | 234 | Desalt | 3 | Sidewinder | H-Fibroin | Assembly |
| SP_235 | 235 | PAGE | 3 | Sidewinder | H-Fibroin | Assembly |
| SP_236 | 236 | Desalt | 3 | Sidewinder | H-Fibroin | Assembly |
| SP_237 | 237 | PAGE | 3 | Sidewinder | H-Fibroin | Assembly |
| SP_238 | 238 | Desalt | 3 | Sidewinder | H-Fibroin | Assembly |
| SP_239 | 239 | PAGE | 3 | Sidewinder | H-Fibroin | Assembly |
| SP_240 | 240 | Desalt | 3 | Sidewinder | H-Fibroin | Assembly |
| SP_241 | 241 | PAGE | 3 | Sidewinder | H-Fibroin | Assembly |
| SP_242 | 242 | Desalt | 4 | Sidewinder | Parallel_mScar | Assembly |
| SP_243 | 243 | PAGE | 4 | Sidewinder | Parallel_mScar | Assembly |
| SP_244 | 244 | Desalt | 4 | Sidewinder | Parallel_mScar | Assembly |
| SP_245 | 245 | PAGE | 4 | Sidewinder | Parallel_mScar | Assembly |
| SP_246 | 246 | Desalt | 4 | Sidewinder | Parallel_mScar | Assembly |
| SP_247 | 247 | PAGE | 4 | Sidewinder | Parallel_mScar | Assembly |
| SP_248 | 248 | Desalt | 4 | Sidewinder | Parallel_mScar | Assembly |
| SP_249 | 249 | PAGE | 4 | Sidewinder | Parallel_mScar | Assembly |
| SP_250 | 250 | Desalt | 4 | Sidewinder | Parallel_mScar | Assembly |
| SP_251 | 251 | PAGE | 4 | Sidewinder | Parallel_mScar | Assembly |
| SP_252 | 252 | Desalt | 4 | Sidewinder | Parallel_mScar | Assembly |
| SP_253 | 253 | PAGE | 4 | Sidewinder | Parallel_mScar | Assembly |
| SP_254 | 254 | Desalt | 4 | Sidewinder | Parallel_mScar | Assembly |
| SP_255 | 255 | PAGE | 4 | Sidewinder | Parallel_mScar | Assembly |
| SP_256 | 256 | Desalt | 4 | Sidewinder | Parallel_mScar | Assembly |
| SP_257 | 257 | PAGE | 4 | Sidewinder | Parallel_mScar | Assembly |
| SP_258 | 258 | Desalt | 4 | Sidewinder | Parallel_mScar | Assembly |
| SP_259 | 259 | PAGE | 4 | Sidewinder | Parallel_mScar | Assembly |
| SP_260 | 260 | Desalt | 4 | Sidewinder | Parallel_mScar | Assembly |
| SP_261 | 261 | PAGE | 4 | Sidewinder | Parallel_mScar | Assembly |
| SP_262 | 262 | Desalt | 4 | Sidewinder | Parallel_mGL | Assembly |
| SP_263 | 263 | PAGE | 4 | Sidewinder | Parallel_mGL | Assembly |
| SP_264 | 264 | Desalt | 4 | Sidewinder | Parallel_mGL | Assembly |
| SP_265 | 265 | PAGE | 4 | Sidewinder | Parallel_mGL | Assembly |
| SP_266 | 266 | Desalt | 4 | Sidewinder | Parallel_mGL | Assembly |
| SP_267 | 267 | PAGE | 4 | Sidewinder | Parallel_mGL | Assembly |
| SP_268 | 268 | Desalt | 4 | Sidewinder | Parallel_mGL | Assembly |
| SP_269 | 269 | PAGE | 4 | Sidewinder | Parallel_mGL | Assembly |
| SP_270 | 270 | Desalt | 4 | Sidewinder | Parallel_mGL | Assembly |
| SP_271 | 271 | PAGE | 4 | Sidewinder | Parallel_mGL | Assembly |
| SP_272 | 272 | Desalt | 4 | Sidewinder | Parallel_mGL | Assembly |
| SP_273 | 273 | PAGE | 4 | Sidewinder | Parallel_mGL | Assembly |
| SP_274 | 274 | Desalt | 4 | Sidewinder | Parallel_mGL | Assembly |
| SP_275 | 275 | PAGE | 4 | Sidewinder | Parallel_mGL | Assembly |
| SP_276 | 276 | Desalt | 4 | Sidewinder | Parallel_mGL | Assembly |
| SP_277 | 277 | PAGE | 4 | Sidewinder | Parallel_mGL | Assembly |
| SP_278 | 278 | Desalt | 4 | Sidewinder | Parallel_mGL | Assembly |
| SP_279 | 279 | PAGE | 4 | Sidewinder | Parallel_mGL | Assembly |
| SP_280 | 280 | Desalt | 4 | Sidewinder | Parallel_mGL | Assembly |
| SP_281 | 281 | PAGE | 4 | Sidewinder | Parallel_mGL | Assembly |
| SP_282 | 282 | Desalt | 4 | Sidewinder | Parallel_AeBlue | Assembly |
| SP_283 | 283 | PAGE | 4 | Sidewinder | Parallel_AeBlue | Assembly |
| SP_284 | 284 | Desalt | 4 | Sidewinder | Parallel_AeBlue | Assembly |
| SP_285 | 285 | PAGE | 4 | Sidewinder | Parallel_AeBlue | Assembly |
| SP_286 | 286 | Desalt | 4 | Sidewinder | Parallel_AeBlue | Assembly |
| SP_287 | 287 | PAGE | 4 | Sidewinder | Parallel_AeBlue | Assembly |
| SP_288 | 288 | Desalt | 4 | Sidewinder | Parallel_AeBlue | Assembly |
| SP_289 | 289 | PAGE | 4 | Sidewinder | Parallel_AeBlue | Assembly |
| SP_290 | 290 | Desalt | 4 | Sidewinder | Parallel_AeBlue | Assembly |
| SP_291 | 291 | PAGE | 4 | Sidewinder | Parallel_AeBlue | Assembly |
| SP_292 | 292 | Desalt | 4 | Sidewinder | Parallel_AeBlue | Assembly |
| SP_293 | 293 | PAGE | 4 | Sidewinder | Parallel_AeBlue | Assembly |
| SP_294 | 294 | Desalt | 4 | Sidewinder | Parallel_AeBlue | Assembly |
| SP_295 | 295 | PAGE | 4 | Sidewinder | Parallel_AeBlue | Assembly |
| SP_296 | 296 | Desalt | 4 | Sidewinder | Parallel_AeBlue | Assembly |
| SP_297 | 297 | PAGE | 4 | Sidewinder | Parallel_AeBlue | Assembly |
| SP_298 | 298 | Desalt | 4 | Sidewinder | Parallel_AeBlue | Assembly |
| SP_299 | 299 | PAGE | 4 | Sidewinder | Parallel_AeBlue | Assembly |
| SP_300 | 300 | Desalt | 4 | Sidewinder | Parallel_AeBlue | Assembly |
| SP_301 | 301 | PAGE | 4 | Sidewinder | Parallel_AeBlue | Assembly |
| SP_302 | 302 | Cartridge | 5 | Sidewinder | Library | Assembly |
| SP_303 | 303 | Cartridge | 5 | Sidewinder | Library | Assembly |
| SP_304 | 304 | Cartridge | 5 | Sidewinder | Library | Assembly |
| SP_305 | 305 | Cartridge | 5 | Sidewinder | Library | Assembly |
| SP_306 | 306 | Cartridge | 5 | Sidewinder | Library | Assembly |
| SP_307 | 307 | Cartridge | 5 | Sidewinder | Library | Assembly |
| SP_308 | 308 | Cartridge | 5 | Sidewinder | Library | Assembly |
| SP_309 | 309 | Cartridge | 5 | Sidewinder | Library | Assembly |
| SP_310 | 310 | Cartridge | 5 | Sidewinder | Library | Assembly |
| SP_311 | 311 | Cartridge | 5 | Sidewinder | Library | Assembly |
| SP_312 | 312 | Cartridge | 5 | Sidewinder | Library | Assembly |
| SP_313 | 313 | Cartridge | 5 | Sidewinder | Library | Assembly |
| SP_314 | 314 | Cartridge | 5 | Sidewinder | Library | Assembly |
| SP_315 | 315 | Cartridge | 5 | Sidewinder | Library | Assembly |
| SP_316 | 316 | Cartridge | 5 | Sidewinder | Library | Assembly |
| SP_317 | 317 | Cartridge | 5 | Sidewinder | Library | Assembly |
| SP_318 | 318 | Cartridge | 5 | Sidewinder | Library | Assembly |
| SP_319 | 319 | Cartridge | 5 | Sidewinder | Library | Assembly |
| SP_320 | 320 | Cartridge | 5 | Sidewinder | Library | Assembly |
| SP_321 | 321 | Cartridge | 5 | Sidewinder | Library | Assembly |
| SP_322 | 322 | Cartridge | 5 | Sidewinder | Library | Assembly |
| SP_323 | 323 | Cartridge | 5 | Sidewinder | Library | Assembly |
| SP_324 | 324 | Cartridge | 5 | Sidewinder | Library | Assembly |
| SP_325 | 325 | Cartridge | 5 | Sidewinder | Library | Assembly |
| SP_326 | 326 | Cartridge | 5 | Sidewinder | Library | Assembly |
| SP_327 | 327 | Cartridge | 5 | Sidewinder | Library | Assembly |
| SP_328 | 328 | Cartridge | 5 | Sidewinder | Library | Assembly |
| SP_329 | 329 | Cartridge | 5 | Sidewinder | Library | Assembly |
| SP_330 | 330 | Cartridge | 5 | Sidewinder | Library | Assembly |
| SP_331 | 331 | Cartridge | 5 | Sidewinder | Library | Assembly |
| SP_332 | 332 | Desalt | 10 | Sidewinder | USER Digest 3WJ | Assembly |
| SP_333 | 333 | Desalt | 10 | Sidewinder | USER Digest 3WJ | Assembly |
| SP_334 | 334 | Desalt | 10 | Sidewinder | USER Digest 3WJ | Assembly |
| SP_335 | 335 | Desalt | 10 | Sidewinder | USER Digest 3WJ | Assembly |
| SP_336 | 336 | Desalt | 10 | Sidewinder | USER Digest 3WJ | Assembly |
| SP_337 | 337 | Desalt | 2 | Sidewinder/PCA | LUX ABC | Amplification |
| SP_338 | 338 | Desalt | 2 | Sidewinder/PCA | LUX ABC | Amplification |
| SP_339 | 339 | Desalt | 2 | Sidewinder/PCA | LUX ABC | Amplification |
| SP_340 | 340 | Desalt | 2 | Sidewinder/PCA | LUX ABC | Amplification |
| SP_341 | 341 | Desalt | 2 | Sidewinder/PCA | LUX ABC | Amplification |
| SP_342 | 342 | Desalt | 8 | Sidewinder/PCA | mGL + mScar | Amplification |
| SP_343 | 343 | Desalt | 8 | Sidewinder/PCA | mGL + mScar | Amplification |
| SP_344 | 344 | Desalt | 8 | Sidewinder/PCA | mGL + mScar | Amplification |
| SP_345 | 345 | Desalt | 8 | Sidewinder/PCA | mGL + mScar | Amplification |
| SP_346 | 346 | Desalt | 3 | Sidewinder | ApoE | Amplification |
| SP_347 | 347 | Desalt | 3 | Sidewinder | ApoE | Amplification |
| SP_348 | 348 | Desalt | 3 | Sidewinder | H-Fibroin | Amplification |
| SP_349 | 349 | Desalt | 3 | Sidewinder | H-Fibroin | Amplification |
| SP_350 | 350 | Desalt | 4 | Sidewinder | Parallel_Universal | Amplification/Cloning |
| SP_351 | 351 | Desalt | 4 | Sidewinder | Parallel_Universal | Amplification/Cloning |
| SP_352 | 352 | Desalt | 4 | Sidewinder | Parallel_mScar | Amplification/Cloning |
| SP_353 | 353 | Desalt | 4 | Sidewinder | Parallel_mScar | Amplification/Cloning |
| SP_354 | 354 | Desalt | 4 | Sidewinder | Parallel_mGL | Amplification/Cloning |
| SP_355 | 355 | Desalt | 4 | Sidewinder | Parallel_mGL | Amplification/Cloning |
| SP_356 | 356 | Desalt | 4 | Sidewinder | Parallel_AeBlue | Amplification/Cloning |
| SP_357 | 357 | Desalt | 4 | Sidewinder | Parallel_AeBlue | Amplification/Cloning |
| SP_358 | 358 | Desalt | 4 | Sidewinder | Parallel_Universal_BB | Amplification/Cloning |
| SP_359 | 359 | Desalt | 4 | Sidewinder | Parallel_Universal_BB | Amplification/Cloning |
| SP_360 | 360 | Desalt | 4,5 | Sidewinder | Parallel_Individuall_BB, | Amplification/Cloning |
| | | | | | Library_BB | |
| SP_361 | 361 | Desalt | 4, 5 | Sidewinder | Parallel_Individuall_BB, | Amplification/Cloning |
| | | | | | Library_BB | |
| SP_362 | 362 | Desalt | 5 | Sidewinder | Library | Amplification/Cloning |
| SP_363 | 363 | Desalt | 5 | Sidewinder | Library | Amplification/Cloning |
| SP_364 | 364 | Desalt | 5 | Sidewinder | Library | Amplification/Cloning |
| SP_365 | 365 | Desalt | 5 | Sidewinder | Library | Amplification/Cloning |
| SP_366 | 366 | Desalt | 5 | Sidewinder | Library | Amplification/Cloning |
| SP_367 | 367 | Desalt | 8 | 4bp overhang | LUX ABC | Assembly |
| SP_368 | 368 | Desalt | 8 | 4bp overhang | LUX ABC | Assembly |
| SP_369 | 369 | Desalt | 8 | 4bp overhang | LUX ABC | Assembly |
| SP_370 | 370 | Desalt | 8 | 4bp overhang | LUX ABC | Assembly |
| SP_371 | 371 | Desalt | 8 | 4bp overhang | LUX ABC | Assembly |
| SP_372 | 372 | Desalt | 8 | 4bp overhang | LUX ABC | Assembly |
| SP_373 | 373 | Desalt | 8 | 4bp overhang | LUX ABC | Assembly |
| SP_374 | 374 | Desalt | 8 | 4bp overhang | LUX ABC | Assembly |
| SP_375 | 375 | Desalt | 8 | 4bp overhang | LUX ABC | Assembly |
| SP_376 | 376 | Desalt | 8 | 4bp overhang | LUX ABC | Assembly |
| SP_377 | 377 | Desalt | 8 | 4bp overhang | LUX ABC | Assembly |
| SP_378 | 378 | Desalt | 8 | 4bp overhang | LUX ABC | Assembly |
| SP_379 | 379 | Desalt | 8 | 4bp overhang | LUX ABC | Assembly |
| SP_380 | 380 | Desalt | 8 | 4bp overhang | LUX ABC | Assembly |
| SP_381 | 381 | Desalt | 8 | 4bp overhang | LUX ABC | Assembly |
| SP_382 | 382 | Desalt | 8 | 4bp overhang | LUX ABC | Assembly |
| SP_383 | 383 | Desalt | 8 | 4bp overhang | LUX ABC | Assembly |
| SP_384 | 384 | Desalt | 8 | 4bp overhang | LUX ABC | Assembly |
| SP_385 | 385 | Desalt | 8 | 4bp overhang | LUX ABC | Assembly |
| SP_386 | 386 | Desalt | 8 | 4bp overhang | LUX ABC | Assembly |
| SP_387 | 387 | Desalt | 8 | 10bp overhang | LUX ABC | Assembly |
| SP_388 | 388 | Desalt | 8 | 10bp overhang | LUX ABC | Assembly |
| SP_389 | 389 | Desalt | 8 | 10bp overhang | LUX ABC | Assembly |
| SP_390 | 390 | Desalt | 8 | 10bp overhang | LUX ABC | Assembly |
| SP_391 | 391 | Desalt | 8 | 10bp overhang | LUX ABC | Assembly |
| SP_392 | 392 | Desalt | 8 | 10bp overhang | LUX ABC | Assembly |
| SP_393 | 393 | Desalt | 8 | 10bp overhang | LUX ABC | Assembly |
| SP_394 | 394 | Desalt | 8 | 10bp overhang | LUX ABC | Assembly |
| SP_395 | 395 | Desalt | 8 | 10bp overhang | LUX ABC | Assembly |
| SP_396 | 396 | Desalt | 8 | 10bp overhang | LUX ABC | Assembly |
| SP_397 | 397 | Desalt | 8 | 10bp overhang | LUX ABC | Assembly |
| SP_398 | 398 | Desalt | 8 | 10bp overhang | LUX ABC | Assembly |
| SP_399 | 399 | Desalt | 8 | 10bp overhang | LUX ABC | Assembly |
| SP_400 | 400 | Desalt | 8 | 10bp overhang | LUX ABC | Assembly |
| SP_401 | 401 | Desalt | 8 | 10bp overhang | LUX ABC | Assembly |
| SP_402 | 402 | Desalt | 8 | 10bp overhang | LUX ABC | Assembly |
| SP_403 | 403 | Desalt | 8 | 10bp overhang | LUX ABC | Assembly |
| SP_404 | 404 | Desalt | 8 | 10bp overhang | LUX ABC | Assembly |
| SP_405 | 405 | Desalt | 8 | 10bp overhang | LUX ABC | Assembly |
| SP_406 | 406 | Desalt | 8 | 10bp overhang | LUX ABC | Assembly |
| SP_407 | 407 | Desalt | 8 | Gibson | LUX ABC | Assembly |
| SP_408 | 408 | Desalt | 8 | Gibson | LUX ABC | Assembly |
| SP_409 | 409 | Desalt | 8 | Gibson | LUX ABC | Assembly |
| SP_410 | 410 | Desalt | 8 | Gibson | LUX ABC | Assembly |
| SP_411 | 411 | Desalt | 8 | Gibson | LUX ABC | Assembly |
| SP_412 | 412 | Desalt | 8 | Gibson | LUX ABC | Assembly |
| SP_413 | 413 | Desalt | 8 | Gibson | LUX ABC | Assembly |
| SP_414 | 414 | Desalt | 8 | Gibson | LUX ABC | Assembly |
| SP_415 | 415 | Desalt | 8 | Gibson | LUX ABC | Assembly |
| SP_416 | 416 | Desalt | 8 | Gibson | LUX ABC | Assembly |
| SP_417 | 417 | Desalt | 8 | Gibson | LUX ABC | Assembly |
| SP_418 | 418 | Desalt | 8 | Gibson | LUX ABC | Assembly |
| SP_419 | 419 | Desalt | 8 | Gibson | LUX ABC | Assembly |
| SP_420 | 420 | Desalt | 8 | Gibson | LUX ABC | Assembly |
| SP_421 | 421 | Desalt | 8 | Gibson | LUX ABC | Assembly |
| SP_422 | 422 | Desalt | 8 | Gibson | LUX ABC | Assembly |
| SP_423 | 423 | Desalt | 8 | Gibson | LUX ABC | Assembly |
| SP_424 | 424 | Desalt | 8 | Gibson | LUX ABC | Assembly |
| SP_425 | 425 | Desalt | 8 | Gibson | LUX ABC | Assembly |
| SP_426 | 426 | Desalt | 8 | Gibson | LUX ABC | Assembly |
| SP_427 | 427 | Desalt | 8 | Gibson | LUX ABC | Assembly |
| SP_428 | 428 | Desalt | 8 | Gibson | LUX ABC | Assembly |
| SP_429 | 429 | Desalt | 8 | Gibson | LUX ABC | Assembly |
| SP_430 | 430 | Desalt | 8 | Gibson | LUX ABC | Assembly |
| SP_431 | 431 | Desalt | 8 | Gibson | LUX ABC | Assembly |
| SP_432 | 432 | Desalt | 8 | Gibson | LUX ABC | Assembly |
| SP_433 | 433 | Desalt | 8 | Gibson | LUX ABC | Assembly |
| SP_434 | 434 | Desalt | 8 | Gibson | LUX ABC | Assembly |
| SP_435 | 435 | Desalt | 8 | Gibson | LUX ABC | Assembly |
| SP_436 | 436 | Desalt | 8 | Gibson | LUX ABC | Assembly |
| SP_437 | 437 | Desalt | 8 | Gibson | LUX ABC | Assembly |
| SP_438 | 438 | Desalt | 8 | Gibson | LUX ABC | Assembly |
| SP_439 | 439 | Desalt | 8 | Gibson | LUX ABC | Assembly |
| SP_440 | 440 | Desalt | 8 | Gibson | LUX ABC | Assembly |
| SP_441 | 441 | Desalt | 8 | Gibson | LUX ABC | Assembly |
| SP_442 | 442 | Desalt | 8 | Gibson | LUX ABC | Assembly |
| SP_443 | 443 | Desalt | 8 | Gibson | LUX ABC | Assembly |
| SP_444 | 444 | Desalt | 8 | Gibson | LUX ABC | Assembly |
| SP_445 | 445 | Desalt | 8 | Gibson | LUX ABC | Assembly |
| SP_446 | 446 | Desalt | 8 | Gibson | LUX ABC | Assembly |
| SP_447 | 447 | Desalt | 11 | 4bp overhang, 10bp | ApoE | Assembly |
| | | | | overhang | | |
| SP_448 | 448 | Desalt | 11 | 4bp overhang, 10bp | ApoE | Assembly |
| | | | | overhang | | |
| SP_449 | 449 | Desalt | 11 | 4bp overhang, 10bp | ApoE | Assembly |
| | | | | overhang | | |
| SP_450 | 450 | Desalt | 11 | 4bp overhang, 10bp | ApoE | Assembly |
| | | | | overhang | | |
| SP_451 | 451 | Desalt | 11 | 4bp overhang, 10bp | ApoE | Assembly |
| | | | | overhang | | |
| SP_452 | 452 | Desalt | 11 | 4bp overhang, 10bp | ApoE | Assembly |
| | | | | overhang | | |
| SP_453 | 453 | Desalt | 11 | 4bp overhang, 10bp | ApoE | Assembly |
| | | | | overhang | | |
| SP_454 | 454 | Desalt | 11 | 4bp overhang, 10bp | ApoE | Assembly |
| | | | | overhang | | |
| SP_455 | 455 | Desalt | 11 | 4bp overhang, 10bp | ApoE | Assembly |
| | | | | overhang | | |
| SP_456 | 456 | Desalt | 11 | 4bp overhang, 10bp | ApoE | Assembly |
| | | | | overhang | | |
| SP_457 | 457 | Desalt | 11 | 4bp overhang, 10bp | ApoE | Assembly |
| | | | | overhang | | |
| SP_458 | 458 | Desalt | 11 | 4bp overhang, 10bp | ApoE | Assembly |
| | | | | overhang | | |
| SP_459 | 459 | Desalt | 11 | 4bp overhang | ApoE | Assembly |
| SP_460 | 460 | Desalt | 11 | 4bp overhang | ApoE | Assembly |
| SP_461 | 461 | Desalt | 11 | 4bp overhang | ApoE | Assembly |
| SP_462 | 462 | Desalt | 11 | 4bp overhang | ApoE | Assembly |
| SP_463 | 463 | Desalt | 11 | 4bp overhang | ApoE | Assembly |
| SP_464 | 464 | Desalt | 11 | 4bp overhang | ApoE | Assembly |
| SP_465 | 465 | Desalt | 11 | 4bp overhang | ApoE | Assembly |
| SP_466 | 466 | Desalt | 11 | 4bp overhang | ApoE | Assembly |
| SP_467 | 467 | Desalt | 11 | 4bp overhang | ApoE | Assembly |
| SP_468 | 468 | Desalt | 11 | 4bp overhang | ApoE | Assembly |
| SP_469 | 469 | Desalt | 11 | 4bp overhang | ApoE | Assembly |
| SP_470 | 470 | Desalt | 11 | 4bp overhang | ApoE | Assembly |
| SP_471 | 471 | Desalt | 11 | 10bp overhang | ApoE | Assembly |
| SP_472 | 472 | Desalt | 11 | 10bp overhang | ApoE | Assembly |
| SP_473 | 473 | Desalt | 11 | 10bp overhang | ApoE | Assembly |
| SP_474 | 474 | Desalt | 11 | 10bp overhang | ApoE | Assembly |
| SP_475 | 475 | Desalt | 11 | 10bp overhang | ApoE | Assembly |
| SP_476 | 476 | Desalt | 11 | 10bp overhang | ApoE | Assembly |
| SP_477 | 477 | Desalt | 11 | 10bp overhang | ApoE | Assembly |
| SP_478 | 478 | Desalt | 11 | 10bp overhang | ApoE | Assembly |
| SP_479 | 479 | Desalt | 11 | 10bp overhang | ApoE | Assembly |
| SP_480 | 480 | Desalt | 11 | 10bp overhang | ApoE | Assembly |
| SP_481 | 481 | Desalt | 11 | 10bp overhang | ApoE | Assembly |
| SP_482 | 482 | Desalt | 11 | 10bp overhang | ApoE | Assembly |
| SP_483 | 483 | Desalt | 11 | PCA | ApoE | Assembly |
| SP_484 | 484 | Desalt | 11 | PCA | ApoE | Assembly |
| SP_485 | 485 | Desalt | 11 | PCA | ApoE | Assembly |
| SP_486 | 486 | Desalt | 11 | PCA | ApoE | Assembly |
| SP_487 | 487 | Desalt | 11 | PCA | ApoE | Assembly |
| SP_488 | 488 | Desalt | 11 | PCA | ApoE | Assembly |
| SP_489 | 489 | Desalt | 11 | PCA | ApoE | Assembly |
| SP_490 | 490 | Desalt | 11 | PCA | ApoE | Assembly |
| SP_491 | 491 | Desalt | 11 | PCA | ApoE | Assembly |
| SP_492 | 492 | Desalt | 11 | PCA | ApoE | Assembly |
| SP_493 | 493 | Desalt | 11 | PCA | ApoE | Assembly |
| SP_494 | 494 | Desalt | 11 | PCA | ApoE | Assembly |
| SP_495 | 495 | Desalt | 11 | Gibson | ApoE | Assembly |
| SP_496 | 496 | Desalt | 11 | Gibson | ApoE | Assembly |
| SP_497 | 497 | Desalt | 11 | Gibson | ApoE | Assembly |
| SP_498 | 498 | Desalt | 11 | Gibson | ApoE | Assembly |
| SP_499 | 499 | Desalt | 11 | Gibson | ApoE | Assembly |
| SP_500 | 500 | Desalt | 11 | Gibson | ApoE | Assembly |
| SP_501 | 501 | Desalt | 11 | Gibson | ApoE | Assembly |
| SP_502 | 502 | Desalt | 11 | Gibson | ApoE | Assembly |
| SP_503 | 503 | Desalt | 11 | Gibson | ApoE | Assembly |
| SP_504 | 504 | Desalt | 11 | Gibson | ApoE | Assembly |
| SP_505 | 505 | Desalt | 11 | Gibson | ApoE | Assembly |
| SP_506 | 506 | Desalt | 11 | Gibson | ApoE | Assembly |
| SP_507 | 507 | Desalt | 11 | Gibson | ApoE | Assembly |
| SP_508 | 508 | Desalt | 11 | Gibson | ApoE | Assembly |
| SP_509 | 509 | Desalt | 11 | Gibson | ApoE | Assembly |
| SP_510 | 510 | Desalt | 11 | Gibson | ApoE | Assembly |
| SP_511 | 511 | Desalt | 11 | Gibson | ApoE | Assembly |
| SP_512 | 512 | Desalt | 11 | Gibson | ApoE | Assembly |
| SP_513 | 513 | Desalt | 11 | Gibson | ApoE | Assembly |
| SP_514 | 514 | Desalt | 11 | Gibson | ApoE | Assembly |
| SP_515 | 515 | Desalt | 11 | Gibson | ApoE | Assembly |
| SP_516 | 516 | Desalt | 11 | Gibson | ApoE | Assembly |
| SP_517 | 517 | Desalt | 11 | Gibson | ApoE | Assembly |
| SP_518 | 518 | Desalt | 11 | Gibson | ApoE | Assembly |
| SP_519 | 519 | Desalt | 11 | 4bp overhang, 10bp | H-Fibroin | Assembly |
| | | | | overhang | | |
| SP_520 | 520 | Desalt | 11 | 4bp overhang, 10bp | H-Fibroin | Assembly |
| | | | | overhang | | |
| SP_521 | 521 | Desalt | 11 | 4bp overhang, 10bp | H-Fibroin | Assembly |
| | | | | overhang | | |
| SP_522 | 522 | Desalt | 11 | 4bp overhang, 10bp | H-Fibroin | Assembly |
| | | | | overhang | | |
| SP_523 | 523 | Desalt | 11 | 4bp overhang, 10bp | H-Fibroin | Assembly |
| | | | | overhang | | |
| SP_524 | 524 | Desalt | 11 | 4bp overhang | H-Fibroin | Assembly |
| SP_525 | 525 | Desalt | 11 | 4bp overhang | H-Fibroin | Assembly |
| SP_526 | 526 | Desalt | 11 | 4bp overhang | H-Fibroin | Assembly |
| SP_527 | 527 | Desalt | 11 | 4bp overhang | H-Fibroin | Assembly |
| SP_528 | 528 | Desalt | 11 | 4bp overhang | H-Fibroin | Assembly |
| SP_529 | 529 | Desalt | 11 | 10bp overhang | H-Fibroin | Assembly |
| SP_530 | 530 | Desalt | 11 | 10bp overhang | H-Fibroin | Assembly |
| SP_531 | 531 | Desalt | 11 | 10bp overhang | H-Fibroin | Assembly |
| SP_532 | 532 | Desalt | 11 | 10bp overhang | H-Fibroin | Assembly |
| SP_533 | 533 | Desalt | 11 | 10bp overhang | H-Fibroin | Assembly |
| SP_534 | 534 | Desalt | 11 | PCA | H-Fibroin | Assembly |
| SP_535 | 535 | Desalt | 11 | PCA | H-Fibroin | Assembly |
| SP_536 | 536 | Desalt | 11 | PCA | H-Fibroin | Assembly |
| SP_537 | 537 | Desalt | 11 | PCA | H-Fibroin | Assembly |
| SP_538 | 538 | Desalt | 11 | PCA | H-Fibroin | Assembly |
| SP_539 | 539 | Desalt | 11 | Gibson | H-Fibroin | Assembly |
| SP_540 | 540 | Desalt | 11 | Gibson | H-Fibroin | Assembly |
| SP_541 | 541 | Desalt | 11 | Gibson | H-Fibroin | Assembly |
| SP_542 | 542 | Desalt | 11 | Gibson | H-Fibroin | Assembly |
| SP_543 | 543 | Desalt | 11 | Gibson | H-Fibroin | Assembly |
| SP_544 | 544 | Desalt | 11 | Gibson | H-Fibroin | Assembly |
| SP_545 | 545 | Desalt | 11 | Gibson | H-Fibroin | Assembly |
| SP_546 | 546 | Desalt | 11 | Gibson | H-Fibroin | Assembly |
| SP_547 | 547 | Desalt | 11 | Gibson | H-Fibroin | Assembly |
| SP_548 | 548 | Desalt | 11 | Gibson | H-Fibroin | Assembly |

### Heteroduplex Annealing

Oligos were suspended by hand in 1× TE buffer at pH 8.0 (Corning, ThermoFisher Scientific) to a final concentration of 100 μM based on manufacturers reported weight. To ensure adequate resuspension of the dried oligos, if the volume required to for a final concentration of 100 μM was less than 50 uL of TE buffer according to the manufacturer's reported weight, oligos would be resuspended in a volume of 50 uL of buffer resulting in a lower final concertation. The concentration of all oligos were additionally measured using the Qubit ssDNA Assay Kit (Invitrogen, ThermoFisher Scientific) and final concentration calculations were based upon these measurements.

Sidewinder fragments were generated from resuspended stock oligos by annealing coding oligo to the barcode oligo to form a heteroduplex. To prepare the Sidewinder fragment, the volume of coding oligo required for 2 μM in a 50 μL reaction was first phosphorylated alone in a 25 μL reaction using 1uL of T4PNK (New England Biolabs) in 1×T4 ligase buffer at 37° C. for 1 hr, followed by an enzyme deactivation at 80° C. for 10 minutes. The corresponding volume of stock barcode oligo needed for 1 uM in 50 μL was then added to the phosphorylated coding oligo and final volume is topped off to 50 μL using 1× T4 ligase buffer.

Heteroduplexes were then annealed together in a PCR tube consisting of an initial denaturation of 98° C. for 10 minutes, followed by a gradual decrease in temperature down to 25° C. at −1° C. per minute. Once fragments are annealed, they were kept at 4° C. until use and have been stably used months after initial heteroduplex formation.

### Heteroduplex Gel Extraction

PAGE gel extraction of annealed heteroduplexes was performed using 8% TBE gel (Invitrogen, ThermoFisher Scientific) and run at 200v for 35 minutes. Gel extraction was done according to published DNA nanotechnology protocol.

### Sidewinder Assembly Conditions

Processed Sidewinder fragments were combined into a single reaction mix at equimolar concentrations at ~1 nM to conduct the Sidewinder assembly. Two avenues were utilized for assembly conditions for the Sidewinder assembly. Assemblies were conducted in 70 μL reactions in 1× HiFi Taq buffer (New England Biolabs). For all assemblies except the h-fibroin assembly, a “cycling” protocol was used of 85° C. for 5 minutes, followed by the addition of 2.8 μL of HiFi Taq ligase (New England Biolabs), then the reaction then cycles between 85° C. for 1 minute and 50° C. for 2 minutes for 100 cycles. These cycles were then followed by 50° C. for 1 hr. The second assembly protocol which was used for the h-fibroin assembly is 13 nM fragments in 70 μL reaction in 1× HiFi Taq buffer (New England Biolabs) 85° C. for 5 minutes followed by −0.1° C. per 6s down to 50° C., addition of 2.8 μL of HiFi Taq ligase, and incubate at 50° C. temperature overnight. The Sidewinder characterization assemblies in FIG. 1 were also conducted with the second assembly protocol with a ramp down to ligation temperature of 72° C.

Reactions which characterized choice of ligase and toehold length (FIG. 7) used the non-cycling assembly protocol at a ligation temperature according to manufacturer's recommendation in the corresponding ligase buffer.

### Conventional Assembly Comparison Conditions

Fragments for the 4 bp 2WJ, 10 bp 2WJ and Gibson assemblies were generated using oligos. The 4 bp 2WJ and 10 bp 2WJ utilized the same coding oligo sequence as the corresponding fragment in the Sidewinder assembly. A new complementary oligo was ordered to generate the desired overhangs: 10 bp 2WJ complement oligo was designed by removing the barcode sequences form the barcode oligo. 4 bp 2WJ was designed to use the terminal 4 bases of the Sidewinder toehold. Gibson oligos were designed to compose an analogous segment with 20 bp of homology to the partner fragment on either end. PCA does not conduct assemblies using fragments but instead uses individual oligos which were designed with 20 bases of overlap to the partnered oligo (FIG. 8A).

The 4 bp 2WJ, 10 bp 2WJ and Gibson oligos were processed to mirror the Sidewinder fragment processing. Oligos were mixed in an equal 1 μM ratio, both oligos phosphorylated with T4 PNK (New England Biolabs) in a 50 μL reaction in 1× T4 Ligase buffer (New England Biolabs) then annealed. Gibson oligos are not phosphorylated. Fragments are PAGE extracted and concentrations measured with Qubit 1× dsDNA High Sensitivity Assay Kit, (Invitrogen, ThermoFisher Scientific).

Fragments were assembled at the same concentration of the corresponding Sidewinder assembly. The 4 bp 2WJ, and 10 bp 2WJ were assembled at 16° C. overnight in 1× T4 ligase buffer with 1 μL T4 Ligase according to manufacturer's recommendation for ligating sticky ends (New England Biolabs). Gibson assembly was conducted at 50° C. for Ihr using NEBuilder HiFi DNA assembly Master Mix (NEB). PCA was preformed using PrimeSTAR GXL Polymerase (Takara Bio) under a published protocol which was demonstrated to be optimized for multi-fragment assemblies.

### PCR Amplification and Purification

Either PrimeSTAR GXL Polymerase or repliQa HiFi ToughMix (Quantabio) were used for amplification of 3WJ assemblies. Only 1 uL of unpurified 3WJ assembly from the previous ligation step is sufficient template in a 50 μL PCR reaction. PCR reaction conditions were established according to manufacturer recommendations and predicted Im of primers.

Post PCR amplification, purification of the PCR reaction was done using a QIAquick PCR Purification Kit (Qiagen). Multiple 50 μL PCR reactions can be passed simultaneously through the same purification column to increase the final concentration of the purified 2WJ assembly. Alternatively, gel extraction of the target band can be done. Gel extraction results in an even more highly pure product for downstream sequencing or cloning as seen with the High GC assembly. Gel extraction was done prior to sequencing for all assemblies except for the parallel assembly. The Monarch DNA Gel Extraction Kit (New England Biolabs) was used according to the manufacturers protocol.

### DNA Gel Imaging

The Sidewinder characterization gel in FIG. 1 is a 6% TBE-UREA Denature Gel (Novex, ThermoFisher Scientific) run at constant 180v for 30 minutes in 1× TBE buffer. Samples were mixed with an equal volume of 2× stain free TBE-Urea loading buffer and heat shocked at 90° C. for 15 min prior to loading gel.

All other gel images are 1-2% agarose gels stained with Sybr Safe (Invitrogen, ThermoFisher Scientific) run at 135V for 25 minutes in 0.5× TBE buffer. The 1 kb+ladder (New England Biolabs) was used in FIGS. 2, 3B, 4, and 5. The Low Molecular Weight Ladder (New England Biolabs) was used in FIG. 3E. All main figure agarose gels depict 50 ng DNA loaded in each lane as measured by Qubit 1× dsDNA High Sensitivity Assay Kit, (Invitrogen, ThermoFisher Scientific). 4 bp 2WJ, 10 bp 2WJ and Gibson lanes depict 1 uL loaded as amplification was insufficient to produce 50 ng post purification in some cases.

### Cloning and Transformation

Sidewinder constructs to be expressed in bacteria were amplified using dU containing primers and repliQa polymerase (QuantaBio). Corresponding vectors were amplified using dU containing primers. Purified products were treated with 1 uL USER (New England Biolabs) in 1× CutSmart buffer and subsequently repurified with PCR Kleen Purification Spin Column (Bio-Rad) and assembled according to published protocols in 1× T4 ligase buffer and 2.5 μL T4 ligase. 2 μL of assembled product was electroporated into electrocompetent DH10b cells, recovered in 2 mL Luria-Bertani (LB) media for 1 hour, and plated on LB-agar plates with the corresponding antibiotics. All final constructs can be found in Table 2.

TABLE 2
Sequences for target constructs and plasmid
backbones utilized in experiments

| | SEQ ID NO: |
|---|---|
| 40-piece_LuxABC | 549 |
| 20-piece_mGL + mScar-Fusion-cassette | 550 |
| High-GC_ApoE | 551 |
| High-Repeat_H-Fibroin | 552 |
| Parallel_Assembly_AeBlue | 553 |
| Parallel_Assembly_mGL | 554 |
| Parallel_Assembly_mScar | 555 |
| Library_EGFP_Scaffold | 556 |
| CmR_P15a_Vector_BB | 557 |
| Inducible_pTac-Full_WT_T7pol_Vector_BB | 558 |

### Sequencing Analysis

Final assemblies were processed as described and the purified samples were used for sequencing. Oxford Nanopore Sequencing was used to get long, full-molecule reads required to determine the percentage of complete 3WJ assemblies for FIGS. 2, 3, & 4. PacBio sequencing was used to get high-confidence per-base whole molecule sequencing for the Sidewinder Library in FIG. 5.

Assemblies validated through Nanopore sequencing used Plasmidsaurus Premium PCR Sequencing services. Assemblies validated through PacBio used Azenta sequencing services. To validate the analysis pipeline, each individual raw read for the Sidewinder 40-piece assembly was viewed and assigned manually, allowing us to know the exact identity of each read without any pre-bias due to filtering. For each of the subsequent assemblies, the verified fragment level analysis pipeline was used to generate the pie charts. In this pipeline, read sequences were aligned to fragment references using blastn, where every read was aligned to every fragment reference. The read is assigned as “unusable” if no hits to any fragment are returned. A “correct” assembly was assigned when all fragments were in the correct order of the gene sequence. Correct assemblies with all fragments were deemed “complete”, whereas those with not all fragments were deemed “partial”. For Nanopore, all remaining reads were checked manually to determine the nature of the assembly.

The fragment level analysis has reads sorted into four main categories. In some embodiments, a “Sidewinder product” is a construct which results from the ligation of the toeholds at the 3WJ, characterized by a seamless sequence transition between the 5′ end of one fragment and the 3′ end of another fragment. A correct assembly is a seamless transition between all partnered fragments in the correct order whereas an incorrect assembly is the seamless transition between non-partnered fragments. In addition to Sidewinder products, PCR artifacts and sequencing artifacts would be expected. PCR artifacts result from mis-priming during PCR. These were identified by the sequence transition between two non-partnered fragments joined, not at the assembly junction, but instead at the internal portion of one of the fragments, indicating that a primer or unreacted fragment oligo mis-primed and was elongated during PCR (FIGS. 9A-9B). Mis-priming can be reduced by biasing the PCR template towards a higher proportion of full length 3WJ assembly as well as by using alternative methods for removing the 3WJ that don't depend on PCR amplification (FIGS. 10A-10B). Sequencing artifacts are due to systematic failures in base calling or sequencing preparation procedure that would not result from assembly. Lastly, in protocols where the barcode oligo is also phosphorylated, “barcode artifacts” were see where, at a low frequency, the ends of the 3WJ become unintentionally ligated to itself and appear in the final sequence.

For the junction analysis checks, using the same datasets, an analysis was conducted specifically on the junction areas in the reads. A junction is defined as 25 base pairs of both the 3′ and 5′ ends of a Sidewinder junction. For the PacBio data, the number was chosen to be 18 base pairs to avoid degenerate bases being included in junctions. For each sequencing run, a list was generated of all possible Sidewinder junctions, including results of both correct and incorrect ligations, and aligned them to the raw fastq files via blastn using sensitive parameters (task=blastn_short-word_size 7-reward 1-penalty-3-gapopen 5-gapextend 2). Resulting junctions were filtered with bitscore thresholds that were chosen to avoid false positive BLAST hits while maximally retaining possible mis-ligations. Reads containing mis-ligations were collected and examined manually to verify whether they are true mis-ligations or false-positives. The junctions for all sequencing runs were generated and analyzed as described above and visualized with custom Python scripts.

Further analysis of the PacBio sequencing data was conducted to achieve base-level resolution for SNP and diversity analysis of the Sidewinder library. Reads were aligned to reference sequences with the Smith-Waterman algorithm from EMBOSS with a match score of 5, mismatch penalty of 4, gap open penalty of 10 and a gap extend penalty of 0.5. To characterize gene-level mutation profiles, reads with incorrect lengths (+20 bp from reference sequence), reads with missing fragments, or reads not aligned with target sequence reference are removed from this analysis. Only reads with an average phred score of Q39.5 or above are kept for this downstream analysis and only bases with a phred score of Q40 are included for base-level mutation analysis in order to minimize errors introduced during sequencing.

The sequencing data generated in this study have been deposited in the NCBI Sequence Read Archive with the accession number PRJNA1201800, the content of which is incorporated herein by reference in its entirety.

### Library Construction

A sequence of 11 N-degenerate bases was placed before the promoter on the first fragment in the assembly in order to be able to track individual constructs with defined mutation profiles throughout the assembly, sequencing, and transformation. This sequence only appears in the coding oligo and does not have a complement in the barcode oligo.

To ensure efficient retention of diversity required for library construction provided by degenerate bases and additional coding oligos, the library construction was done using a modified protocol prior to assembly. Both barcode oligos and coding oligos were ordered using cartridge purification but otherwise oligo processing followed the same protocol as for other assemblies. For fragments which require multiple coding oligos to cover all mutation profiles, oligos are processed and fragments are annealed in their own tube as if they were distinct fragments. After heteroduplex formation, fragments were not PAGE extracted. Final fragment concentrations are assumed to be the same across each of the fragments at 1 μM heteroduplex. Equimolar fragment concentrations were utilized in the final Sidewinder assembly. To achieve this, analogous fragments (i.e. all F4 fragments with each different coding oligo) were mixed immediately prior to assembly into a single tube, vortexed, spun down and then this pool was treated as an individual fragment to be added to the assembly mix. Assembly and amplification were then carried out as described.

Pre-clonal sequencing was done using purified amplicon of the final assembled product. Post-clonal sequencing was conducted by cloning the Sidewinder library assembly as described in the Cloning and Transformation Method. The post recovery culture was then grown overnight in 500 mL LB with 20 ug/mL chloramphenicol. Then overnight culture was then miniprepped in batches 10 mL and each elution pooled and sent for sequencing.

### Library Data Presentation

Empirical codon level mutation profile ratios were determined by calculating the ratio between bases associated with the specified codon choices. The average absolute deviation was calculated by taking the absolute value of the difference between the empirical proportion a codon appears from the theoretical proportion and averaging this difference for all 37 codon options in the library FIG. 5G.

Library coverage was determined by considering the identity of mutated positions for all 17 positions in the gene and assigning this combination to one of the 442,368 variants. To calculate the coverage of every possible combination of N mutants depicted in FIG. 5K, it was first determined the number of ways one could combine any number of mutation positions. For example, there are 643 ways to combine any 2 mutation positions in this library, 6,971 ways for 3 mutation positions and so on. It was then calculated how many of these possible combinations were seen for every N 1-17 where 17 is the coverage for the entire gene library with 442,368 possible variations.

### Combinatorial Fluorescence Library Screening

The combinatorial library generated using Sidewinder to introduce mutations into functional protein assemblies was screened by encapsulating transformed clones into hydrogel microparticles using a droplet generator. These encapsulated clones were subsequently analyzed using the SONY SH800S fluorescence-activated cell sorter (FACS), equipped with four excitation lasers (405 nm, 488 nm, 561 nm, and 638 nm) and six detectors capable of detecting emissions ranging from 400 nm to 780 nm. Sorting conditions were optimized by adjusting the gain settings, with the forward scatter (FSC) set to 1% and the back scatter (BSC) set to 25%. Detector gain was set to 25% for all channels, except for the FL1 and FL4 channels, which were adjusted to 30%. The sort delay was calibrated to 14 before initiating sorting. Colonies encapsulated in hydrogels were suspended in phosphate-buffered saline pH 7.4 (Gibco, ThermoFisher Scientific) and analyzed at a sample pressure of 4, with an event speed ranging from 200 to 300 events per second. Populations were gated based on their spectral properties and sorted into individual wells of 96-well plates for subsequent expansion and spectral characterization.

Over 300 of these sorted clones were screened using a monochromator on the Tecan infinite 200 pro using a 3d fluorescence intensity scan from excitation 250 nm to 600 nm and emission from 400 nm to 700 nm. 6 clones with potentially unique spectra were then chosen and cloned them into an p15a pT7 vector and cloned into pTac T7 polymerase strain. Clones were induced overnight in 3 mL at 100 μM IPTG and 10 ug/mL tetracycline, centrifuged, and resuspended in 50 μL 1×PBS. Final excitation spectra were captured with emission at either 560 nm or 620 nm and excitation from 300 nm to 520 nm or from 400 nm to 580 nm respectively. Final emission spectra were captured with excitation at either 420 nm or 460 nm and emission from 460 nm to 680 nm or from 460 nm to 624 nm respectively.

Overnight cultures grown at 3 mL at 100 μM IPTG and 10 μg/mL tetracycline were OD normalized to OD 0.2 and 2 μL were spotted 100 μM IPTG and 10 μg/mL tetracycline LB agar plates. Images were taken using FITC (470 nm and 525 nm) and TRTC (530 nm and 605 nm) overlayed with Trans on an ECHO Revolve.

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. As used in this specification and the appended claims, the singular forms “a,” “an,” and “the” include plural references unless the context clearly dictates otherwise. Any reference to “or” herein is intended to encompass “and/or” unless otherwise stated.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

1. A composition, comprising:
n fragments, wherein n is an integer greater than 2,
wherein each fragment comprises a first polynucleotide strand and a second polynucleotide strand,
wherein each (i)th fragment comprises a first barcode, a first toehold, a second barcode, and a second toehold, wherein 1<i<n;
wherein the first fragment comprises a first terminal region, a second barcode, and a first toehold,
wherein the (n)th fragment comprises a first barcode, a second toehold, and a second terminal region,
wherein for each (i)th fragment, wherein 1<i<n:
the first polynucleotide strand comprises a 5′ overhang and a 3′ overhang;
the 5′ overhang of the first polynucleotide strand comprises the first barcode;
the 3′ overhang of the first polynucleotide strand comprises the second barcode;
the first barcode of the (i)th fragment is complementary to the second barcode of the (i−1)th fragment;
the first toehold of the (i)th fragment is complementary to the second toehold of the (i+1)th fragment;
the second barcode of the (i)th fragment is complementary to the first barcode of the (i+1)th fragment; and
the second toehold of the (i)th fragment is complementary to the first toehold of the (i−1)th fragment.

2. A composition, comprising:
n fragments, wherein n is an integer greater than 2,
wherein each fragment comprises a first barcode, a first toehold, a second barcode, and a second toehold,
wherein each fragment comprises a first polynucleotide strand and a second polynucleotide strand,
wherein the first polynucleotide strand comprises a 5′ overhang and a 3′ overhang,
wherein the 5′ overhang of the first polynucleotide strand comprises the first barcode, and
wherein the 3′ overhang of the first polynucleotide strand comprises the second barcode,
wherein for each (i)th fragment, wherein 1<i<n:
the first barcode of the (i)th fragment is complementary to the second barcode of the (i−1)th fragment;
the first toehold of the (i)th fragment is complementary to the second toehold of the (i+1)th fragment;
the second barcode of the (i)th fragment is complementary to the first barcode of the (i+1)th fragment; and
the second toehold of the (i)th fragment is complementary to the first toehold of the (i−1)th fragment;
wherein the first barcode of the first fragment is complementary to the second barcode of the (n)th fragment, and
wherein the second toehold of the first fragment is complementary to the first toehold of the (n)th fragment.

3. The composition of claim 1, wherein for each (i)th fragment, wherein 1<i<n:
the first barcode of the (i)th fragment is not complementary to the first barcode of any of the n fragments; and
the first barcode of the (i)th fragment is not complementary to the second barcode of any (k)th fragment, wherein k is an integer not equal to (i-1).

4. The composition of claim 1,
wherein the 3′ overhang of the first polynucleotide strand comprises the first toehold,
wherein the first toehold is 5′ of the second barcode,
wherein the second polynucleotide strand comprises a 3′ overhang, and
wherein the 3′ overhang of the second polynucleotide strand comprises the second toehold.

5. The composition of claim 1,
wherein the 5′ overhang of the first polynucleotide strand comprises the second toehold,
wherein the second toehold is 3′ of the first barcode,
wherein the second polynucleotide strand comprises a 5′ overhang, and
wherein the 5′ overhang of the second polynucleotide strand comprises the first toehold.

6. (canceled)

7. The composition of claim 1, wherein:
at least 80%, 85%, 90%, 95%, 99%, or 100% of the fragments comprise a payload segment;
at least 80%, 85%, 90%, 95%, 99%, or 100% of the fragments comprise a toehold-flanked internal payload segment;
wherein the payload segment comprises the sequence of the first toehold and/or the second toehold; and/or
wherein the payload segment does not comprise the sequence of the first barcode or the second barcode.

8. The composition of claim 1, wherein the first fragment, the (i)th fragment, the (n)th fragment, one or more of the n fragments, the first toehold, the second toehold, the first barcode, and/or the second barcode:
is at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 110, 120, 130, 140, 150, 160, 170, 180, 190, 200, 210, 220, 230, 240, 250, 1-5, 1-10, 10-100, 10-250, 25-50, 25-100, 25-250, 50-100, 50-200, 50-250, 75-100, 75-200, 75-250, 100-150, 100-200, 100-250, 150-200, 150-250, or 200-250, nucleotides in length;
comprises a GC content of about 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, 20%-50%, 20%-75%, 20%-100%, 30%-60%, 30%-75%, 30%-100%, 40%-60%, 40%-75%, 40%-100%, 50%-75%, 50%-100%, 60%-75%, 60%-100%, or 75%-100%;
comprises a melting temperature (Tₘ) of about 35° C., 36° C., 37° C., 38° C., 39° C., 40° C., 41° C., 42° C., 43° C., 44° C., 45° C., 46° C., 47° C., 48° C., 49° C., 50° C., 51° C., 52° C., 53° C., 54° C., 55° C., 56° C., 57° C., 58° C., 59° C., 60° C., 61° C., 62° C., 63° C., 64° C., 65° C., 66° C., 67° C., 68° C., 69° C., 70° C., 71° C., 72° C., 73° C., 74° C., 75° C., 35° C.-55° C., 35° C.-75° C., 35° C.-100° C., 45° C.-55° C., 45° C.-75° C., 45° C.-100° C., 55° C.-75° C., 55° C.-100° C., 65° C.-75° C., 65° C.-100° C., or 75° C.-100° C.;
comprises DNA;
comprises RNA; and/or
comprises one or more nucleic acid analogs selected from the group consisting of RNA, 2′-O-methyl RNA, locked nucleic acid (LNA), peptide nucleic acid (PNA), morpholino, phosphorodiamidate morpholino oligomer (PMO), HNA, FANA, TNA, ANA, GNA, CeNA, UNA, L-DNA, or any combination thereof.

9. The composition of claim 1, wherein:
the melting temperature (Tₘ) of the first barcode and the second barcode is at least about 5° C., 6° C., 7° C., 8° C., 9° C., 10° C., 11° C., 12° C., 13° C., 14° C., 15° C., 16° C., 17° C., 18° C., 19° C., 20° C., 21° C., 22° C., 23° C., 24° C., or 25° C., higher than the Im of the first toehold and the second toehold;
wherein a first barcode comprises the sequence of the first 1, 2, 3, 4, 5, 6, 7, 8, 9, 10, 11, 12, 13, 14, 15, 16, 17, 18, 19, or 20, nucleotides, of any one of SEQ ID Nos: 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, and 300; and/or
wherein a second barcode comprises the sequence of the final 1, 2, 3, 4, 5, 6, 7, 8, 9, 10, 11, 12, 13, 14, 15, 16, 17, 18, 19, or 20, nucleotides, of any one of SEQ ID Nos: 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, and 300.

10. (canceled)

11. The composition of claim 1,
wherein the first barcode of the (i)th fragment forms a pair with the second barcode of the (i−1)th fragment,
wherein the second barcode of the (i)th fragment forms a pair with the first barcode of the (i+1)th fragment, and
wherein each pair is optimized for maximum mutual specificity within a pair and absolute exclusivity across different pairs.

12. The composition of claim 1, wherein n is at least 3, 4, 5, 6, 7, 8, 9, 10, 11, 12, 13, 14, 15, 16, 17, 18, 19, 20, 22, 24, 26, 28, 30, 32, 34, 36, 38, 40, 42, 44, 46, 48, 50, 55, 60, 65, 70, 75, 80, 85, 90, 95, 100, 125, 150, 175, 200, 225, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, 1000, 10-25, 10-50, 10-75, 10-100, 10-500, 10-1000, 25-50, 25-75, 25-100, 25-500, 25-1000, 50-75, 50-100, 50-500, 50-1000, 75-100, 75-500, 75-1000, 100-500, 100-1000, or 500-1000.

13. The composition of claim 1,
wherein, upon incubation in a reaction mixture, the n fragments are capable of joining together via at least one three-way junction (3WJ) intermediate to generate an intermediate product.

14. The composition of claim 13, wherein a ligase is capable of ligating nicks on the second polynucleotide strands of said intermediate product to generate an assembled product.

15. (canceled)

16. (canceled)

17. The composition of claim 14, wherein the assembled product comprises a final synthetic sequence, and wherein the final synthetic sequence does not comprise the sequence of the first barcode or the second barcode of any of the n fragments.

18. (canceled)

19. The composition of claim 17, wherein the final synthetic sequence is at least about 500 bases, 750 bases, 1 kb, 2 kb, 3 kb, 4 kb, 5 kb, 6 kb, 7 kb, 8 kb, 9 kb, 10 kb, 15 kb, 20 kb, 25 kb, 50 kb, 75 kb, 100 kb, 250 kb, 500 kb, 750 kb, or 1 MB, in length.

20. (canceled)

21. The composition of claim 1,
wherein for each (i)th fragment, the first toehold of the (i)th fragment is not complementary to the second toehold of any (k)th fragment, wherein k is an integer not equal to (i+1); or
wherein for at least one (i)th fragment, the first toehold of the (i)th fragment is complementary to the second toehold of one or more (k)th fragments, wherein k is an integer not equal to (i+1).

22. The composition of claim 7,
(a) wherein the first fragment:
is an invariant fragment, wherein all instances of the invariant first fragment in the composition are identical; or
is a variant fragment, wherein two or more instances of the variant first fragment in the composition differ with respect to the sequence of the internal payload segment;
(b) wherein at least one (i)th fragment is an invariant fragment, wherein all instances of the invariant (i)th fragment in the composition are identical;
(c) wherein at least one (i)th fragment is a variant fragment, wherein two or more instances of the variant (i)th fragment in the composition differ with respect to the sequence of the internal payload segment; and/or
(d) wherein the (n)th fragment:
is an invariant fragment, wherein all instances of the invariant (n)th fragment in the composition are identical; or
is a variant fragment, wherein two or more instances of the variant (n)th fragment in the composition differ with respect to the sequence of the internal payload segment.

23. (canceled)

24. The composition of claim 1, wherein the composition comprises y sets of n fragments, and wherein:
the value of n is the same between at least two of the y sets;
the value of n is the different between at least two of the y sets;
the first barcode and the second barcode of each set are not complementary to the first barcode and the second barcode of any other set;
upon incubation of the y sets together in a single reaction mixture, each set of n fragments is capable of, in parallel, joining together via three-way junction (3WJ) intermediates to generate y intermediate products;
and/or
y is an integer greater than 1.

25. The composition of claim 17, wherein the final synthetic sequence comprises one or more payload genes, wherein the one or more payload genes encode one or more RNA payload(s) and/or one or more payload protein(s).

26-47. (canceled)

48. A method, comprising:
providing the n fragments of claim 1;
incubating the n fragments in a reaction mixture under reaction conditions such that:
the first barcode of the (i)th fragment hybridizes to the second barcode of the (i−1)th fragment; and
the second barcode of the (i)th fragment hybridizes to the first barcode of the (i+1)th fragment,
thereby joining together the n fragments via three-way junction (3WJ) intermediates to generate an intermediate product; and
ligating nicks on the second polynucleotide strands to generate an assembled product.

49-85. (canceled)

86. A kit, comprising:
the n fragments of claim 1,
a non-thermostable ligase, a thermostable ligase, a chemical coupling agent, a polymerase, a primer capable of binding the first terminal region (or a complement thereof), a primer capable of binding the second terminal region (or a complement thereof), or any combination thereof; and/or
a ligation buffer, optionally comprising:
HiFi Taq buffer.

87. (canceled)
